(19) United States
(12) Patent Application Publication
(10) Pub. No.: US 2023/0331737 A1
(43) Pub. Date: Oct. 19, 2023

(54) MTORC1 MODULATORS AND USES THEREOF

(71) Applicant: Aeovian Pharmaceuticals, Inc., Berkeley, CA (US)

(72) Inventors: John KINCAID, Pleasanton, CA (US); Alexandre FROIDBISE, Gardouch (FR); Guillaume EPPE, Toulouse (FR)

(21) Appl. No.: 18/157,224

(22) Filed: Jan. 20, 2023

Related U.S. Application Data
(63) Continuation of application No. PCT/US21/42644, filed on Jul. 21, 2021.
(60) Provisional application No. 63/054,767, filed on Jul. 21, 2020.

(51) Int. Cl. C07D 491/18

(52) U.S. Cl. CPC C07D 491/18

(57) ABSTRACT

The disclosure provides compounds and salts that show high selectivity and inhibitory activity for mTORC1 and uses thereof for the treatment of disease.

### CROSS-REFERENCE

This application is a continuation of International Patent Application PCT/US2021/042644, filed Jul. 21, 2021, which claims priority to U.S. Application No. 63/054,767 filed Jul. 21, 2020, each of which is incorporated by reference herein in its entirety.

### BACKGROUND OF THEE INVENTION

The therapeutic potential of rapamycin has been established in many chronic diseases, from Alzheimer's and Parkinson's disease to diabetes and cardiovascular disease. However, the prohibitive safety profile of rapamycin for chronic treatment has limited its use for the treatment of various diseases. Rapamycin, an FDA approved compound, inhibits mTOR signaling, leading to extension of lifespan in a number of species, yet it can induce adverse effects, such as peripheral edema, hypercholesterolemia, muscosal ulcerations, abdominal pain, headache, nausea, diarrhea, pain, constipation, hypertriglyceridemia, hypertension, increased creatinine, fever, urinary tract infection, anemia, arthralgia, and thrombocytopenia. Given the complications associated with rapamycin, therapeutic alternatives are needed.

### SUMMARY OF THE INVENTION

In an aspect, the present disclosure provides a compound represented by the Formula

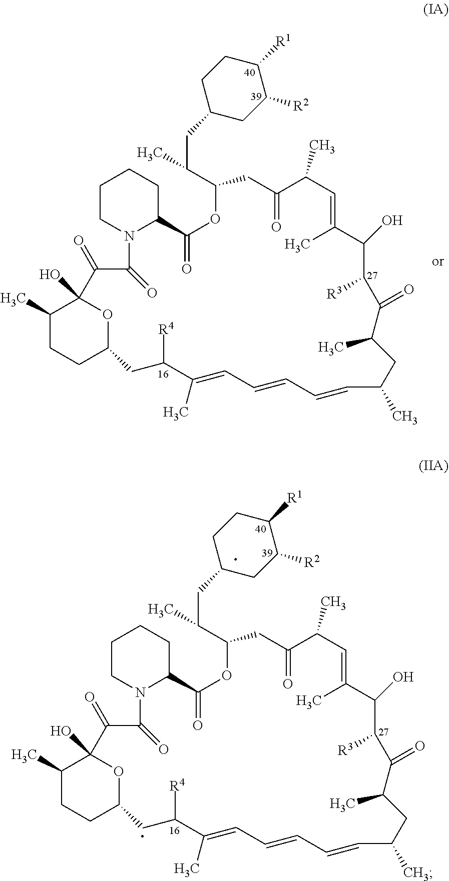

or a salt of either one thereof, wherein:
R¹ is selected from

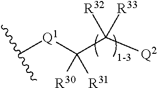

and —OCH₃;
R² is selected from hydrogen, hydroxy, and an optionally substituted C₁-C₆ alkoxy group, wherein substituents on the C₁-C₆ alkoxy group are independently selected at each occurrence from hydroxy, halogen, cyano, nitro, C₂-C₆ alkoxy group, optionally substituted carbocycle and optionally substituted heterocycle, wherein substituents on the carbocycle or heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
R³ is selected from hydrogen, hydroxy, and optionally substituted C₁-C₆ alkoxy group, wherein the substituents on the C₁-C₆ alkoxy group are independently selected at each occurrence from hydroxy, halogen, cyano, nitro, C₂-C₆ alkoxy group, optionally substituted carbocycle and optionally substituted heterocycle, wherein substituents on the carbocycle or heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
R⁴ is selected from

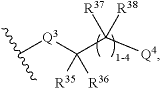

and —O—CH(CH₃)₂;
T is an optionally substituted 3-6-membered heterocycloalkyl wherein substituents are independently selected from hydroxy, halogen, cyano, nitro, C₁-C₆ alkyl, haloalkyl, hydroxy C₁-C₆ alkyl, alkoxy, and alkoxy C₁-C₆ alkyl;
Q¹ and Q³ are independently selected from —O—, —OC(═O)NR⁴¹—, —S—, and —NR⁴⁰—;
Q² is selected from optionally substituted C₃₋₆ carbocycle, optionally substituted 3-8-membered heterocycle, —OR³⁴, —(O—CH₂—(CH₂)ₚ)ₙ—W, and —N(R³⁹)₂, wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
Q⁴ is selected from optionally substituted C₃₋₆ carbocycle, optionally substituted 3-8-membered heterocycle, and —OR⁴², wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
R³⁰, R³¹, R³⁵, and R³⁶ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl;
each R³², R³³, R³⁷, and R³⁸ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl;
each R³⁴ is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle;
each R³⁹ is selected from hydrogen, C₁-C₆ alkyl, haloalkyl, and alkoxy C₁-C₆ alkyl;
each R⁴⁰ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle; and
each R⁴¹ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle;
each R⁴² is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle;
each p is selected from 1 or 2;
n is selected from 2-4; and
W is selected from —OH and —OCH₃.

In certain aspects, the disclosure provides a compound represented by Formula (III-A) or (III-C):

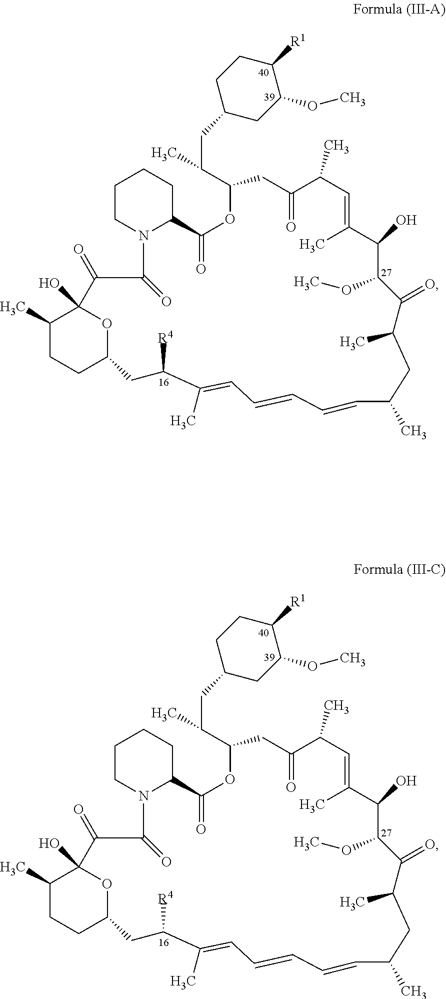

or a salt thereof, wherein:
R^{1′} is selected from —OH,

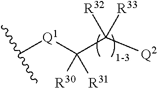

and —OCH₃;
R⁴ is selected from

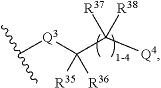

—O—(CH₂)₀₋₁T and —O—CH(CH₃)₂;
T is an optionally substituted 3-6-membered heterocycloalkyl wherein substituents are independently selected from hydroxy, halogen, cyano, nitro, C₁-C₆ alkyl, haloalkyl, hydroxy C₁-C₆ alkyl, alkoxy, and alkoxy C₁-C₆ alkyl;
Q¹ and Q³ are independently selected from —O—, —OC(═O)NR⁴¹—, —S—, and —NR⁴⁰—;
Q² is selected from optionally substituted C₃₋₆ carbocycle, optionally substituted 3-8-membered heterocycle, —OR³⁴, —(O—CH₂—(CH₂)ₚ)ₙ—W, and —N(R³)₂, wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
Q⁴ is selected from optionally substituted C₃₋₆ carbocycle, optionally substituted 3-8-membered heterocycle, and —OR⁴², wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
R³⁰, R³¹, R³⁵, and R³⁶ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl;
each R³², R³³, R³⁷, and R³⁸ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl;
each R³⁴ is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle;
each R³⁹ is selected from hydrogen, C₁-C₆ alkyl, haloalkyl, and alkoxy C₁-C₆ alkyl;
each R⁴⁰ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle; and
each R⁴¹ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle;
each R⁴² is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle;
each p is selected from 1 or 2;
n is selected from 2-4; and
W is selected from —OH and —OCH₃.

In certain aspects, the present disclosure provides a compound of Formula (IB), (IC), (ID), (IE), (IIB), (IIC), (III-A), (III-B), (III-C), (III-D), (III-E), (III-F), (III-G), or (III-H) or a salt of any one thereof.

In certain aspects, the present disclosure provides a pharmaceutical formulation comprising a compound of Formula (IA), (IB), (IC), (ID), (IE), (IIA), (IIB), (IIC), (III-A), (III-B), (III-C), (III-D), (III-E), (III-F), (III-G), or (III-H) or a salt of any one thereof and a pharmaceutically acceptable excipient.

In certain aspects, the present disclosure provides methods for treating an mTORopathy using a pharmaceutical formulation of a compound of Formula (IA), (IB), (IC), (ID), (IE), (IIA), (IIB), (IIC), (III-A), (III-B), (III-C), (III-D), (III-E), (III-F), (III-G), or (III-H).

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counterions. Salts can form from or incorporate one or more deprotonated acidic groups (e.g. carboxylic acids), one or more protonated basic groups (e.g. amines), or both (e.g. zwitterions).

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to saturated hydrocarbon groups, including straight-chain alkyl and branched-chain alkyl groups that contain from 1 to 6 carbons. The term —C_{x-y}alkylene-refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

The terms “C_{x-y}alkenyl” and “C_{x-y}alkynyl” refer to unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene-refers to a substituted or unsubstituted alkynylene chain with from x to y carbons in the alkynylene chain. For example, —C₂₋₆alkynylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. Alkylene chain may be optionally substituted by one or more substituents such as those substituents described herein.

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. Alkenylene chain may be optionally substituted by one or more substituents such as those substituents described herein.

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. Alkynylene chain may be optionally substituted by one or more substituents such as those substituents described herein.

The term “carbocycle” as used herein refers to a saturated, unsaturated or aromatic ring in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein. Bicyclic carbocycles may be fused, bridged or spiro-ring systems.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. In an exemplary embodiment, a heterocycle, e.g., pyridyl, may be fused to a saturated or unsaturated ring, e.g., cyclohexane, cyclopentane, or cyclohexene. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—

NH₂), —R^{b}—OR^{a}, —R^{b}—OC(O)—R^{a}, —R^{b}—OC(O)—OR^{a}, —R^{b}—OC(O)—N(R^{a})₂, —R^{b}—NR^{a})₂, —R^{b}—C(O)R^{a}, —R^{b}—C(O)OR^{a}, —R^{b}—C(O)N(R^{a})₂, R^{b}—O—R^{c}—C(O)N)₂, R^{b}—NR^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—
NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—
NH₂), —R^{b}—OR, —R^{b}—OC(O)—R^{a}, —R^{b}—OC(O)—OR, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)R^{a} (where t is 1 or 2), —R^{b}—S(O)ₜOR^{a} (where t is 1 or 2) and —R^{b}—S(O)_{L}N(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain. It will be understood by those skilled in the art that substituents can themselves be substituted, if appropriate.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms ““administer”, “administered”, “administers” and “administering” a compound should be understood to mean providing a compound of the invention or a prodrug of a compound of the invention to the individual in need.

The term “effective amount” or “therapeutically effective amount” refers to that amount of a compound or salt described herein that is sufficient to affect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term can also apply to a dose that can induce a particular response in target cells, e.g., reduction of proliferation or down regulation of activity of a target protein. The specific dose can vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “selective inhibition” or “selectively inhibit” as referred to a biologically active agent refers to the agent's ability to preferentially reduce the target signaling activity as compared to off-target signaling activity, via direct or interact interaction with the target.

### Introduction

The mechanistic target of rapamycin (mTOR) signaling pathway integrates both intracellular and extracellular signals and serves as a central regulator of cell metabolism, growth, proliferation and survival. In particular, mTOR complex 1 (mTORC1) positively regulates cell growth and proliferation by promoting many anabolic processes, including biosynthesis of proteins, lipids and organelles, and by limiting catabolic processes such as autophagy. Much of the knowledge about mTORC1 function comes from the use of the bacterial macrolide rapamycin.

Rapamycin is believed to inhibit mTORC1 directly and mTORC2 indirectly upon chronic treatment. Recent evidence has revealed that inhibition of mTORC1 is responsible for effects related to lifespan extension, while inhibition of mTORC2 is uncoupled from longevity and is responsible for several of the adverse effects of rapamycin, such as impaired insulin sensitivity, glucose homeostasis, and lipid dysregulation.

Studies of rapamycin and related compounds reveal that these compounds form binary complexes with FKB binding proteins such as FKBP12 and FKBP51. This binary complex can allosterically inhibit the functionality of mTORC1 by binding to the FRB domain of mTOR. FKBP12 and FKBP51 direct binding assays provide a method to assess the relative binding affinity of rapamycin and related compounds to the specified FKBP. While not wishing to be bound by any particular mechanistic theory, it may be preferred that binding of a rapamycin and related compounds to an FKB protein, e.g., FKBP12 or FKBP51, is similar, equivalent or stronger relative to rapamycin binding to said FKB protein.

The ternary complex formation assay provides a method to assess the relative binding affinity of the rapamycin/FKB binary complex to the FRB domain of mTOR. Different binding affinities for mTOR exhibited by rapamycin and related compounds/FKB complexes may result in different pharmacology and safety profiles relative to rapamycin, everolimus, and related compounds.

In certain aspects, the disclosure provides compounds and salts thereof, and methods of use for the treatment of diseases. In certain aspects, the compounds described herein display similar direct binding properties, e.g., similar or improved FKB binding, relative to known compounds, such as rapamycin and everolimus. In certain aspects, the compounds described herein display altered ternary binding affinity, e.g. diminished binding affinity to the FRB domain of mTOR, relative to known compounds, such as rapamycin or everolimus.

In certain embodiments, compounds or salts of the disclosure are evaluated for direct binding to FKBP12 and/or FKBP51. In certain embodiments, compounds or salts of the disclosure are evaluated for ternary complex formation with MTORC1 and FKBP12. In certain embodiments, a compound or salt thereof has potent binding to FKBP12 and/or FKBP51.

### Compounds

In some aspects, the present disclosure provides a compound represented by the Formula (IA) or (IIA):

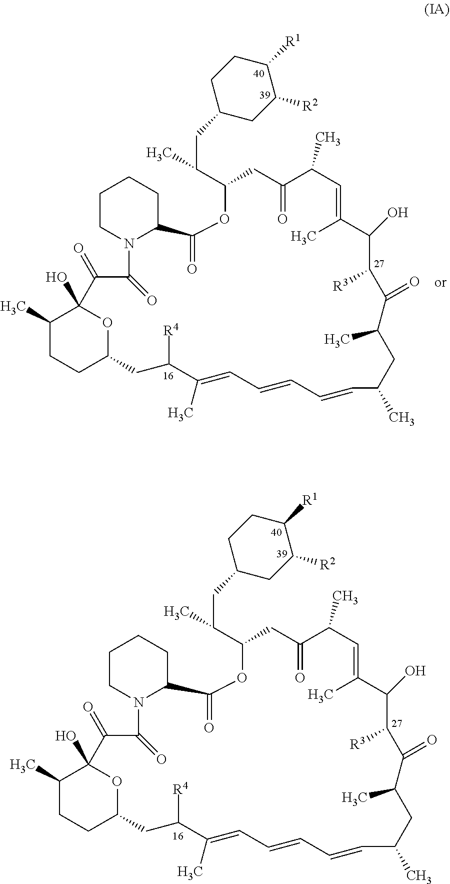

(IIA); or a salt of either one thereof, wherein:
R¹ is selected from

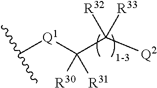

and —OCH₃;
R² is selected from hydrogen, hydroxy, and an optionally substituted C₁-C₆ alkoxy group, wherein substituents on the C₁-C₆ alkoxy group are independently selected at each occurrence from hydroxy, halogen, cyano, nitro, C₂-C₆ alkoxy group, optionally substituted carbocycle and optionally substituted heterocycle, wherein substituents on the carbocycle or heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
R³ is selected from hydrogen, hydroxy, and optionally substituted C₁-C₆ alkoxy group, wherein the substituents on the C₁-C₆ alkoxy group are independently selected at each occurrence from hydroxy, halogen, cyano, nitro, C₂-C₆ alkoxy group, optionally substituted carbocycle and optionally substituted heterocycle, wherein substituents on the carbocycle or heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
R⁴ is selected from

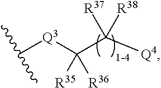

—O—(CH₂)₀₋₁T and —O—CH(CH₃)₂;
T is an optionally substituted 3-6-membered heterocycloalkyl wherein substituents are independently selected from hydroxy, halogen, cyano, nitro, C₁-C₆ alkyl, haloalkyl, hydroxy C₁-C₆ alkyl, alkoxy, and alkoxy C₁-C₆ alkyl;
Q¹ and Q³ are independently selected from —O—, —OC(═O)NR⁴¹—, —S—, and —NR⁴⁰—;
Q² is selected from optionally substituted C₃₋₆ carbocycle, optionally substituted 3-8-membered heterocycle, —OR³⁴, —(O—CH₂—(CH₂)ₚ)ₙ—W, and —N(R³⁹)₂, wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
Q⁴ is selected from optionally substituted C₃₋₆ carbocycle, optionally substituted 3-8-membered heterocycle, and —OR⁴², wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
R³⁰, R³¹, R³⁵, and R³⁶ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl;
each R³², R³³, R³⁷, and R³⁸ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl;
each R³⁴ is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle;
each R³⁹ is selected from hydrogen, C₁-C₆ alkyl, haloalkyl, and alkoxy C₁-C₆ alkyl;
each R⁴⁰ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle; and
each R⁴¹ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle;
each R⁴² is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle;
each p is selected from 1 or 2;
n is selected from 2-4; and
W is selected from —OH and —OCH₃.

In some embodiments, the compound or salt of Formula (IA) is represented by the structure of Formula (IB), (IC), (ID), or (IE), or a salt any one of thereof. In some embodiments, the structure of Formula (IB) is represented by

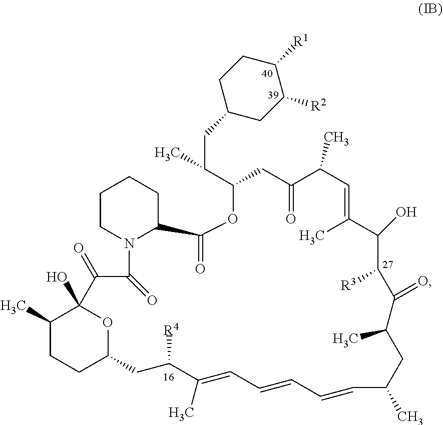

or a salt thereof. In some embodiments, the structure of Formula (IC) is represented by

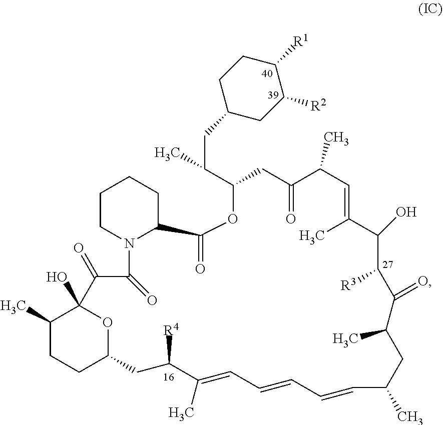

or a salt thereof. In some embodiments, the structure of Formula (ID) is represented by

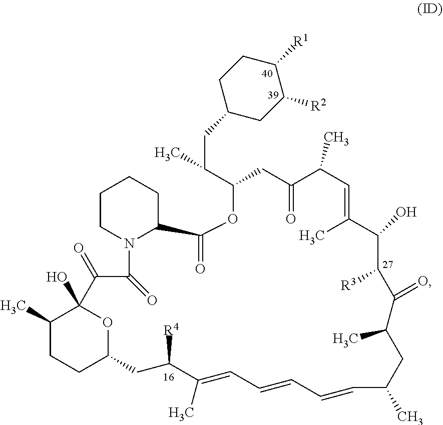

or a salt thereof. In some embodiments, the structure of Formula (IE) is represented by

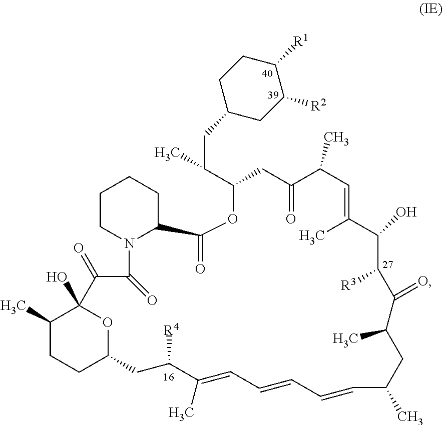

or a salt thereof.

In some embodiments, the compound or salt of Formula (IIA) is represented by the structure of Formula (IIB) or Formula (IIC). In some embodiments, the structure of Formula (IIB) is represented by

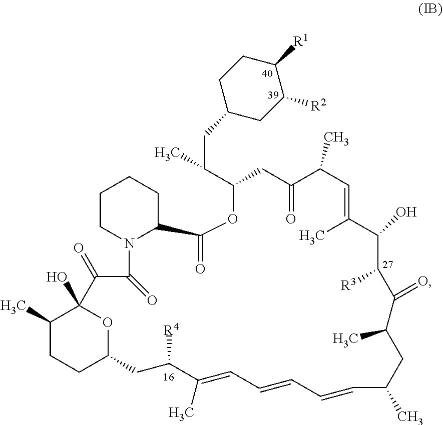

or a salt thereof. In some embodiments, the structure of Formula (IIC) may be represented by

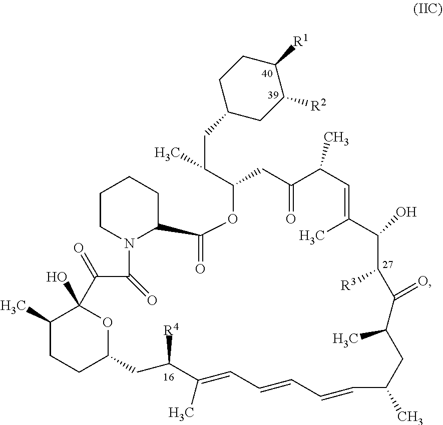

or a salt thereof.

In certain embodiments, a compound of the disclosure may be selected from Formulas (III-A), (III-B), (III-C), (III-D), (III-E), (III-F), (III-G), and (III-H):

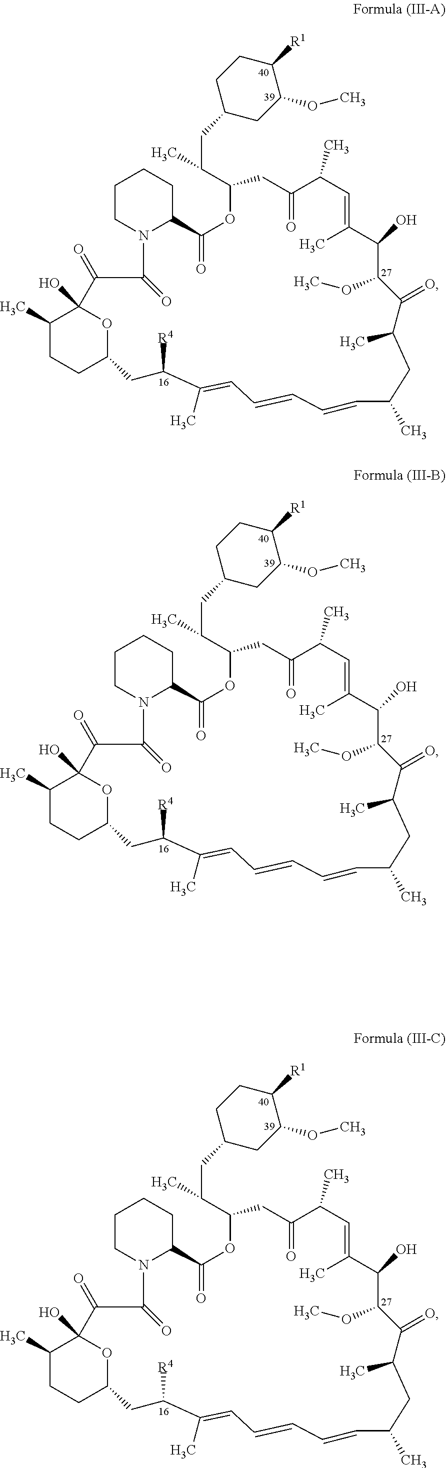

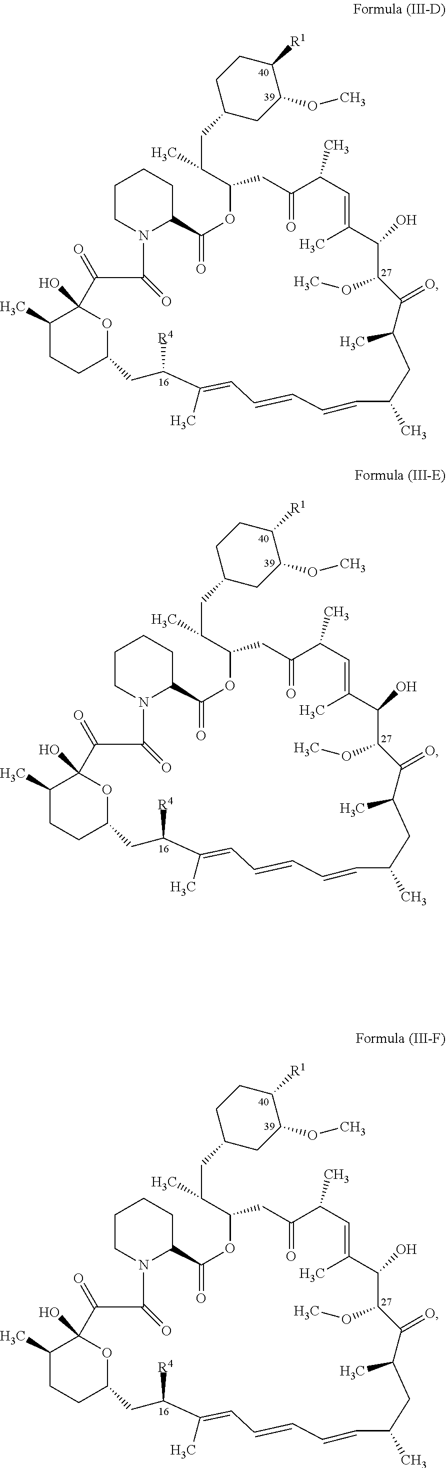

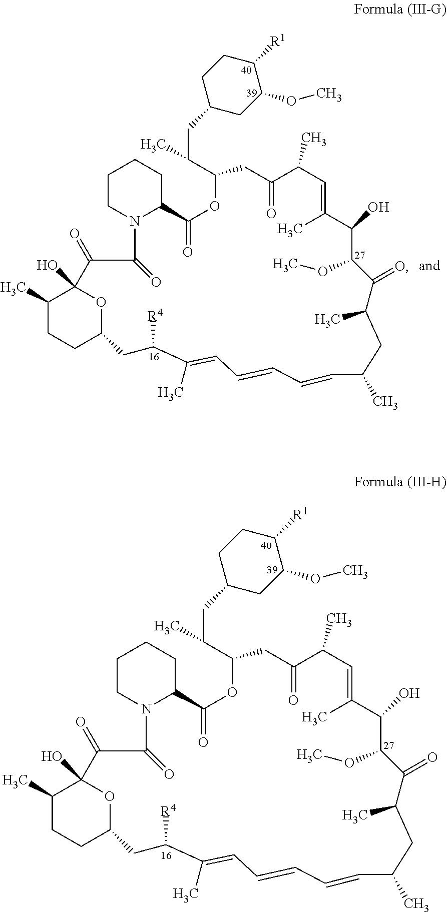

or a salt of any one thereof wherein:
R¹ is selected from

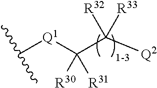

and —OCH₃;
R⁴ is selected from

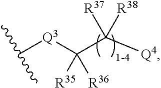

—O—(CH₂)₀₋₁T and —O—CH(CH₃)₂;
T is an optionally substituted 3-6-membered heterocycloalkyl wherein substituents are independently selected from hydroxy, halogen, cyano, nitro, C₁-C₆ alkyl, haloalkyl, hydroxy C₁-C₆ alkyl, alkoxy, and alkoxy C₁-C₆ alkyl;
Q¹ and Q³ are independently selected from —O—, —OC(═O)NR⁴¹—, —S—, and —NR⁴⁰—;
Q² is selected from optionally substituted C₃₋₆ carbocycle, optionally substituted 3-8-membered heterocycle, —OR³⁴, —(O—CH₂—(CH₂)ₚ)ₙ—W, and —N(R³⁹)₂, wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
Q⁴ is selected from optionally substituted C₃₋₆ carbocycle, optionally substituted 3-8-membered heterocycle, and —OR⁴², wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
R³⁰, R³¹, R³⁵, and R³⁶ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl;
each R³², R³³, R³⁷, and R³⁸ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl;
each R³⁴ is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle;
each R³⁹ is selected from hydrogen, C₁-C₆ alkyl, haloalkyl, and alkoxy C₁-C₆ alkyl;
each R⁴⁰ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle; and
each R⁴¹ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle;
each R⁴² is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle;
each p is selected from 1 or 2;
n is selected from 2-4; and
W is selected from —OH and —OCH₃.

In certain embodiments, a compound of the disclosure may be selected from a compound represented by Formula (III-A) or (III-C):

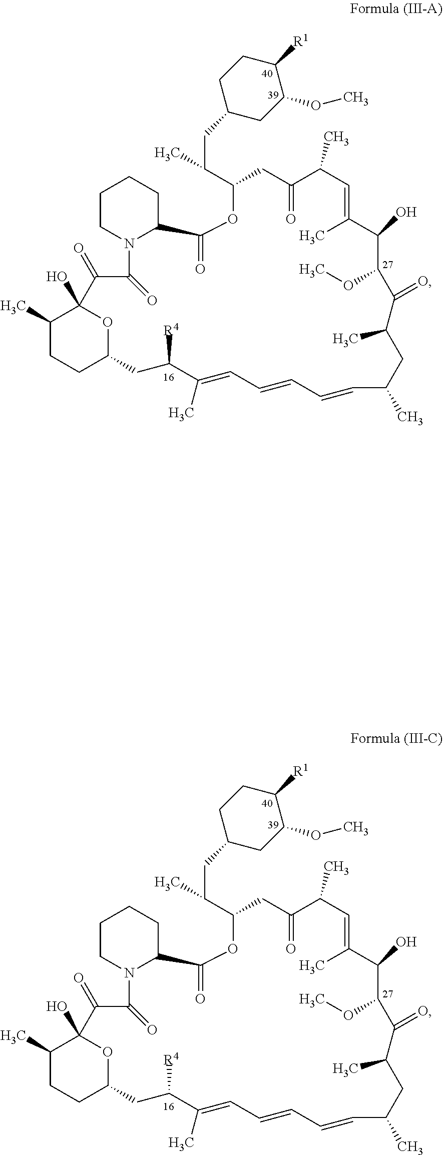

or a salt thereof, wherein:
R^{1′} is selected from —OH,

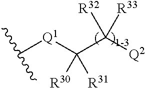

and —OCH₃;
R⁴ is selected from

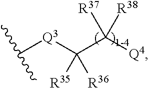

—O—(CH₂)₀₋₁-T and —O—CH(CH₃)₂;
T is an optionally substituted 3-6-membered heterocycloalkyl wherein substituents are independently selected from hydroxy, halogen, cyano, nitro, C₁-C₆ alkyl, haloalkyl, hydroxy C₁-C₆ alkyl, alkoxy, and alkoxy C₁-C₆ alkyl;
Q¹ and Q³ are independently selected from —O—, —OC(═O)NR⁴¹—, —S—, and —NR⁴⁰—;
Q² is selected from optionally substituted C₃₋₆ carbocycle, optionally substituted 3-8-membered heterocycle, —OR³⁴, —(O—CH₂—(CH₂)ₚ)ₙ—W, and —N(R³⁹)₂, wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
Q⁴ is selected from optionally substituted C₃₋₆ carbocycle, optionally substituted 3-8-membered heterocycle, and —OR⁴², wherein-substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
R³⁰, R³¹, R³⁵, and R³⁶ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl;
each R³², R³³, R³⁷, and R³⁸ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl;
each R³⁴ is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle;
each R³⁹ is selected from hydrogen, C₁-C₆ alkyl, haloalkyl, and alkoxy C₁-C₆ alkyl;
each R⁴⁰ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle; and
each R⁴¹ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle;
each R⁴² is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle;
each p is selected from 1 or 2;
n is selected from 2-4; and
W is selected from —OH and —OCH₃.

In certain embodiments, a compound of the disclosure may be selected from a compound represented by Formula (III-C).

In certain embodiments, a compound of the disclosure may be selected from a compound represented by Formula (III-A).

In some embodiments, for a compound or salt of Formula (III-A) or (III-C), R^{1′} is selected from:

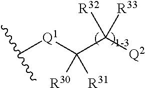

### and —OCH₃.

In some embodiments, for a compound or salt of Formula (III-A) or (III-C), R^{1′} is —OH.

In some embodiments, for a compound or salt of Formula (III-A) or (III-C), R^{1′} is selected from:

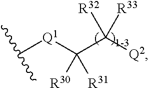

wherein Q¹ is 0.

In some embodiments, for a compound or salt of Formula (III-A) or (III-C), R^{1′} is selected from:

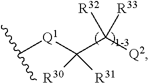

wherein Q² is selected from optionally substituted 5-7 membered heterocycle, —OH, or C₁-C₆ alkoxy.

In some embodiments, for a compound or salt of Formula (III-A) or (III-C), R^{1′} is selected from:

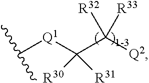

wherein Q² is selected from optionally substituted 5-6 membered heterocycle, —OH, or C₁-C₆ alkoxy.

In some embodiments, for a compound or salt of Formula (III-A) or (III-C), R^{1′} is selected from:

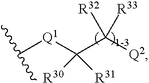

wherein Q² is selected from optionally substituted 5-6 membered heterocycle. The optional substituents of the 5-6 membered heterocycle may be selected from hydroxy, hydroxy C₁-C₆ alkyl, C₁-C₆ alkyl, and alkoxy.

In some embodiments, for a compound or salt of Formula (III-A) or (III-C), R³⁰, R³¹, R³², and R³³ are independently selected at each occurrence from hydrogen and hydroxy. In some embodiments, for a compound or salt of Formula (III-A) or (III-C), R³⁰, R³¹, R³², and R³³ are each hydrogen.

In some embodiments, for a compound or salt of Formula (III-A) or (III-C), R⁴ is selected from

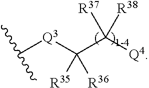

In some embodiments, R⁴ is selected from

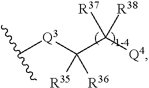

wherein Q³ is —O—.

In some embodiments, for a compound or salt of Formula (III-A) or (III-C), R³⁵, R³⁶, R³⁷, and R³⁸ are independently selected at each occurrence from hydrogen, hydroxy, hydroxy C₁-C₆ alkyl and C₁-C₆ alkyl.

In some embodiments, for a compound or salt of Formula (III-A) or (III-C), R³¹, R³⁶, R³⁷, and R³⁸ are independently selected at each occurrence from hydrogen.

In some embodiments, for a compound or salt of Formula (III-A) or (III-C), Q⁴ is selected from optionally substituted C₃₋₆ carbocycle, optionally substituted 3-7-membered heterocycle, and —OR⁴².

In some embodiments, for a compound or salt of Formula (III-A) or (III-C), R⁴² is selected from hydrogen, optionally substituted C₁-C₆ alkyl, wherein the optional substituents are selected from hydroxy, and C₁-C₆ alkoxy.

In some embodiments, for a compound or salt of Formula (III-A) or (III-C), R is selected from:

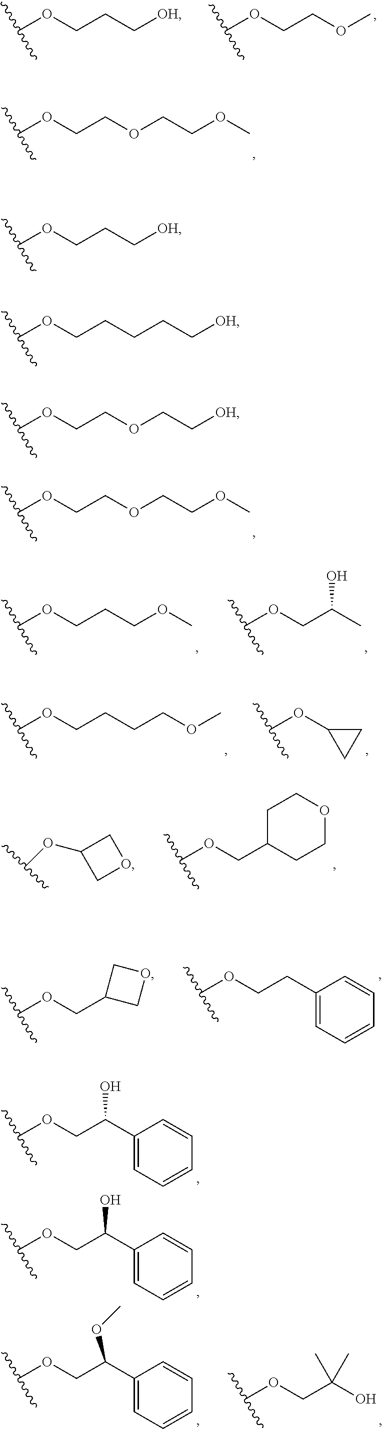

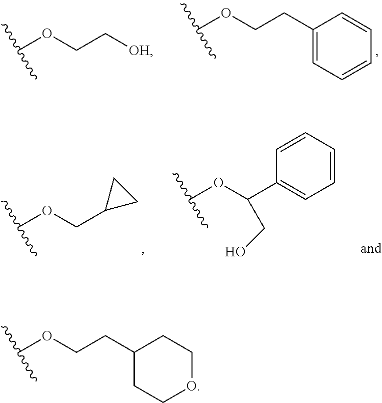 [SMILES: *OCCO ; *OCCc1ccccc1 ; *OCC1CC1 ; *OC(CO)c1ccccc1 ; *OCCC1CCOCC1]

In some embodiments for a compound or salt of Formula (III-A) or (III-C), R is selected from:

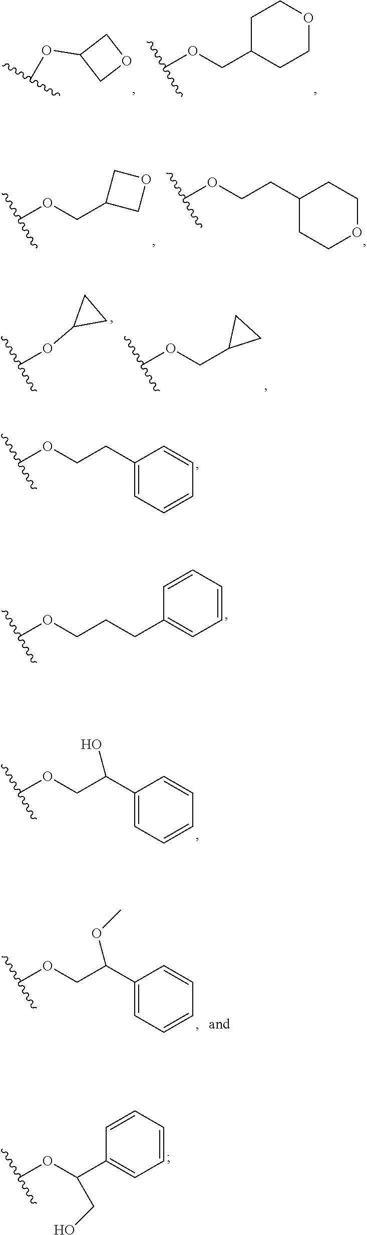

In some embodiments, for a compound or salt of Formula (III-A) or (III-C), R⁴ is selected from

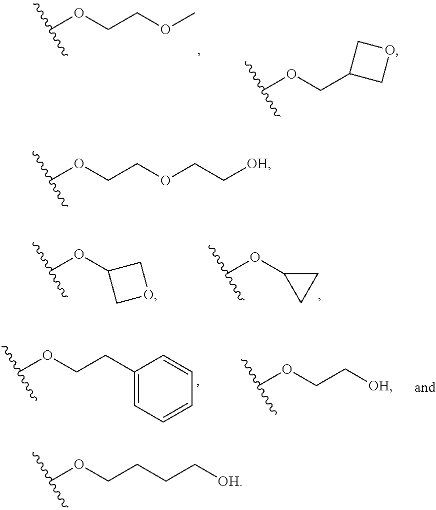

In some embodiments, for a compound or salt of Formula (IA), (IB), (IC), (ID), (IE), (IIA), (IIB), (IIC), (III-A), (III-B), (III-C), (III-D), (III-E), (III-F), (III-G), or (III-H), R¹ is

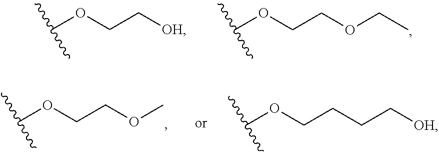

### R⁴ is not

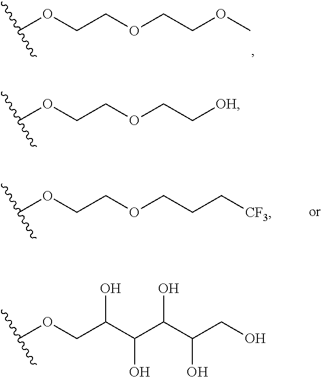

In some embodiments, for a compound or salt of Formula (IA), (IB), (IC), (ID), (IE), (IIA), (IIB), (IIC), (III-A), (III-B), (III-C), (III-D), (III-E), (III-F), (III-G), or (III-H), R¹ is not

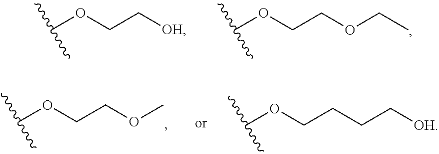

In some embodiments, for a compound or salt of Formula (IA), (IB), (IC), (ID), (IE), (IIA), (IIB), (IIC), (III-A), (III-B), (III-C), (III-D), (III-E), (III-F), (III-G), or (III-H), R¹ is hydroxy. In some embodiments, for a compound or salt of Formula (IA), (IB), (IC), (ID), (IE), (IIA), (IIB), (IIC), (III-A), (III-B), (III-C), (III-D), (III-E), (III-F), (III-G), or (III-H), R¹ is not hydroxy.

In some embodiments, for a compound or salt of Formula (IA), (IB), (IC), (ID), (IE), (IIA), (IIB), or (IIC), R² is selected from optionally substituted C₁-C₆ alkoxy group. In some embodiments, R² is a C₁-C₆ alkoxy. In some embodiments, R² is —OCH₃.

In some embodiments, for a compound or salt of Formula (IA), (IB), (IC), (ID), (IE), (IIA), (IIB), or (IIC), R³ is a C₁-C₆ alkoxy. In some embodiments, R³ is a C₁-C₃ alkoxy. In some embodiments, R³ is a C₁ alkoxy group. In some embodiments, R³ is a —OCH₃.

In some embodiments, for a compound or salt of Formula (IA), (IB), (IC), (ID), (IE), (IIA), (IIB), (IIC), (III-A), (III-B), (III-C), (III-D), (III-E), (III-F), (III-G), or (III-H), R¹ is selected from:

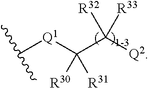

In some embodiments, R¹ is selected from:

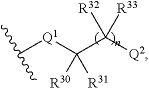

wherein n is 0, 1, 2, 3, 4 or 5. In some embodiments, n of

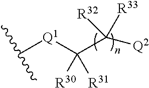

is 0, 1, 2, or 3. In some embodiments, n of

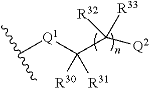

is 0, 1, or 2. In some embodiments, n of

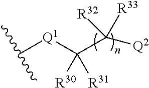

is 0. In some embodiments, n of

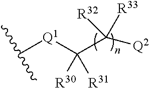

is 1. In some embodiments, n of

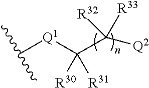

is 2.

In some embodiments for a compound or salt of Formula (IA), (IB), (IC), (ID), (IE), (IIA), (IIB), (IIC), (III-A), (III-B), (III-C), (III-D), (III-E), (III-F), (III-G), or (III-H), Q² is selected from optionally substituted phenyl, optionally substituted 5-7-membered heterocycle, and —N(R³⁹)₂, wherein substituents on phenyl and 5-7-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), when Q¹ is —O—, Q² is selected from optionally substituted phenyl, optionally substituted 5-7-membered heterocycle, and —N(R³⁹)₂, wherein substituents on phenyl and 5-7-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q² is selected from optionally substituted phenyl and optionally substituted 5- or 6-membered heterocycle wherein substituents on phenyl and 5- or 6-membered heterocycle are independently selected from hydroxy, halogen, cyano, nitro, C₁-C₆ alkyl, haloalkyl; hydroxy C₁-C₆ alkyl, alkoxy, and alkoxy C₁-C₆ alkyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q² is selected from optionally substituted phenyl and optionally substituted 5- or 6-membered saturated heterocycle wherein substituents on phenyl and 5- or 6-membered saturated heterocycle are independently selected from hydroxy, halogen, cyano, nitro, C₁-C₆ alkyl, haloalkyl, hydroxy C₁-C₆ alkyl, C₁-C₆ alkoxy, and C₁-C₆ alkoxy C₁-C₆ alkyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q² is selected from optionally substituted phenyl, optionally substituted piperidine, optionally substituted morpholine, optionally substituted piperazine, optionally substituted pyrrolidine, optionally substituted pyrazolidine, optionally substituted oxazolidine, and optionally substituted isooxazolidine, wherein substituents on phenyl, morpholine, piperidine, pyrrolidine, pyrazolidine, oxazolidine, isooxazolidine, and piperazine are independently selected from hydroxy, halogen, cyano, nitro, C₁-C₆ alkyl, haloalkyl, hydroxy C₁-C₆ alkyl, alkoxy, and alkoxy C₁-C₆ alkyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q² is selected from optionally substituted phenyl, optionally substituted piperidine, optionally substituted morpholine, and optionally substituted piperazine, wherein substituents on phenyl, morpholine, piperidine, and piperazine are independently selected from hydroxy, halogen, cyano, nitro, C₁-C₆ alkyl, haloalkyl, hydroxy C₁-C₆ alkyl, alkoxy, and alkoxy C₁-C₆ alkyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q¹ of R¹ is selected from —O— and —OC(═O)NR⁴¹—. In some embodiments, Q¹ of R¹ is selected from —O— and —OC(═O)NR⁴¹—, and R⁴¹ is selected from hydrogen and C₁-C₃ alkyl group wherein the substituents are independently selected at each occurrence from halogen, hydroxy, carbocycle and heterocycle. In some embodiments, the carbocycle of optionally substituted C₁-C₃ alkyl group of R⁴¹ is a C₃₋₆ carbocycle, e.g., phenyl. In some embodiments, the heterocycle of optionally substituted C₁-C₃ alkyl group of R¹ is 3- to 6-membered heterocycle, e.g., a 5- or 6-membered heteroaryl ring In some embodiments, Q¹ of R is selected from —O— and —OC(═O)NR⁴¹—, and R⁴¹ is selected from hydrogen and C₁-C₃ alkyl group wherein the substituents are independently selected at each occurrence from halogen or hydroxy. In some embodiments, Q¹ of R¹ is selected from —O— and —OC(═O)NR⁴¹—, and R⁴¹ is selected from hydrogen and C₁-C₃ alkyl group. In some embodiments, Q¹ of R¹ is selected from —O— and —OC(═O)NR⁴¹—, and R⁴¹ is selected from hydrogen and C₁ alkyl group. In some embodiments, Q¹ of R¹ is —OC(═O)NR⁴¹—, and R⁴¹ is selected from hydrogen and C₁₋₃ alkyl group.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q¹ of R¹ is selected from —O—, —OC(═O)NH—, and —OC(═O)N(CH₃)—. In some embodiments, Q¹ of R¹ is from —O—. In some embodiments, Q¹ of R¹ is —OC(═O)NH—. In some embodiments, Q¹ of R¹ is and —OC(═O)N(CH₃)—. In some embodiments, Q¹ of R¹ is and —OC(═O)N(CH₂CH₃)—. In some embodiments, Q¹ of R¹ is and —OC(═O)N(CH₂CH₂CH₃)—. In some embodiments, Q¹ of R¹ is and —OC(═O)N(CH₂CH₂CH₂CH₃)—.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), each of R³⁰, R³¹, R³² and R³³ of R¹ is independently selected from hydrogen, hydroxy, halogen, cyano, nitro, and C₁-C₆ alkyl. In some embodiments, each of R³⁰, R³¹, R³² and R³³ of R¹ is independently selected from hydrogen, hydroxy, halogen, cyano, nitro, and C₁-C₃ alkyl. In some embodiments, each of R³⁰, R³¹, R³² and R³³ of R¹ is independently selected from hydrogen, hydroxy, and C₁-C₃ alkyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), each of R³⁰, R³¹, R³² and R³³ of R¹ is independently selected from hydrogen, hydroxy, and methyl. In some embodiments, one of R³⁰, R³¹, R³² and R³³ of R¹ is hydroxy or methyl and the rest of R³⁰, R³¹, R³² and R³³ are each hydrogen. In some embodiments, one of R³⁰, R³¹, R³² and R³³ of R¹ is hydroxy and the rest of R³⁰, R³¹, R³² and R³³ are each hydrogen. In some embodiments, each R³⁰, R³¹, R³² and R³³ of R is hydrogen.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q² of R¹ is selected from optionally substituted C₃₋₆ carbocycle, optionally substituted 5-7-membered heterocycle, —OR³⁴, —(O—CH₂—(CH₂)ₚ)ₐ—W, and —N(R³⁹)₂, wherein substituents on C₃₋₆ carbocycle and 5-7-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl. In some embodiments, Q² of R¹ is selected from optionally substituted phenyl, optionally substituted 5-7-membered heterocycle, —OR³⁴, —(O—CH₂—(CH₂)ₚ)ₙ—W, and —N(R³⁹)₂, wherein substituents on phenyl and 5-7-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q² of R is selected from optionally substituted 5-7-membered heterocycle, and —OR³⁴. In some embodiments, Q² of R¹ is selected from —OR³⁴, and R³⁴ is selected from hydrogen and optionally substituted C₁-C₆ alkyl. In some embodiments, Q² of R¹ is selected from —OR³⁴, and R³⁴ is selected from hydrogen and C₁-C₆ alkyl. In some embodiments, Q² of R¹ is selected from —OR³⁴, and R³⁴ is selected from hydrogen, methyl, ethyl and propyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q² of R¹ is selected from optionally substituted carbocycle or optionally substituted heterocycle. In some embodiments, the carbocycle of Q² of R¹ may be selected from:

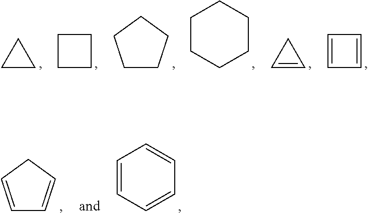

any one of which is optionally substituted. In some embodiments, the heterocycle of Q² of R¹ may be selected from:

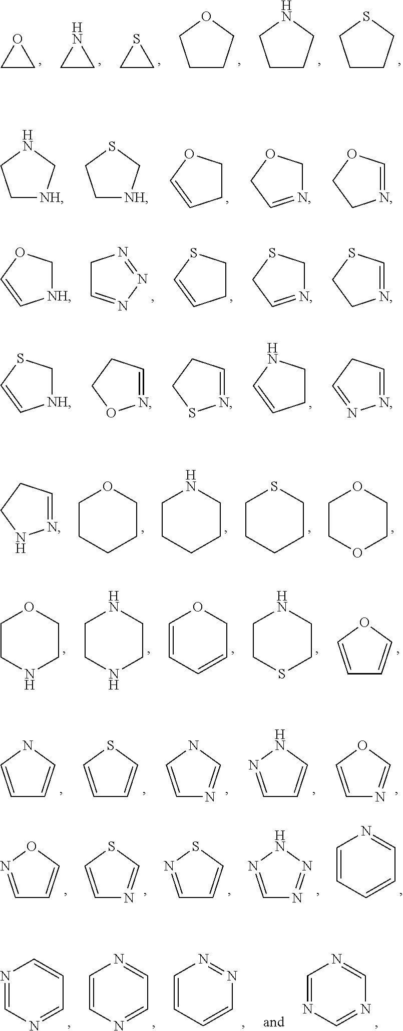

any one of which is optionally substituted.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q² of R¹ is optionally substituted carbocycle. In some embodiments, substituents on carbocycle are independently selected from hydroxy, halogen, cyano, nitro, C₁-C₆ alkyl, haloalkyl, hydroxy C₁-C₆ alkyl, alkoxy, and alkoxy C₁-C₆ alkyl. In some embodiments, Q² of R¹ is optionally substituted C₃₋₆ carbocycle. In some embodiments, substituents on C₃₋₆ carbocycle are independently selected from hydroxy, halogen, cyano, nitro, C₁-C₆ alkyl, haloalkyl, hydroxy C₁-C₆ alkyl, alkoxy, and alkoxy C₁-C₆ alkyl. In some embodiments, C₃₋₆ carbocycle is substituted with one substituent selected from hydroxy, halogen, cyano, nitro, C₁-C₆ alkyl, haloalkyl, hydroxy C₁-C₆ alkyl, alkoxy, and alkoxy C₁-C₆ alkyl. In some embodiments, C₃₋₆ carbocycle is substituted with one substituent selected from hydroxy, C₁-C₆ alkyl, hydroxy C₁-C₆ alkyl, alkoxy, and alkoxy C₁-C₆ alkyl. In some embodiments, Q² of R¹ is optionally substituted phenyl. In some embodiments, substituents on phenyl of Q² of R¹ of are independently selected from hydroxy, halogen, cyano, nitro, C₁-C₆ alkyl, haloalkyl, hydroxy C₁-C₆ alkyl, alkoxy, and alkoxy C₁-C₆ alkyl. In some embodiments, phenyl of Q² of R¹ is substituted with one substituent selected from hydroxy, halogen, cyano, nitro, C₁-C₆ alkyl, haloalkyl, hydroxy C₁-C₆ alkyl, alkoxy, and alkoxy C₁-C₆ alkyl.

In some embodiments for a compound or salt of Formula (IA), (IB), (IC), (ID), (IE), (IIA), (IIB), (IIC), (III-A), (III-B), (III-C), (III-D), (III-E), (III-F), (III-G), or (III-H), Q² of R¹ is optionally substituted 5-7-membered heterocycle. In some embodiments, substituents on 5-7-membered heterocycle of Q² of R¹ are independently selected from hydroxy, halogen, cyano, nitro, C₁-C₆ alkyl, haloalkyl, hydroxy C₁-C₆ alkyl, alkoxy, and alkoxy C₁-C₆ alkyl. In some embodiments, substituents on 5-7-membered heterocycle of Q² of R¹ are independently selected from hydroxy, C₁-C₆ alkyl, hydroxy C₁-C₆ alkyl, alkoxy, and alkoxy C₁-C₆ alkyl. In some embodiments, 5-7-membered heterocycle of Q² of R¹ is substituted one substituent selected from hydroxy, halogen, cyano, nitro, C₁-C₆ alkyl, haloalkyl, hydroxy C₁-C₆ alkyl, alkoxy, and alkoxy C₁-C₆ alkyl. In some embodiments, 5-7-membered heterocycle of Q² of R¹ is substituted two substituents independently selected at each occurrence from hydroxy, halogen, cyano, nitro, C₁-C₆alkyl, haloalkyl, hydroxy C₁-C₆ alkyl, alkoxy, and alkoxy C₁-C₆ alkyl. In some embodiments, 5-7-membered heterocycle of Q² of R¹ is substituted with one, two, or three substituents independently selected at each occurrence from hydroxy, halogen, cyano, nitro, C₁-C₆ alkyl, haloalkyl, hydroxy C₁-C₆ alkyl, alkoxy, and alkoxy C₁-C₆ alkyl. In some embodiments, 5-7-membered heterocycle of Q² of R¹ is substituted with one or two substituents independently selected at each occurrence from hydroxy, halogen, cyano, nitro, C₁-C₆ alkyl, haloalkyl, hydroxy C₁-C₆ alkyl, alkoxy, and alkoxy C₁-C₆ alkyl. In some cases, the C₁-C₆ alkyl of the independently selected at each occurrence C₁-C₆ alkyl of the 5-7-membered heterocycle of Q² of R¹ may be substituted with a substituent independently selected at each occurrence from hydroxy, C₁-C₆ alkyl, and alkoxy.

In some embodiments for a compound or salt of Formula (IA), (IB), (IC), (ID), (IE), (IIA), (IIB), (IIC), (III-A), (III-B), (III-C), (III-D), (III-E), (III-F), (III-G), or (III-H), Q² of R¹ is —OR³⁴. In some embodiments, Q² of R is —OR³⁴, and R³⁴ is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, C₁-C₆ alkoxy, carbocycle and heterocycle. In some embodiments, the optionally substituted carbocycle of R³⁴ of —OR³⁴ is a C₃₋₆ carbocycle. In some embodiments, the optionally substituted heterocycle of R³⁴ of —OR³⁴ is a 3-7-membered hetercycle.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), R¹ is selected from

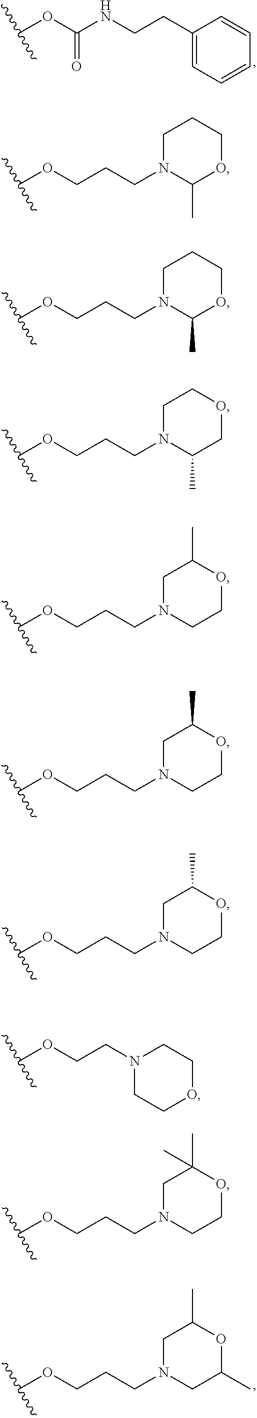

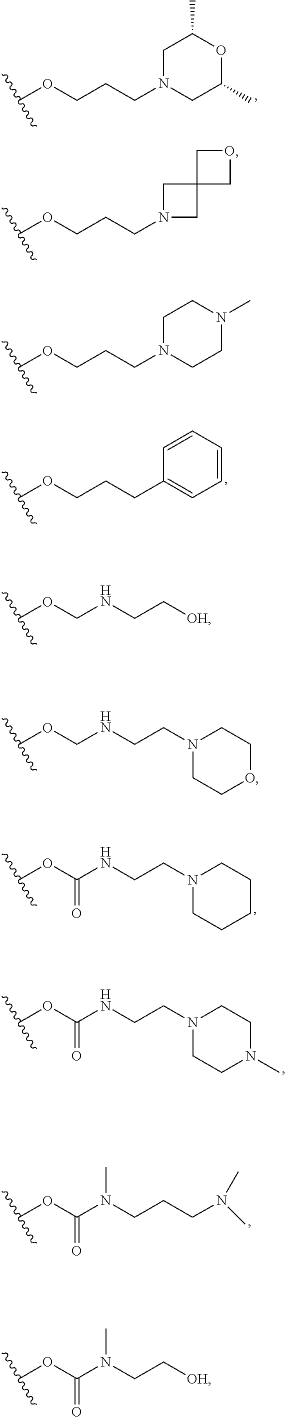

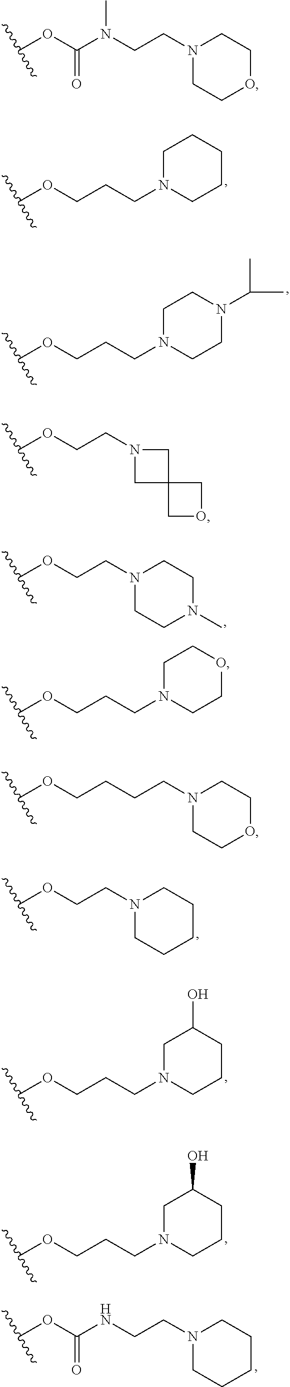

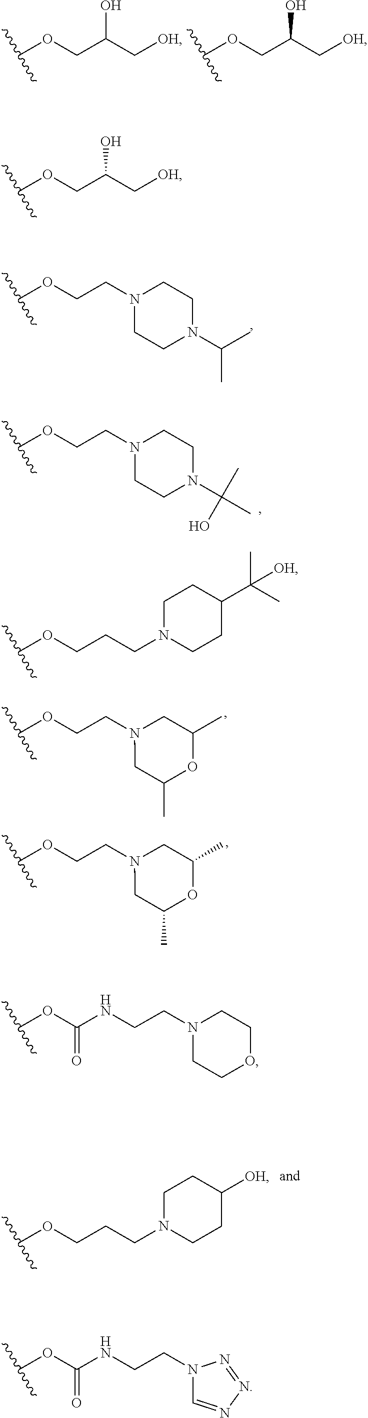

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), R¹ is selected from:

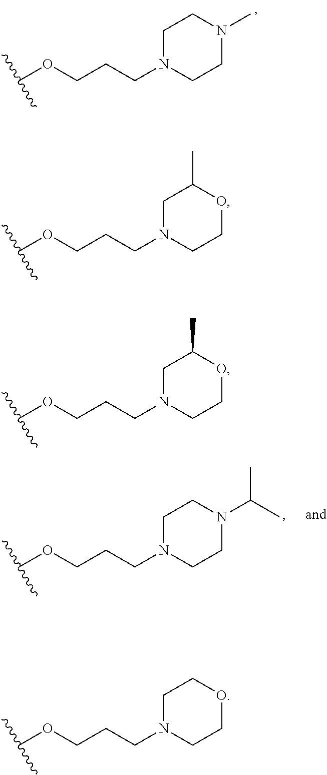 [SMILES: *OCCCN1CCN(C)CC1 ; *OCCCN1CCOC(C)C1 ; *OCCCN1CCO[C@H](C)C1 ; *OCCCN1CCN(C(C)C)CC1 ; *OCCCN1CCOCC1]

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), R¹ is

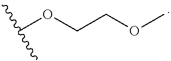 [SMILES: *OCCOC]

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), R¹ is

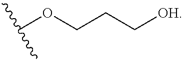 [SMILES: *OCCCO]

In some embodiments for a compound of Formula (IA), (IB), (IC), (ID), (IE), (IIA), R³⁴ of —OR³⁴ may be selected from:

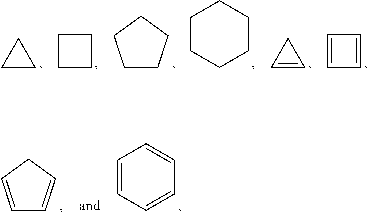

any one of which is optionally substituted.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), the heterocycle of R³⁴ of —OR³⁴ may be selected from:

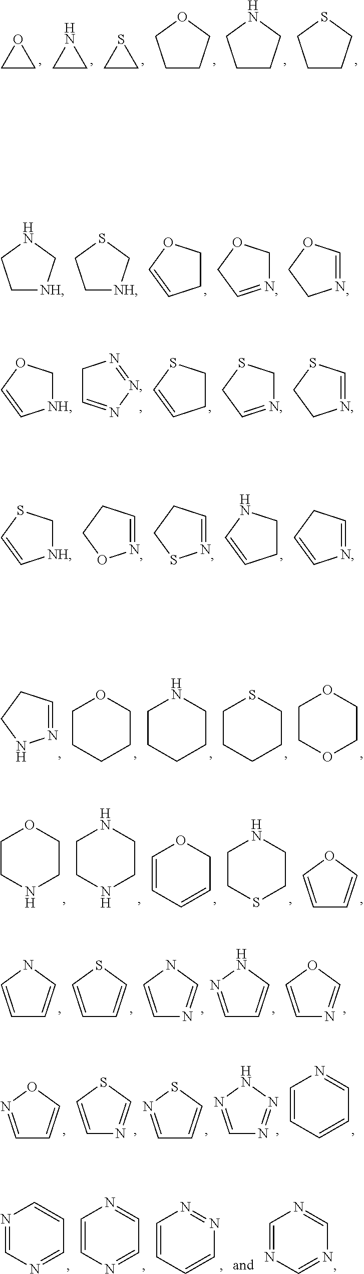

any one of which is optionally substituted.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q² of R¹ is —OR³⁴, and R³⁴ is selected from hydrogen, C₁-C₆ alkyl, carbocycle, and heterocycle. In some embodiments, the carbocycle of R³⁴ of —OR³⁴ is a C₃₋₆ carbocycle. In some embodiments, Q² of R¹ is selected from —OR³⁴, and R³⁴ is selected from hydrogen and optionally substituted C₁-C₆ alkyl. In some embodiments, Q² of R¹ is selected from —OR³⁴, and R³⁴ is selected from hydrogen and C₁-C₆ alkyl. In some embodiments, Q² of R¹ is selected from —OR³⁴, and R³⁴ is selected from hydrogen, methyl, ethyl and propyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), R¹ is selected from

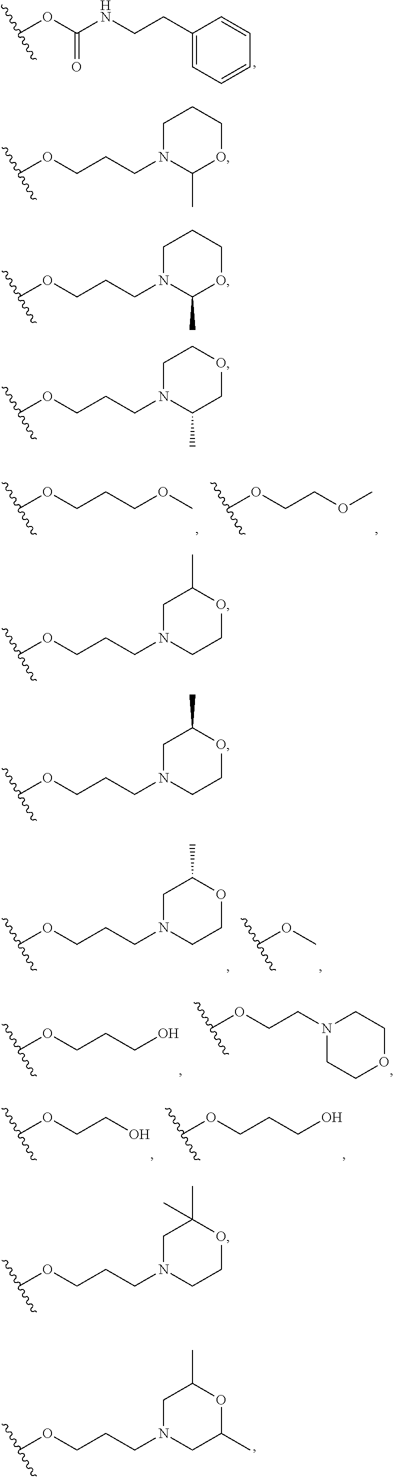

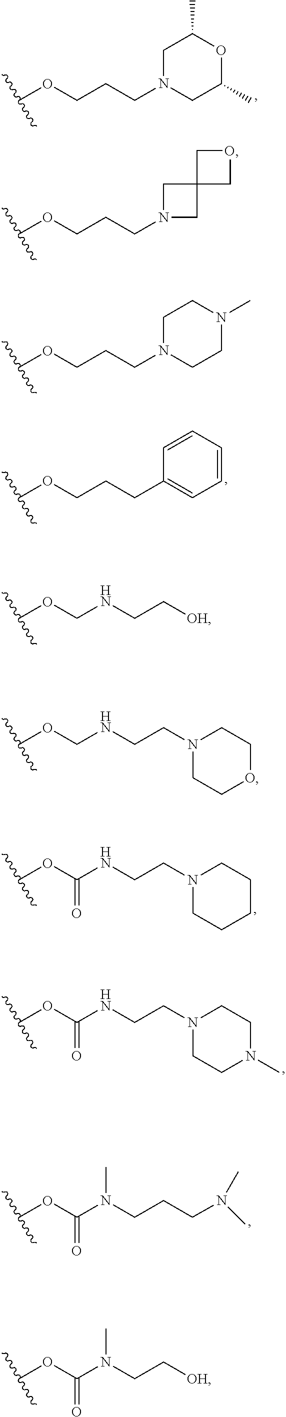

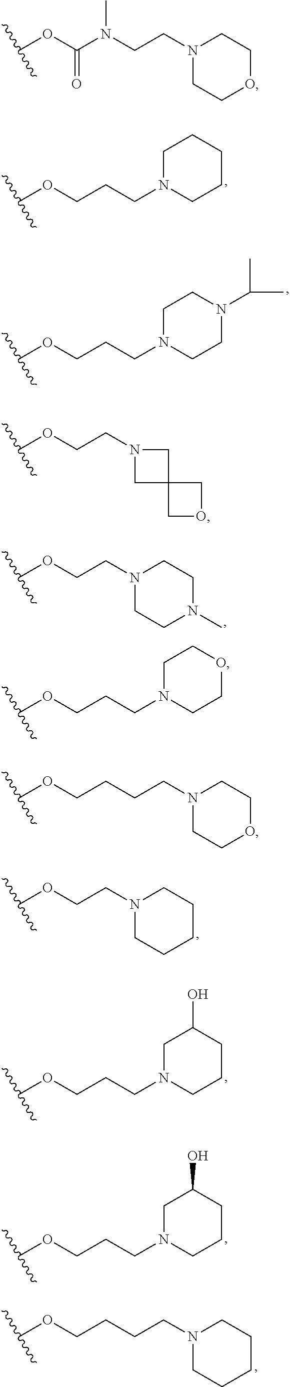

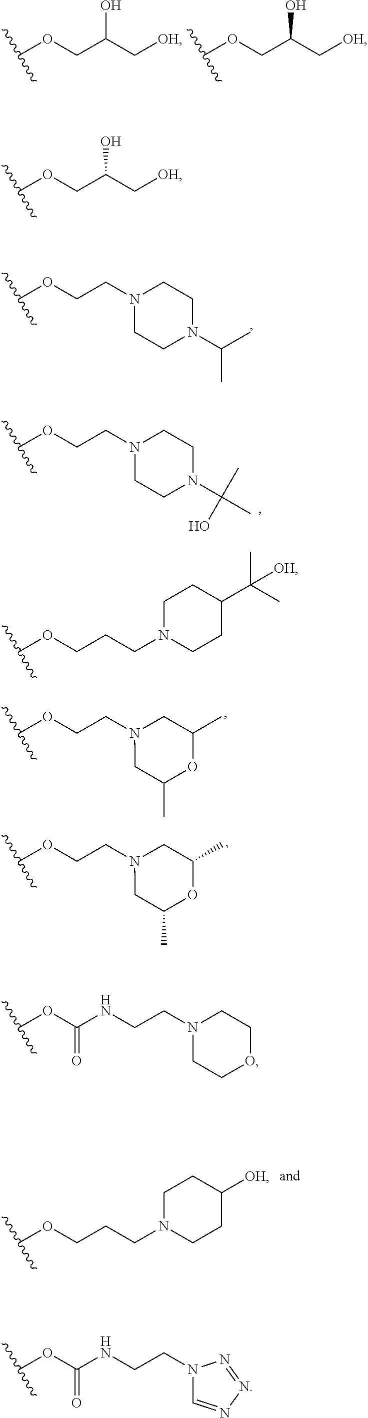

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), R¹ is selected from:

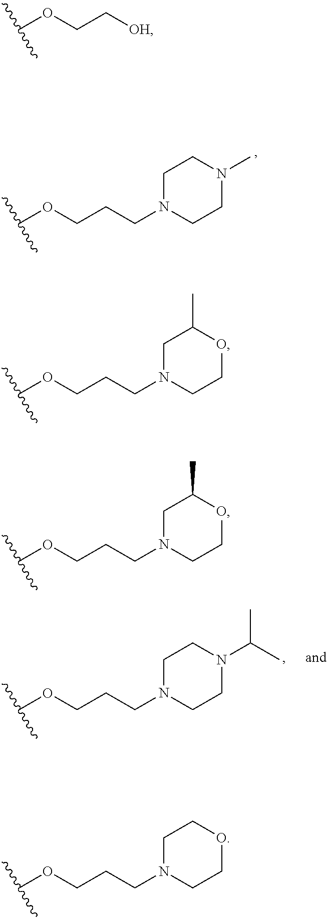 [SMILES: *OCCO ; *OCCCN1CCN(C)CC1 ; *OCCCN1CCOC(C)C1 ; *OCCCN1CCO[C@H](C)C1 ; *OCCCN1CCN(C(C)C)CC1 ; *OCCCN1CCOCC1]

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), R⁴ is selected from

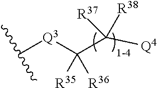

and —O—(CH₂)₀₋₁T.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), R⁴ is —O—(CH₂)₀₋₁T. In some embodiments, T of —O—(CH₂)₀₋₁T is an optionally substituted 3-6-membered heterocycloalkyl wherein substituents are independently selected from hydroxy, C₁-C₆ alkyl, hydroxy C₁-C₆ alkyl, alkoxy, and alkoxy C₁-C₆ alkyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), R⁴ is selected from

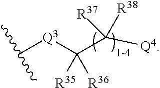

In some embodiments, Q³ of R⁴ is —O—.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), each of R³⁵, R³⁶, R³⁷ and R³⁸ of R⁴ are independently selected from hydrogen, hydroxy, halogen, cyano, nitro, and C₁-C₃ alkyl. In some embodiments, each of R³⁵, R³⁶, R³⁷ and R³⁸ of R⁴ are independently selected from hydrogen, hydroxy, and methyl.

In some embodiments for a compound of Formula (IA), (IB), (IC), (ID), (IE), (IIA), (IIB), (II), (III-A), (III-B), (III-C), (III-D), (III-E), (III-F), (III-G), or (III-H), one or two of R³, R³⁶, R³⁷ and R³⁸ of R⁴ is selected from hydroxy and methyl and the rest of R³⁵, R³⁶, R³⁷ and R³⁸ are each hydrogen.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), each of R³⁵, R³⁶, R³⁷, and R³⁸ are independently selected from hydrogen, hydroxy, halogen, cyano, nitro, C₁-C₆ alkyl, haloalkyl, hydroxy C₁₋₆ alkyl, alkoxy, and alkoxy C₁-C₆ alkyl, wherein no more than three of R³⁵, R³⁶, R³⁷, and R³⁸ are hydroxy, halogen, cyano, nitro, C₁-C₆ alkyl, haloalkyl, hydroxy C₁₋₆ alkyl, alkoxy, and alkoxy C₁-C₆ alkyl and the others are hydrogen.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), each of R³⁵, R³⁶, R³⁷, and R³⁸ are independently selected from hydrogen, hydroxy, halogen, cyano, nitro, C₁-C₆ alkyl, haloalkyl, hydroxy C₁₋₆ alkyl, alkoxy, and alkoxy C₁-C₆ alkyl, wherein no more than three of R³⁵, R³⁶, R³⁷, and R³⁸ are hydroxy.

In some embodiments for a compound of Formula (IA), (IB), (IC), (ID), (IE), (IA), (IIB), (IIC), (III-A), (III-B), (III-C), (III-D), (III-E), (III-F), (III-G), or (III-H), each of R³⁵, R³⁶, R³⁷, and R³⁸ are independently selected from hydrogen, hydroxy, halogen, cyano, nitro, C₁-C₆ alkyl, haloalkyl, hydroxy C₁₋₆ alkyl, alkoxy, and alkoxy C₁-C₆ alkyl, wherein no more than two of R³⁵, R³⁶, R³⁷, and R³⁸ are hydroxy.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q⁴ of R⁴ is selected from optionally substituted phenyl, and —OR⁴², wherein substituents on phenyl are independently selected from hydroxy, halogen, cyano, nitro, C₁-C₆ alkyl, haloalkyl, hydroxy C₁-C₆ alkyl, alkoxy, and alkoxy C₁-C₆ alkyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q⁴ of R⁴ is selected from phenyl and —OR⁴², and R⁴² is selected from hydrogen and optionally substituted C₁-C₆ alkyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), Q⁴ of R⁴ is selected from phenyl and —OR⁴², and R⁴² is selected from hydrogen, methyl, hydroxyethyl, and methoxyethyl.

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), R⁴ is selected from:

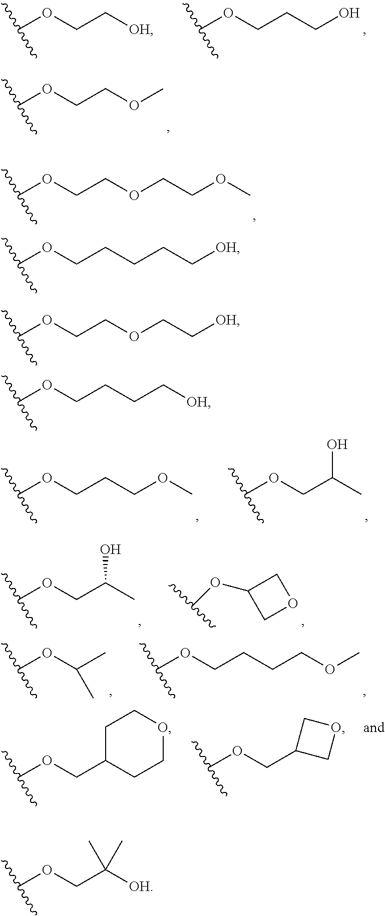

In some embodiments for a compound of Formula (IA), (IB), (IC), (ID), (IE), (IIA), (IIB), (IIC), (III-A), (III-B), (III-C), (III-D), (III-E), (III-F), (III-G), or (III-H), R⁴ is selected

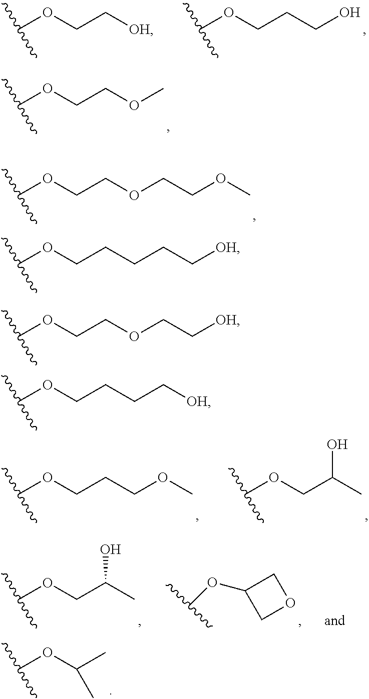

In certain embodiments, for a compound of Formula (IA), (IB), (IC), (ID), (IE), (IIA), (IIB), (IIC), (III-A), (III-B), (III-C), (III-D), (III-E), (III-F), (III-G), or (III-H):
R¹ is selected from

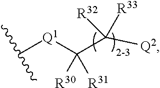

and —OCH₃;
R² is selected from optionally substituted C₁-C₆ alkoxy group, such as R² is a C₁-C₆ alkoxy group, and preferably R² is —OCH₃;
R³ is selected from an optionally substituted C₁-C₆ alkoxy group, such as R² is a C₁-C₆ alkoxy group, and preferably R² is —OCH₃;
   R⁴ is selected from

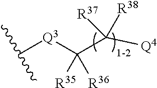

and —O—(CH₂)₀₋₁T;
T is an optionally substituted 4-6-membered heterocycloalkyl wherein substituents are independently selected from hydroxy, halogen, cyano, nitro, C₁-C₆ alkyl, haloalkyl, hydroxy C₁-C₆ alkyl, alkoxy, and alkoxy C₁-C₆ alkyl, such as T is selected from optionally substituted oxetane and optionally substituted pyran;
Q¹ and Q³ are independently selected from —O—, —OC(═O)NR⁴¹—, —S—, and —NR⁴⁰—, preferably Q¹ and Q³ are each —O—;
Q² is selected from optionally substituted C₃₋₆ carbocycle, optionally substituted 3-8-membered heterocycle, —OR³⁴, and —N(R³⁹)₂, wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl, preferably Q² is —OR³⁴;
Q⁴ is selected from optionally substituted C₃₋₆ carbocycle, optionally substituted 3-8-membered heterocycle, and —OR⁴², wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl, preferably Q⁴ is —OR⁴²;
R³⁰, R³¹, R³⁵, and R³⁶ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl, and preferably each of R³⁰, R³¹, R³⁵, and R³⁶ is hydrogen;
each R³², R³³, R³⁷, and R³⁸ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl, wherein no more than two of R³⁵, R³⁶ R³⁷, and R³⁸ are hydroxy, such as preferably each of R³², R³³, R³⁷, and R³⁸ are hydrogen;
R³⁴ is selected from hydrogen and optionally substituted C₁-C₆ alkyl, hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle, preferably R³⁴ is hydrogen or CH₃;
each R³⁹ is selected from hydrogen, C₁-C₆ alkyl, haloalkyl, and alkoxy C₁-C₆ alkyl;
each R⁴ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle;
each R⁴¹ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle; and
each R⁴² is selected from hydrogen, optionally substituted C₁-C₂ alkyl, optionally substituted carbocycle, and optionally substituted heterocycle, wherein the substituents on C₁-C₂ alkyl, carbocycle, and heterocycle are independently selected at each occurrence from hydroxy, halogen, cyano, nitro, C₁-C₂ alkoxy, carbocycle and heterocycle.

In certain embodiments, for a compound of Formula (IA), (IB), (IC), (ID), (IE), (IIA), (IIB), (IIC), (III-A), (III-B), (III-C), (III-D), (III-E), (III-F), (III-G), or (III-H):
R¹ is selected from

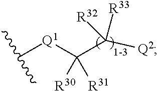

R² is selected from optionally substituted C₁-C₆ alkoxy, such as R² is a C₁-C₆ alkoxy group, and preferably R² is —OCH₃;
R³ is selected from an optionally substituted C₁-C₆ alkoxy, such as R² is a C₁-C₆ alkoxy group, and preferably R² is —OCH₃;
   R⁴ is selected from

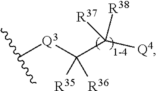

—O—(CH₂)₀₋₁T and —O—CH(CH₃)₂;
T is an optionally substituted 3-6-membered heterocycloalkyl wherein substituents are independently selected from hydroxy, halogen, cyano, nitro, C₁-C₆ alkyl, haloalkyl, hydroxy C₁-C₆ alkyl, alkoxy, and alkoxy C₁-C₆ alkyl;
Q¹ selected from —OC(═O)NR⁴¹—, —S—, and —NR⁴⁰—, preferably Q¹ is —OC(═O)NR⁴¹—;
Q³ is selected from —O—, —OC(═O)NR⁴¹—, —S—, and —NR⁴⁰—, preferably Q³ is —O—;
Q² is selected from optionally substituted phenyl, optionally substituted 3-6-membered saturated heterocycle, —OR³⁴, and —N(R³⁹)₂, wherein substituents on phenyl and 3-6-membered saturated heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
Q⁴ is selected from optionally substituted C₃₋₆ carbocycle, optionally substituted 3-8-membered heterocycle, and —OR⁴², wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl, preferably Q⁴ is —OR⁴²;
R³⁰, R³¹, R³⁵, and R³⁶ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl, and preferably each of R³⁰, R³¹, R³⁵, and R³⁶ is hydrogen;
each R³², R³³, R³⁷, and R³⁸ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl, wherein no more than two of R³⁵, R³⁶, R³⁷, and R³⁸ are hydroxy, such as preferably each of R³², R³³, R³⁷, and R³⁸ are hydrogen;
each R³⁴ is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle, preferably R³⁴ is hydrogen or CH₃;
each R³⁹ is selected from hydrogen, C₁-C₆ alkyl, haloalkyl, and alkoxy C₁-C₆ alkyl;
each R⁴⁰ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle;
each R⁴¹ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle; and
each R⁴² is selected from hydrogen, optionally substituted C₁-C₂ alkyl, optionally substituted carbocycle, and optionally substituted heterocycle, wherein the substituents on C₁-C₂ alkyl, carbocycle, and heterocycle are independently selected at each occurrence from hydroxy, halogen, cyano, nitro, C₁-C₂ alkoxy, carbocycle and heterocycle.

In certain embodiments, for a compound or salt of Formula (IA), (IB), (IC), (ID), (IE), (IIA), (IIB), (IIC), (III-A), (III-B), (III-C), (III-D) (III-E), (III-F), (III-G), or (III-H),
R¹ is selected from

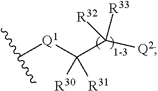

R² is selected from optionally substituted C₁-C₆ alkoxy group, such as R² is a C₁-C₆ alkoxy group, and preferably R² is —OCH₃;
R³ is selected from an optionally substituted C₁-C₆ alkoxy group, such as R² is a C₁-C₆ alkoxy group, and preferably R² is —OCH₃;
R⁴ is selected from

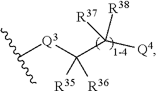

—O—(CH₂)₀₋₁T and —O—CH(CH₃)₂;
   T is an optionally substituted 3-6-membered heterocycloalkyl wherein substituents are independently selected from hydroxy, halogen, cyano, nitro, C₁-C₆ alkyl, haloalkyl, hydroxy C₁-C₆ alkyl, alkoxy, and alkoxy C₁-C₆ alkyl;
   Q¹ selected from —O—, —OC(═O)NR⁴¹—, and —NR⁴⁰—, preferably Q¹ is —O—;
   Q³ is selected from —O—, —OC(═O)NR⁴¹—, —S—, and —NR⁴⁰—, preferably Q³ is —O—;
   Q² is selected from optionally substituted phenyl, optionally substituted 3-8-membered saturated heterocycle, and —N(R³⁹)₂, wherein substituents on phenyl and 3-8-membered saturated heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl;
   Q⁴ is selected from optionally substituted C₃₋₆ carbocycle, optionally substituted 3-8-membered heterocycle, and —OR⁴², wherein substituents on C₃₋₆ carbocycle and 3-8-membered heterocycle are independently selected from hydroxy, halogen, cyano, nitro, C₁-C₆ alkyl, haloalkyl, hydroxy C₁-C₆ alkyl, alkoxy, and alkoxy C₁-C₆ alkyl, preferably Q⁴ is —OR⁴²;
   R³⁰, R³¹, R³⁵, and R³⁶ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl, and preferably each of R³⁰, R³¹, R³⁵, and R³⁶ is hydrogen;
   each R³², R³³, R³⁷, and R³⁸ are independently selected from hydrogen, hydroxy, halogen, cyano, nitro, C₁-C₆ alkyl, haloalkyl, hydroxy C₁-C₆ alkyl, alkoxy, and alkoxy C₁-C₆ alkyl, wherein no more than two of R³⁵, R³⁶, R³⁷, and R³⁸ are hydroxy, such as preferably each of R³², R³³, R³⁷, and R³⁸ are hydrogen;
   each R³⁴ is selected from hydrogen, optionally substituted C₁-C₆ alkyl, optionally substituted carbocycle, and optionally substituted heterocycle, wherein the substituents on C₁-C₆ alkyl, carbocycle, and heterocycle are independently selected at each occurrence from hydroxy, halogen, cyano, nitro, C₁-C₆ alkoxy, carbocycle and heterocycle, preferably R³⁴ is hydrogen or CH₃;
   each R³⁹ is selected from hydrogen, C₁-C₆ alkyl, haloalkyl, and alkoxy C₁-C₆ alkyl;
   each R⁴⁰ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle; and
   each R⁴¹ is selected from hydrogen and an optionally substituted C₁-C₆ alkyl group, wherein the substituents are independently selected at each occurrence from hydroxy, halogen, cyano, nitro, C₂-C₆ alkoxy group, carbocycle and heterocycle; and
   each R⁴² is selected from hydrogen, optionally substituted C₁-C₂ alkyl, optionally substituted carbocycle, and optionally substituted heterocycle, wherein the substituents on C₁-C₂ alkyl, carbocycle, and heterocycle are independently selected at each occurrence from hydroxy, halogen, cyano, nitro, C₁-C₂ alkoxy, carbocycle and heterocycle.

In certain embodiments, for a compound or salt of Formula (III-A), R¹ and R⁴ may be selected from Table 1. In some cases, R¹ may be selected from Table 1. In some cases, R⁴ may be selected from Table 1.

In certain embodiments, for a compound or salt of Formula (III-B), R¹ and R⁴ may be selected from Table 2. In some cases, R¹ may be selected from Table 2. In some cases, R⁴ may be selected from Table 2.

In certain embodiments, for a compound or salt of Formula (III-C), R¹ and R⁴ may be selected from Table 3. In some cases, R¹ may be selected from Table 3. In some cases, R⁴ may be selected from Table 3.

In certain embodiments, for a compound or salt of Formula (III-D), R¹ and R⁴ may be selected from Table 4. In some cases, R¹ may be selected from Table 4. In some cases, R⁴ may be selected from Table 4.

In certain embodiments, for a compound or salt of Formula (III-E), R¹ and R⁴ may be selected from Table 5. In some cases, R¹ may be selected from Table 5. In some cases, R⁴ may be selected from Table 5.

In certain embodiments, for a compound or salt of Formula (III-F), R¹ and R⁴ may be selected from Table 6. In some cases, R¹ may be selected from Table 6. In some cases, R⁴ may be selected from Table 6.

In certain embodiments, for a compound or salt of Formula (III-G), R¹ and R⁴ may be selected from Table 7. In some cases, R¹ may be selected from Table 7. In some cases, R⁴ may be selected from Table 7.

In certain embodiments, for a compound or salt of Formula (III-H), R¹ and R⁴ may be selected from Table 8. In some cases, R¹ may be selected from Table 8. In some cases, R⁴ may be selected from Table 8.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

When stereochemistry is not specified, molecules with stereocenters described herein include isomers, such as enantiomers and diastereomers, mixtures of enantiomers, including racemates, mixtures of diastereomers, and other mixtures thereof, to the extent they can be made by one of ordinary skill in the art by routine experimentation. In certain embodiments, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration.

For any Formula described herein with depicted stereochemistry at a particular position, the intended stereochemistry of a substituent is that depicted in the Formula. For example, a compound of Formula (III-A) where R⁴ is

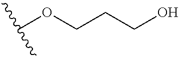 [SMILES: *OCCCO]

would have the following stereochemistry at R⁴:

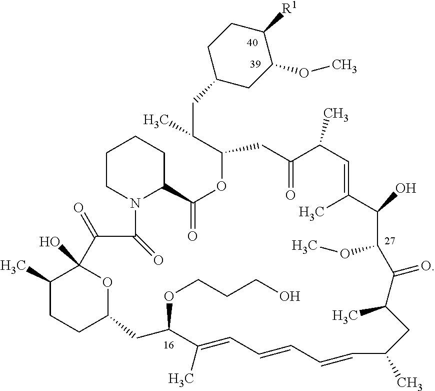

Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

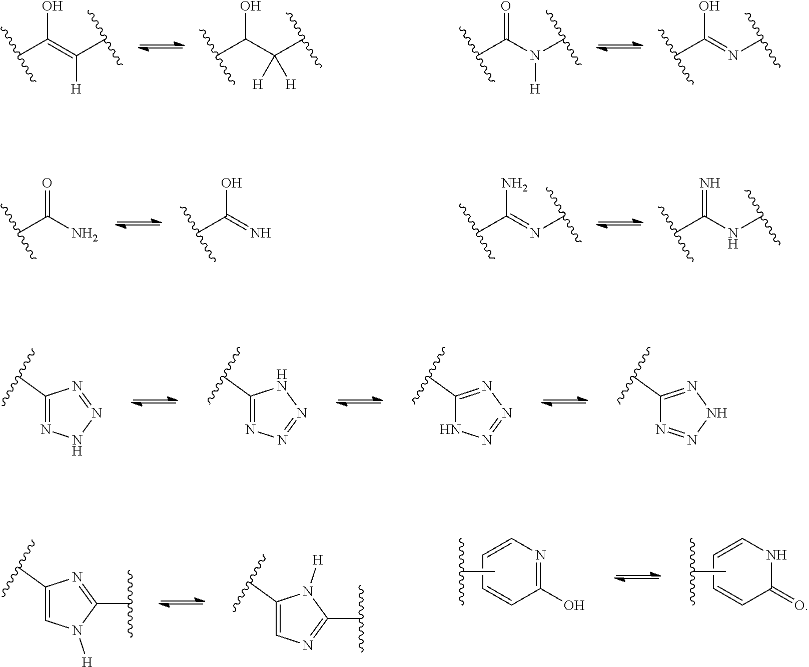

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ¹⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed Chrom., 6:283-286 (1992); 1. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

A compound or salt of any one of Formula (IA), (IB), (IC), (ID), (IE), (IIA), (IIB), (IIC), (III-A), (III-B), (III-C), (III-D), (III-E), (III-F), (III-G), or (III-H), may be formulated in any suitable pharmaceutical formulation. A pharmaceutical formulation of the present disclosure typically contains an active ingredient (e.g., compound or salt of any one of Formula (IA), (IB), (IC), (ID), (IE), (IIA), (IIB), (IIC), (III-A), (III-B), (III-C), (III-D), (III-E), (III-F), (III-G), or (III-H), and one or more pharmaceutically acceptable excipients or carriers, including but not limited to: inert solid diluents and fillers, diluents, sterile aqueous solution and various organic solvents, permeation enhancers, antioxidants, solubilizers, and adjuvants.

In certain embodiments, a pharmaceutical formulation of the disclosure comprises a mixture of diastereomers. The pharmaceutical formulation may include one major diastereomer which accounts for 50 wt % or more of the mixture of diastereomers in the formulation and one or more minor diastereomers which individually or in combination account for less than 50 wt % of the mixture of diastereomers. A pharmaceutical formulation may comprise 51 wt % or more of the major diastereomer, such as from about 60 wt % to 95 wt %, such as 70 wt % to 95 wt %, such as 80 wt % to 95 wt % of the major diastereomer and one or more minor diastereomers bringing the percentage to 100 wt %. For example, a pharmaceutical comprises 80 wt % of the compound of 525 of Table 3 and 20 wt % of the compound 126 of Table 1. As another example, a pharmaceutical formulation comprises a mixture of diastereomers with 80 wt % of compound 601 of Table 4, 10 wt % of compound 201 of Table 2, 8 wt % of compound 401 of Table 3, and 2 wt % of compound 2 of Table 1.

In certain embodiments, the pharmaceutical formulation comprises a compound or salt of the disclosure in a mixture of diastereomers with a major diastereomer and one or more minor diastereomers, wherein the one or more minor diastereomers account for about 0.5 wt % to about 20 wt % of the mixture of diastereomers in the pharmaceutical formulation. For example, a pharmaceutical formulation comprises from about 1 wt % to about 40 wt %, such as about 1 wt % to about 30 wt %, such as about 1 wt % to about 20 wt %, such as about 2 wt % to about 10 wt %, such as about 5 wt % to about 10 wt % of a minor diastereomer or a combination of minor diastereomers.

In certain embodiments, the pharmaceutical formulation comprises a compound or salt of the disclosure in a mixture of diastereomers wherein the major diastereomer accounts for 90 wt % or more, 95 wt % or more of even 98 wt % or more of the mixture of diastereomers. In certain embodiments, a compound or salt of Formula (IA), (IB), (IC), (ID), (IE), (IIA), (IIB), (IIC), (III-A), (III-B), (III-C), (III-D), (III-E), (III-F), (III-G), or (III-H), is formulated with an agent that inhibits degradation of the compound or salt. In certain embodiments, the compound or salt is formulated with one or more antioxidants. Acceptable antioxidants include, but are not limited to, citric acid, d,I-α-tocopherol, BHA, BHT, monothioglycerol, ascorbyl palmitate, ascorbic acid, and propyl gallate. In certain embodiments, the formulation contains from 0.1 to 30%, from 0.5 to 25%, from 1 to 20%, from 5 to 15%, or from 7 to 12% (wt/wt) CCI-779, from 0.5 to 50%, from 1 to 40%, from 5 to 35%, from 10 to 25%, or from 15 to 20% (wt/wt) water soluble polymer, from 0.5 to 10%, 1 to 8%, or 3 to 5% (wt/wt) surfactant, and from 0.001% to 1%, 0.01% to 1%, or 0.1% to 0.5% (wt/wt) antioxidant. In certain embodiments, the antioxidants of the formulations of this invention will be used in concentrations ranging from 0.001% to 3% wt/wt.

In certain embodiments, a compound or salt of Formula (IA), (IB), (IC), (ID), (IE), (IIA), (IIB), (IIC), (III-A), (III-B), (III-C), (III-D), (III-E), (III-F), (III-G), or (III-H), is formulated with a pH modifying agent to maintain a pH of about 4 to about 6. Acceptable pH modifying agents include, but are not limited to citric acid, sodium citrate, dilute HCl, and other mild acids or bases capable of buffering a solution containing a compound or a salt of the disclosure to a pH in the range of about 4 to about 6.

In certain embodiments, a compound or salt of Formula (IA), (IB), (IC), (ID), (IE), (IIA), (IIB), (IIC), (III-A), (III-B), (III-C), (III-D), (III-E), (III-F), (III-G), or (III-H), is formulated with a chelating agent or other material capable of binding metal ions, such as ethylene diamine tetra acetic acid (EDTA) and its salts are capable of enhancing the stability of a compound or salt of Formula (IA), (IB), (IC), (ID), (IE), (IIA), (IIB), (IIC), (III-A), (III-B), (III-C), (III-D), (III-E), (III-F), (III-G), or (III-H).

Pharmaceutical formulations may be provided in any suitable form, which may depend on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In some embodiments, the dosage form is formulated for oral administration. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form.

The amount of compound or salt of any one of Formula (IA), (IB), (IC), (ID), (IE), (IIA), (IIB), (IIC), (III-A), (III-B), (III-C), (III-D), (III-E), (III-F), (III-G), or (III-H) will be dependent on the mammal being treated, the severity of the disorder or condition, the rate of administration, the disposition of the compound or salt of any one of Formula (IA), (IB), (IC), (ID), (IE), (IIA), (IIB), (IIC), (III-A), (III-B), (III-C), (III-D), (III-E), (III-F), (III-G), or (III-H), and the discretion of the prescribing physician.

In some embodiments, pharmaceutically acceptable carriers of Formula (IA), (IB), (IC), (ID), (IE), (IA), (IIB), (IIC), (III-A), (III-B), (III-C), (III-D), (III-E), (III-F), (III-G), or (III-H), can include a physiologically acceptable compound that is an antioxidant.

In some embodiments, the disclosure provides a pharmaceutical composition for oral administration containing at least one compound or salt of any one of Formula (IA), (IB), (IC), (ID), (IE), (IIA), (IIB), (IIC), (III-A), (III-B), (III-C), (III-D), (III-E), (III-F), (III-G), or (III-H), and a pharmaceutical excipient suitable for oral administration. The composition may be in the form of a solid, liquid, gel, semi-liquid, or semi-solid. In some embodiments, the composition further comprises a second agent.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound or salt of any one of Formula (IA), (IB), (IC), (ID), (IE), (IIA), (IIB), (IC), (III-A), (III-B), (III-C), (III-D), (III-E), (III-F), (III-G), or (III-H), moistened with an inert liquid diluent.

In some embodiments, the disclosure provides a pharmaceutical composition for injection containing a compound or salt of any one of Formula (IA), (IB), (IC), (ID), (IE), (IIA), (IIB), (IIC), (III-A), (III-B), (III-C), (III-D), (III-E), (III-F), (III-G), or (III-H), disclosed herein and a pharmaceutical excipient suitable for injection. Components and amounts of agents in the composition are as described herein.

In certain embodiments, the compound or salt of any one of Formula (IA), (IB), (IC), (ID), (IE), (IIA), (IIB), (IIC), (III-A), (III-B), (III-C), (III-D), (III-E), (III-F), (III-G), or (III-H), may be formulated for injection as aqueous or oil suspensions, emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Pharmaceutical compositions may also be prepared from a compound or salt of any one of Formula (IA), (IB), (IC), (ID), (IE), (IIA), (IIB), (IIC), (III-A), (III-B), (III-C), (III-D), (III-E), (III-F), (III-G), or (III-H), and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

The disclosure also provides kits. The kits may include a compound or salt of any one of Formula (IA), (IB), (IC), (ID), (IE), (IIA), (IIB), (IIC), (III-A), (III-B), (III-C), (III-D), (III-E), (III-F), (III-G), or (III-H), and one or more additional agents in suitable packaging with written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, the compound or salt of any one of Formula (IA), (IB), (IC), (ID), (IE), (IIA), (IIB), (IIC), (III-A), (III-B), (III-C), (III-D), (III-E), (III-F), (III-G), or (III-H), and the agent are provided as separate compositions in separate containers within the kit. In some embodiments, the compound or salt of any one of Formula (IA), (IB), (IC), (ID), (IE), (IIA), (IIB), (IIC), (III-A), (III-B), (III-C), (III-D), (III-E), (III-F), (III-G), or (III-H), and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

### Applications

In one aspect, the present disclosure provides a method of inhibiting mTORC1, comprising administering a compound or salt of any one of Formula (IA), (IB), (IC), (ID), (IE), (IIA), (IIB), (IIC), (III-A), (III-B), (III-C), (III-D), (III-E), (III-F), (III-G), or (III-H). In one aspect, the present disclosure provides a method of inhibiting mTORC1 without appreciably modulating mTORC2, comprising administering a compound or salt of any one of Formula (IA), (IB), (IC), (ID), (IE), (IIA), (IIB), (IIC), (III-A), (III-B), (III-C), (III-D), (III-E), (III-F), (III-G), or (III-H). In certain embodiments, the compounds and salt of the disclosure do not appreciably inhibit mTORC2.

While not being bound to any particular mechanism, a compound or salt of any one of Formula (IA), (IB), (IC), (ID), (IE), (IIA), (IIB), (IIC), (III-A), (III-B), (III-C), (III-D), (III-E), (III-F), (III-G), or (III-H) may show reduced side effects relative to rapamycin. In particular, compounds or salts of the disclosure may not appreciably impact the gastrointestinal and/or cardiac systems. In certain embodiments the compounds of the disclosure may be administered in larger dosing amounts or over longer periods of time than the prescribed dosing amounts or timeframes for rapamycin. For example of the intended timeframes, a compound or salt of any one of Formula (IA), (IB), (IC), (ID), (IE), (IIA), (IIB), (IIC), (III-A), (III-B), (III-C), (III-D), (III-E), (III-F), (III-G), or (III-H) may be administered daily, every other day, once a week, once every two weeks over a period of time, such as 2 months or more, 4 months or more, 6 months or more, 1 year or more, or even two years or more. For example of the intended dosing, a compound or salt of any one of Formula (IA), (IB), (IC), (ID), (IE), (IIA), (IIB), (IIC), (III-A), (III-B), (III-C), (III-D), (III-E), (III-F), (III-G), or (III-H) may be administered in dose, 30% or greater, 50% greater, 80% or greater than rapamycin indicated dosing for the same indication.

In certain embodiments, a compound or salt of any one of Formula (IA), (IB), (IC), (ID), (IE), (IIA), (IIB), (IIC), (III-A), (III-B), (III-C), (III-D), (III-E), (III-F), (III-G), or (III-H) is administered to a subject in need thereof for the treatment and/or prevention of a tauopathy (including but not limited to Alzheimer's disease, Parkinson's disease, progressive supranuclear palsy (PSP), corticobasal degeneration, corticobasal syndrome, frontotemporal dementia, frontotemporal lobar degeneration (FTLD) including but not limited to FTLD-17, behavior variant FTD, primary progressive aphasia (semantic, agrammatic or logopenic variants), argyrophilic grain disease, Pick's disease, globular glial tauopathies, primary age-related tauopathy (including neurofibrillary tangle dementia), chronic traumatic encephalopathy (CTE)-traumatic brain injury and aging-related tau astrogliopathy), an mTORopathy (including but not limited to tuberous sclerosis complex (TSC)), an mTORopathy associated with epileptic seizures, focal cortical dysplasia (FCD), ganglioglioma, hemimegalencephaly, neurofibromatosis 1, Sturge-Weber syndrome, Cowden syndrome, PMSE (Polyhydramnios, Megalencephaly, Symptomatic Epilepsy)), familial multiple discoid fibromas (FMDF), an epilepsy/epileptic seizures (both genetic and acquired forms of the disease such as familial focal epilepsies, epileptic spasms, infantile spasms (IS), status epilepticus (SE), temporal lobe epilepsy (PLE) and absence epilepsy), rare diseases associated with a dysfunction of mTORC1 activity (e.g., lymphangioleiomyomatosis (LAM), Leigh's syndrome, Friedrich's ataxia, Diamond-Blackfan anemia, etc.), metabolic diseases (e.g., obesity, Type II diabetes, etc.), autoimmune and inflammatory diseases (e.g., Systemic Lupus Erythematosus (SLE), multiple sclerosis (MS) psoriasis, etc.), cancer, a fungal infection, a proliferative disease, the maintenance of immunosuppression, transplant rejection, traumatic brain injury, autism, lysosomal storage diseases and neurodegenerative diseases associated with an mTORC1 hyperactivity (e.g., Parkinson's, Huntington's disease, etc.), aberrant compound accumulation, dysfunction of the autophagy mechanisms, and generally including but not limited to disorders that can be modulated by selective inhibition of the mTORC1 pathway.

In certain embodiments, a compound or salt of any one of Formula (IA), (IB), (IC), (ID), (IE), (IIA), (IIB), (IIC), (III-A), (III-B), (III-C), (III-D), (III-E), (III-F), (III-G), or (III-H) is administered to a subject in need thereof for treatment and/or prevention of a tauopathy selected from the group consisting of: progressive supranuclear palsy, dementia pugilistica (chronic traumatic encephalopathy), frontotemporal dementia, lytico-bodig disease (parkinson-dementia complex of guam), tangle-predominant dementia (with nfts similar to ad, but without plaques), ganglioglioma and gangliocytoma, meningioangiomatosis, subacute sclerosing panencephalitis, lead encephalopathy, tuberous sclerosis, Pick's disease, corticobasal degeneration (tau proteins are deposited in the form of inclusion bodies within swollen or “ballooned” neurons), Alzheimer's disease, Parkinson's disease, Huntington's disease, frontotemporal dementia, and frontotemporal lobar degeneration.

In certain embodiments, a compound or salt of any one of Formula (IA), (IB), (IC), (ID), (IE), (IIA), (IIB), (IIC), (III-A), (III-B), (III-C), (III-D), (III-E), (III-F), (III-G), or (III-H) is administered to a subject in need thereof for the treatment and/or prevention of a tauopathy selected from the group consisting of: Alzheimer's disease, Parkinson's disease, progressive supranuclear palsy (PSP), corticobasal degeneration, corticobasal syndrome, frontotemporal dementia, frontotemporal lobar degeneration (FTLD) including but not limited to FTLD-17, behavior variant FTD, primary progressive aphasia (semantic, agrammatic or logopenic variants), argyrophilic grain disease, Pick's disease, globular glial tauopathies, primary age-related tauopathy (including neurofibrillary tangle dementia), chronic traumatic encephalopathy (CTE)-traumatic brain injury and aging-related tau astrogliopathy.

In certain embodiments, a compound or salt of any one of Formula (IA), (IB), (IC), (ID), (IE), (IIA), (IIB), (IIC), (III-A), (III-B), (III-C), (III-D), (III-E), (III-F), (III-G), or (III-H) is administered to a subject in need thereof for the treatment and/or prevention of a mTORopathy. The mTORopathy may be, for example, Tuberous Sclerosis, Focal Cortical Dysplasia, or a PTEN (Phosphatase and tensin homolog) disease, etc. The mTORopathy may be a disease or disorder described elsewhere herein.

In certain embodiments, a compound or salt of any one of Formula (IA), (IB), (IC), (ID), (IE), (IIA), (IIB), (IIC), (III-A), (III-B), (III-C), (III-D), (III-E), (III-F), (III-G), or (III-H) is administered to a subject in need thereof for the treatment and/or prevention of cancer. Non-limiting examples of cancers can include Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer, Rectal cancer; Renal cell carcinoma (kidney cancer); Renal pelvis and ureter, transitional cell cancer, Rhabdomyosarcoma; Salivary gland cancer; Sarcoma, Ewing family of tumors; Sarcoma, Kaposi; Sarcoma, soft tissue; Sarcoma, uterine; Sézary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer, Vaginal cancer; Waldenström macroglobulinemia; Wilms tumor and any combination thereof.

In certain embodiments, a compound or salt of any one of Formula (IA), (IB), (IC), (ID), (IE), (IIA), (IIB), (IIC), (III-A), (III-B), (III-C), (III-D), (III-E), (III-F), (III-G), or (III-H) is administered to a subject in need thereof for the treatment and/or prevention of seizures and/or seizure related disorders. The seizure related disorders may include but not limited to: West syndrome, Focal Cortical Dysplasia (FCD), tuberous sclerosis complex (TSC), childhood absence epilepsy, benign focal epilepsies of childhood, juvenile myoclonic epilepsy (QME), temporal lobe epilepsy, frontal lobe epilepsy, refractory epilepsy, Lennox-Gastaut syndrome, occipital lobe epilepsy, 5 Proteus syndrome, hemi-megalencephaly syndrome (HMEG), megalencephaly syndrome (MEG), megalencephaly-capillary malformation (MCAP), megalencephalypolymicrogyria-polydactyly-hydrocephalus syndrome (MPPH) and PTEN disorders.

A compound according any therapeutic compound disclosed herein for use in the treatment and/or prevention of disorders that include the processes of fibrosis and/or inflammation (e.g., liver and kidney disorders). The disorders may include but not limited to liver fibrosis (which may occur in end-stage liver disease); liver cirrhosis; liver failure due to toxicity; non-alcohol-associated hepatic steatosis or NASH; and alcohol-associated steatosis. Another example may be kidney fibrosis, which may occur as a result of acute kidney injury or diabetic nephropathy can induce kidney fibrosis and inflammation.

A compound according any therapeutic compound disclosed herein for use in the treatment and/or prevention of disorders that include the processes of fibrosis and/or inflammation (e.g., liver and kidney disorders). The disorders may include but not limited to liver fibrosis (which may occur in end-stage liver disease); liver cirrhosis; liver failure due to toxicity; non-alcohol-associated hepatic steatosis or NASH; and alcohol-associated steatosis. Another example may be kidney fibrosis, which may occur as a result of acute kidney injury, chronic kidney disease, or diabetic nephropathy can induce kidney fibrosis and inflammation. The disorder may include polycystic kidney disease, ischemia/reperfusion injury, transplantation, adriamycin nephropathy, unilateral ureteral obstruction (UUO), glomerulopathy, IgA nephropathy, focal segmental glomerulosclerosis (FSGS), Lupus mesangial proliferative nephritis.

A compound according any therapeutic compound disclosed herein for use in the treatment and/or prevention of acute or chronic organ or tissue transplant rejection, for example, heart, lung, combined heart-lung, liver, kidney, pancreatic, skin or corneal transplants, prevention of graft-versus-host disease, such as following bone marrow transplantation, etc.

A compound according any therapeutic compound disclosed herein for use in the treatment and/or prevention of autoimmune diseases and/or and inflammatory conditions include in particular inflammatory conditions with an etiology that may include an autoimmune component such as arthritis (for example rheumatoid arthritis, arthritis chronica progrediente and arthritis deformans) and rheumatic diseases. Examples may include autoimmune hematological disorders (including e. g. hemolytic anemia, aplastic anemia, pure red cell anaemia and idiopathic thrombocytopenia), systemic lupus erythematosus, polychondritis, scleroderma, Wegener granulamatosis, dermatomyositis, chronic active hepatitis, myasthenia gravis, psoriasis, Steven-Johnson syndrome, idiopathic sprue, autoimmune inflammatory bowel disease (including e. g. ulcerative colitis and Crohn's disease) endocrine ophthalmopathy, Graves disease, sarcoidosis, multiple sclerosis, primary biliary cirrhosis, juvenile diabetes (diabetes mellitus type I), uveitis (anterior and posterior), keratoconjunctivitis sicca and vernal keratoconjunctivitis, interstitial lung fibrosis, psoriatic arthritis, glomerulonephritis (with and without nephrotic syndrome, e.g. including idiopathic nephrotic syndrome or minimal change nephropathy) and juvenile dermatomyositis.

A compound according any therapeutic compound disclosed herein for use in the treatment and/or prevention of mitochondrial diseases or disorders.

A compound according any therapeutic compound disclosed herein for use in the treatment and/or prevention of smooth muscle cell proliferation migration leading to vessel intimal thickening, blood vessel obstruction, obstructive coronary atherosclerosis, or restenosis.

In certain embodiments, a compound or salt of Formula (IA), (IB), (IC), (ID), (IE), (IIA), (IIB), (IIC), (III-A), (III-B), (III-C), (III-D), (III-E), (III-F), (III-G), or (III-H) or a compound of any one of Tables 1, 2, 3, 4, 5, 6, 7, or 8 is administered to a subject in need thereof for the treatment and/or prevention of diabetic nephropathy, kidney-related complications of type 1 diabetes and type 2 diabetes, autosomal dominant polycystic kidney disease (ADPKD), autosomal recessive polycystic kidney disease (ARPKD), kidney diseases associated with cyst formation or cystogenesis, focal segmental glomerulosclerosis (FSGS) and other diseases associated with sclerosis of the kidney (glomerulopathy, IgA nephropathy, Lupus mesangial proliferative nephritis), laminopathies, age-related macular degeneration (AMD), diabetic macular edema, diabetic retinopathy, glaucoma, age related retinal disease, immune system senescence, respiratory tract infections, urinary tract infections, heart failure, osteoarthritis, pulmonary arterial hypertension (PAH), and/or chronic obstructive pulmonary disease (COPD).

In certain embodiments, a compound or salt of Formula (IA), (IB), (IC), (ID), (IE), (IIA), (IIB), (IIC), (III-A), (III-B), (III-C), (III-D), (III-E), (III-F), (III-G), or (III-H) or a compound of any one of Tables 1, 2, 3, 4, 5, 6, 7, or 8 is administered to a subject in need thereof for the treatment and/or prevention of Lymphangioleiomyomatosis (LAM) and/or polycystic kidney disease.

In certain embodiments, the disclosure provides a method of treating disease characterized by hyperactivation of mTORC1. The following references include methods for evaluating mTORC (e.g., mTORC1) activity: T. O'Reilly et al., Translational Oncology, v3, i2, p 65-79, (2010); J. Peralba, Clinical Cancer Research, v9, i8, p 2887-2892 (2003); D. R. Moore et al., Acla Physiologica, v201, i3, p 365-372 (2010); M. Dieterlen., Clinical Cytometry, v82B, i3, p 151-157, (2012); the contents of each of which are incorporated by reference herein.

In certain embodiments, the disclosure provides a method of treating age-related diseases. It may be established that modulation of mTORC1 signalling may prolong lifespan and may delay the onset of age-related diseases across a wide array of organisms, ranging from flies to mammals, thus possibly providing therapeutic options for preventing or treating age-related diseases in humans. In a recent clinical study Mannick et al. (mTOR inhibition improves immune function in the elderly, Sci Transl Med. 2014 Dec. 24; 6(268):268ra179. doi: 10.1126/scitranslmed.3009892) may have showed that mTOR inhibition improves the immune function in the elderly.

In certain embodiments, the disclosure provides a method of treating mitochondrial diseases. Mitochondrial myopathy and mitochondrial stress may be mitochondrial disorders as described in Chinnery, P. F. (2015); EMBO Mol. Med. 7, 1503-1512; Koopman, W. J. et al., 10 (2016); EMBO Mol. Med. 8, 311-327 and Young, M. J., and Yound and Copeland, W. C. (2016); Curr. Opin. Genet. Dev. 38, 52-62.

In certain embodiments, the disclosure provides a method of treating diseases of impaired autophagy. In some cases they may include impaired autophagies that result in mitochondrial damage, lysosomal storage diseases, cancer, Crohn's disease, etc. In some cases the impaired autophagies may be as described in Jiang P. & Mizushima, N., Autophagy and human diseases, Cell Research volume 24, p. 69-79 (2014).

In certain embodiments, the disclosure provides a method of treating limbic predominate age-related tar DNA-binding protein 43 (TDP-43) encephalopathy. In some cases, the compounds herein may be used to treat a condition or disease associated with misfolded TDP-43. In some cases, the compounds herein may be used to treat a TDP-43 associated neurodegenerative disease.

In certain embodiments, a compound or salt of the disclosure is used to induce heterodimerization of FKBP12 and the FRB domain of mTOR. Chemical Induction of Dimerization (CID) can be employed as a biological tool to spatially manipulate specific molecules, e.g., peptides and polypeptides, within cells at precise times to control a particular activity. Uses of CID include experimental investigations to elucidate cellular systems and therapeutic uses to regulate cell-based therapies. Exemplary uses include activation of cells used to promote engraftment, to treat diseases or conditions, or to control or modulate the activity of therapeutic cells that express chimeric antigen receptors or recombinant T cell receptors. Compounds of the disclosure maybe used in the development of inducible systems or molecular switches to control cell signaling.

The use of rapamycin as a dimerizing agent is limited by side effects associated with mTOR inhibition. mTOR inhibition can lead to reductions in cell growth and proliferation as well as possible immunosuppression. In contrast, compounds of the present disclosure may present an advantage over rapamycin due to the high selectivity for mTOR1 over mTOR2. mTOR2 inhibition is associated with the negative side effects affiliated with rapamycin. As the presently described compounds are selective from mTOR1 and have minimal impact on mTOR2.

In certain embodiments, the disclosure provides a method of approximating or multimerizing two or more polypeptides within a cell, comprising administering a compound with an pIC50 of 8.0 or greater, 8.5 or greater, or even 9.0 or greater for mTOR1 and a pIC50 of 7.0 or less, 6.5 or less, or even 6 or less for mTOR2. In certain embodiments, the disclosure provides a method of inducing heterodimerization of FKBP12 and the FRB domain of mTOR in a cell, comprising contacting the cell with a compound with a pIC50 of 8.0 or greater, 8.5 or greater, or even 9.0 or greater for mTOR1 and a pIC50 of 7.0 or less, 6.5 or less, or even 6 or less for mTOR2. In certain embodiments, the compound is any one of the compounds described herein, e.g., a compound of Formula (IA), (IB), (IC), (ID), (IE), (IIA), (IIB), (IIC), (III-A), (III-B), (III-C), (III-D), (III-E), (III-F), (III-G), or (III-H) or a compound of any one of Tables 1, 2, 3, 4, 5, 6, 7, or 8. In certain embodiments the cell is in vitro. In certain embodiments, the cell is in vivo.

The term “multimerize” or multimerization refers to the dimerization of two peptides or polypeptides, or the multimerization of more than two peptides or polypeptides, for example, the dimerization of FKBP12 and the FRB domain of mTOR.

Inducible FKBP12/FRB-based multimerization systems can also be incorporated into chimeric antigen receptor (CAR) T cells which can be used, for example, in immunotherapy applications. One type of immunotherapy is adoptive cell transfer in which a subject's immune cells are collected and modified ex vivo, e.g., CAR-modified T cells, to provide for specific and targeted tumor cell killing when the modified cells are returned to the body. T Cells from a patient's blood may be extracted and genetically engineered to express CARs on the cell surface. The components of a CAR typically include an extracellular, antibody-derived single chain variable fragment (scFv), which specifically recognizes a target tumor cell antigen, and one or more multicellular T-cell-derived signaling sequences fused to the scFv. Binding of the scFv region to an antigen results in activation of the T cell through the signaling domains of the CAR. In certain embodiments, a compound of the disclosure may be administered to a cell to activate a CAR-T cell with an FKBP12/FRB-based multimerization system. In certain embodiments, the disclosure provides a method of activating the growth of a cell, e.g., CAR-T cell, containing an FKBP protein fusion and an FRB fusion protein by contacting the cell with a compound of Formula (IA), (IB), (IC), (ID), (IE), (IIA), (IIB), (IIC), (III-A), (III-B), (III-C), (III-D), (III-E), (III-F), (III-G), or (III-H) or a compound of any one of Tables 1, 2, 3, 4, 5, 6, 7, or 8.

In some instances, it is beneficial to increase the activity of a therapeutic cell. For example, co-stimulating polypeptides may be used to enhance the activation of T Cells, and of CAR-expressing T cells against antigens, which would increase the potency of the adoptive immunotherapy. These treatments are used, for example, to treat tumors for elimination, and to treat cancer and blood disorders, but these therapies may have negative side effects. Overzealous on-target effects, such as those directed at large tumor masses, can lead to cytokine storms associated with tumor lysis syndrome (TLS), cytokine release syndrome (CRS) or macrophage activation syndrome (MAS). In some instances of therapeutic cell-induced adverse events, there is a need for rapid and near complete elimination of the therapeutic cells. If there is a need to reduce the number of transferred CAR-T cells, an inducing ligand may be administered to the subject being treated, thereby inducing apoptosis specifically of the modified T cells. For example, multimeric versions of the ligand binding domains FRB and/or FKBP12 or variants thereof, such as those described in WO 2020/076738, fused to caspase proteins and expressed in a modified therapeutic cell can serve as scaffolds that permit the spontaneous dimerization and activation of the caspase units upon recruitment through the FRB and/or FKBP12 with a chemical inducing agent such as a compound of Formula (IA), (IB), (IC), (ID), (IE), (IIA), (IIB), (IIC), (III-A), (III-B), (III-C), (III-D), (III-E), (III-F), (III-G), or (III-H) or a compound of any one of Tables 1, 2, 3, 4, 5, 6, 7, or 8. In certain embodiments, the disclosure provides a method of inhibiting the growth of a cell containing an FKBP protein fusion and an FRB fusion protein by contacting the cell with a compound a compound of Formula (IA), (IB), (IC), (ID), (IE), (IIA), (IIB), (IIC), (III-A), (III-B), (III-C), (III-D), (III-E), (III-F), (III-G), or (III-H) or a compound of any one of Tables 1, 2, 3, 4, 5, 6, 7, or 8.

The following examples are offered to illustrate, but not to limit the claimed invention. It will be recognized that these preparation methods are illustrative and not limiting. Using the teaching provided herein, numerous other methods of producing the rapamycin analogs described herein will be available to one of skill in the art.

### EXAMPLES

### Illustrative Synthetic Schemes

Illustrative synthetic routes to prepare a compound of Formula (IA), (IB), (IC), (ID), (IE), (IIA), (IIB), (IIC), (III-A), (III-B), (III-C), (III-D), (III-E), (III-F), (III-G), or (III-H), or a compound of any one of Tables 1, 2, 3, 4, 5, 6, 7, or 8 shown and described herein are exemplary only and are not intended, nor are they to be construed, to limit the scope of the present disclosure in any manner whatsoever. Those skilled in the art will be able to recognize modifications of the disclosed synthetic schemes and to devise alternate routes based on the disclosed examples provided herein; all such modifications and alternate routes are within the scope of the claims.

The chemical entities described herein can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in Schemes 1-32 the steps in some cases may be performed in a different order than the order shown in Schemes 1-32. Numberings or R groups in each scheme do not necessarily correspond to that of the claims or other schemes or tables herein. In some embodiments, C16 modification may be performed before C40 modification. In some embodiments, C40 modification may be performed before C16 modification. In some embodiments, C28 modification may be performed before/after C16 and/or C40 modification.

Compounds of the disclosure with C40 and/or C28 modifications including stereochemical inversions at these positions may be prepared as previously described, for example, in PCT Publication Nos. WO 95/14023 and WO 01/14387.

In certain embodiments, compounds of the disclosure are prepared from one of the following compounds as a starting material: rapamycin, everolimus, and/or 27-o-desmethyl rapamycin.

In some embodiments, compounds of Tables 1 to 8 may be prepared according to schemes 1 to 32. The compounds of tables 1 to 8 may have the core structure of Formula (III-A), Formula (III-B), Formula (III-C), Formula (III-D), Formula (III-E), Formula (III-F), Formula (III-G), or Formula (III-H) as shown below with the R¹ and R⁴ illustrated in table 1 to 8.

The compound nomenclature below was generated using Dotmatics ELN.

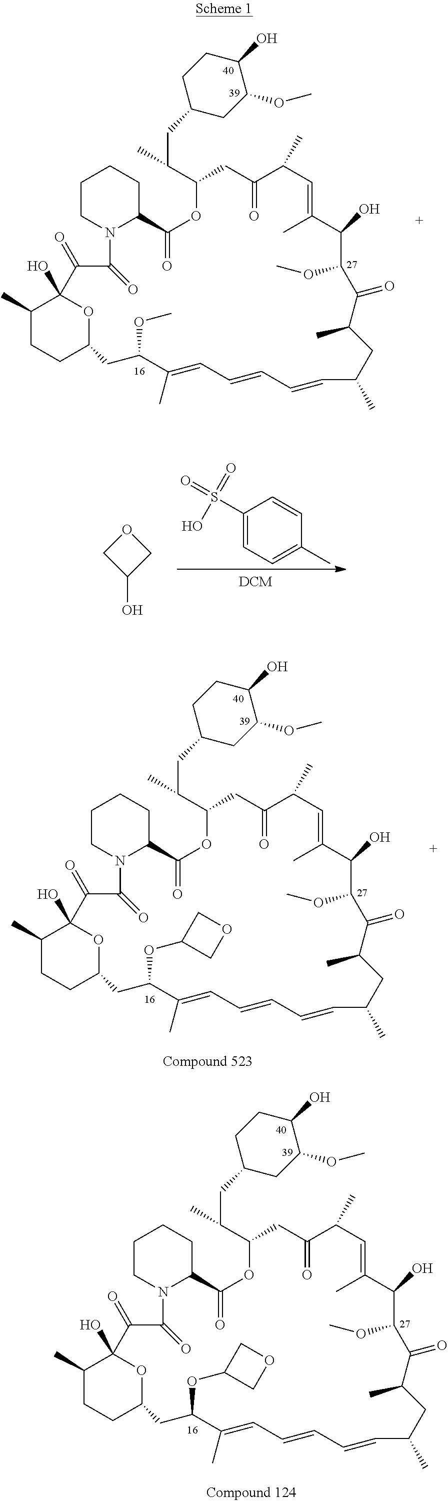

Oxetan-3-ol (5.8 mL, 87.5 mmol) was added to a solution of rapamycin (2.00 g, 2.19 mmol) in anhydrous DCM (87 mL). The mixture was cooled to −40° C. and 4-methylbenzenesulfonic acid (1.88 g, 10.9 mmol) was added. The mixture was stirred 90 minutes at room temperature. The mixture was diluted with DCM and neutralized by a saturated solution of NaHCO3. The phases were separated. The organic phase was washed with water (40 mL), dried, filtered and concentrated to dryness. The resulting crude mixture was purified by reverse phase chromatography (Uptisphere Strategy C18-Hq 10 um 250×30.0 mm CH₃CN:H₂O gradient 70:30 to 100:0, 277 nm). The main fraction (872 mg) was purified by SFC separation to afford (1R,9S,12S,15R,16E,18R,19R,21R,23S,24E,26E,28E,30S,32S,35R)-1,18-dihydroxy-12-[(2R)-1-[(1S,3R,4R)-4-hydroxy-3-methoxycyclohexyl]propan-2-yl]-19-methoxy-15,17,21,23,29,35-hexamethyl-30-(oxetan-3-yloxy)-11,36-dioxa-4-azatricyclo[30.3.1.04,9]hexatriaconta-16,24,26,28-tetraene-2,3,10,14,20-pentone (169 mg, 9%, white amorphous solid, compound 523) and (1R,9S,12S,15R,16E,18R,19R,21R,23S,24E,26E,28E,30R,32S,35R)-1,18-dihydroxy-12-[(2R)-1-[(1S,3R,4R)-4-hydroxy-3-methoxycyclohexyl]propan-2-yl]-19-methoxy-15,17,21,23,29,35-hexamethyl-30-(oxetan-3-yloxy)-11,36-dioxa-4-azatricyclo[30.3.1.04,9]hexatriaconta-16,24,26,28-tetraene-2,3,10,14,20-pentone (56 mg, 3%, white amorphous solid, compound 124).

SFC separation: Column: Princeton 2 Ethylpyridine 5 μm 60 A. Column size: 3 cm I.D.×15 cm L. Mobile phase: Carbon dioxide/Isopropanol (CO₂/IpOH) 80/20. Flowrate: 100 ml/min. Pressure: 100 Bar. Wavelength: UV 277 nm. SFC Equipment: Waters SFC200.

Compound 523: ¹H NMR (DMSO-d₆, 600 MHz): δ (ppm) 5.84-6.54 (m, 5H), 5.47 (dd, J=14.9, 9.8 Hz, 1H), 5.23 (d, J=4.5 Hz, 1H), 5.08 (br d, J=10.1 Hz, 1H), 4.90-5.02 (m, 2H), 4.47-4.72 (m, 3H), 4.28-4.45 (m, 3H), 3.97-4.08 (m, 2H), 3.88 (d, J=5.0 Hz, 1H), 3.69-3.79 (m, 1H), 3.41-3.49 (m, 1H), 3.00-3.36 (m, 9H), 2.66-2.89 (m, 2H), 2.34-2.47 (m, 2H), 2.13-2.33 (m, 1H), 1.46-2.11 (m, 19H), 1.10-1.46 (m, 7H), 0.65-1.09 (m, 19H), 0.59 (q, J=12.0 Hz, 1H). LCMS: MNa+ (ion type), 978.3 (ion m/z).

Compound 124: ¹H NMR (DMSO-d₆, 600 MHz): δ (ppm) 5.84-6.54 (m, 5H), 5.47 (dd, J=14.9, 9.8 Hz, 1H), 5.23 (d, J=4.5 Hz, 1H), 5.08 (br d, J=10.1 Hz, 1H), 4.90-5.02 (m, 2H), 4.47-4.72 (m, 3H), 4.28-4.45 (m, 3H), 3.97-4.08 (m, 2H), 3.88 (d, J=5.0 Hz, 1H), 3.69-3.79 (m, 1H), 3.41-3.49 (m, 1H), 3.00-3.36 (m, 9H), 2.66-2.89 (m, 2H), 2.34-2.47 (m, 2H), 2.13-2.33 (m, 1H), 1.46-2.11 (m, 19H), 1.10-1.46 (m, 7H), 0.65-1.09 (m, 19H), 0.59 (q, J=12.0 Hz, 1H). LCMS: MNa+ (ion type), 978.3 (ion m/z).

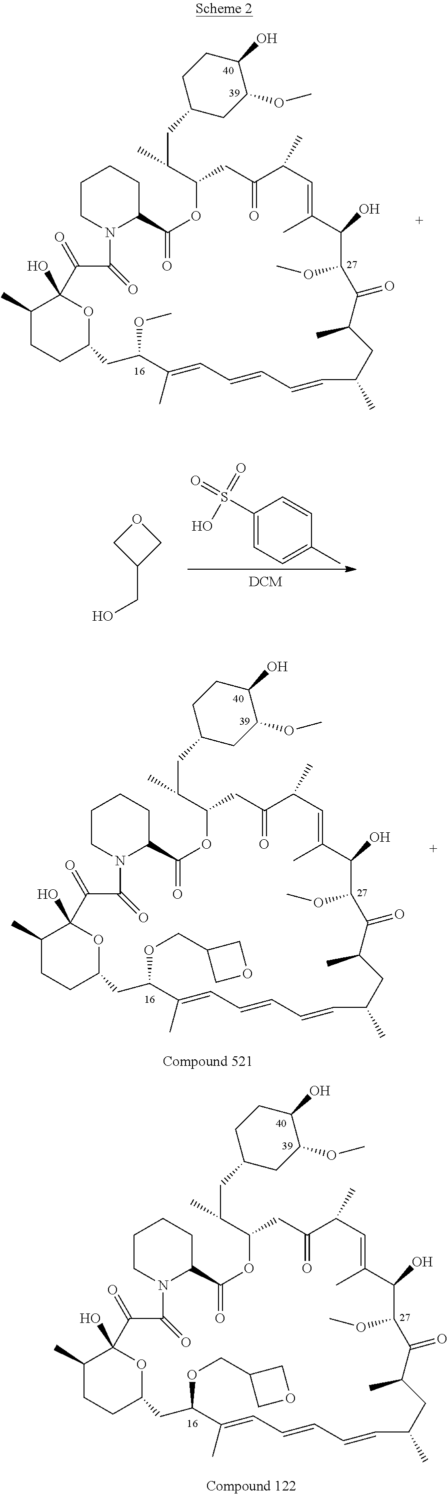

Oxetan-3-ylmethanol (8.03 g, 86.6 mmol) was added to a solution of rapamycin (2.00 g, 2.19 mmol) in anhydrous DCM (87 mL). The mixture was cooled to 0° C. and 4-methylbenzenesulfonic acid (1.88 g, 10.9 mmol) added. The mixture was stirred for five hours at 0° C. The mixture was diluted with DCM and neutralized by a saturated solution of NaHCO₃. The phases were separated. The organic phase was washed with water (40 mL), dried, filtered and concentrated to dryness. The resulting crude mixture was purified by reverse phase chromatography (Uptisphere Strategy C18-Hq 10 um 250×30.0 mm CH₃CN:H₂O gradient 50:50 to 100:0, 277 nm). The main fraction (900 mg) was purified by SFC separation to afford (1R,9S,12S,15R,16E,18R,19R,21R,23S,24E,26E,28E,30S,32S,35R)-1,18-dihydroxy-12-[(2R)-1-[(1S,3R,4R)-4-hydroxy-3-methoxycyclohexyl]propan-2-yl]-19-methoxy-15,17,21,23,29,35-hexamethyl-30-[(oxetan-3-yl)methoxy]-11,36-dioxa-4-azatricyclo[30.3.1.04,9]hexatriaconta-16,24,26,28-tetraene-2,3,10,14,20-pentone (102.9 mg, 5%, white amorphous solid, compound 521) and (1R,9S,12S,15R,16E,18R,19R,21R,23S,24E,26E,28E,30R,32S,35R)-1,18-dihydroxy-12-[(2R)-1-[(1S,3R,4R)-4-hydroxy-3-methoxycyclohexyl]propan-2-yl]-19-methoxy-15,17,21,23,29,35-hexamethyl-30-[(oxetan-3-yl)methoxy]-11,36-dioxa-4-azatricyclo[30.3.1.04,9]hexatriaconta-16,24,26,28-tetraene-2,3,10,14,20-pentone (22.9 mg, 1%, white amorphous solid, compound 122).

SFC separation method: Column: Princeton 2 Ethylpyridine 5 μm 60. Column size: 3 cm I.D.×15 cm L. Mobile phase: CO2/IpOH 78/22. Flowrate: 100 ml/min. Pressure: 100 Bar. Wave length: UV 277 nm. SFC Equipment: Waters SFC200.

Compound 521: ¹H NMR (DMSO-d₆, 600 MHz): δ (ppm) 6.32-6.50 (m, 2H), 6.05-6.28 (m, 3H), 5.47 (dd, J=14.9, 9.6 Hz, 1H), 5.24 (br d, J=2.3 Hz, 1H), 5.09 (br d, J=10.1 Hz, 1H), 4.96-5.02 (m, 1H), 4.91-4.95 (m, 1H), 4.47-4.66 (m, 3H), 4.18-4.33 (m, 2H), 3.97-4.08 (m, 2H), 3.92 (d, J=4.7 Hz, 1H), 3.78 (dd, J=11.7, 2.1 Hz, 1H), 3.01-3.48 (m, 13H), 2.83 (ddd, J=11.1, 8.7, 4.5 Hz, 1H), 2.74 (dd, J=17.7, 2.6 Hz, 1H), 2.35-2.46 (m, 2H), 2.17-2.26 (m, 1H), 1.46-2.13 (m, 19H), 0.55-1.44 (m, 27H). LCMS: MNa+ (ion type), 992.4 (ion m/z).

Compound 124: ¹H NMR (DMSO-d₆, 600 MHz): δ ppm 6.31-6.71 (m, 2H), 5.85-6.28 (m, 3H), 4.75-5.70 (m, 5H), 4.45-4.69 (m, 3H), 4.15-4.40 (m, 2H), 3.67-4.12 (m, 4H), 3.50-3.64 (m, 1H), 3.36-3.49 (m, 2H), 3.00-3.24 (m, 6H), 2.52-2.90 (m, 4H), 1.82-2.47 (m, 6H), 0.67-1.80 (m, 44H), 0.51-0.63 (m, 1H). LCMS: MNa+ (ion type), 992.4 (ion m/z).

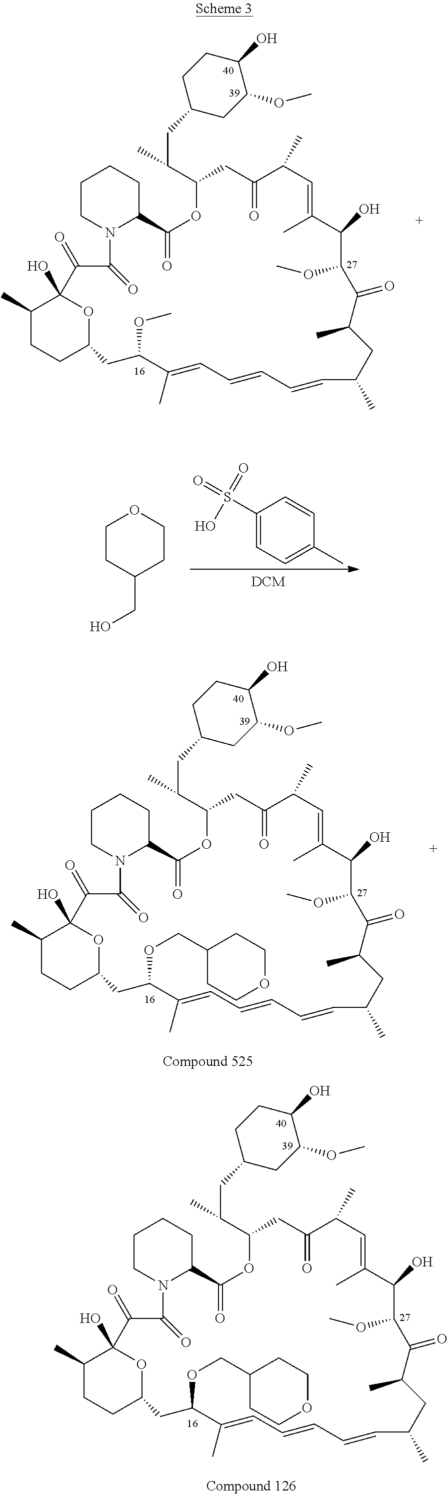

(Oxan-4-yl)methanol (10.06 g, 86.6 mmol) was added to a solution of rapamycin (2.00 g, 2.19 mmol) in anhydrous DCM (87 mL). The mixture was cooled to −40° C. and 4-methylbenzenesulfonic acid (1.88 g, 10.9 mmol) added. The mixture was allowed to reach −10° C. and stirred for one hour at −10° C. The mixture was diluted with DCM and neutralized by a saturated solution of NaHCO3. The phases were separated. The organic phase was washed with water (40 mL), dried, filtered and concentrated to dryness. The resulting crude mixture was purified by reverse phase chromatography (Uptisphere Strategy C18-Hq 10 um 250×30.0 mm CH₃CN:H₂O gradient 60:40 to 100:0 in 40 min, 277 nm). The main fraction (450 mg) was purified by SFC separation to afford (1R,9S,12S,15R,16E,18R,19R,21R,23S,24E,26E,28E,30S,32S,35R)-1,18-dihydroxy-12-[(2R)-1-[(1S,3R,4R)-4-hydroxy-3-methoxycyclohexyl]propan-2-yl]-19-methoxy-15,17,21,23,29,35-hexamethyl-30-[(oxan-4-yl)methoxy]-11,36-dioxa-4-azatricyclo[30.3.1.04,9]hexatriaconta-16,24,26,28-tetraene-2,3,10,14,20-pentone (109 mg, 5%, amorphous white solid, compound 525) and (1R,9S,12S,15R,16E,18R,19R,21R,23S,24E,26E,28E,30R,32S,35R)-1,18-dihydroxy-12-[(2R)-1-[(1S,3R,4R)-4-hydroxy-3-methoxycyclohexyl]propan-2-yl]-19-methoxy-15,17,21,23,29,35-hexamethyl-30-[(oxan-4-yl)methoxy]-11,36-dioxa-4-azatricyclo[30.3.1.04,9]hexatriaconta-16,24,26,28-tetraene-2,3,10,14,20-pentone (6 mg, 0.5%, amorphous white solid, compound 126).

SFC separation: Column: Princeton 2 Ethylpyridine 5 μm 60 A, Column size: 3 cm I.D.×15 cm L. Mobile phase: CO₂/IpOH 83/17. Flowrate: 100 ml/mi. Pressure: 100 Bar. Wave length: UV 277 nm. SFC Equipment: Waters SFC200

Compound 525: ¹H NMR (DMSO-d₆, 600 MHz): δ ppm 6.27-6.52 (m, 2H), 6.04-6.25 (m, 3H), 5.46 (dd, J=14.9, 9.8 Hz, 1H), 5.23 (s, 1H), 5.13-5.39 (m, 1H), 5.09 (br d, J=10.1 Hz, 1H), 4.96-5.00 (m, 1H), 4.91-4.95 (m, 1H), 4.34-4.64 (m, 1H), 3.99-4.08 (m, 2H), 3.94 (d, J=4.5 Hz, 1H), 3.79-3.86 (m, 2H), 3.65-3.75 (m, 1H), 3.23-3.37 (m, 6H), 3.12-3.21 (m, 6H), 3.07 (dd, J=9.1, 6.3 Hz, 1H), 2.94 (dd, J=9.1, 6.2 Hz, 1H), 2.83 (ddd, J=11.1, 8.6, 4.3 Hz, 1H), 2.73 (dd, J=17.7, 2.6 Hz, 1H), 2.34-2.45 (m, 2H), 2.17-2.26 (m, 1H), 1.93-2.14 (m, 3H), 1.11-1.92 (m, 28H), 0.89-1.08 (m, 6H), 0.87 (d, J=6.6 Hz, 3H), 0.80-0.85 (m, 3H), 0.78 (d, J=6.7 Hz, 3H), 0.70-0.76 (m, 3H), 0.60 (m, 1H). LCMS: MNa+ (ion type), 1020.5 (ion m/z).

Compound 126: LCMS: MNa+ (ion type), 1020.5 (ion m/z).

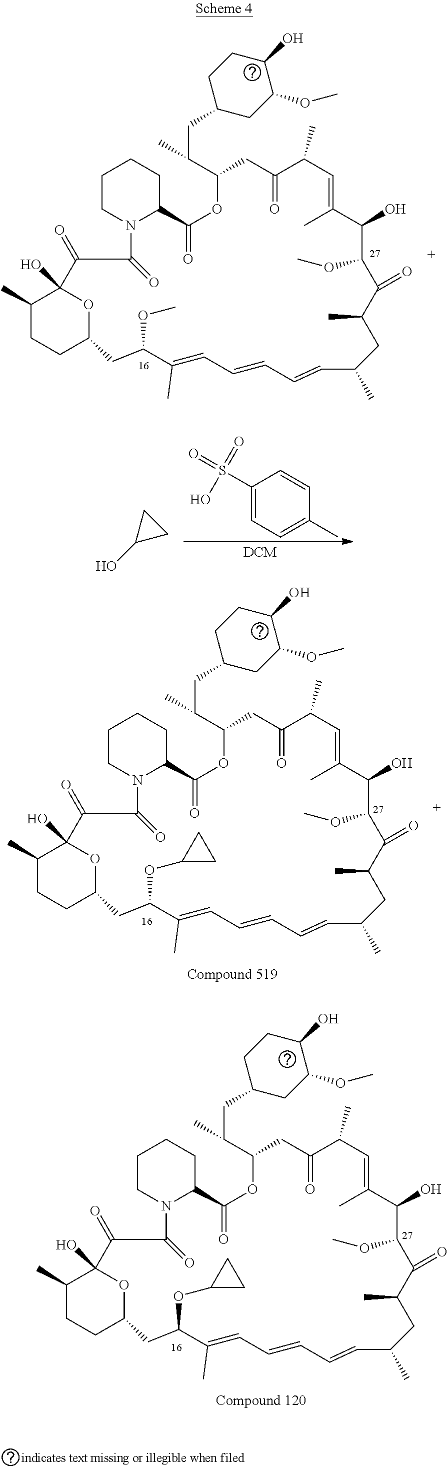

Cyclopropanol (5.47 mL, 86.64 mmol) was added to a solution of rapamycin (2.00 g, 2.19 mmol) in anhydrous DCM (87 mL). The mixture was cooled to −20° C. and 4-methylbenzenesulfonic acid (1.88 g, 10.9 mmol) added. The mixture stirred for 2 hours at −20° C. The mixture was allowed to reach room temperature over an hour. The mixture was diluted with DCM and neutralized by a saturated solution of NaHCO3. The phases were separated. The organic phase was washed with water (40 mL), dried, filtered and concentrated to dryness. The resulting crude mixture was purified by reverse phase chromatography (Uptisphere Strategy C18-Hq 10 um 250×30.0 mm CH3CN:H2O gradient 70:30 to 100:0 in 33 min, 277 nm). The main fraction (940 mg) was purified by SFC separation to afford (1R,9S,12S,15R,16E,18R,19R,21R,23S,24E,26E,28E,30R,32S,35R)-30-(cyclopropoxy)-1,18-dihydroxy-12-[(1R)-2-[(1S,3R,4R)-4-hydroxy-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (132 mg, 6.5%, amorphous white solid, compound 519) and (1R,9S,12S,15R,16E,18R,19R,21R,23S,24E,26E,28E,30S,32S,35R)-30-(cyclopropoxy)-1,18-dihydroxy-12-[(1R)-2-[(1S,3R,4R)-4-hydroxy-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (14 mg, 0.6%, amorphous white solid, compound 120).

SFC separation: Column: Princeton 2 Ethylpyridine 5 μm 60 A. Column size: 3 cm I.D.×15 cm L. Mobile phase: CO2/IpOH 80/20. Flowrate: 70 ml/min. Pressure: 100 Bar. Wave length: UV 277 nm. SFC Equipment: Waters SFC200

Compound 519: ¹H NMR (DMSO-d₆, 600 MHz): δ (ppm) 6.35-6.49 (m, 2H), 6.19-6.29 (m, 1H), 6.09-6.18 (m, 2H), 5.46 (dd, J=15.0, 9.5 Hz, 1H), 5.17-5.32 (m, 1H), 5.09 (br d, J=10.1 Hz, 1H), 4.95-5.02 (m, 1H), 4.93 (br d, J=5.3 Hz, 1H), 4.56 (br s, 1H), 3.98-4.06 (m, 1H), 3.92 (d, J=4.7 Hz, 1H), 3.84-3.90 (m, 1H), 3.82 (dd, J=11.8, 2.0 Hz, 1H), 3.39-3.45 (m, 1H), 3.30 (s, 3H), 3.25-3.29 (m, 2H), 3.16-3.22 (m, 1H), 3.16 (s, 3H), 3.04 (tt, J=6.0, 3.1 Hz, 1H), 2.79-2.91 (m, 1H), 2.73 (dd, J=17.6, 2.5 Hz, 1H), 2.34-2.46 (m, 2H), 1.78-2.34 (m, 6H), 1.63-1.77 (m, 10H), 1.01-1.62 (m, 13H), 0.98 (d, J=6.6 Hz, 2H), 0.92-1.01 (m, 2H), 0.87 (d, J=6.5 Hz, 3H), 0.83 (d, J=6.5 Hz, 3H), 0.81-0.86 (m, 1H), 0.75-0.81 (m, 3H), 0.69-0.75 (m, 3H), 0.60 (q, J=11.9 Hz, 1H), 0.27-0.52 (m, 4H). LCMS: MNa+ (ion type), 962.3 (ion m/z).

Compound 120: ¹H NMR (DMSO-d₆, 600 MHz): δ (ppm) 5.91-6.72 (m, 5H), 5.61-5.73 (m, 1H), 4.86-5.45 (m, 4H), 4.35-4.75 (m, 1H), 4.05 (br d, J=1.6 Hz, 1H), 3.69-3.99 (m, 3H), 3.51-3.63 (m, 1H), 2.98-3.42 (m, 10H), 2.63-2.93 (m, 2H), 2.51-2.59 (m, 2H), 2.21-2.34 (m, 1H), 1.85-2.19 (m, 3H), −0.05-1.83 (m, 47H). LCMS: MNa+ (ion type), 962.3 (ion m/z).

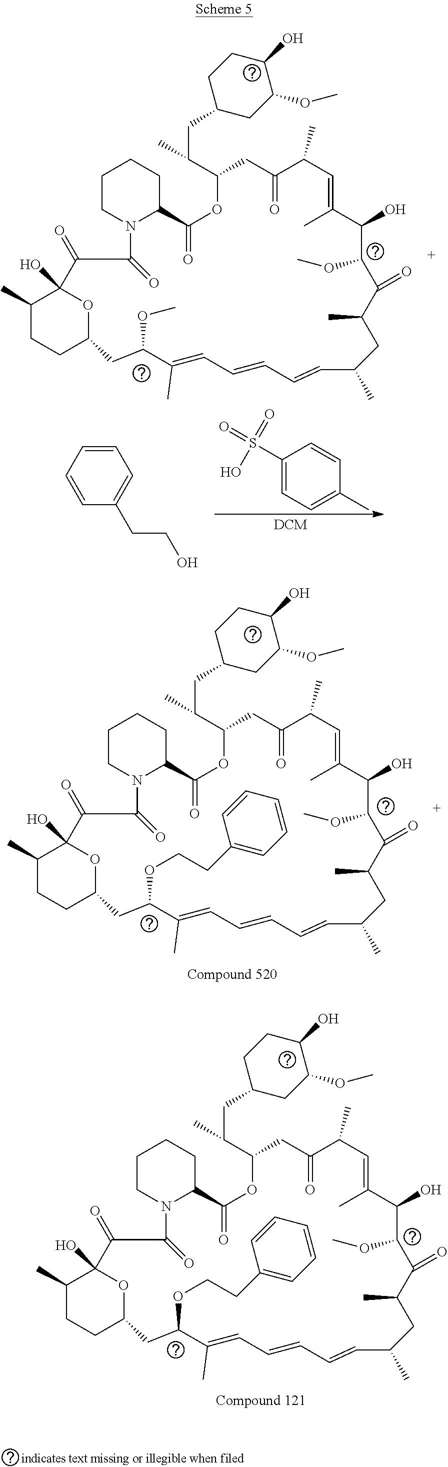

2-phenylethanol (10 mL, 86.6 mmol) was added to a solution of rapamycin (2.00 g, 2.19 mmol) in anhydrous DCM (87 mL). The mixture was cooled to −20° C. and 4-methylbenzenesulfonic acid (1.88 g, 10.9 mmol) added. The mixture stirred for 1 hour at 0° C. The mixture was allowed to reach room temperature over an hour. The mixture was diluted with DCM and neutralized by a saturated solution of NaHCO₃. The phases were separated. The organic phase was washed with water (40 mL), dried, filtered and concentrated to dryness. The resulting crude mixture was purified by reverse phase chromatography (Uptisphere Strategy C18-Hq 10 um 250×30.0 mm CH₃CN:H₂O gradient 60:40 to 100:0 in 25 min, 277 nm). The main fraction (1.76 g) was purified by SFC separation to afford (1R,9S,12S,15R,16E,18R,19R,21R,23S,24E,26E,28E,30S,32S,35R)-1,18-dihydroxy-12-[(1R)-2-[(1S,3R,4R)-4-hydroxy-3-methoxy-cyclohexyl]-1-methyl-ethyl]-19-methoxy-15,17,21,23,29,35-hexamethyl-30-(2-phenylethoxy)-11,36-dioxa-4-azatricyclo[30.3.1.04,9]hexatriaconta-16,24,26,28-tetraene-2,3,10,14,20-pentone (95.2 mg, 4.2%, amorphous white solid, compound 520) and ((1R,9S,12S,15R,16E,18R,19R,21R,23S,24E,26E,28E,30R,32S,35R)-1,18-dihydroxy-12-[(1R)-2-[(I S,3R,4R)-4-hydroxy-3-methoxy-cyclohexyl]-1-methyl-ethyl]-19-methoxy-15,17,21,23,29,35-hexamethyl-30-(2-phenylethoxy)-11,36-dioxa-4-azatricyclo[30.3.1.04,9]hexatriaconta-16,24,26,28-tetraene-2,3,10,14,20-pentone (42 mg, 1.9%, amorphous white solid, compound 121).

SFC separation: Column: Princeton 2 Ethylpyridine 5 μm 60 A. Column size: 3 cm I.D.×15 cm L. Mobile phase: CO2/IpOH 80/20. Flowrate: 100 ml/min. Pressure: 100 Bar. Wave length: UV 277 nm. SFC Equipment: Waters SFC200.

Compound 520: ¹H NMR (DMSO-d₆, 600 MHz): δ (ppm) 7.13-7.31 (m, 5H), 6.30-6.42 (m, 21H), 6.05-6.24 (m, 3H), 5.45 (dd, J=14.8, 9.7 Hz, 1H), 5.17-5.28 (m, 1H), 5.08 (br d, J=10.1 Hz, 1H), 4.94-5.05 (m, 1H), 4.93 (br d, J=5.4 Hz, 1H), 4.57 (d, J=4.5 Hz, 1H), 3.89-4.06 (m, 3H), 3.68-3.75 (m, 1H), 3.40-3.48 (m, 2H), 3.31-3.33 (m, 4H), 3.25 (br dd, J=10.1, 6.6 Hz, 1H), 3.16-3.21 (m, 2H), 3.15 (s, 3H), 2.71-2.85 (m, 4H), 2.33-2.44 (m, 2H), 2.16-2.33 (m, 1H), 2.05-2.12 (m, 1H), 1.96-2.05 (m, 1H), 1.80-1.91 (m, 2H), 1.74-1.78 (m, 1H), 1.73 (s, 3H), 1.59-1.70 (m, 5H), 1.57-1.59 (m, 3H), 0.91-1.56 (m, 16H), 0.85 (d, J=6.6 Hz, 3H), 0.82 (d, J=6.5 Hz, 41), 0.76 (d, J=6.7 Hz, 3H), 0.72 (d, J=6.7 Hz, 3H), 0.55-0.62 (m, 1H). LCMS: MNa+ (ion type), 1026.5 (ion m/z).

Compound 121: 1H NMR (600 MHz, DMSO-d6) Shift 7.16-7.28 (m, 5H), 6.55 (s, 1H), 5.88-6.50 (m, 4H), 5.53-5.71 (m, 1H), 5.21-5.26 (m, 1H), 5.08-5.13 (m, 1H), 4.96-5.00 (m, 1H), 4.52-4.58 (m, 1H), 3.77-4.11 (m, 4H), 3.54 (br d, J=13.06 Hz, 1H), 3.35-3.48 (m, 2H), 3.20-3.29 (m, 4H), 3.10-3.20 (m, 4H), 2.93-3.08 (m, 1H), 2.70-2.84 (m, 4H), 2.51-2.55 (m, 1H), 2.31-2.47 (m, 1H), 2.20-2.30 (m, 1H), 2.15 (br d, J=13.79 Hz, 1H), 1.87-2.05 (m, 2H), 1.07-1.83 (m, 26H), 0.53-0.99 (m, 18H). LCMS: MNa+ (ion type), 1026.4 (ion m/z).

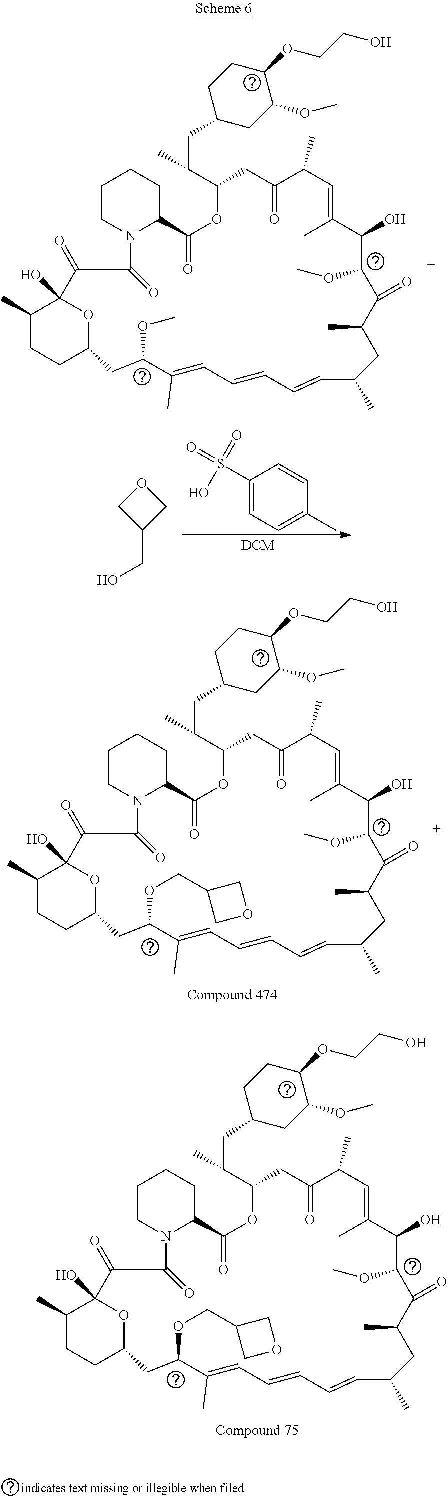

Oxetan-3-ylmethanol (3.83 g, 41 mmol) was added to a solution of (1R,9S,12S,15R,16E,18R,19R,21R,23S,24E,26E,28E,30S,32S,35R)-1,18-dihydroxy-12-{(2R)-1-[(1S,3R,4R)-4-(2-hydroxyethoxy)-3-methoxycyclohexyl]propan-2-yl}-19,30-dimethoxy-15,17,21,23,29,35-hexamethyl-11,36-dioxa-4-azatricyclo[30.3.1.0˜4,9˜]hexatriaconta-16,24,26,28-tetraene-2,3,10,14,20-pentone (1 g, 1.04 mmol) in anhydrous DCM (41.7 mL). The mixture was cooled down to 0° C. and 4-methylbenzenesulfonic acid (0.88 g, 5.11 mmol) was added. The mixture was stirred for 6 hours at 0° C., diluted with DCM and neutralized by a saturated solution of NaHCO₃. The phases were separated. The organic phase was washed with water (60 mL), dried, filtered and concentrated to dryness. The resulting crude mixture was purified by reverse phase chromatography (Uptisphere Strategy C18-Hq 10 um 250×30.0 mm CH₃CN:H₂O gradient 60:40 to 100:0 in 25 min, 277 nm). The main fraction (395 mg) was purified by SFC separation to afford (1R,9S,12S,15R,16E,18R,19R,21R,23S,24E,26E,28E,30S,32S,35R)-1,18-dihydroxy-12-[(1R)-2-[(1S,3R,4R)-4-(2-hydroxyethoxy)-3-methoxy-cyclohexyl]-1-methyl-ethyl]-19-methoxy-15,17,21,23,29,35-hexamethyl-30-(oxetan-3-ylmethoxy)-11,36-dioxa-4-azatricyclo[30.3.1.04,9]hexatriaconta-16,24,26,28-tetraene-2,3,10,14,20-pentone (75.5 mg, 7.1%, amorphous white solid, compound 474) and (1R,9S,12S,15R,16E,18R,19R,21R,23S,24E,26E,28E,30R,32S,35R)-1,18-dihydroxy-12-[(1R)-2-[(1S,3R,4R)-4-(2-hydroxyethoxy)-3-methoxy-cyclohexyl]-1-methyl-ethyl]-19-methoxy-15,17,21,23,29,35-hexamethyl-30-(oxetan-3-ylmethoxy)-11,36-dioxa-4-azatricyclo[30.3.1.04,9]hexatriaconta-16,24,26,28-tetraene-2,3,10,14,20-pentone (48 mg, 3.7%, amorphous white solid, compound 75).

SFC separation: Column: Princeton 2 Ethylpyridine 5 μm 60 A. Column size: 3 cm I.D.×15 cm L. Mobile phase: CO2/IpOH 80/20. Flowrate: 50 ml/min. Pressure: 100 Bar. Wave length: UV 277 nm. SFC Equipment: Waters SFC200.

Compound 474: ¹H NMR (DMSO-d₆, 600 MHz): δ (ppm) 6.45 (s, 1H), 6.40 (dd, J=14.7, 11.2 Hz, 1H), 6.18-6.26 (m, 1H), 6.10-6.17 (m, 2H), 5.47 (dd, J=15.0, 9.5 Hz, 1H), 5.25 (d, J=4.5 Hz, 1H), 5.09 (br d, J=10.1 Hz, 1H), 4.95-5.00 (m, 1H), 4.93 (br d, J=5.7 Hz, 1H), 4.61 (dd, J=7.8, 5.9 Hz, 2H), 4.44 (t, J=5.4 Hz, 1H), 4.25 (td, J=6.0, 2.0 Hz, 2H), 3.96-4.06 (m, 2H), 3.92 (d, J=4.7 Hz, 1H), 3.78 (br d, J=13.5 Hz, 1H), 3.39-3.55 (m, 6H), 3.32-3.34 (m, 1H), 3.33 (s, 3H), 3.24-3.29 (m, 1H), 3.16-3.23 (m, 1H), 3.15 (s, 3H), 3.02-3.12 (m, 2H), 2.97 (ddd, J=11.1, 8.8, 4.5 Hz, 1H), 2.73 (br d, J=15.1 Hz, 1H), 2.30-2.48 (m, 2H), 2.19-2.34 (m, 1H), 2.09 (br d, J=13.5 Hz, 1H), 1.79-2.06 (m, 5H), 1.74 (s, 3H), 1.62-1.70 (m, 5H), 0.00 (d, J=6.9 Hz, 3H), 0.90-1.60 (m, 15H), 0.87 (d, J=6.6 Hz, 3H), 0.83 (d, J=6.5 Hz, 3H), 0.79-0.82 (m, 1H), 0.78 (d, J=6.7 Hz, 3H), 0.73 (d, J=6.7 Hz, 3H), 0.65 (q, J=11.8 Hz, 1H). LCMS: MNa+ (ion type), 1036.5 (ion m/z).

Compound 75: ¹H NMR (DMSO-d₆, 600 MHz): δ (ppm) 6.58 (s, 1H), 6.38-6.50 (m, 1H), 6.11-6.25 (m, 3H), 6.06 (br d, J=11.0 Hz, 1H), 5.64 (dd, J=14.5, 8.4 Hz, 1H), 5.25 (br d, J=4.5 Hz, 2H), 5.09-5.14 (m, 1H), 4.99 (br d, J=6.0 Hz, 1H), 4.60 (dt, J=7.8, 6.3 Hz, 2H), 4.41-4.45 (m, 1H), 4.32 (t, J=5.8 Hz, 1H), 4.26 (t, J=5.9 Hz, 1H), 4.05 (t, J=4.4 Hz, 1H), 3.91-4.02 (m, 1H), 3.88 (d, J=4.8 Hz, 1H), 3.85 (dd, J=10.1, 1.9 Hz, 1H), 3.57 (br d, J=13.8 Hz, 1H), 3.38-3.54 (m, 6H), 3.32-3.34 (m, 1H), 3.31 (s, 3H), 3.18 (s, 3H), 3.05-3.13 (m, 2H), 2.99-3.05 (m, 1H), 2.92-2.99 (m, 1H), 2.78 (br dd, J=17.5, 2.6 Hz, 1H), 2.52-2.73 (m, 2H), 2.23-2.38 (m, 1H), 2.06-2.19 (m, 1H), 1.86-2.05 (m, 31H), 1.71-1.76 (m, 1H), 1.69 (s, 3H), 1.66 (s, 3H), 1.21-1.65 (m, 12H), 1.03-1.14 (m, 3H), 0.99 (d, J=6.6 Hz, 3H), 0.98-1.02 (m, 1H), 0.94 (br d, J=6.7 Hz, 3H), 0.86-0.92 (m, 2H), 0.85 (d, J=6.6 Hz, 3H), 0.80 (d, J=6.7 Hz, 3H), 0.74 (d, J=6.6 Hz, 3H), 0.62 (q, J=11.8 Hz, 1H). LCMS: MNa+ (ion type), 1036.5 (ion m/z).

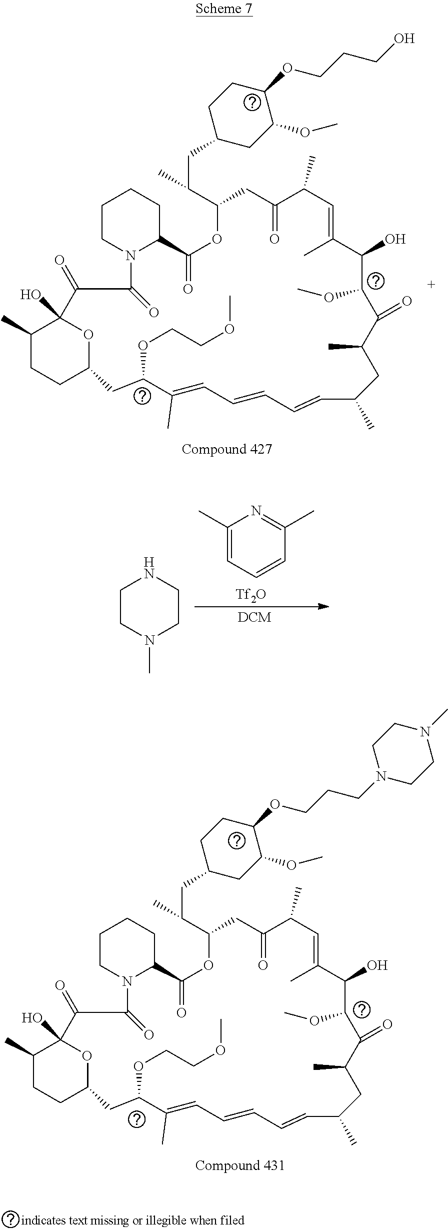

A solution of (1R,9S,12S,15R,16E,18R,19R,21R,23S,24E,26E,28E,30S,32S,35R)-1,18-dihydroxy-12-[(1R)-2-[(1S,3R,4R)-4-(3-hydroxypropoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (compound 427, 350 mg, 0.344 mmol) in anhydrous DCM (2.87 mL) was cooled to −78° C. under argon. Then 2,6-dimethylpyridine (171 uL, 1.48 mmol) was added. The solution was stirred for few minutes and trifluoromethylsulfonyl trifluoromethanesulfonate (116 uL, 0.689 mmol) was added. The mixture was stirred 10 min at −78° C. The bath was removed and 1-methylpiperazine (191 uL, 1.72 mmol) was added. The reaction mixture was stirred while allowed to reach room temperature over 20 minutes. The mixture was diluted with DCM, concentrated and purified by silica gel flash column chromatography (0 to 10% of (MeOH:Triethylamine 1:1) in ethyl acetate. The isolated fractions of interest were purified a second time by silica gel flash column chromatography (0 to 20% of MeOH in DCM to afford the compound of interest (1R,9S,12S,15R,16E,18R,19R,21R,23S,24E,26E,28E,30S*,32S,35R)-1,18-dihydroxy-19-methoxy-30-(2-methoxyethoxy)-12-[(1R)-2-[(1S,3R,4R)-3-methoxy-4-[3-(4-methylpiperazin-1-yl)propoxy]cyclohexyl]-1-methyl-ethyl]-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (368 mg, 77%, compound 431).

Compound 431: ¹H NMR (DMSO-d₆, 600 MHz): δ (ppm) 5.87-6.57 (m, 5H), 5.46 (dd, J=14.9, 9.6 Hz, 1H), 5.25 (d, J=4.5 Hz, 1H), 5.09 (br d, J=10.1 Hz, 1H), 4.98 (dt, J=7.8, 4.0 Hz, 1H), 4.93 (br d, J=5.6 Hz, 1H), 4.01 (br t, J=4.1 Hz, 2H), 3.94 (d, J=4.5 Hz, 1H), 3.78 (dd, J=11.8, 1.7 Hz, 1H), 3.07-3.62 (m, 19H), 2.87-3.04 (m, 2H), 2.73 (br dd, J=17.7, 2.6 Hz, 1H), 2.13-2.66 (m, 11H), 2.06-2.11 (m, 1H), 1.81-2.05 (m, 5H), 1.47-1.78 (m, 17H), 1.34-1.44 (m, 2H), 0.98 (br d, J=6.5 Hz, 13H), 0.80-0.88 (m, 7H), 0.77 (d, J=6.7 Hz, 3H), 0.73 (d, J=6.6 Hz, 3H), 0.65 (q, J=11.8 Hz, 1H). LCMS: MH+ (ion type), 1098.5 (ion m/z).

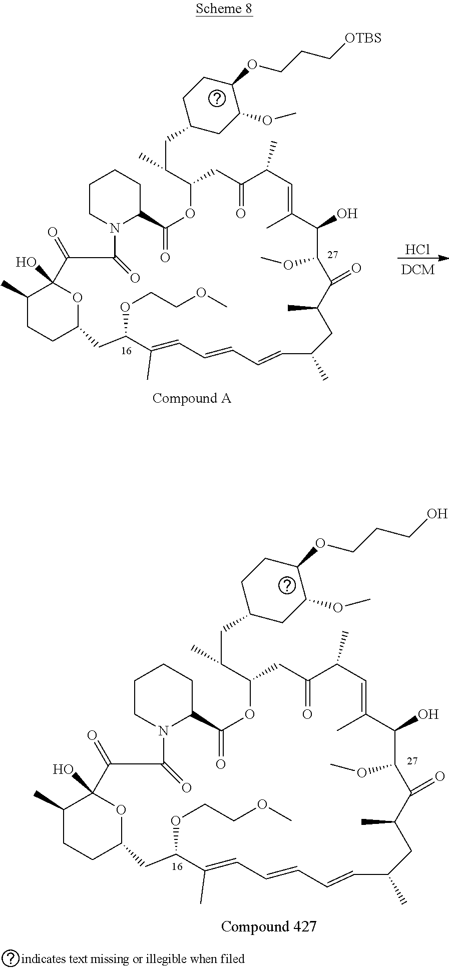

(1R,9S,12S,15R,16E,18R,19R,21R,23S,24E,26E,28E,30S,32S,35R)-12-[(1R)-2-[(1S,3R,4R)-4-[3-[tert-butyl(dimethyl)silyl]oxypropoxy]-3-methoxy-cyclohexyl]-1-methyl-ethyl]-1,18-dihydroxy-19-methoxy-30-(2-methoxyethoxy)-15,17,21,23,29,35-hexamethyl-11,36-dioxa-4-azatricyclo[30.3.1.04,9]hexatriaconta-16,24,26,28-tetraene-2,3,10,14,20-pentone (compound A, 600 mg, 0.531 mmol) was dissolved in anhydrous THF (6 mL) and stirred at room temperature. A 1 M aqueous solution of chlorohydric acid (53 uL, 0.0531 mmol) was added. After two hours at room temperature, water and DCM were added, the layers were separated and the aqueous was extracted twice with DCM. The combined organic layer was concentrated and purified by silica gel flash column chromatography (AcOEt/Cyclohexane 0/100 to 100/0) to afford (1R,9S,12S,15R,16E,18R,19R,21R,23S,24E,26E,28E,30S,32S,35R)-1,18-dihydroxy-12-[(1R)-2-[(1S,3R,4R)-4-(3-hydroxypropoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.04,9]hexatriaconta-16,24,26,28-tetraene-2,3,10,14,20-pentone (250 mg, 57%, compound 427, white amorphous solid)

Compound 427: 1H NMR (600 MHz, CHLOROFORM-d, 300K) δ ppm 5.79-6.44 (m, 4H), 5.02-5.60 (m, 4H), 4.78 (br s, 1H), 4.17 (d, J=5.9 Hz, 1H), 2.95-3.97 (m, 26H), 2.54-2.91 (m, 4H), 2.05-2.39 (m, 5H), 0.78-2.01 (m, 43H), 0.62-0.76 (m, 1H). LCMS: MNa+ (ion type), 1038.5 (ion m/z).

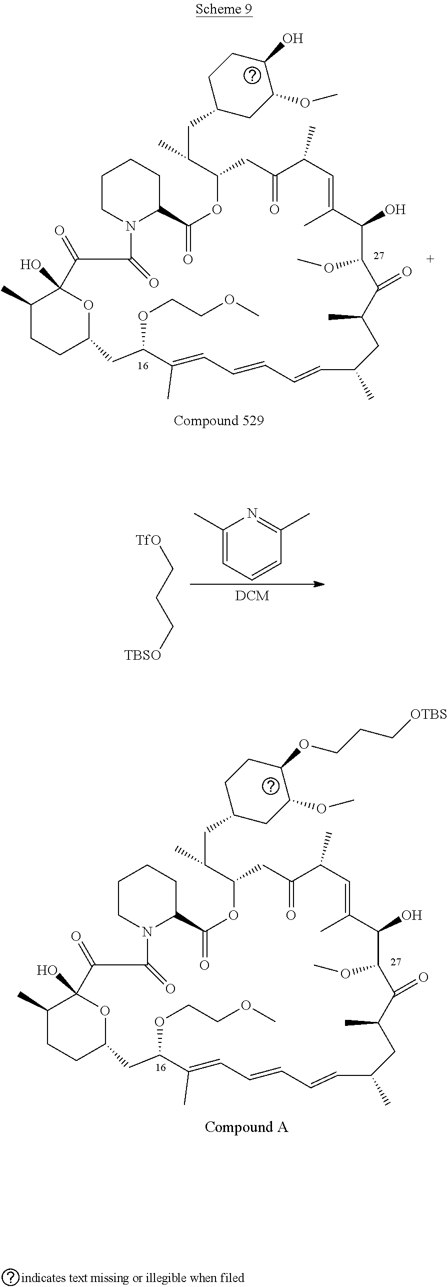

(1R,9S,12S,15R,16E,18R,19R,21R,23S,24E,26E,28E,30S,32S,35R)-1,18-dihydroxy-12-[(1R)-2-[(1S,3R,4R)-4-hydroxy-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.04,9]hexatriaconta-16,24,26,28-tetraene-2,3,10,14,20-pentone (compound 529, 1.00 g, 1.04 mmol) was dissolved in chlorobenzene (10 mL) with N-ethyl-N-isopropyl-propan-2-amine (0.58 mL, 3.34 mmol) and 3-[tert-butyl(dimethyl)silyl]oxypropyl trifluoromethanesulfonate (1009 mg, 3.13 mmol) under argon. The reaction mixture was heated at 50° C. for two hours. Extra N-ethyl-N-isopropyl-propan-2-amine (0.58 mL, 3.34 mmol) and 3-[tert-butyl(dimethyl)silyl]oxypropyl trifluoromethanesulfonate (1009 mg, 3.13 mmol) were charged after two hours and four hours of reaction. After 6 hours at 50° C., the mixture was allowed to reach room temperature. It was then diluted with DCM and water. The layers were separated and the organic was washed with a saturated aqueous solution of NaCl. The gathered organic layers were concentrated and purified by silica gel flash column chromatography (AcOEt/Cyclohexane 0/100 to 30/70) to afford (1R,9S,12S,15R,16E,18R,19R,21R,23S,24E,26E,28E,30S,32S,35R)-12-[(1R)-2-[(1S,3R,4R)-4-[3-[tert-butyl(dimethyl)silyl]oxypropoxy]-3-methoxy-cyclohexyl]-1-methyl-ethyl]-1,18-dihydroxy-19-methoxy-30-(2-methoxyethoxy)-15,17,21,23,29,35-hexamethyl-11,36-dioxa-4-azatricyclo[30.3.1.04,9]hexatriaconta-16,24,26,28-tetraene-2,3,10,14,20-pentone (545 mg, 44% compound A, white amorphous solid).

Compound A: ¹H NMR (DMSO-d₆, 600 MHz): δ (ppm) 5.86-6.55 (m, 5H), 5.46 (dd, J=15.0, 9.7 Hz, 1H), 5.25 (d, J=4.5 Hz, 1H), 5.09 (br d, J=10.1 Hz, 1H), 4.90-5.00 (m, 2H), 3.70-4.36 (m, 4H), 3.60-3.68 (m, 2H), 3.28-3.59 (m, 11H), 3.03-3.28 (m, 9H), 2.89-3.03 (m, 2H), 2.73 (br dd, J=17.5, 2.4 Hz, 1H), 1.79-2.46 (m, 9H), 0.48-1.78 (m, 49H), −0.11-0.16 (m, 6H). LCMS: MNa+ (ion type), 1152.6 (ion m/z).

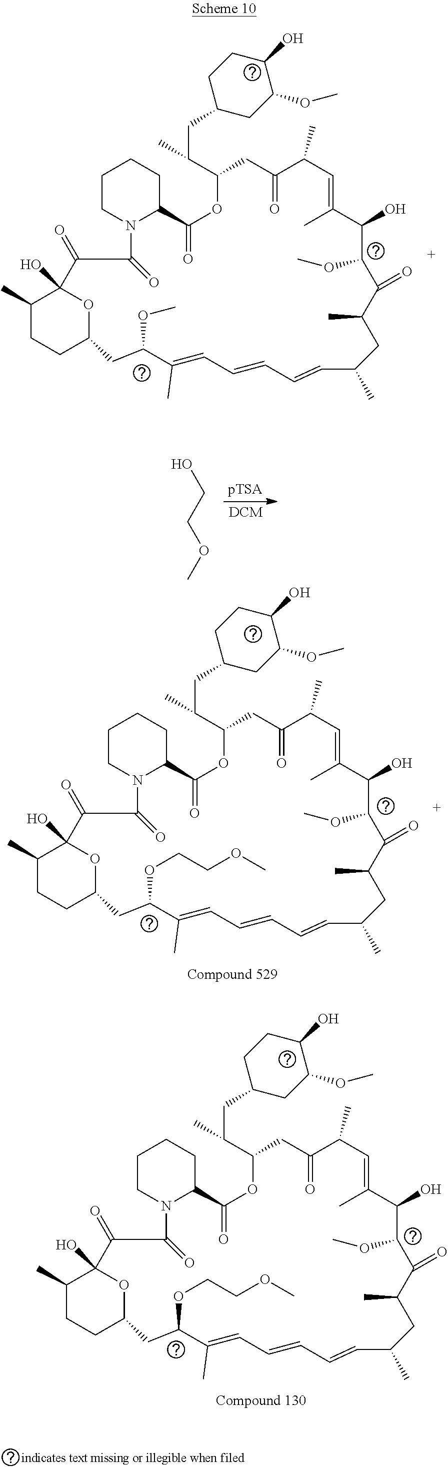

Rapamycin (2.0 g, 2.19 mmol), pTSA monohydrate (1.88 g, 10 mmol), DCM (27 mL) and 2-methoxyethanol (63 mL) were charged in a 100 mL flask and stirred for 1 h at room temperature. The mixture was diluted with EtOAc and NaHCO₃ aqueous solution. The layers were separated and the aqueous layer extracted with EtOAc. The combined organic phases were washed with water, concentrated and purified by SFC purification to afford two fractions. FC purification condition: Instrument: Waters SFC80; Stationary Phase: Princeton 2-ethylpyridine 20×150 mm 5 μm;

Mobile phase: CO2/IpOH 83/17; Flowrate: 100 mL/min; Detection: 277 nm; Pressure: 50 bar 1185 mg of sample were dissolved in 65 mL of IpOH.

Compound 529: (1R,9S,12S,15R,16E,18R,19R,21R,23S,24E,26E,28E,30S,32S,35R)-1,18-dihydroxy-12-[(1R)-2-[(1S,3R,4R)-4-hydroxy-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.04,9]hexatriaconta-16,24,26,28-tetraene-2,3,10,14,20-pentone (12.6 g, 45%, compound 529, white amorphous solid).

Compound 529: ¹H NMR (DMSO-d₆, 600 MHz): δ (ppm) 6.44 (s, 1H), 6.39 (dd, J=14.7, 11.3 Hz, 1H), 6.18-6.25 (m, 1H), 6.07-6.16 (m, 2H), 5.46 (dd, J=15.0, 9.7 Hz, 1H), 5.24 (br s, 1H), 5.09 (br d, J=10.1 Hz, 1H), 4.98 (ddd, J=8.5, 4.6, 2.9 Hz, 1H), 4.93 (br d, J=5.4 Hz, 1H), 4.43-4.71 (m, 1H), 3.98-4.09 (m, 2H), 3.94 (d, J=4.7 Hz, 1H), 3.76-3.81 (m, 1H), 3.09-3.48 (m, 17H), 2.83 (ddd, J=11.1, 8.7, 4.4 Hz, 1H), 2.73 (dd, J=17.5, 2.6 Hz, 1H), 2.34-2.45 (m, 2H), 2.16-2.27 (m, 1H), 1.80-2.14 (m, 6H), 1.46-1.78 (m, 14H), 1.35-1.45 (m, 2H), 1.11-1.33 (m, 4H), 0.91-1.09 (m, 6H), 0.67-0.89 (m, 13H), 0.55-0.64 (m, 1H). LCMS: MN-4+(ion type), 975.5 (ion m/z).

Compound 130: (1R,9S,12S,15R,16E,18R,19R,21R,23S,24E,26E,28E,30R,32S,35R)-1,18-dihydroxy-12-[(1R)-2-[(1S,3R,4R)-4-hydroxy-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.04,9]hexatriaconta-16,24,26,28-tetraene-2,3,10,14,20-pentone (350 mg, 17%, compound 130, white amorphous solid).

Compound 130: ¹H NMR (DMSO-d₆, 600 MHz) δ 5.9-6.6 (m, 5H), 5.61 (dd, 1H, J=8.4, 14.6 Hz), 5.2-5.3 (m, 2H), 5.11 (ddd, 1H, J=2.8, 4.8, 9.0 Hz), 4.99 (dd, 1H, J=1.0, 6.1 Hz), 4.57 (br dd, 1H, J=1.2, 1.9 Hz), 4.04 (br d, 1H, J=3.8 Hz), 3.9-4.0 (m, 1H), 3.90 (d, 1H, J=4.5 Hz), 3.82 (dd, 1H, J=1.9, 9.7 Hz), 3.55 (br d, 1H, J=13.6 Hz), 3.0-3.5 (m, 12H), 2.5-2.9 (m, 4H), 2.2-2.3 (m, 1H), 1.8-2.2 (m, 4H), 0.5-1.8 (m, 46H); LCMS: MNa+ (ion type), 980.6 (ion m/z).

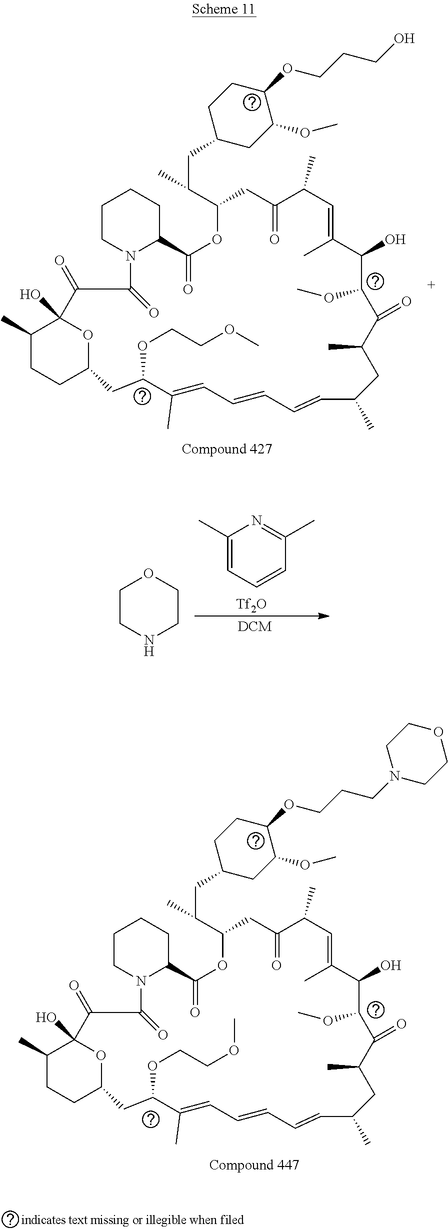

(1R,9S,12S,15R,16E,18R,19R,21R,23S,24E,26E,28E,30S,32S,35R)-1,18-dihydroxy-12-[(1R)-2-[(1S,3R,4R)-4-(3-hydroxypropoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (compound 14, 237 mg, 0.233 mmol) was dissolved in anhydrous DCM (1.94 mL). 2,6-dimethylpyridine (116 uL, 1.00 mmol) was then added and the mixture was cooled down to −78° C., before trifluoromethylsulfonyl trifluoromethanesulfonate (78 uL, 0.466 mmol) was added. The reaction was stirred at −78° C. for 1 hour. The ice bath was removed and morpholine (102 uL, 1.17 mmol) was added. The reaction mixture was stirred while allowed to reach room temperature over 20 minutes. The mixture was diluted with DCM, concentrated and purified by silica gel flash column chromatography (0 to 10% of (MeOH:Triethylamine 1:1) in ethyl acetate. The isolated fractions of interest were purified a second time by silica gel flash column chromatography (0 to 20% of MeOH in DCM to afford (1R,9S,12S,15R,16E,18R,19R,21R,23S,24E,26E,28E,30S,32S,35R)-1,18-dihydroxy-19-methoxy-30-(2-methoxyethoxy)-12-[(1R)-2-[(1S,3R,4R)-3-methoxy-4-(3-morpholinopropoxy)cyclohexyl]-1-methyl-ethyl]-15,17,21,23,29,35-hexamethyl-11,36-dioxa-4-azatricyclo[30.3.1.04,9]hexatriaconta-16,24,26,28-tetraene-2,3,10,14,20-pentone (118.2 mg, 41%, Compound 447, white amorphous solid).

Compound 447: ¹H NMR (DMSO-d₆, 600 MHz): δ (ppm) 6.45 (d, J=1.5 Hz, 1H), 6.39 (dd, J=14.7, 11.2 Hz, 1H), 6.08-6.25 (m, 3H), 5.46 (dd, J=14.8, 9.7 Hz, 1H), 5.25 (d, J=4.5 Hz, 1H), 5.09 (br d, J=10.1 Hz, 1H), 4.95-5.05 (m, 1H), 4.91-4.95 (m, 1H), 3.97-4.08 (m, 2H), 3.86-3.95 (m, 1H), 3.76-3.82 (m, 1H), 3.51-3.59 (m, 6H), 3.33-3.50 (m, 5H), 3.32 (s, 3H), 3.25-3.28 (m, 1H), 3.23 (s, 3H), 3.11-3.16 (m, 3H), 2.92-3.04 (m, 2H), 2.73 (br dd, J=17.8, 2.5 Hz, 1H), 2.34-2.45 (m, 2H), 2.27-2.34 (m, 6H), 1.83-2.23 (m, 7H), 1.71-1.77 (m, 3H), 0.89-1.70 (m, 26H), 0.85-0.88 (m, 3H), 0.83 (d, J=6.5 Hz, 3H), 0.81-0.84 (m, 1H), 0.77 (d, J=6.7 Hz, 3H), 0.73 (d, J=6.6 Hz, 3H), 0.60-0.69 (m, 1H). LCMS: MH+ (ion type), 1085.6 (ion m/z).

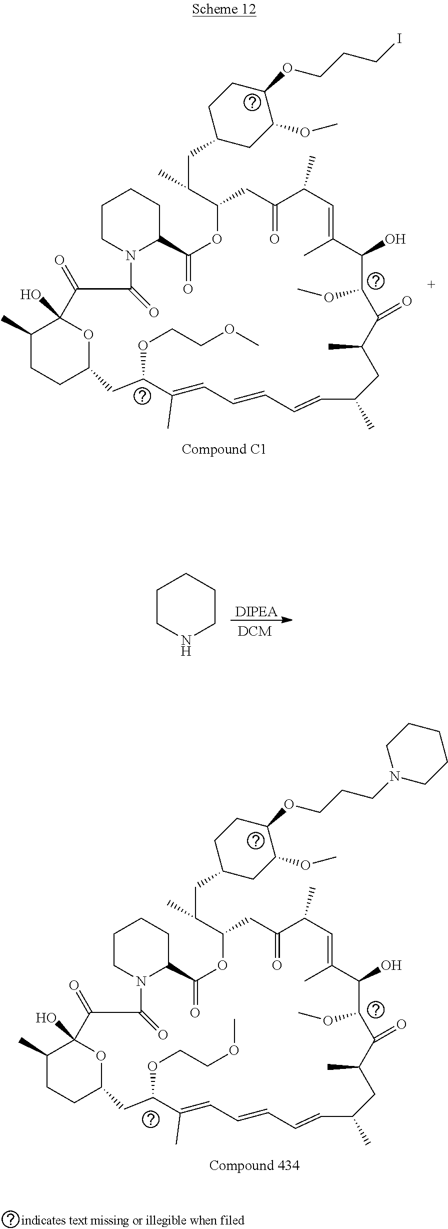

To a solution of (1R,9S,12S,15R,16E,18R,19R,21R,23S,24E,26E,28E,30S,32S,35R)-1,18-dihydroxy-12-[(1R)-2-[(1S,3R,4R)-4-(3-iodopropoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (compound C1) (326 mg, 0.289 mmol) in anhydrous DCM (1.8 mL) was added N-ethyl-N-isopropyl-propan-2-amine (152 uL, 0.868 mmol) then piperidine (34 uL, 0.347 mmol). The reaction mixture was stirred for 4.5 hours at room temperature under argon. The reaction mixture was diluted with DCM and quenched with aqueous saturated NH4Cl solution (pH=6). The organic phase was washed with water and dried. The crude was then purified by silica gel flash column chromatography (100/0 to 70/30 of EtOAc/MeOH:Et3N (50:50). The fraction of interest were then purified by silica gel flash column chromatography (100/0 to 80/20 of DCM/MeOH) to afford the desired product (1R,9S,12S,15R,16E,18R,19R,21R,23S,24E,26E,28E,30S,32S,35R)-1,18-dihydroxy-19-methoxy-30-(2-methoxyethoxy)-12-[(1R)-2-[(1S,3R,4R)-3-methoxy-4-[3-(1-piperidyl)propoxy]cyclohexyl]-1-methyl-ethyl]-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (115.5 mg, 36%, compound 434).

Compound 434: MS (ES+, m/z): 1083.7 [M+Na]+. 1H NMR (600 MHz, DMSO-d6) Shift 5.87-6.57 (m, 5H), 5.42-5.68 (m, 1H), 5.24 (d, J=4.40 Hz, 1H), 4.89-5.16 (m, 3H), 3.86-4.08 (m, 3H), 3.67-3.81 (m, 1H), 3.33-3.61 (m, 7H), 3.31-3.33 (m, 3H), 3.12-3.27 (m, 9H), 2.63-3.08 (m, 7H), 2.22-2.43 (m, 3H), 2.07-2.12 (m, 1H), 1.83-2.01 (m, 5H), 1.51-1.76 (m, 18H), 1.36-1.44 (m, 3H), 1.20-1.20 (m, 1H), 1.10-1.33 (m, 7H), 0.94-1.05 (m, 6H), 0.87 (s, 14H). LCMS: MH+ (ion type), 1083.6 (ion m/z).

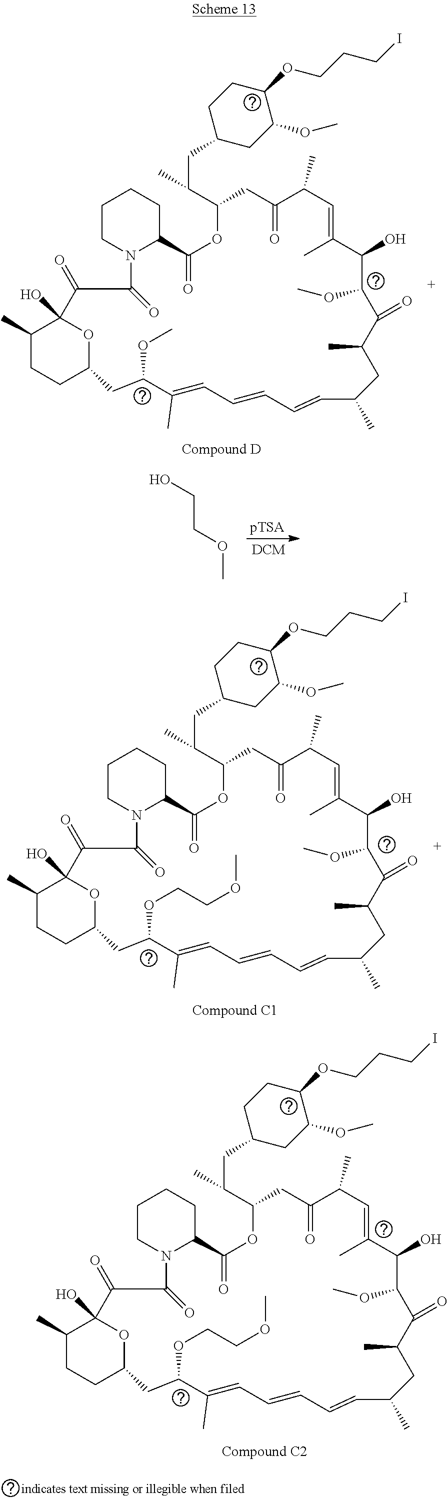

To a solution of (1R,9S,12SR,15R,16E,18R,19R,21R,23S,24E,26E,28E,30S,32S,35R)-1,18-dihydroxy-12-[(1R)-2-[(1S,3R,4R)-4-(3-iodopropoxy)-3-methoxy-cyclohexyl]-1-methyl-ethyl]-19,30-dimethoxy-15,17,21,23,29,35-hexamethyl-11,36-dioxa-4-azatricyclo[30.3.1.04,9]hexatriaconta-16,24,26,28-tetraene-2,3,10,14,20-pentone (6.83 g, 6.31 mmol) in DCM (77 mL) was added 2-methoxyethanol (161 mL, 2.31 mol) then 4-methylbenzenesulfonic acid (5.43 g, 31.5 mmol). The reaction mixture was stirred for 1 h at room temperature and subsequently neutralized with a saturated aqueous solution of NaHCO₃. The two phases were separated. The organic phase was washed with NaCl, dried and concentrated to dryness. Purification of the crude mixture by reverse phase chromatography (Uptisphere Strategy C18-Hq 10 um 250×30.0 mm CH₃CN:H2O gradient 75:25 to 100:0, 277 nm). The main fraction (2.88 g) was purified by SFC separation to afford (1R,9S,12S,15R,16E,18R,19R,21R,23S,24E,26E,28E,30S,32S,35R)-1,18-dihydroxy-12-[(1R)-2-[(1S,3R,4R)-4-(3-iodopropoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (2.11 g, 29%, compound C₁) and (1R,9S,12S,15R,16E,18R,19R,21R,23S,24E,26E,28E,30R,32S,35R)-1,18-dihydroxy-12-[(1R)-2-[(1S,3R,4R)-4-(3-iodopropoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.04,9]hexatriaconta-16,24,26,28-tetraene-2,3,10,14,20-pentone (850 mg, 12%) compound C2. SFC separation: Column: Princeton 2 Ethylpyridine 5 μm 60 A Column size: 3 cm I.D.×15 cm L; Mobile phase: CO2/IpOH 80/20; Flowrate: 100 ml/min; Pressure: 100 Bar Wave length: UV 277 nm SFC Equipment: Waters SFC200.

Compound C1: ¹H NMR (600 MHz, DMSO-d₆) δ 5.87-6.54 (m, 5H), 5.40-5.70 (m, 1H), 4.86-5.35 (m, 4H), 3.99-4.06 (m, 2H), 3.92-3.99 (m, 1H), 3.71-3.82 (m, 2H), 3.47-3.57 (m., 3H), 3.31-3.32 (m, 3H), 3.09-3.27 (m, 10H), 2.94-3.06 (m, 3H), 2.66-2.85 (m, 2H), 2.35-2.48 (m, 2H), 2.22 (br d, J=7.34 Hz, 1H), 2.07-2.12 (m, 1H), 2.01-2.05 (m, 1H), 1.84-1.99 (m, 6H), 1.74 (s, 2H), 1.52-1.70 (m, 11H), 1.33-1.45 (m, 2H), 1.21-1.33 (m, 3H), 1.10-1.20 (m, 2H), 0.94-1.09 (m, 8H), 0.76-0.88 (m, 9H), 0.71-0.75 (m, 3H), 0.64-0.68 (m, 1H). LCMS: MNa+ (ion type), 1148.6 (ion m/z).

Compound C2: ¹H NMR (DMSO-d₆, 600 MHz) δ 5.8-6.6 (m, 5H), 5.61 (dd, 1H, J=8.3, 14.5 Hz), 5.2-5.3 (m, 2H), 5.1-5.2 (m, 1H), 4.99 (br d, 1H, J=5.1 Hz), 3.8-4.1 (m, 4H), 2.9-3.6 (m, 22H), 2.5-2.8 (m, 3H), 2.2-2.4 (m, 1H), 2.1-2.2 (m, 1H), 1.8-2.1 (m, 5H), 0.5-1.8 (m, 42H), LCMS: MNa+ (ion type), 1148.4 (ion m/z).

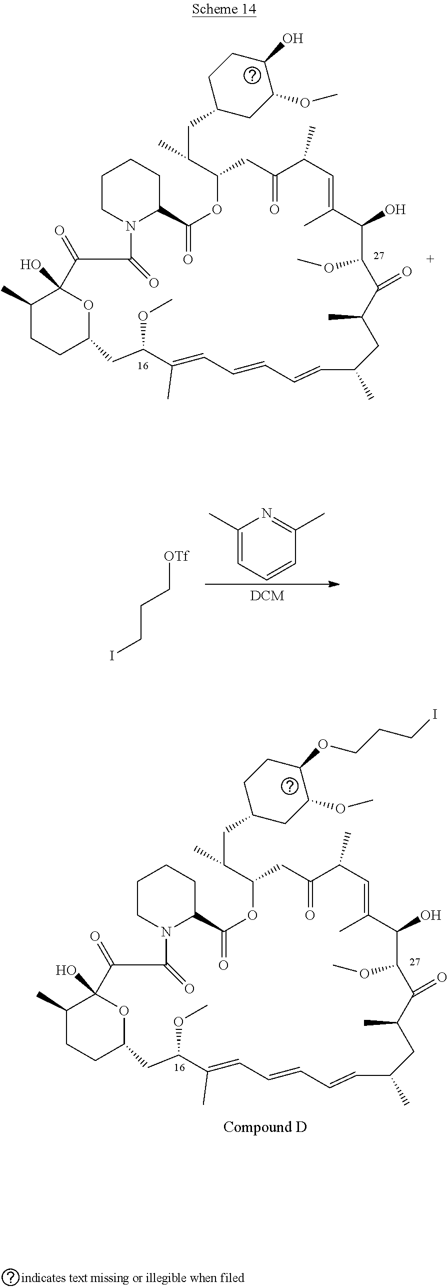

Under Ar, rapamycin (3.00 g, 3.28 mmol) was dissolved in Anhydrous Toluene (20.25 mL) after which N-ethyl-N-isopropyl-propan-2-amine (5.3 mL, 30.1 mmol) and 3-iodopropyl trifluoromethanesulfonate (5.41 g, 16.4 mmol) were added. The mixture was then heated at 60° C. for 2.5 hours. The crude mixture was then concentrated under vacuum and purified by silica gel flash column chromatography (Cyclohexane/EtOAc from 100/0 to 50/50) to yield (1R,9S,12S,15R,16E,18R,19R,21R,23S,24E,26E,28E,30S,32S,35R)-1,18-dihydroxy-12-[(1R)-2-[(1S,3R,4R)-4-(3-iodopropoxy)-3-methoxy-cyclohexyl]-1-methyl-ethyl]-19,30-dimethoxy-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (3.07 g, 47%, compound D).

Compound D: MS (ES+, m/z): 1104.5 [M+Na]+. 1H NMR (DMSO, 600 MHz): δ (ppm) 6.43 (d, 1H), 6.41-6.38 (m, 1H), 6.25-6.09 (m, 3H), 5.46 (dd, J=14.9, 9.6 Hz, 1H), 5.25 (d, J=4.5 Hz, 1H), 5.10 (d, J=10.2 Hz, 1H), 5.00-4.92 (m, 2H), 4.28 (d, 1H), 4.03-3.98 (m, 2H), 3.94 (d, J=4.6 Hz, 1H), 3.64-3.60 (m, 1H), 3.53 (dtt, J=19.7, 9.9, 5.5 Hz, 2H), 3.44 (d, J=13.5 Hz, 1H), 3.33-3.28 (m, 5H), 3.10 (d, J=63.6 Hz, 9H), 2.73 (dd, J=17.7, 2.6 Hz, 1H), 2.43-2.36 (m, 2H), 2.25-1.80 (m, 7H), 1.75-0.89 (m, 31H), 0.89-0.71 (m, 10H), 0.69-0.60 (m, 1H). LCMS: MNa+ (ion type), 1104.5 (ion m/z).

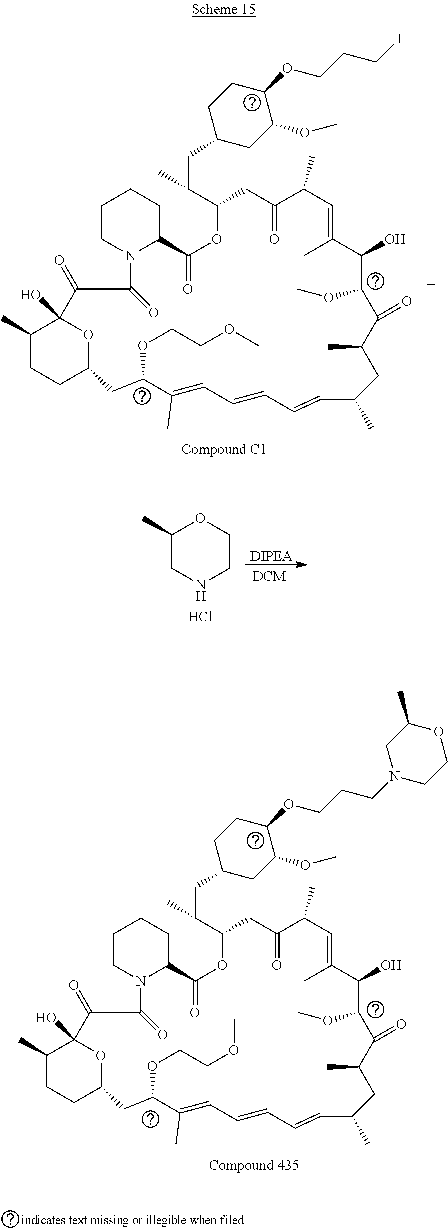

(1R,9S,12S,15R,16E,18R,19R,21R,23S,24E,26E,28E,30S,32S,35R)-1,18-dihydroxy-12-[(1R)-2-[(1S,3R,4R)-4-(3-iodopropoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (350 mg, 0.311 mmol, compound C1) was dissolved in dry DCM (1.5 mL). A solution of (2R)-2-methylmorpholine hydrochloride (51 mg, 0.373 mmol) and N-ethyl-N-isopropyl-propan-2-amine (228 uL, 1.31 mmol) in dry DCM (0.6 mL) was added and the solution stirred at rt for 20 hours. The reaction mixture was diluted with DCM and quenched with saturated NH₄Cl. The resulting mixture was washed with water, dried, concentrated and purified over silica gel flash column chromatography (100/0 to 70/30 of EtOAc/MeOH:Et₃N (50:50). The fractions of interest were purified over silica gel flash column chromatography (DCM/MeOH, 100/0 to 90/10) to yield (1R,9S,12S,15R,16E,18R,19R,21R,23S,24E,26E,28E,30S,32S,35R)-1,18-dihydroxy-19-methoxy-30-(2-methoxyethoxy)-12-[(1R)-2-[(1S,3R,4R)-3-methoxy-4-[3-[(2R)-2-methylmorpholin-4-yl]propoxy]cyclohexyl]-1-methyl-ethyl]-15,17,21,23,29,35-hexamethyl-11,36-dioxa-4-azatricyclo[30.3.1.04,9]hexatriaconta-16,24,26,28-tetraene-2,3,10,14,20-pentone (72.3 mg, 21%, compound 435).

Compound 435: MS (ES+, m/z): 1099.7 [M+Na]+. ¹H NMR (DMSO-d₆, 600 MHz): δ (ppm) 6.45 (d, J=1.3 Hz, 1H), 6.39 (dd, J=14.6, 11.2 Hz, 1H), 6.18-6.25 (m, 1H), 6.08-6.16 (m, 2H), 5.46 (dd, J=14.9, 9.6 Hz, 1H), 5.25 (d, J=4.5 Hz, 1H), 5.09 (br d, J=10.1 Hz, 1H), 4.96-5.00 (m, 1H), 4.91-4.95 (m, 1H), 3.98-4.06 (m, 2H), 3.93 (d, J=4.7 Hz, 1H), 3.76-3.81 (m, 1H), 3.70 (dd, J=11.2, 1.6 Hz, 1H), 3.34-3.56 (m, 7H), 3.32 (s, 3H), 3.24-3.29 (m, 2H), 3.23 (s, 3H), 3.17-3.21 (m, 1H), 3.15 (s, 3H), 2.90-3.05 (m, 2H), 2.55-2.76 (m, 3H), 2.33-2.45 (m, 2H), 2.30 (br t, J=7.2 Hz, 2H), 2.16-2.25 (m, 1H), 2.09 (br d, J=13.2 Hz, 1H), 1.98-2.06 (m, 1H), 1.77-1.97 (m, 5H), 1.73 (s, 3H), 1.65-1.68 (m, 2H), 1.64 (s, 3H), 1.45-1.62 (m, 8H), 1.35-1.45 (m, 2H), 1.24-1.33 (m, 3H), 1.08-1.20 (m, 3H), 1.04-1.08 (m, 2H), 1.02 (d, J=6.3 Hz, 3H), 0.98 (d, J=6.6 Hz, 3H), 0.88-0.96 (m, 2H), 0.87 (d, J=6.6 Hz, 3H), 0.83 (d, J=6.5 Hz, 3H), 0.77 (d, J=6.7 Hz, 31), 0.73 (d, J=6.6 Hz, 3H), 0.65 (q, J=11.9 Hz, 1H). LCMS: MH+ (ion type), 1099.6 (ion m/z).

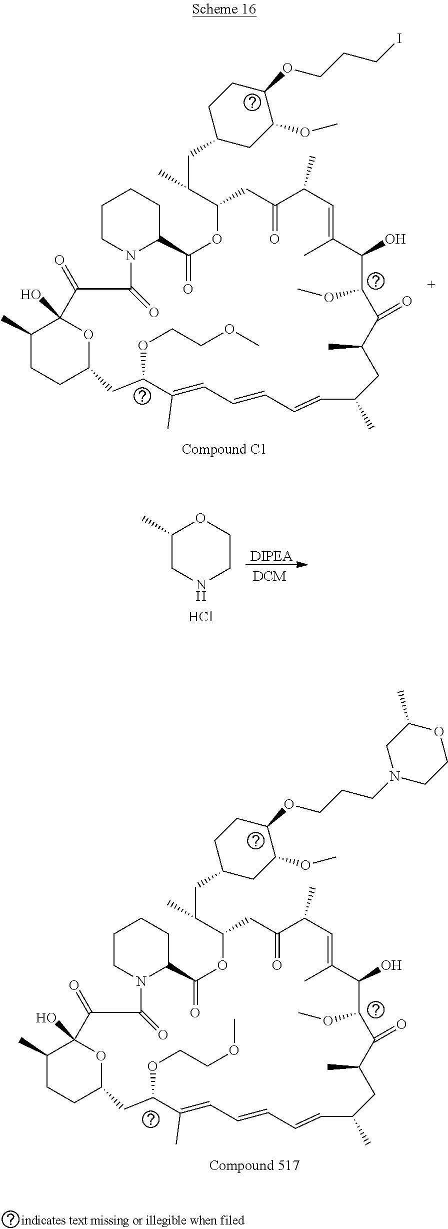

(1R,9S,12S,15R,16E,18R,19R,21R,23S,24E,26E,28E,30S,32S,35R)-1,18-dihydroxy-12-[(1R)-2-[(1S,3R,4R)-4-(3-iodopropoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (150 mg, 0.1332 mmol, compound 17) was dissolved in dry DCM (0.63 mL). A solution of (2S)-2-methylmorpholine hydrochloride (21.99 mg, 0.159 mmol) and N-ethyl-N-isopropyl-propan-2-amine (97.7 uL 0.559 mmol) in dry DCM (0.4 mL) was added and the solution stirred at rt for 20 hours. The reaction mixture was diluted with DCM and quenched with saturated NH4Cl. The resulting mixture was washed with water, dried, concentrated and purified over silica gel flash column chromatography (100/0 to 70/30 of EtOAc/MeOH:Et3N (50:50). The fractions of interest were purified over silica gel flash column chromatography (DCM/MeOH, 100/0 to 90/10) to yield (1R,9S,12S,15R,16E,18R,19R,21R,23S,24E,26E,28E,30S,32S,35R)-1,18-dihydroxy-19-methoxy-30-(2-methoxyethoxy)-12-[(1R)-2-[(1S,3R,4R)-3-methoxy-4-[3-[(2S)-2-methylmorpholin-4-yl]propoxy]cyclohexyl]-1-methyl-ethyl]-15,17,21,23,29,35-hexamethyl-11,36-dioxa-4-azatricyclo[30.3.1.04,9]hexatriaconta-16,24,26,28-tetraene-2,3,1.0,14,20-pentone (80 mg, 54%, compound 517).

Compound 517: MS (ES+, m/z): 1099.7 [M+Na]+. Rt=2.29 min. 1H NMR (600 MHz, DMSO-d6) Shift 6.04-6.50 (m, 1H), 5.40-5.70 (m, 1H), 5.20-5.28 (m, 1H), 4.86-5.16 (m, 1H), 3.62-4.08 (m, 5H), 3.32-3.56 (m, 11H), 3.11-3.28 (m, 9Hf), 2.92-3.04 (m, 2H), 2.58-2.74 (m, 3H), 2.35-2.45 (m, 2H), 2.18-2.33 (m, 3H), 1.79-2.12 (m, 8H), 1.73 (s, 2H), 1.52-1.70 (m, 14H), 1.31-1.44 (m, 3H), 1.21-1.31 (m, 5H), 1.18 (br dd, (J=4.84, 11.59 Hz, 1H), 0.94-1.11 (m, 11H), 0.72-0.88 (m, 13H), 0.53-0.71 (m, 2H). LCMS: MH+ (ion type), 1099.7 (ion m/z).

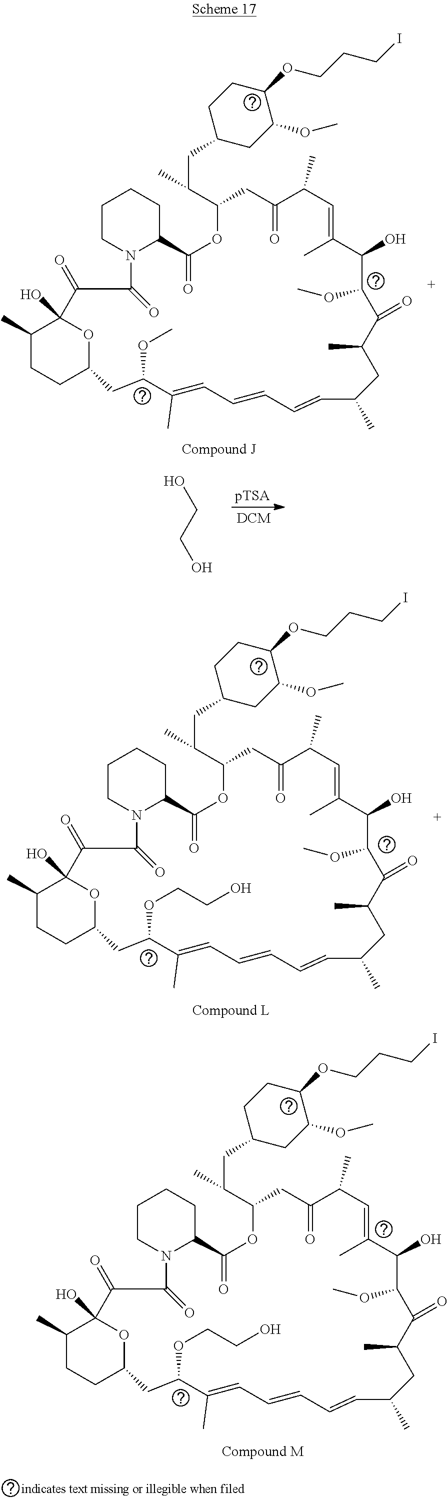

(1R,9S,12SR,15R,16E,18R,19R,21R,23S,24E,26E,28E,30S,32SR,35R)-1,18-dihydroxy-12-[(1R)-2-[(1S,3R,4R)-4-(3-iodopropoxy)-3-methoxy-cyclohexyl]-1-methyl-ethyl]-19,30-dimethoxy-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (2.00 g, 1.85 mmol) in anhydrous DCM (18.5 mL) was added ethylene glycol (34 mL, 0.61 mol) then 4-methylbenzenesulfonic acid (1.59 g, 9.24 mmol). The reaction mixture was stirred at room temperature for 3 hours and subsequently neutralized with a saturated aqueous solution of NaHCO₃. The two phases were separated. The organic phase was washed with NaCl, dried and concentrated to dryness. Purification of the crude mixture by reverse phase chromatography (Uptisphere Strategy C18-Hq 10 um 250×30.0 mm CH3CN:H2O gradient 70:30 to 100:0, 277 nm). Two isolated fractions were purified by SFC to afford (1R,9S,12S,15R,16E,18R,19R,21R,23S,24E,26E,28E,30S,32S,35R)-1,18-dihydroxy-30-(2-hydroxyethoxy)-12-[(1R)-2-[(1S,3R,4R)-4-(3-iodopropoxy)-3-methoxy-cyclohexyl]-1-methyl-ethyl]-19-methoxy-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (400 mg, 19%, compound L) and (1R,9S,12S,15R,16E,18R,19R,21R,23S,24E,26E,28E,30R,32S,35R)-1,18-dihydroxy-30-(2-hydroxyethoxy)-12-[(1R)-2-[(1S,3R,4R)-4-(3-iodopropoxy)-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (500 mg, 19%, compound M).

SFC separation: Column: Princeton 2 Ethylpyridine 5 μm 60 A. Column size: 3 cm I.D.×15 cm L. Mobile phase: CO2/IpOH 80/20. Flowrate: 50 ml/min. Pressure: 100 Bar. Wave length: UV 277 nm. SFC Equipment: Waters SFC200.

Compound L: 1H NMR (DMSO-d6, 600 MHz): δ (ppm) 6.43 (d, J=1.0 Hz, 1H), 6.40 (dd, J=14.6, 11.2 Hz, 1H), 6.07-6.25 (m, 3H), 5.46 (dd, J=14.9, 9.6 Hz, 1H), 5.25 (br d, J=4.1 Hz, 1H), 5.09 (br d, J=10.1 Hz, 1H), 4.95-5.00 (m, 1H), 4.92-4.94 (m, 1H), 4.44-4.53 (m, 1H), 3.99-4.09 (m, 2H), 3.94 (d, J=4.5 Hz, 1H), 3.75-3.81 (m, 1H), 3.49-3.57 (m, 2H), 3.40-3.48 (m, 3H), 3.32 (s, 3H), 3.30-3.30 (m, 2H), 3.19-3.28 (m, 3H), 3.15 (s, 3H), 3.12-3.16 (m, 1H), 3.02-3.07 (m, 1H), 2.97 (ddd, J=11.0, 8.8, 4.5 Hz, 1H), 2.73 (br dd, J=17.5, 2.6 Hz, 1H), 2.34-2.44 (m, 2H), 2.18-2.33 (m, 11H), 1.78-2.15 (m, 8H), 1.74 (s, 3H), 1.67-1.72 (m, 2H), 1.65 (s, 3H), 1.48-1.64 (m, 5H), 1.01-1.43 (m, 10H), 0.98 (d, J=6.6 Hz, 3H), 0.87 (d, J=6.5 Hz, 3H), 0.84-0.85 (m, 1H), 0.83 (d, J=6.5 Hz, 3H), 0.78 (d, J=6.7 Hz, 3H), 0.73 (d, J=6.7 Hz, 3H), 0.62-0.69 (m, 1H). LCMS: MNa+ (ion type), 1134.3 (ion m/z).

Compound M: MS (ES+, m/z): 1134.3 [M+Na]+. Rt=5.34 min. ¹H NMR (DMSO-d₆, 600 MHz): δ (ppm) 6.51 (s, 1H), 6.40-6.48 (m, 1H), 6.13-6.22 (m, 2H), 6.04 (br d, J=11.2 Hz, 1H), 5.63 (br dd, J=14.2, 8.2 Hz, 1H), 5.05-5.45 (m, 8H), 4.99 (br d, J=5.9 Hz, 1H), 4.51 (br t, J=5.4 Hz, 1H), 4.04 (br t, J=4.2 Hz, 1H), 3.99-4.03 (m, 1H), 3.89 (d, J=4.7 Hz, 1H), 3.83 (br dd, J=9.6, 1.2 Hz, 1H), 3.41-3.59 (m, 5H), 3.31-3.32 (m, 1H), 3.31 (br s, 3H), 3.29-3.29 (m, 2H), 3.21-3.25 (m, 2H), 3.18 (s, 3H), 3.05-3.10 (m, 1H), 2.93-3.05 (m, 2H), 2.75-2.80 (m, 1H), 2.67-2.74 (m, 1H), 2.52-2.59 (m, 1H), 2.21-2.32 (m, 1H), 1.87-2.18 (m, 7H), 1.46-1.78 (m, 13H), 1.08-1.45 (m, 6H), 0.99 (br d, J=6.6 Hz, 3H), 0.95-0.98 (m, 1H), 0.93 (br d, J=6.7 Hz, 3H), 0.85 (br d, J=6.6 Hz, 3H), 0.80 (br d, J=6.7 Hz, 3H), 0.75 (br d, J=6.6 Hz, 3H), 0.59-0.67 (m, 1H). LCMS: MNa+ (ion type), 1134.3 (ion m/z).

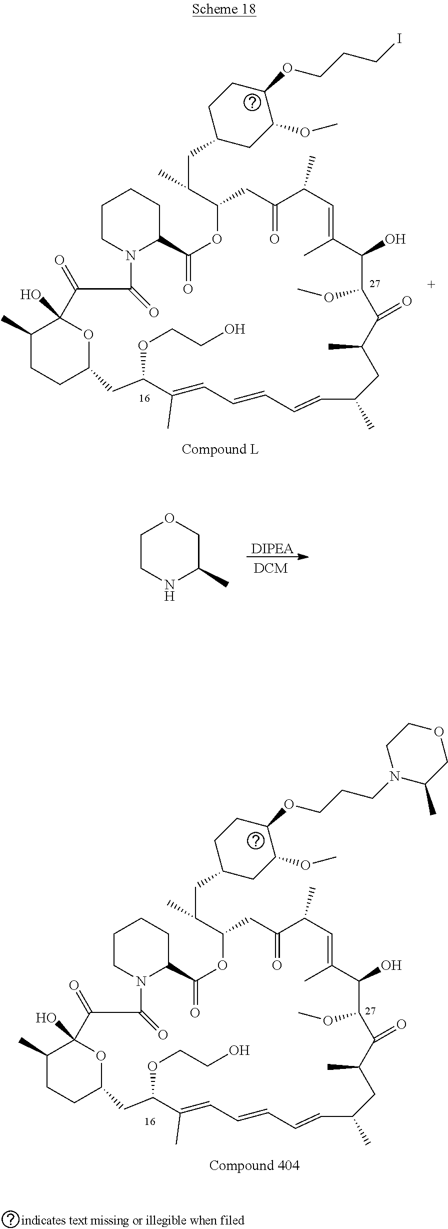

(1R,9S,12S,15R,16E,18R,19R,21R,23S,24E,26E,28E,30S,32S,35R)-1,18-dihydroxy-30-(2-hydroxyethoxy)-12-[(1R)-2-[(1S,3R,4R)-4-(3-iodopropoxy)-3-methoxy-cyclohexyl]-1-methyl-ethyl]-19-methoxy-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (350 mg, 0.315 mmol) was dissolved in DCM-Anhydrous (1,1444 mL) with N-ethyl-N-isopropyl-propan-2-amine (220 uL, 1.26 mmol) and (3R)-3-methylmorpholine (97%, 43 uL, 0.378 mmol). The reaction mixture was stirred at room temperature for 24 hours. The mixture was diluted with DCM. An aqueous saturated solution of NH₄Cl was added to adjust the pH to 7. The resulting mixture was washed with water, dried, concentrated and purified over silica gel flash column chromatography (100/0 to 90/10 of EtOAc/MeOH:Et3N (50:50). The fractions of interest were purified over silica gel flash column chromatography (DCM/MeOH, 100/0 to 90/10) to yield (1R,9S,12S,15R,16E,18R,19R,21R,23S,24E,26E,28E,30S,32S,35R)-1,18-dihydroxy-30-(2-hydroxyethoxy)-19-methoxy-12-[(1R)-2-[(1S,3R,4R)-3-methoxy-4-[3-[(3R)-3-methylmorpholin-4-yl]propoxy]cyclohexyl]-1-methyl-ethyl]-15,17,21,23,29,35-hexamethyl-11,36-dioxa-4-azatricyclo[30.3.1.04,9]hexatriaconta-16,24,26,28-tetraene-2,3,10,14,20-pentone (101.3 mg, 29%, Compound 404).

Compound 404: MS (ES+, m/z): 1085.6 [M+Na]+. 1H NMR (600 MHz, DMSO-d6) Shift 5.89-6.62 (m, 5H), 5.43-5.67 (m, 1H), 5.19-5.28 (m, 1H), 4.91-5.14 (m, 3H), 4.43-4.57 (m, 1H), 3.43-4.07 (m, 12H), 3.31-3.34 (m, 3H), 3.11-3.28 (m, 7H), 2.93-3.10 (m, 4H), 2.64-2.81 (m, 3H), 1.84-2.44 (m, 12H), 1.73 (s, 3H), 1.51-1.70 (m, 12H), 1.36-1.44 (m, 2H), 1.22-1.35 (m, 4H), 1.13-1.19 (m, 1H), 0.94-1.11 (m, 8H), 0.81-0.92 (m, 9H), 0.77 (d, J=6.75 Hz, 2H), 0.73 (d, J=6.60 Hz, 2H), 0.63-0.70 (m, 1H). LCMS: MH+ (ion type), 1085.6 (ion m/z).

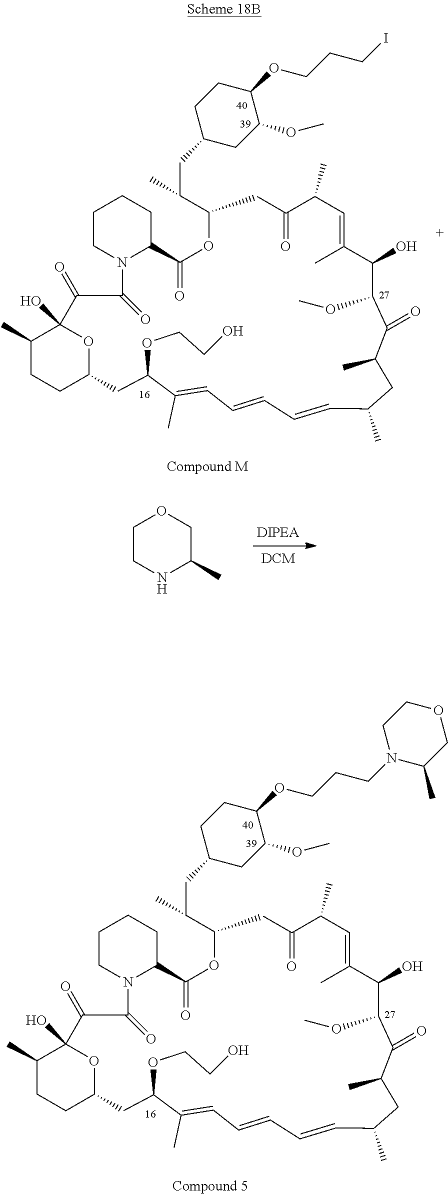

(1R,9S,12S,15R,16E,18R,19R,21R,23S,24E,26E,28E,30R,32S,35R)-1,18-dihydroxy-30-(2-hydroxyethoxy)-12-[(1R)-2-[(1S,3R,4R)-4-(3-iodopropoxy)-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (350 mg, 0.315 mmol) was dissolved in DCM-Anhydrous (1,1444 mL) with N-ethyl-N-isopropyl-propan-2-amine (220 uL, 1.26 mmol) and (3R)-3-methylmorpholine (97%, 43 uL, 0.378 mmol). The reaction mixture was stirred at room temperature for 72 hours. The mixture was diluted with DCM. An aqueous saturated solution of NH₄Cl was added to adjust the pH to 7. The resulting mixture was washed with water, dried, concentrated and purified over silica gel flash column chromatography (100/0 to 90/10 of EtOAc/MeOH:Et3N (50:50). The fractions of interest were purified over silica gel flash column chromatography (DCM/MeOH, 100/0 to 90/10) to yield (1R,9S,12S,15R,16E,18R,19R,21R,23S,24E,26E,28E,30R,32S,35R)-1,18-dihydroxy-30-(2-hydroxyethoxy)-19-methoxy-12-[(1R)-2-[(1S,3R,4R)-3-methoxy-4-[3-[(3R)-3-methylmorpholin-4-yl]propoxy]cyclohexyl]-1-methyl-ethyl]-15,17,21,23,29,35-hexamethyl-11,36-dioxa-4-azatricyclo[30.3.1.04,9]hexatriaconta-16,24,26,28-tetraene-2,3,10,14,20-pentone (52 mg, 15%, Compound 404).

Compound 5: MS (ES+, m/z): 1085.6 [M+Na]+. 6.63-5.85 (m, 5H), 5.74-5.51 (m, 1H), 5.45-5.05 (m, 3H), 5.04-4.72 (m, 2H), 4.58-4.41 (m, 11H), 4.18-3.39 (m, 9Hf), 3.33-3.30 (m, 3H), 3.26-3.09 (m, 4H), 3.09-2.89 (m, 3H), 2.82-2.62 (m, 3H), 2.33-1.85 (m, 7H), 1.80-1.44 (m, 11H), 1.42-1.21 (m, 5H), 1.19-0.60 (m, 17H) LCMS: MH+ (ion type), 1085.6 (ion m/z).

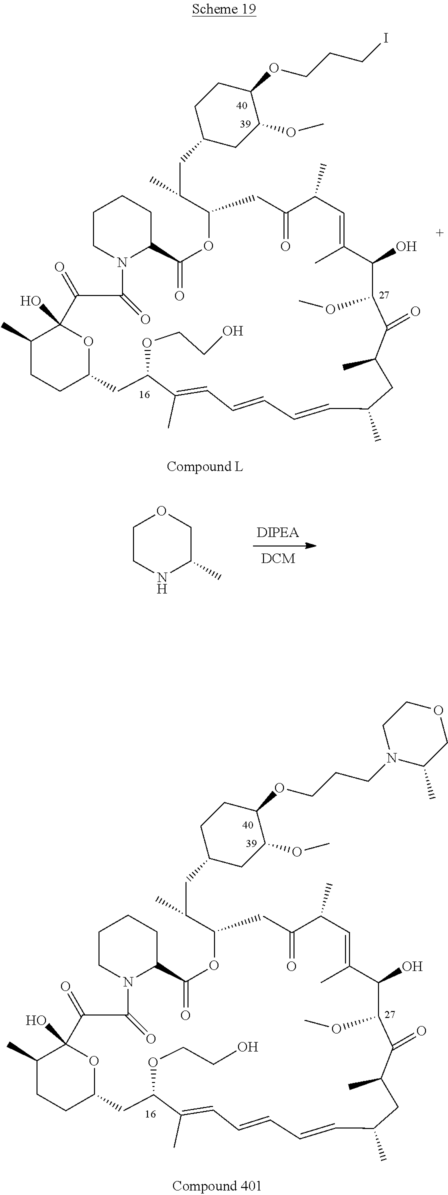

(1R,9S,12S,15R,16E,18R,19R,21R,23S,24E,26E,28E,30S,32S,35R)-1,18-dihydroxy-30-(2-hydroxyethoxy)-12-[(1R)-2-[(1S,3R,4R)-4-(3-iodopropoxy)-3-methoxy-cyclohexyl]-1-methyl-ethyl]-19-methoxy-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (143 mg, 0.129 mmol) was dissolved in DCM-Anhydrous (1,1444 mL) with N-ethyl-N-isopropyl-propan-2-amine (90 uL, 0.514 mmol) and (3S)-3-methylmorpholine (18 uL, 0.154 mmol). The reaction mixture was stirred at room temperature for 48 hours. The mixture was diluted with DCM. An aqueous saturated solution of NH4Cl was added to adjust the pH to 7. The resulting mixture was washed with water, dried, concentrated and purified over silica gel flash column chromatography (100/0 to 90/10 of EtOAc/MeOH:Et3N (50:50). The fractions of interest were purified over silica gel flash column chromatography (DCM/MeOH, 100/0 to 90/10) to yield (1R,9S,12S,15R,16E,18R,19R,21R,23S,24E,26E,28E,30S,32S,35R)-1,18-dihydroxy-30-(2-hydroxyethoxy)-19-methoxy-12-[(1R)-2-[(1S,3R,4R)-3-methoxy-4-[3-[(3S)-3-methylmorpholin-4-yl]propoxy]cyclohexyl]-1-methyl-ethyl]-15,17,21,23,29,35-hexamethyl-11,36-dioxa-4-azatricyclo[30.3.1.04,9]hexatriaconta-16,24,26,28-tetraene-2,3,10,14,20-pentone (118.4 mg, 85%, Compound 401).

Compound 401: MS (ES+, m/z): 1085.6 [M+Na]+. Rt=1.34 min. 1H NMR (600 MHz, DMSO-d6) Shift 5.90-6.58 (m, 5H), 5.39-5.67 (m, 1H), 5.20-5.27 (m, 1H), 4.84-5.15 (m, 3H), 4.43-4.57 (m, 1H), 3.40-4.11 (m, 12H), 3.31-3.34 (m, 3H), 3.12-3.29 (m, 7H), 2.93-3.08 (m, 3H), 2.63-2.77 (m, 3H), 1.85-2.41 (m, 12H), 1.46-1.79 (m, 16H), 1.22-1.42 (m, 6H), 0.65-1.05 (m, 23H). LCMS: MH+ (ion type), 1085.6 (ion m/z).

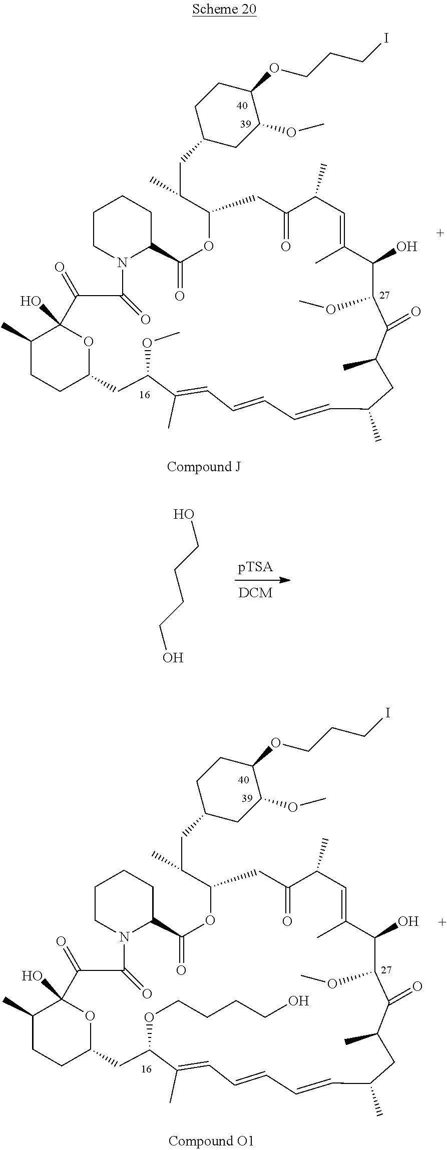

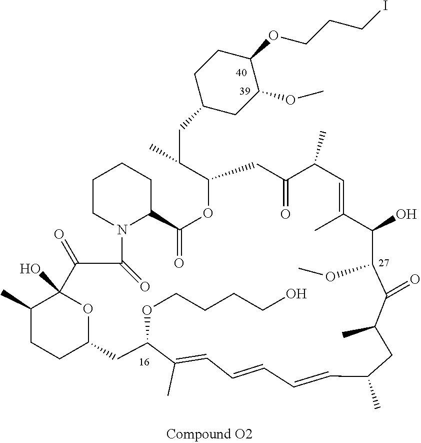

(1R,9S,12SR,15R,16E,18R,19R,21R,23S,24E,26E,28E,30S,32SR,35R)-1,18-dihydroxy-12-[(1R)-2-[(1S,3R,4R)-4-(3-iodopropoxy)-3-methoxy-cyclohexyl]-1-methyl-ethyl]-19,30-dimethoxy-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (5.00 g, 4.62 mmol) was dissolved in anhydrous DCM (184 mL) and butane-1,4-diol (16 mL, 182 mmol). 4-methylbenzenesulfonic acid (3.97 g, 23 mmol) was added and the reaction mixture stirred at room temperature for 4 hours. It was then neutralized with a saturated aqueous solution of NaHCO₃ and the two phases were separated. The organic phase was washed with brine, dried and concentrated to dryness. Purification of the obtained crude by reverse phase chromatography (Uptisphere Strategy C18-Hq 10 um 250×30.0 mm CH3CN:H2O gradient 70:30 to 100:0, 277 nm) afforded one main fraction (2.2 g) that was purified by SFC to afford two fractions.

SFC separation: Column: Princeton 2 Ethylpyridine Sm 60 A. Column size: 3 cm I.D.×15 cm L. Mobile phase: CO2/IpOH 83/17. Flowrate: 50 ml/min. Pressure: 100 Bar.Wave length: UV 277 nm. SFC Equipment: Waters SFC200.

Compound O1: (1R,9S,12S,15R,16E,18R,19R,21R,23S,24E,26E,28E,30S,32S,35R)-1,18-dihydroxy-30-(4-hydroxybutoxy)-12-[(1R)-2-[(1S,3R,4R)-4-(3-iodopropoxy)-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (969 mg, 16%, compound O1).

Compound O1: ¹H NMR (DMSO-d₆, 600 MHz): δ (ppm) 6.44 (s, 1H), 6.37-6.42 (m, 1H), 6.17-6.24 (m, 1H), 6.07-6.16 (m, 2H), 5.45 (dd, J=14.9, 9.8 Hz, 1H), 5.25 (d, J=4.5 Hz, 1H), 5.09 (br d, J=10.3 Hz, 1H), 4.98 (br s, 1H), 4.93 (br s, 1H), 4.30-4.40 (m, 1H), 4.02 (br d, J=12.0 Hz, 2H), 3.94 (d, J=4.5 Hz, 1H), 3.72 (br d, J=13.5 Hz, 1H), 3.48-3.57 (m, 2H), 3.41-3.47 (m, 1H), 3.35-3.40 (m, 2H), 3.32 (s, 3H), 3.30-3.30 (m, 2H), 3.16-3.28 (m, 3H), 3.15 (s, 3H), 3.07-3.11 (m, 1H), 3.01-3.06 (m, 1H), 2.93-3.00 (m, 1H), 2.73 (br d, J=15.3 Hz, 1H), 2.36-2.48 (m, 2H), 2.18-2.33 (m, 1H), 2.07-2.14 (m, 1H), 1.99-2.06 (m, 1H), 1.88-1.98 (m, 4H), 1.83 (br s, 1H), 1.74 (s, 3H), 1.52-1.68 (m, 9H), 1.35-1.52 (m, 7H), 1.24 (br s, 4H), 1.17 (br t, J=7.1 Hz, 2H), 1.00-1.10 (m, 3H), 0.98 (br d, J=6.6 Hz, 3H), 0.88-0.93 (m, 1H), 0.87 (d, J=6.6 Hz, 31H), 0.83 (br d, J=6.5 Hz, 3H), 0.78 (d, J=6.7 Hz, 3H), 0.73 (d, J=6.6 Hz, 3H), 0.59-0.69 (m, 1H) LCMS: MNa+ (ion type), 1162.6 (ion m/z).

Compound O2: (1R,9S,12S,15R,16E,18R,19R,21R,23S,24E,26E,28E,30R,32S,35R)-1,18-dihydroxy-30-(4-hydroxybutoxy)-12-[(1R)-2-[(1S,3R,4R)-4-(3-iodopropoxy)-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (341 mg, 4%, compound O2).

Compound O2: ¹H NMR (600 MHz, DMSO-d6) δ 5.87-6.64 (m, 5H), 5.42-5.68 (m, 1H), 5.20-5.35 (m, 2H), 5.05-5.16 (m, 1H), 4.99 (br d, J=4.84 Hz, 1H), 4.26-4.45 (m, 1H), 3.76-4.09 (m, 4H), 3.44-3.58 (m, 3H), 3.35-3.41 (m, 2H), 3.32-3.33 (m, 11H), 3.12-3.23 (m, 5H), 3.3 (m, 6H) 2.95-3.05 (m, 2H), 2.73-2.83 (m, 1H), 2.5-2.5 (m, 2H), 2.25-2.32 (m, 1H), 1.78-2.19 (m, 6H), 1.21-1.76 (m, 25H), 0.55-1.19 (m, 21H). LCMS: MNa+ (ion type), 1162.5 (ion m/z).

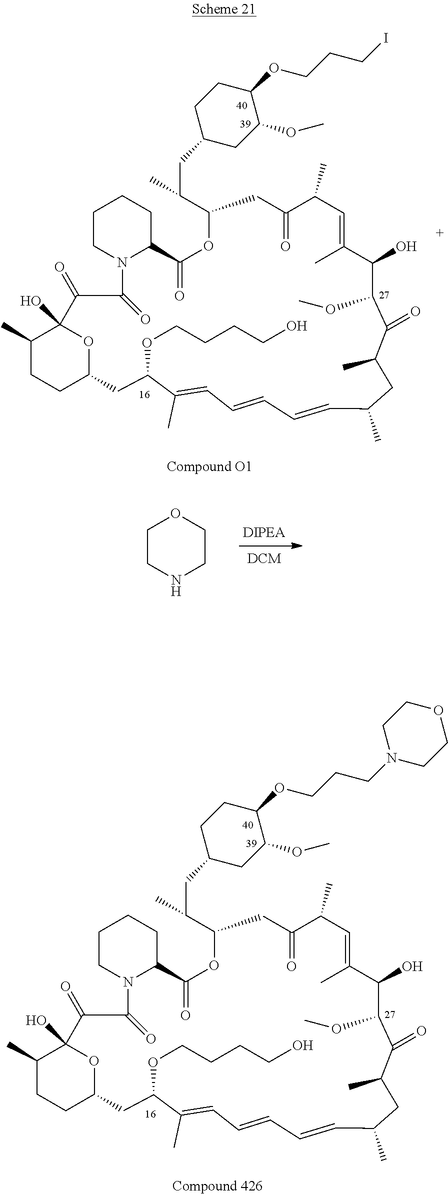

(1R,9S,12SR,15R,16E,18R,19R,21R,23S,24E,26E,28E,30S,32S,35R)-1,18-dihydroxy-30-(4-hydroxybutoxy)-12-[(1R)-2-[(1S,3R,4R)-4-(3-iodopropoxy)-3-methoxy-cyclohexyl]-1-methyl-ethyl]-19-methoxy-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (240 mg, 0.210 mmol) in dry DCM (1,3155 mL) was added N-ethyl-N-isopropyl-propan-2-amine (110 uL, 0.631 mmol) then morpholine (22 uL, 0.253 mmol). The reaction mixture was stirred at room temperature for 24 hours. The mixture was then diluted with DCM and aqueous HCl 1N was added until pH=5. The organic phase was washed with water, dried and concentrated to dryness and purified over silica gel flash column chromatography (100/0 to 85/15 of EtOAc/MeOH:Et3N (50:50). The fractions of interest were purified over silica gel flash column chromatography (DCM/MeOH, 100/0 to 90/10) to yield (1R,9S,12S,15R,16E,18R,19R,21R,23S,24E,26E,28E,30S,32S,35R)-1,18-dihydroxy-30-(4-hydroxybutoxy)-19-methoxy-12-[(1R)-2-[(1S,3R,4R)-3-methoxy-4-(3-morpholinopropoxy)cyclohexyl]-1-methyl-ethyl]-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (107 mg, 46%, Compound 426).

Compound 426: MS (ES+, m/z): 1099.6 [M+H]+. Rt=1.62 min. ¹H NMR (DMSO-d₆, 600 MHz): δ (ppm) 6.43-6.46 (m, 1H), 6.39 (dd, J=14.5, 11.2 Hz, 1H), 6.07-6.24 (m, 3H), 5.42-5.49 (m, 1H), 5.25 (d, J=4.7 Hz, 1H), 5.09 (br d, J=10.1 Hz, 1H), 4.98 (dt, J=7.7, 4.1 Hz, 1H), 4.93 (br d, J=5.3 Hz, 1H), 4.35 (t, J=5.1 Hz, 1H), 3.97-4.07 (m, 2H), 3.94 (d, J=4.7 Hz, 1H), 3.72 (dd, J=11.8, 1.8 Hz, 1H), 3.42-3.57 (m, 7H), 3.34-3.40 (m, 2H), 3.32 (s, 3H), 3.24-3.29 (m, 1H), 3.17-3.23 (m, 2H), 3.15 (s, 3H), 3.09 (dt, J=9.4, 6.1 Hz, 1H), 2.92-3.04 (m, 2H), 2.73 (br dd, J=17.5, 2.4 Hz, 1H), 2.35-2.45 (m, 2H), 2.31 (br t, J=7.2 Hz, 6H), 2.16-2.25 (m, 1H), 1.78-2.14 (m, 6H), 1.74 (s, 3H), 0.89-1.69 (m, 29H), 0.87 (d, J=6.5 Hz, 3H), 0.81-0.84 (m, 1H), 0.83 (d, J=6.3 Hz, 3H), 0.77 (d, J=6.7 Hz, 3H), 0.73 (d, J=6.7 Hz, 3H), 0.61-0.69 (m, 1H). LCMS: MH+ (ion type), 1099.6 (ion m/z).

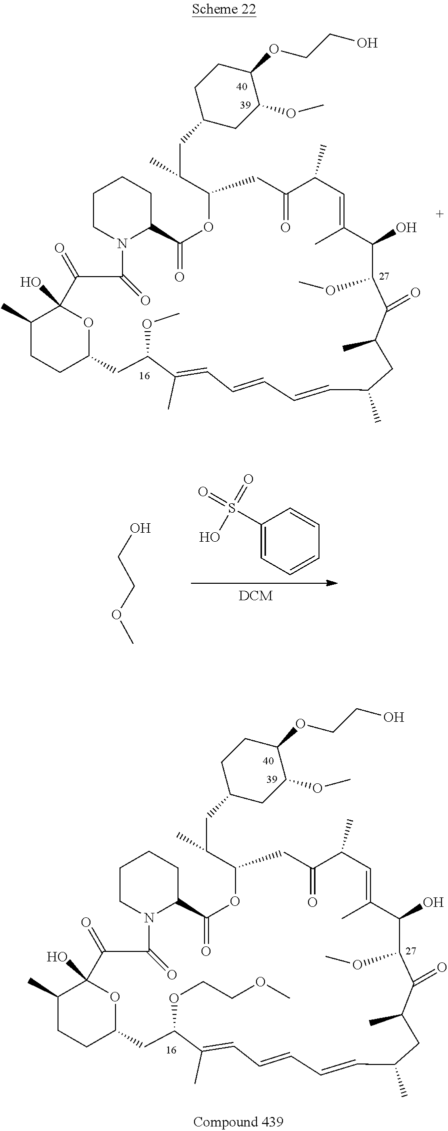

To a solution of everolimus (1.50 g, 1.57 mmol) in anhydrous DCM (19,568 mL) was added 2-methoxyethanol (45 mL, 0.573 mol) then 4-methylbenzenesulfonic acid (1.35 g, 7.83 mmol). The reaction mixture was stirred for 1 h at room temperature. The reaction mixture was neutralized with saturated NaHCO3 aqueous and extracted with DCM. The organic phase was washed with water (60 mL), dried, filtered and concentrated to dryness. The resulting crude mixture was purified by reverse phase chromatography (Uptisphere Strategy C18-Hq 10 um 250×30.0 mm CH3CN:H2O gradient 60:40 to 100:0 in 25 min, 277 nm). The main fraction (768 mg) was purified by SFC separation to afford (1R,9S,12S,15R,16E,18R,19R,21R,23S,24E,26E,28E,30S,32S,35R)-1,18-dihydroxy-12-[(1R)-2-[(1S,3R,4R)-4-(2-hydroxyethoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (compound 439,231 mg, 15%, white amorphous solid).

SFC separation: Column: Princeton 2 Ethylpyridine. 5 μm 60 A. Column size: 3 cm I.D.×15 cm L. Mobile phase: CO2/IpOH 85/15. Flowrate: 50 ml/min. Pressure: 100 Bar. Wave length: UV 277 nm. SFC Equipment: Waters SFC200.

Compound 439: ¹H NMR (DMSO-d₆, 600 MHz): δ (ppm) 5.90-6.49 (m, 5H), 5.42-5.5 (m, 1H), 5.25 (d, J=4.5 Hz, 1H), 5.09 (d, J=10.1 Hz, 1H), 5.00-4.95 (m, 1H), 4.93 (d, J=5.5 Hz, 1H), 4.44 (t, J=5.4 Hz, 1H), 4.08-3.98 (m, 2H), 3.94 (d, J=4.5 Hz, 1H), 3.78 (d, J=13.7 Hz, 1H), 3.56-3.35 (m, 6H), 3.35-3.31 (m, 3H), 3.28-3.11 (m, 7H), 3.08-2.93 (m, 2H), 2.77-2.70 (m, 1H), 2.45-2.34 (m, 2H), 2.21 (s, 1H), 2.14-1.81 (m, 6H), 1.74 (s, 2H), 1.72-1.47 (m, 8H), 1.47-1.21 (m, 5H), 1.20-0.91 (m, 8H), 0.89-0.60 (m, 10H). LCMS: MNa+ (ion type), 1024.7 (ion m/z).

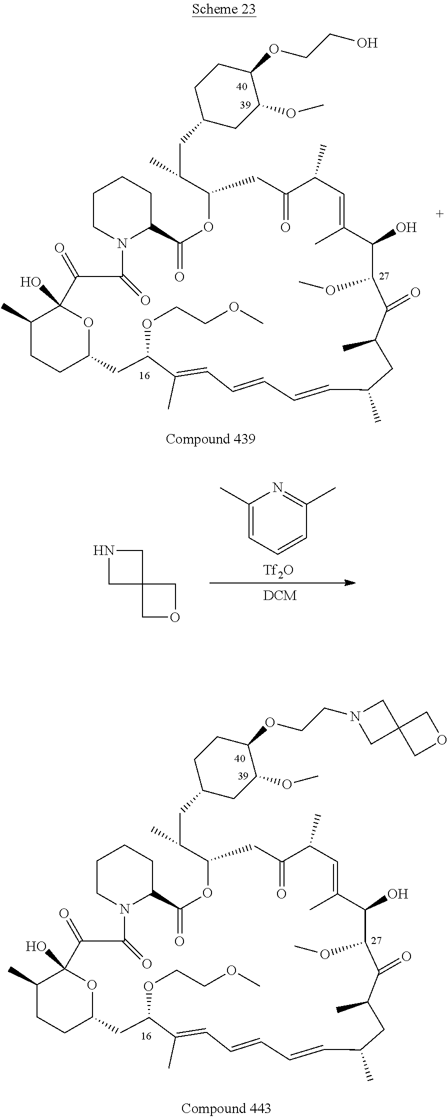

(1R,9S,12S,15R,16E,18R,19R,21R,23S,24E,26E,28E,30S,32S,35R)-1,18-dihydroxy 12-[(1R)-2-[(1S,3R,4R)-4-(2-hydroxyethoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.04,9]hexatriaconta-16,24,26,28-tetraene-2,3,10,14,20-pentone (Compound 29, 250 mg, 0.249 mmol) was diluted in anhydrous DCM (2,079 mL). The mixture was then cooled down to −78° C., before 2,6-dimethylpyridine (0.12 mL, 1.07 mmol) was added and stirred for 10 min. Subsequent addition of trifluoromethylsulfonyl trifluoromethanesulfonate (84 uL, 0.499 mmol) was carried out. The reaction was stirred at −78° C. for 1 hour. The ice bath was removed after addition of 2-oxa-6-azaspiro[3.3]heptane (98%, 0.11 mL, 1.25 mmol). The mixture was allowed to reach room temperature, diluted with DCM, concentrated, purified by silica gel flash column chromatography (100/0 to 90/10 of EtOAc/MeOH:Et₃N (50:50)). The isolated fractions of interest were purified a second time by silica gel flash column chromatography (0 to 20% of MeOH in DCM to afford (1R,9S,12S,15R,16E,18R,19R,21R,23S,24E,26E,28E,30S,32S,35R)-1,18-dihydroxy-19-methoxy-30-(2-methoxyethoxy)-12-[(1R)-2-[(1S,3R,4R)-3-methoxy-4-[2-(2-oxa-6-azaspiro[3.3]heptan-6-yl)ethoxy]cyclohexyl]-1-methyl-ethyl]-15,17,21,23,29,35-hexamethyl-11,36-dioxa-4-azatricyclo[30.3.1.04,9]hexatriaconta-16,24,26,28-tetraene-2,3,10,14,20-pentone (Compound 443, 21.2 mg, 8%, white amorphous solid).

Compound 443: ¹H NMR (DMSO-d₆, 600 MHz): δ (ppm) 6.45 (d, J=1.3 Hz, 1H), 6.39 (dd, J=14.6, 11.2 Hz, 1H), 6.19-6.25 (m, 1H), 6.07-6.17 (m, 2H), 5.46 (dd, J=14.8, 9.5 Hz, 1H), 5.25 (d, J=4.4 Hz, 1H), 5.09 (br d, J=10.0 Hz, 1H), 4.95-5.01 (m, 1H), 4.90-4.96 (m, 1H), 4.57 (s, 4H), 3.96-4.07 (m, 2H), 3.94 (d, J=4.5 Hz, 1H), 3.78 (dd, J=11.8, 2.0 Hz, 1H), 3.33-3.76 (m, 11H), 3.31 (s, 3H), 3.24-3.27 (m, 2H), 3.24 (s, 3H), 3.19-3.23 (m, 2H), 3.15 (s, 3H), 2.92-3.02 (m, 2H), 2.73 (br dd, J=17.8, 2.7 Hz, 1H), 2.36-2.44 (m, 3H), 2.18-2.26 (m, 1H), 2.07-2.13 (m, 1H), 1.82-2.04 (m, 5H), 1.73 (s, 3H), 1.67-1.72 (m, 2H), 1.64 (s, 3H), 1.37-1.58 (m, 7H), 1.25-1.35 (m, 3H), 0.99-1.21 (m, 5H), 0.98 (d, J=6.6 Hz, 3H), 0.87 (d, J=6.5 Hz, 3H), 0.83 (d, J=6.5 Hz, 3H), 0.77 (d, J=6.7 Hz, 3H), 0.73 (d, J=6.6 Hz, 3H), 0.58-0.69 (m, 1H). LCMS: MH+ (ion type), 1083.7 (ion m/z).

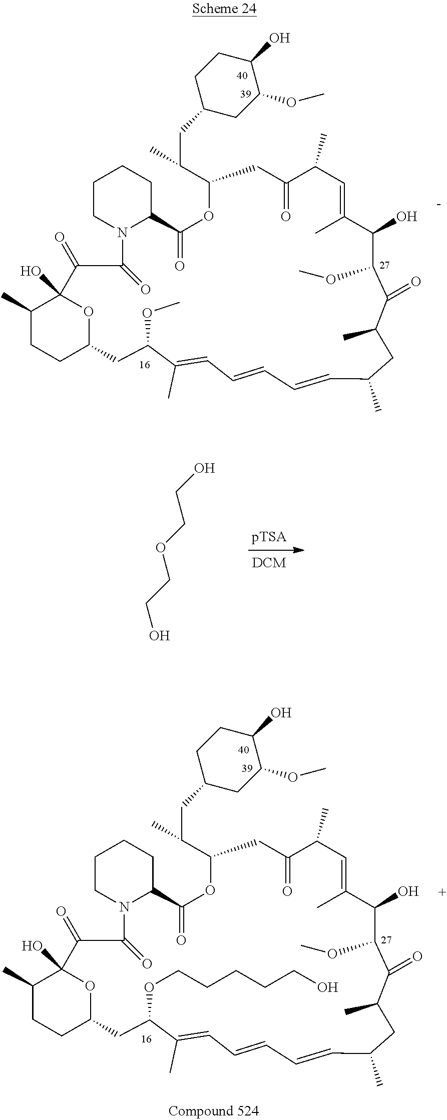

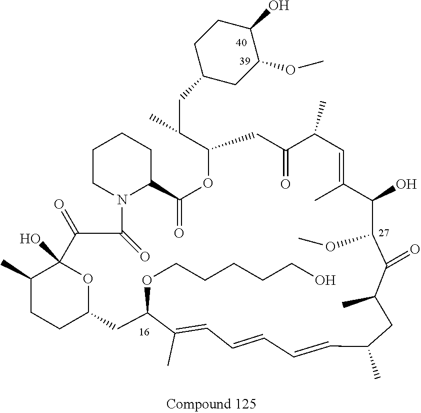

2,2′-oxydiethanol (8.7 mL, 86.6 mmol) was added to a solution of (1R,9S,12S,15R,16E,18R,19R,21R,23S,24E,26E,28E,30S,32S,35R)-1,18-dihydroxy-12-[(1R)-2-[(1S,3R,4R)-4-hydroxy-3-methoxy-cyclohexyl]-1-methyl-ethyl]-19,30-dimethoxy-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (2.00 g, 2.19 mmol) in anhydrous DCM (87 mL). The mixture was cooled to −15° C. and 4-methylbenzenesulfonic acid (1.88 g, 10.9 mmol) was added. The mixture was stirred 60 minutes at −15° C. and 120 minutes at room temperature. The mixture was diluted with DCM and neutralized by a saturated solution of NaHCO3. The phases were separated. The organic phase was washed with water (40 mL), dried, filtered and concentrated to dryness. The resulting crude mixture was purified by reverse phase chromatography (Uptisphere Strategy C18-Hq 10 um 250×30.0 mm CH₃CN:H₂O gradient 70:30 to 100:0, 277 nm). The main fraction (1.1 g) was purified by SFC separation to afford two fractions.

(1R,9S,12S,15R,16E,18R,19R,21R,23S,24E,26E,28E,30S,32S,35R)-1,18-dihydroxy-30-[2-(2-hydroxyethoxy)ethoxy]-12-[(1R)-2-[(1S,3R,4R)-4-hydroxy-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (138 mg, 6%, white amorphous solid, compound 524) and (1R,9S,12S,15R,16E,18R,19R,21R,23S,24E,26E,28E,30R,32S,35R)-1,18-dihydroxy-30-[2-(2-hydroxyethoxy)ethoxy]-12-[(1R)-2-[(1S,3R,4R)-4-hydroxy-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (58 mg, 3%, white amorphous solid, compound 125).

SFC separation: Column: Princeton 2 Ethylpyridine 5 μm 60 A. Column size: 3 cm I.D.×15 cm L. Mobile phase: CO₂/IpOH 82/18. Flowrate: 100 ml/min. Pressure: 100 Bar. Wave length: UV 277 nm. SFC Equipment: Waters SFC200.

Compound 524: ¹H NMR (DMSO-d₆, 600 MHz): δ (ppm) 6.44 (d, J=1.5 Hz, 1H), 6.40 (dd, J=14.5, 11.2 Hz, 1H), 6.18-6.25 (m, 1H), 6.07-6.16 (m, 2H), 5.46 (dd, J=14.8, 9.7 Hz, 1H), 5.24 (d, J=4.5 Hz, 1H), 5.09 (br d, J=10.1 Hz, 1H), 4.96-5.01 (m, 1H), 4.93 (br d, J=4.8 Hz, 1H), 4.47-4.63 (m, 2H), 3.97-4.08 (m, 2H), 3.94 (d, J=4.7 Hz, 11H), 3.79 (dd, J=11.7, 1.9 Hz, 1H), 3.38-3.58 (m, 7H), 3.22-3.37 (m, 6H), 3.11-3.21 (m, 5H), 2.83 (ddd, J=11.2, 8.6, 4.5 Hz, 1H), 2.73 (br dd, J=17.7, 2.6 Hz, 1H), 2.34-2.46 (m, 2H), 2.17-2.27 (m, 1H), 1.83-2.12 (m, 5H), 1.46-1.79 (m, 14H), 0.90-1.44 (m, 13H), 0.68-0.90 (m, 13H), 0.60 (q, J=11.8 Hz, 1H) LCMS: MNa+ (ion type), 1010.4 (ion m/z).

Compound 125: ¹H NMR (DMSO-d₆, 600 MHz): δ (ppm) 5.93-6.63 (m, 5H), 5.61 (dd, J=14.4, 8.5 Hz, 1H), 5.24 (br d, J=4.5 Hz, 1H), 5.07-5.13 (m, 1H), 4.98 (br d, J=4.8 Hz, 1H), 4.46-4.63 (m, 3H), 3.67-4.14 (m, 4H), 2.99-3.60 (m, 18H), 2.79 (br s, 4H), 0.35-2.42 (m, 47H). LCMS: MNa+ (ion type), 1010.4 (ion m/z).

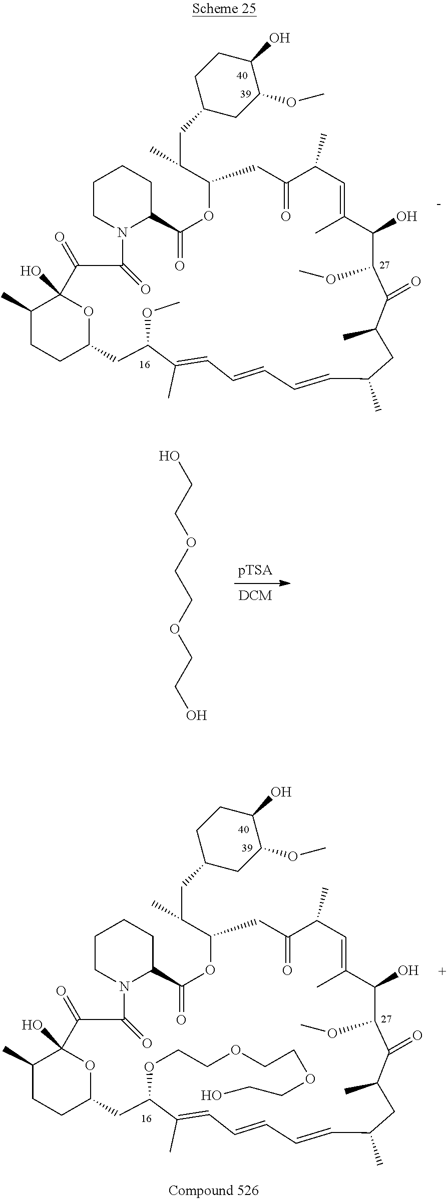

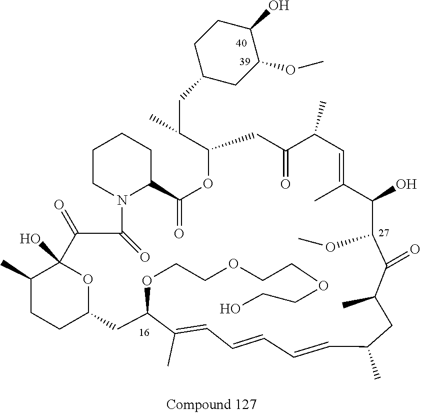

2,2′-[ethane-1,2-diylbis(oxy)]diethanol (18.37 mL, 129 mmol) was added to a solution of (1R,9S,12S,15R,16E,18R,19R,21R,23S,24E,26E,28E,30S,32S,35R)-1,18-dihydroxy-12-{(2R)-1-[(1S,3R,4R)-4-hydroxy-3-methoxycyclohexyl]propan-2-yl}-19,30-dimethoxy-15,17,21,23,29,35-hexamethyl-11,36-dioxa-4-azatricyclo[30.3.1.0˜4,9˜]hexatriaconta-16,24,26,28-tetraene-2,3,10,14,20-pentone (3.00 g, 3.28 mmol) in anhydrous DCM (131 mL). 4-methylbenzenesulfonic acid (2.82 g, 16.4 mmol) was added. The mixture was stirred 60 minutes at room temperature. The mixture was diluted with DCM and neutralized by a saturated solution of NaHCO₃. The phases were separated. The organic phase was washed with water, dried, filtered and concentrated to dryness. The resulting crude mixture was purified by reverse phase chromatography (Uptisphere Strategy C18-Hq 10 um 250×30.0 mm CH₃CN:H₂O gradient 70:30 to 100:0, 277 nm). The main fraction (1.8 g) was purified by SFC separation to afford two fractions.

(1R,9S,12S,15R,16E,18R,19R,21R,23S,24E,26E,28E,30R,32S,35R)-1,18-dihydroxy-30-[2-[2-(2-hydroxyethoxy)ethoxy]ethoxy]-12-[(1R)-2-[(1S,3R,4R)-4-hydroxy-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (444 mg, 13%, white amorphous solid, compound 526) and (1R,9S,12S,15R,16E,18R,19R,21R,23S,24E,26E,28E,30S,32S,35R)-1,18-dihydroxy-30-[2-[2-(2-hydroxyethoxy)ethoxy]ethoxy]-12-[(1R)-2-[(1S,3R,4R)-4-hydroxy-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (288 mg, 3%, white amorphous solid, compound 127).

SFC separation: Column: Princeton 2 Ethylpyridine 5 μm 60 A. Column size: 3 cm I.D.×15 cm L. Mobile phase: CO₂/IpOH 60/40. Flowrate: 100 ml/min. Pressure: 100 Bar. Wave length: UV 277 nm. SFC Equipment: Waters SFC200.

Compound 526: ¹H NMR (DMSO-d₆, 600 MHz) δ 5.9-6.6 (m, 5H), 5.46 (dd, 1H, J=9.6, 14.9 Hz), 5.2-5.3 (m, 1H), 5.09 (br d, 1H, J=10.1 Hz), 5.0-5.1 (m, 1H), 4.93 (br d, 1H, J=6.2 Hz), 4.5-4.6 (m, 2H), 4.0-4.1 (m, 2H), 3.94 (d, 1H, J=4.5 Hz), 3.8-3.8 (m, 1H), 3.4-3.6 (m, 12H), 3.1-3.3 (m, 7H), 2.82 (ddd, 2H, J=4.3, 8.5, 11.2 Hz), 2.73 (dd, 1H, J=2.4, 17.5 Hz), 2.3-2.4 (m, 2H), 2.2-2.3 (m, 1H), 1.8-2.2 (m, 5H), 1.5-1.8 (m, 14H), 0.7-1.5 (m, 28H), 0.59 (q, 1H, J=12.0 Hz) LCMS: MNa+ (ion type), 1054.8 (ion m/z).

Compound 127: ¹H NMR (DMSO-d₆, 600 MHz): δ (ppm) 6.53 (d, J=0.9 Hz, 1H), 6.43 (dd, J=14.0, 11.7 Hz, 1H), 6.13-6.22 (m, 2H), 6.05 (br d, J=10.3 Hz, 1H), 5.60 (dd, J=14.5, 8.4 Hz, 1H), 5.25 (d, J=4.5 Hz, 1H), 5.19-5.24 (m, 1H), 5.10 (ddd, J=8.9, 4.8, 2.7 Hz, 1H), 4.98 (br d, J=5.1 Hz, 1H), 4.57 (d, J=4.3 Hz, 1H), 4.55 (t, J=5.4 Hz, 1H), 4.04 (br d, J=4.1 Hz, 1H), 3.95-4.00 (m, 1H), 3.91 (d, J=4.5 Hz, 1H), 3.80-3.83 (m, 1H), 3.53-3.57 (m, 1H), 3.44-3.52 (m, 8H), 3.40-3.43 (m, 2H), 3.32-3.40 (m, 2H), 3.30-3.31 (m, 1H), 3.30 (s, 3H), 3.18 (s, 3H), 3.12-3.15 (m, 1H), 3.01-3.09 (m, 1H), 2.79-2.86 (m, 1H), 2.75-2.79 (m, 1H), 2.50 (br s, 2H), 2.22-2.31 (m, 1H), 2.15 (br d, J=12.9 Hz, 1H), 1.97-2.04 (m, 1H), 1.87-1.96 (m, 1H), 1.70-1.78 (m, 3H), 1.70 (s, 3H), 1.65 (s, 3H), 1.47-1.63 (m, 7H), 1.32-1.46 (m, 3H), 1.22-1.32 (m, 3H), 1.05-1.21 (m, 4H), 0.98 (br d, J=6.7 Hz, 3H), 0.94-0.97 (m, 1H), 0.92 (d, J=6.6 Hz, 3H), 0.85-0.88 (m, 1H), 0.83 (d, J=6.5 Hz, 3H), 0.79 (br d, J=6.7 Hz, 2H), 0.73 (s, 3H), 0.52-0.60 (m, 1H) LCMS: MNa+ (ion type), 1054.8 (ion m/z).

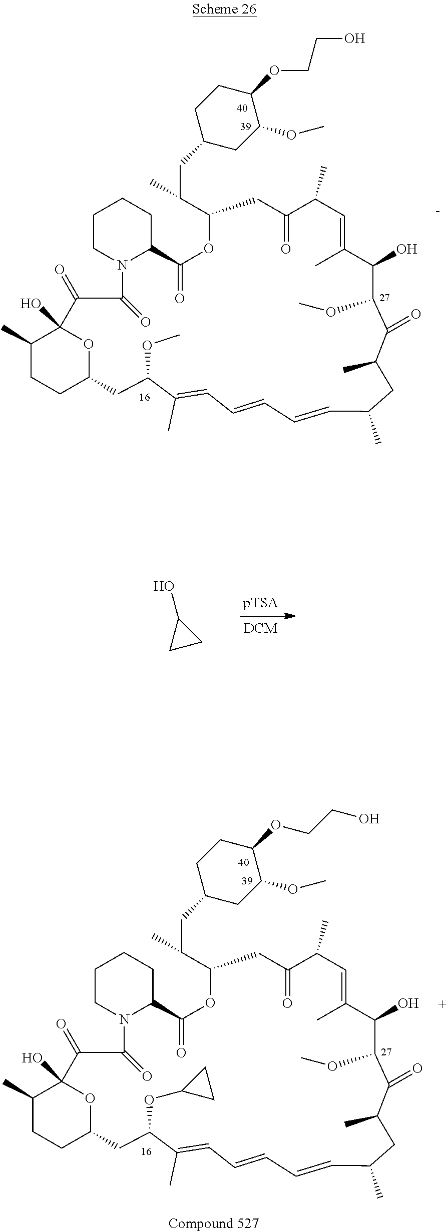

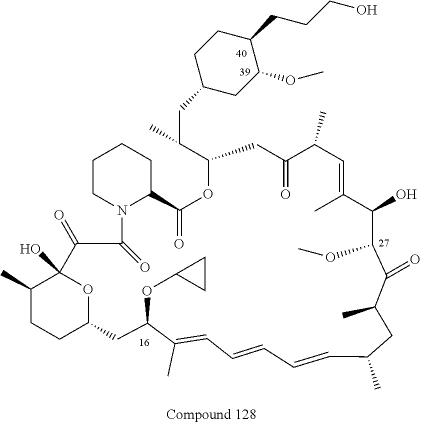

Cyclopropanol (2.6 mL, 41.3 mmol) was added to a solution of (1R,9S,12S,15R,16E,18R,19R,21R,23S,24E,26E,28E,30S,32S,35R)-1,18-dihydroxy-12-{(2R)-1-[(1S,3R,4R)-4-(2-hydroxyethoxy)-3-methoxycyclohexyl]propan-2-yl}-19,30-dimethoxy-15,17,21,23,29,35-hexamethyl-11,36-dioxa-4-azatricyclo[30.3.1.0˜4,9˜]hexatriaconta-16,24,26,28-tetraene-2,3,10,14,20-pentone (1.00 g, 1.04 mmol) in anhydrous DCM (42 mL). The mixture was cooled to −20° C. and 4-methylbenzenesulfonic acid (881 mg, 5.11 mmol) was added. The mixture was stirred 30 minutes at room temperature. The mixture was diluted with DCM and neutralized by a saturated solution of NaHCO3. The phases were separated. The organic phase was washed with water (40 mL), dried, filtered and concentrated to dryness. The resulting crude mixture was purified by reverse phase chromatography (Uptisphere Strategy C18-Hq 10 um 250×30.0 mm CH₃CN:H₂O gradient 70:30 to 100:0, 277 nm). The main fraction was purified by SFC separation to afford two fractions.

(1R,9S,12S,15R,16E,18R,19R,21R,23S,24E,26E,28E,30S,32S,35R)-30-(cyclopropoxy)-1,18-dihydroxy-12-[(1R)-2-[(1S,3R,4R)-4-(2-hydroxyethoxy)-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (381 mg, 36%, white amorphous solid, compound 527) and (1R,9S,12S,15R,16E,18R,19R,21R,23S,24E,26E,28E,30R,32S,35R)-30-(cyclopropoxy)-1,18-dihydroxy-12-[(1R)-2-[(1S,3R,4R)-4-(2-hydroxyethoxy)-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (168 mg, 15%, white amorphous solid, compound 128).

SFC separation: Column: Waters Viridis Ethylpyridine 5 μm 60 A. Column size: 19×250 mm. Mobile phase: CO2/IpOH 80/20. Flowrate: 50 ml/min. Pressure: 100 Bar. Wave length: UV 277 nm. SFC Equipment: Waters SFC200.

Compound 527: ¹H NMR (600 MHz, DMSO-d6) δ 5.91-6.52 (m, 5H), 5.44-5.59 (m, 1H), 5.19-5.32 (m, 1H), 5.03-5.17 (m, 1H), 4.91-5.00 (m, 2H), 4.41-4.46 (m, 1H), 3.99-4.03 (m, 1H), 3.66-3.96 (m, 3H), 3.38-3.55 (m, 5H), 3.31-3.35 (m, 3H), 3.24-3.29 (m, 1H), 3.12-3.21 (m, 31H), 2.95-3.10 (m, 3H), 2.65-2.78 (m, 1H), 2.34-2.48 (m, 2H), 2.18-2.26 (m, 1H), 2.06-2.15 (m, 1H), 1.65-2.04 (m, 13H), 1.50-1.62 (m, 5H), 1.36-1.44 (m, 2H), 1.22-1.33 (m, 4H), 1.10-1.19 (m, 2H), 0.93-1.06 (m, 7H), 0.81-0.91 (m, 6H), 0.62-0.79 (m, 7H), 0.22-0.54 (m, 4H) LCMS: MNa+ (ion type), 1006.6 (ion m/z).

Compound 128: ¹H NMR (600 MHz, DMSO-d6) δ 5.91-6.70 (m, 5H), 5.61-5.74 (m, 1H), 5.18-5.34 (m, 2H), 5.05-5.18 (m, 1H), 4.98 (br d, J=5.43 Hz, 1H), 4.40-4.46 (m, 1H), 4.01-4.09 (m, 1H), 3.80-3.99 (m, 3H), 3.42-3.59 (m, 5H), 3.30-3.39 (m, 4H), 3.13-3.28 (m, 4H), 2.92-3.10 (m, 3H), 2.53-2.86 (m, 3H), 2.25-2.32 (m, 1H), 2.06-2.19 (m, 1H), 1.87-2.03 (m, 3H), 1.65-1.71 (m, 6H), 1.49-1.61 (m, 6H), 1.40-1.45 (m, 1H), 1.20-1.37 (m, 5H), 1.17 (br s, 1H), 1.06-1.15 (m, 4H), 0.93-1.05 (m, 7H), 0.70-0.90 (m, 11H), 0.61-0.66 (m, 1H), 0.22-0.53 (m, 4H) LCMS: MNa+ (ion type), 1006.7 (ion m/z).

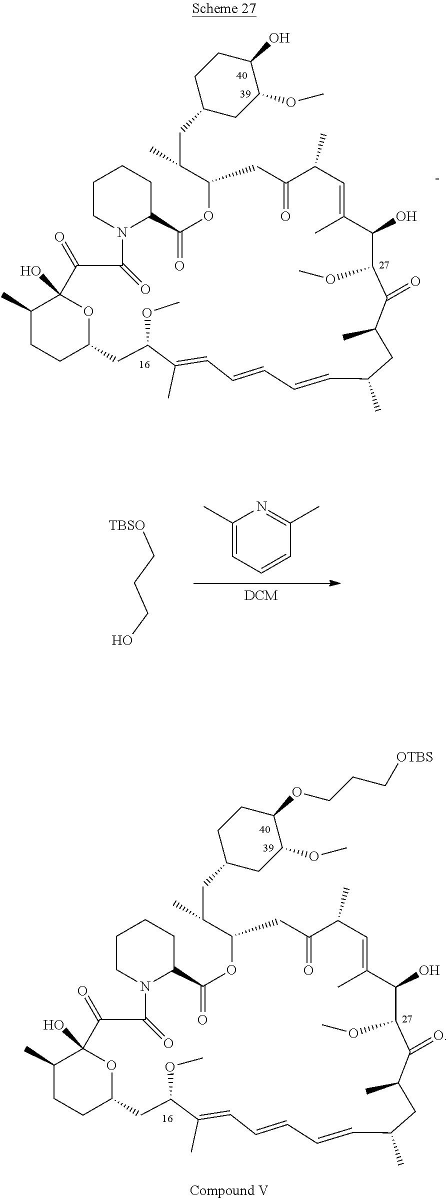

Preparation of (1R,9S,12S,15R,16E,18R,19R,21R,23S,24E,26E,28E,30S,32S,35R)-12-[(1R)-2-[(1S,3R,4R)-4-[3-[tert-butyl(dimethyl)silyl]oxypropoxy]-3-methoxy-cyclohexyl]-1-methyl-ethyl]-1,18-dihydroxy-19,30-dimethoxy-15,17,21,23,29,35-hexamethyl-11,36-dioxa-4-azatricyclo[30.3.1.0 A4,9] hexatriaconta-16,24,26,28-tetraene-2,3,10,14,20-pentone (Compound V). 3-[tert-butyl(dimethyl)silyl]oxypropyl trifluoromethanesulfonate (1799 mg, 5.58 mmol) was added to a mixture of Sirolimus (1.7 g, 1.86 mmol) and N-ethyl-N-isopropyl-propan-2-amine (1.8 mL, 10.2 mmol) previously dissolved in dry Toluene (6.9 mL) under argon. After 3 hours of stirring at 60° C., the crude mixture was concentrated and purified on silica gel by flash column chromatography (Cyclohexane/Ethylacetate 100:0 to 70:30) to afford the desired product as an amorphous white solid (799 mg). Yield 39%. LCMS: MNa+ (ion type), 1108.7 (ion m/z).

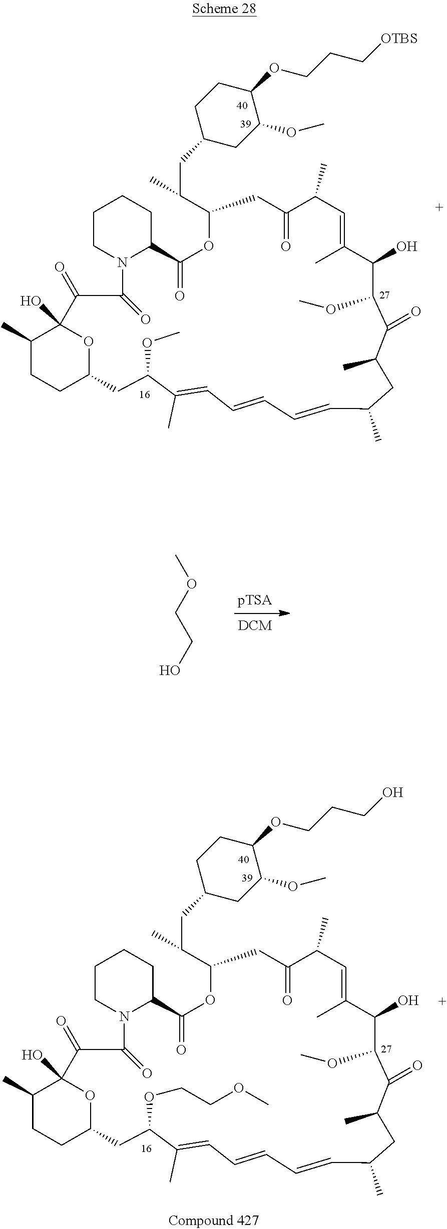

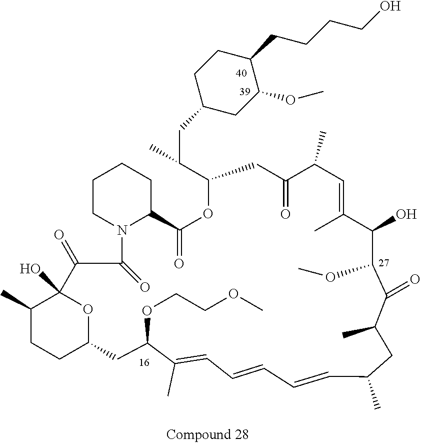

2-methoxyethanol (81 mL, 1.02 mmol) was added to a solution of (1R,9S,12SR,15R,16E,18R,19R,21R,23S,24E,26E,28E,30S,32S,35R)-12-[(1R)-2-[(1S,3R,4R)-4-[3-[tertbutyl(dimethyl)silyl]oxypropoxy]-3-methoxy-cyclohexyl]-1-methyl-ethyl]-1,18-dihydroxy-19,30-dimethoxy-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (3.03 g, 2.79 mmol) in anhydrous DCM (30 mL). 4-methylbenzenesulfonic acid (2.40 g, 13.9 mmol) was added. The mixture was stirred 60 minutes at room temperature. The mixture was diluted with DCM and neutralized by a saturated solution of NaHCO3. The phases were separated. The organic phase was washed with water (40 mL), dried, filtered and concentrated to dryness. The resulting crude mixture was purified by reverse phase chromatography (Uptisphere Strategy C18-Hq 10 um 250×30.0 mm CH₃CN:H₂O gradient 60:40 to 100:0, 277 nm). The main fraction was purified by SFC separation to afford two fractions.

(1R,9S,12S,15R,16E,18R,19R,21R,23S,24E,26E,28E,30S,32S,35R)-1,18-dihydroxy-12-[(1R)-2-[(1S,3R,4R)-4-(3-hydroxypropoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.04,9]hexatriaconta-16,24,26,28-tetraene-2,3,10,14,20-pentone (66 mg, 3%, white amorphous solid, compound 427) and (1R,9S,12S,15R,16E,18R,19R,21R,23S,24E,26E,28E,30R,32S,35R)-1,18-dihydroxy-12-[(1R)-2-[(1S,3R,4R)-4-(3-hydroxypropoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.04,9]hexatriaconta-16,24,26,28-tetraene-2,3,10,14,20-pentone (19.7 mg, 1%, white amorphous solid, compound 28).

SFC separation: Column: Waters Viridis Ethylpyridine 5 μm 60 A. Column size: 19×250 mm. Mobile phase: CO2/IpOH 80/20. Flowrate: 50 ml/min. Pressure: 100 Bar. Wave length: UV 277 nm. SFC Equipment: Waters SFC200.

Compound 427: ¹H NMR (600 MHz, DMSO-d₆, 300K) δ ppm ¹H NMR (DMSO-d₆, 500 MHz): δ (ppm) 6.45 (d, J=1.7 Hz, 1H), 6.32-6.43 (m, 1H), 6.08-6.26 (m, 3H), 5.46 (dd, J=14.8, 9.7 Hz, 1H), 5.26 (d, J=4.4 Hz, 1H), 5.09 (br d, J=10.0 Hz, 1H), 4.98 (dt, J=7.6, 4.0 Hz, 1H), 4.94 (br d, J=5.4 Hz, 1H), 4.30 (br t, J=5.1 Hz, 1H), 3.97-4.08 (m, 2H), 3.94 (d, J=4.6 Hz, 1H), 3.79 (br d, J=11.5 Hz, 1H), 3.53-3.60 (m, 1H), 3.34-3.52 (m, 6H), 3.33 (s, 3H), 3.25-3.30 (m, 2H), 3.24 (s, 3H), 3.16 (s, 3H), 2.89-3.05 (m, 2H), 2.73 (br d, J=15.4 Hz, 1H), 2.30-2.44 (m, 2H), 2.17-2.28 (m, 1H), 2.10 (br d, J=13.0 Hz, 1H), 1.99-2.06 (m, 1H), 1.81-1.98 (m, 4H), 1.74 (s, 3H), 1.66-1.70 (m, 2H), 1.64 (s, 3H), 1.50-1.63 (m, 6H), 1.35-1.47 (m, 2H), 1.21-1.33 (m, 3H), 1.09-1.20 (m, 2H), 1.04 (d, J=5.9 Hz, 3H), 1.00-1.08 (m, 2H), 0.98 (br d, J=6.6 Hz, 3H), 0.91-0.96 (m, 1H), 0.87 (d, J=6.6 Hz, 3H), 0.83 (d, J=6.4 Hz, 3H), 0.79-0.81 (m, 1H), 0.78 (d, J=6.8 Hz, 3H), 0.73 (d, J=6.8 Hz, 3H), 0.65 (q, J=12.0 Hz, 1H)

Compound 28: ¹H NMR (600 MHz, DMSO-d₆, 300K) δ ppm 5.49-6.45 (m, 5H), 4.98-5.57 (m, 5H), 4.10-4.51 (m, 3H), 3.57-3.94 (m, 8H), 3.18-3.57 (m, 16H), 2.97-3.17 (m, 2H), 2.51-2.76 (m, 3H), 1.89-2.42 (m, 8H), 1.24-1.88 (m, 18H), 0.65-1.18 (m, 14H). LCMS: MNa+ (ion type), 1038.7 (ion m/z).

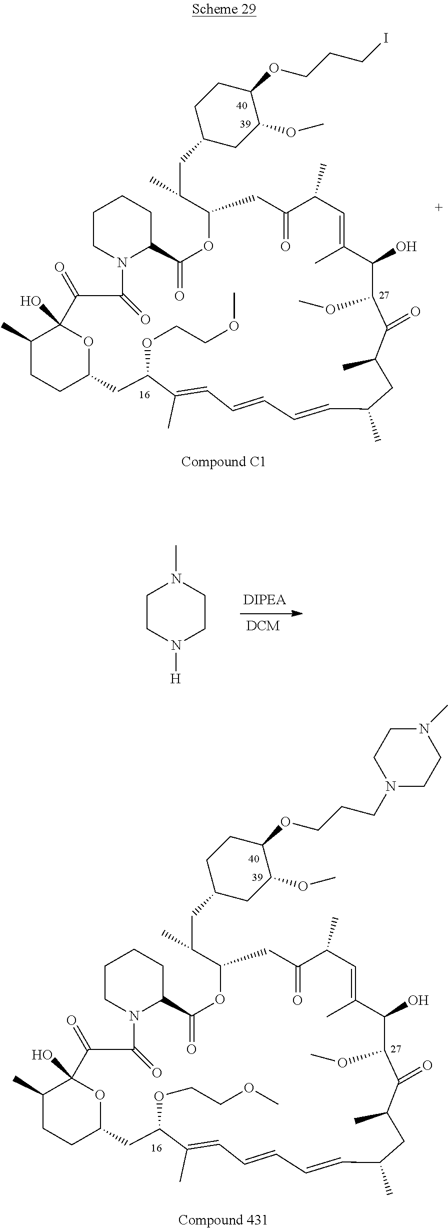

To a solution of (1R,9S,12S,15R,16E,18R,19R,21R,23S,24E,26E,28E,30S,32S,35R)-1,18-dihydroxy-12-[(1R)-2-[(1S,3R,4R)-4-(3-iodopropoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (compound C1) (300 mg, 0.266 mmol) in anhydrous DCM (1.66 mL) was added N-ethyl-N-isopropyl-propan-2-amine (139 uL, 0.799 mmol) then methylpiperazine (36 uL, 0.319 mmol). The reaction mixture was stirred for 6 hours at room temperature under argon. The reaction mixture was diluted with DCM and quenched with aqueous saturated NH4Cl solution (pH=6). The organic phase was washed with water and dried. The crude was then purified by silica gel flash column chromatography (100/0 to 70/30 of EtOAc/MeOH:Et3N (50:50). The fraction of interest were then purified by silica gel flash column chromatography (100/0 to 80/20 of DCM/MeOH) to afford the desired product (1R,9S,12S,15R,16E,18R,19R,21R,23S,24E,26E,28E,30S,32S,35R)-1,18-dihydroxy-19-methoxy-30-(2-methoxyethoxy)-12-[(1R)-2-[(1S,3R,4R)-3-methoxy-4-[3-(4-methylpiperazin-1-yl)propoxy]cyclohexyl]-1-methyl-ethyl]-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (137 mg, 47%, compound 431).

Compound 431: MS (ES+, m/z): 1098.7 [M+H]+. ¹H NMR (600 MHz, DMSO-d₆) δ 5.88-6.53 (m, 5H), 5.42-5.70 (m, 1H), 5.20-5.29 (m, 1H), 5.06-5.15 (m, 1H), 4.92-5.05 (m, 2H), 3.98-4.08 (m, 2H), 3.83-3.96 (m, 1H), 3.67-3.81 (m, 1H), 3.32-3.57 (m, 9H), 3.10-3.28 (m, 9H), 2.91-3.05 (m, 2H), 2.70-2.76 (m, 1H), 2.16-2.48 (m, 13H), 2.16 (br s, 3H), 1.97-2.13 (m, 3H), 1.83-1.96 (m, 4H), 1.73 (s, 2H), 1.52-1.70 (m, 12H), 1.35-1.45 (m, 2H), 1.21-1.33 (m, 4H), 1.14-1.20 (m, 1H), 0.93-1.11 (m, 8H), 0.80-0.89 (m, 6H), 0.72-0.79 (m, 5H), 0.63-0.69 (n, 1H) LCMS: MH+ (ES+), 1098.7 (ion m/z).

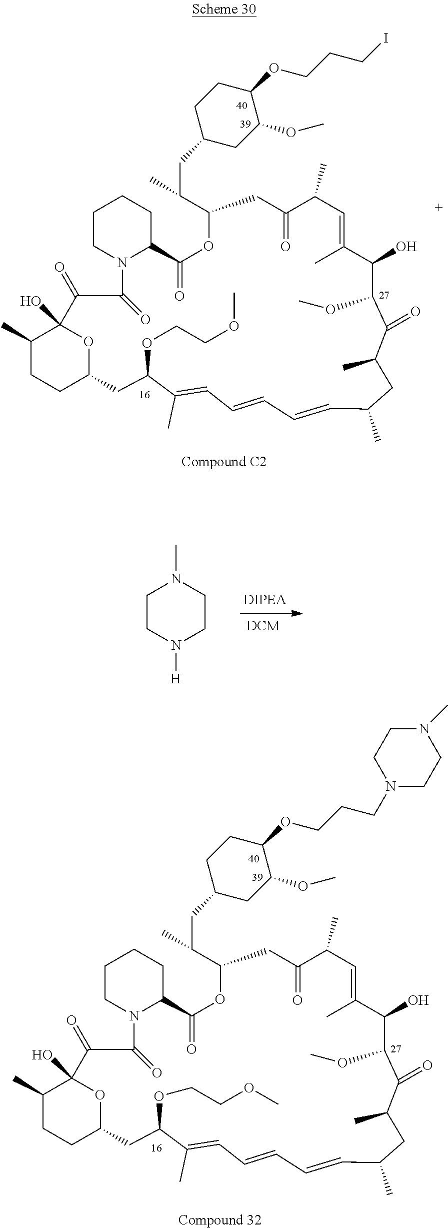

To a solution of ((1R,9S,12S,15R,16E,18R,19R,21R,23S,24E,26E,28E,30R,32S,35R)-1,18-dihydroxy-12-[(1R)-2-[(1S,3R,4R)-4-(3-iodopropoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.04,9]hexatriaconta-16,24,26,28-tetraene-2,3,10,14,20-pentone (compound C1) (502.9 mg, 0.446 mmol) in anhydrous DCM (2.79 mL) was added N-ethyl-N-isopropyl-propan-2-amine (233 uL, 1.34 mmol) then 1-methylpiperazine (60 uL, 0.535 mmol). The reaction mixture was stirred for 3 hours at room temperature under argon. The reaction mixture was diluted with DCM and quenched with aqueous saturated NH4Cl solution (pH=6). The organic phase was washed with water and dried. The crude was then purified by silica gel flash column chromatography (100/0 to 70/30 of EtOAc/MeOH:Et3N (50:50). The fraction of interest were then purified by silica gel flash column chromatography (100/0 to 80/20 of DCM/MeOH) to afford the desired product (1R,9S,12S,15R,16E,18R,19R,21R,23S,24E,26E,28E,30R,32S,35R)-1,18-dihydroxy-19-methoxy-30-(2-methoxyethoxy)-12-[(1R)-2-[(1S,3R,4R)-3-methoxy-4-[3-(4-methylpiperazin-1-yl)propoxy]cyclohexyl]-1-methyl-ethyl]-15,17,21,23,29,35-hexamethyl-11,36-dioxa-4-azatricyclo[30.3.1.04,9]hexatriaconta-16,24,26,28-tetraene-2,3,10,14,20-pentone (78.9 mg, 15%, compound 32).

Compound 32: MS (ES+, m/z): 1098.7 [M+H]+. ¹H NMR (600 MHz, DMSO-d₆) δ 5.89-6.61 (m, 5H), 5.39-5.67 (m, 1H), 5.04-5.38 (m, 4H), 4.89-5.02 (m, 1H), 3.89-4.07 (m, 2H), 3.77-3.87 (m, 1H), 3.65-3.75 (m, 1H), 3.32-3.51 (m, 7H), 2.91-3.07 (m, 4H), 2.64-2.83 (m, 3H), 2.51-2.63 (m, 2H), 2.26-2.44 (m, 9H), 2.07-2.23 (m, 6H), 1.86-2.03 (m, 4H), 1.54-1.72 (m, 18H), 1.47-1.52 (m, 2H), 1.22-1.41 (m, 7H), 1.04-1.11 (m, 5H), 0.90-1.02 (m, 10H), 0.72-0.88 (m, 10H), 0.61-0.68 (m, 1H) LCMS: MH+ (ES+), 1098.7 (ion m/z).

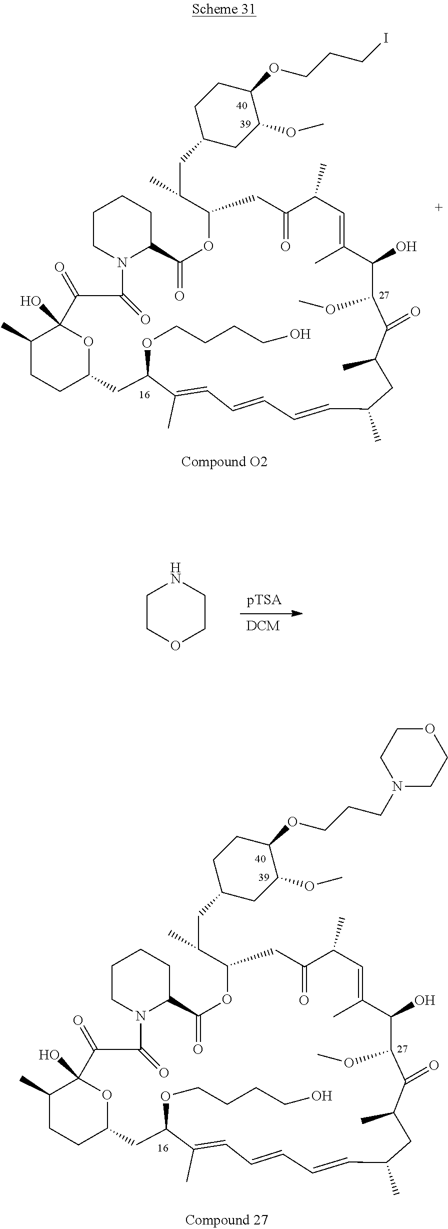

(1R,9S,12SR,15R,16E,18R,19R,21R,23S,24E,26E,28E,30R,32S,35R)-1,18-dihydroxy-30-(4-hydroxybutoxy)-12-[(1R)-2-[(1S,3R,4R)-4-(3-iodopropoxy)-3-methoxy-cyclohexyl]-1-methyl-ethyl]-19-methoxy-15,17,21,23,29,35-hexamethyl-11,36-dioxa-4-azatricyclo[30.3.1.04,9]hexatriaconta-16,24,26,28-tetraene-2,3,10,14,20-pentone (334 mg, 0.29 mmol) in dry DCM (1.33 mL) was added N-ethyl-N-isopropyl-propan-2-amine (113 uL, 0.88 mmol) then morpholine (22 uL, 0.25 mmol). The reaction mixture was stirred at room temperature for 24 hours. The mixture was then diluted with DCM and aqueous HCl 1N was added until pH=5. The organic phase was washed with water, dried and concentrated to dryness and purified over silica gel flash column chromatography (100/0 to 85/15 of EtOAc/MeOH:Et3N (50:50). The fractions of interest were purified over silica gel flash column chromatography (DCM/MeOH, 100/0 to 90/10) to yield (1R,9S,12S,15R,16E,18R,19R,21R,23S,24E,26E,28E,30R,32S,35R)-1,18-dihydroxy-30-(4-hydroxybutoxy)-19-methoxy-12-[(1R)-2-[(1S,3R,4R)-3-methoxy-4-(3-morpholinopropoxy)cyclohexyl]-1-methyl-ethyl]-15,17,21,23,29,35-hexamethyl-11,36-dioxa-4-azatricyclo[30.3.1.04,9]hexatriaconta-16,24,26,28-tetraene-2,3,10,14,20-pentone (110 mg, 34%, Compound 27).

Compound 27: ¹H NMR (600 MHz, DMSO-d6) δ 5.86-6.59 (m, 5H), 5.43-5.64 (m, 1H), 4.89-5.27 (m, 4H), 4.29-4.43 (m, 1H), 3.60-4.09 (m, 4H), 3.45-3.58 (m, 7H), 3.34-3.41 (m, 2H), 3.32 (br d, J=5.28 Hz, 4H), 3.10-3.20 (m, 5H), 2.93-3.01 (m, 2H), 2.61-2.83 (m, 3H), 2.26-2.35 (m, 7H), 0.64-2.20 (m, 53H) LCMS: MH+ (ion type), 1099.6 (ion m/z).

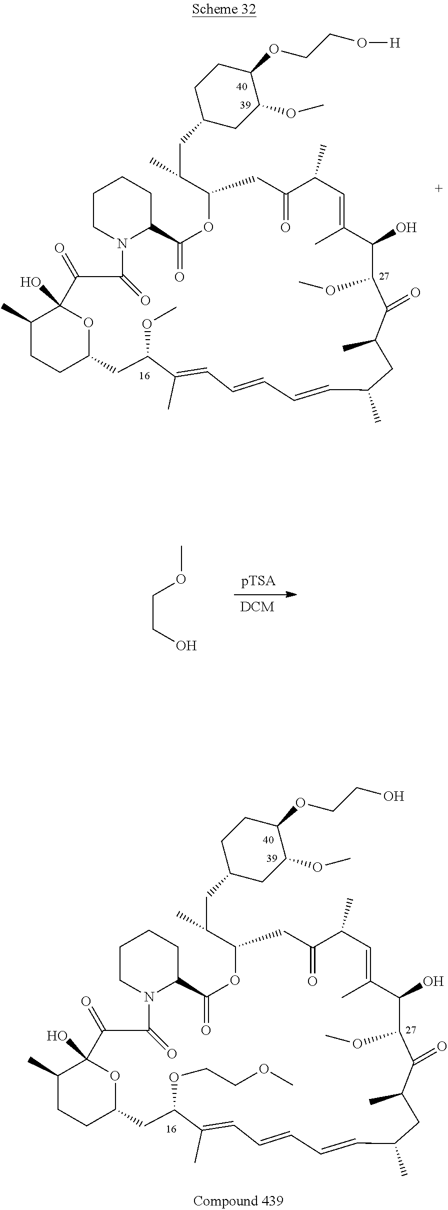

To a solution of everolimus (1.50 g, 1.57 mmol) in anhydrous DCM (19,568 mL) was added 2-methoxyethanol (45 mL, 0.573 mol) then 4-methylbenzenesulfonic acid (1.35 g, 7.83 mmol). The reaction mixture was stirred for 1 h at room temperature. The reaction mixture was neutralized with saturated NaHCO₃ aqueous and extracted with DCM. The organic phase was washed with water (60 mL), dried, filtered and concentrated to dryness. The resulting crude mixture was purified by reverse phase chromatography (Uptisphere Strategy C18-Hq 10 um 250×30.0 mm CH3CN:H2O gradient 60:40 to 100:0 in 25 min, 277 nm). The main fraction (540 mg) was purified by SFC separation to afford (1R,9S,12S,15R,16E,18R,19R,21R,23S,24E,26E,28E,30R,32S,35R)-1,18-dihydroxy-12-[(1R)-2-[(1S,3R,4R)-4-(2-hydroxyethoxy)-3-methoxy-cyclohexyl]-1-methyl-ethyl]-19-methoxy-30-(2-methoxyethoxy)-15,17,21,23,29,35-hexamethyl-11,36-dioxa-4-azatricyclo[30.3.1.0\hat{ }4,9]hexatriaconta-16,24,26,28-tetraene-2,3,10,14,20-pentone (compound 439,231 mg, 15%, white amorphous solid).

SFC separation: Column: Princeton 2 Ethylpyridine. 5 μm 60 A. Column size: 3 cm I.D.×15 cm L. Mobile phase: CO2/IpOH 85/15. Flowrate: 50 ml/min. Pressure: 100 Bar. Wave length: UV 277 nm. SFC Equipment: Waters SFC200.

Compound 439: ¹H NMR (DMSO-d₆, 600 MHz): δ (ppm) 5.87-6.60 (m, 5H), 5.61 (dd, J=14.3, 8.5 Hz, 1H), 5.04-5.53 (m, 3), 4.99 (d, J=5.8 Hz, 1H), 4.95-4.89 (m, 1H), 4.43 (t, J=5.5 Hz, 1H), 3.91-4.15 (2H, m), 3.78-3.92 (m, 1H), 3.62-3.78 (m, 1H), 3.35-3.59 (m, 3H), 3.26-3.34 (m 7H), 3.10-3.26 (m, 3H), 2.89-3.09 (m, 4H), 2.59-2.85 (m, 2H), 2.34-2.43 (m, 1H), 2.20-2.33 (m, 1H), 1.81-2.19 (m, 4H), 1.45-1.81 (m, 6H), 1.19-1.45 (m, 3H), 0.89-1.19 (m, 5H), 0.59-0.89 (5H, m). LCMS: MNa+ (ion type), 1024.6 (ion m/z).

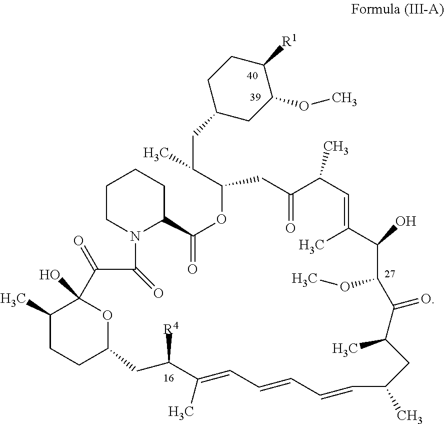

TABLE 1
with Fromula (III-A) core:

| No. # | R¹ | R⁴ |
|---|---|---|
| 1 | 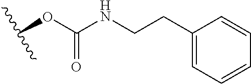 | 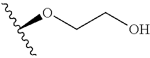 |
| 2 | 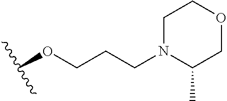 | 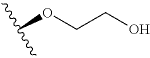 |
| 3 | 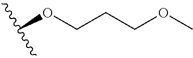 | 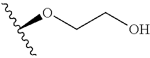 |
| 4 | 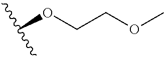 | 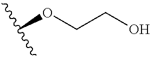 |
| 5 | 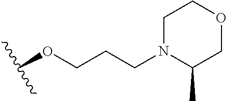 | 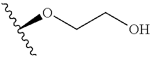 |
| 6 | 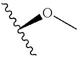 | 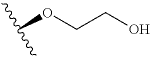 |
| 7 | 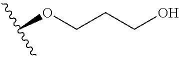 | 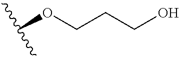 |
| 8 | 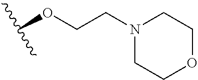 | 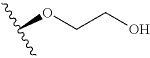 |
| 9 | 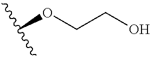 | 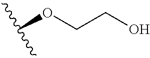 |
| 10 | 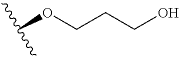 | 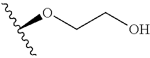 |
| 11 | 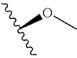 | 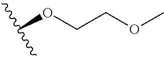 |
| 12 | 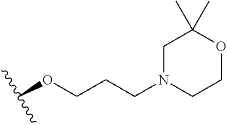 | 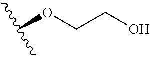 |
| 13 | 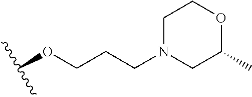 | 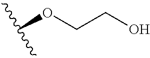 |
| 14 | 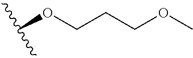 | 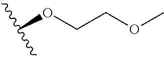 |
| 15 | 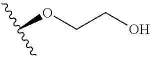 | 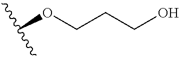 |
| 16 | 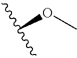 | 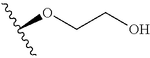 |
| 17 | 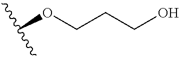 | 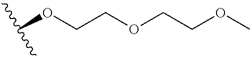 |
| 18 | 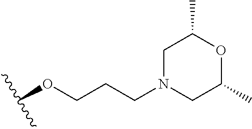 | 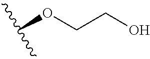 |
| 19 | 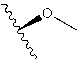 | 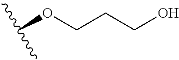 |
| 20 | 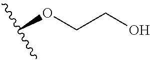 | 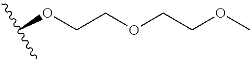 |
| 21 | 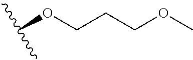 | 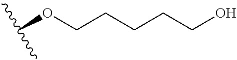 |
| 22 | 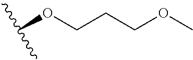 | 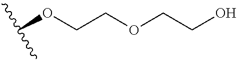 |
| 23 | 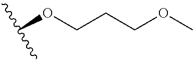 | 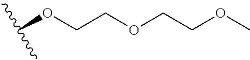 |
| 24 | 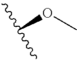 | 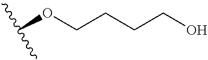 |
| 25 | 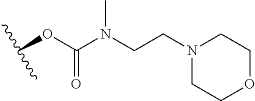 | 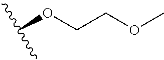 |
| 26 | 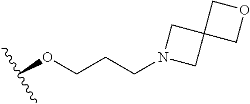 | 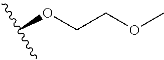 |
| 27 | 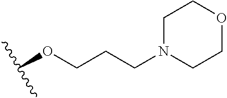 | 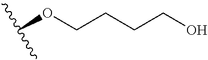 |
| 28 | 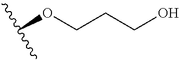 | 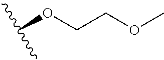 |
| 29 | 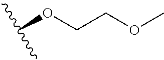 | 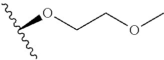 |
| 30 | 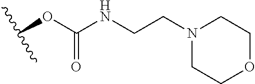 | 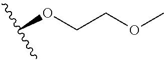 |
| 31 | 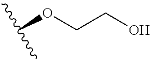 | 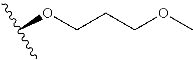 |
| 32 | 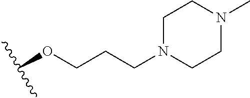 | 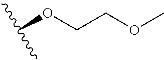 |
| 33 | 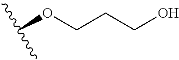 | 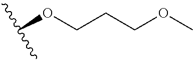 |
| 34 | 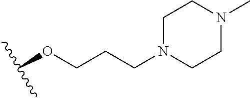 | 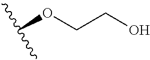 |
| 35 | 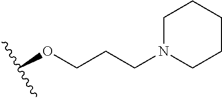 | 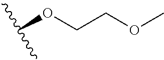 |
| 36 | 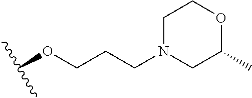 | 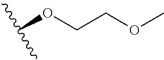 |
| 37 | 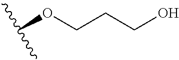 | 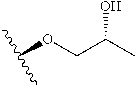 |
| 38 | 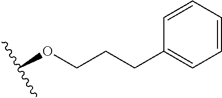 | 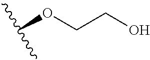 |
| 39 | 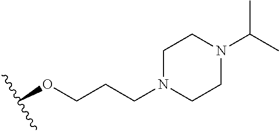 | 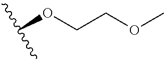 |
| 40 | 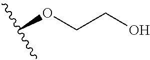 | 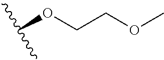 |
| 41 | 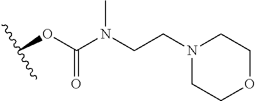 | 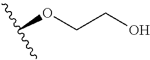 |
| 42 | 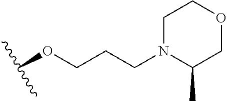 | 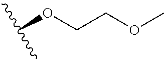 |
| 43 | 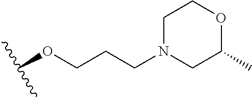 | 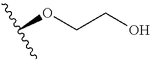 |
| 44 | 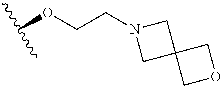 | 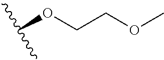 |
| 45 | 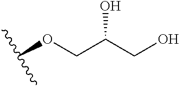 | 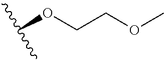 |
| 46 | 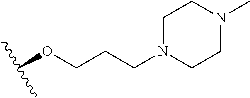 | 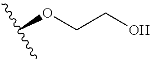 |
| 47 | 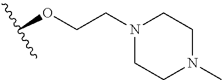 | 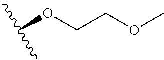 |
| 48 | 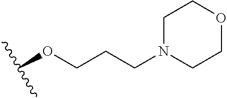 | 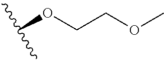 |
| 49 | 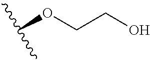 | 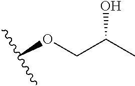 |
| 50 | 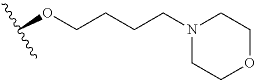 | 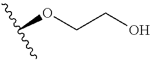 |
| 51 | 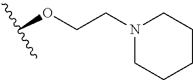 | 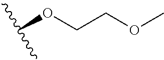 |
| 52 | 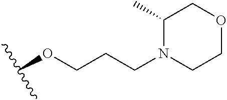 | 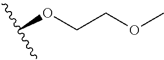 |
| 53 | 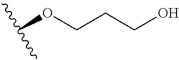 | 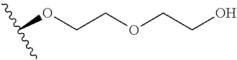 |
| 54 | 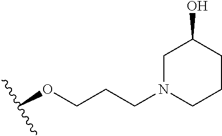 | 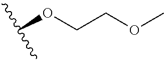 |
| 55 | 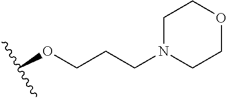 | 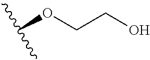 |
| 56 | 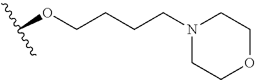 | 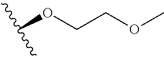 |
| 57 | 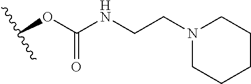 | 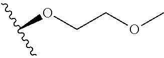 |
| 58 | 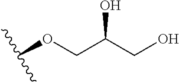 | 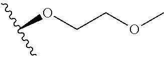 |
| 59 | 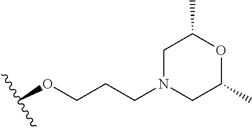 | 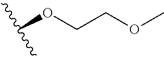 |
| 60 | 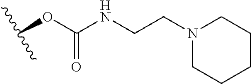 | 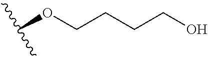 |
| 61 | 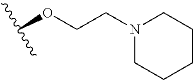 | 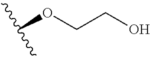 |
| 62 | 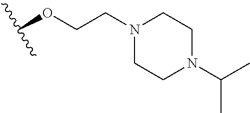 | 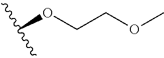 |
| 63 | 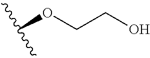 | 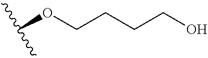 |
| 64 | 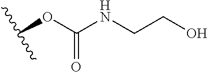 | 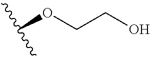 |
| 65 | 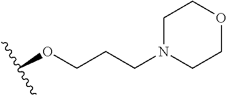 | 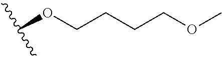 |
| 66 | 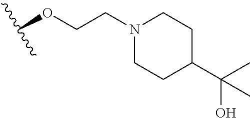 | 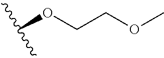 |
| 67 | 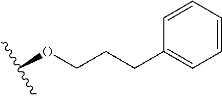 | 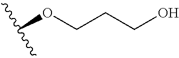 |
| 68 | 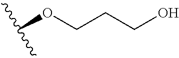 | 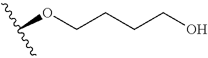 |
| 69 | 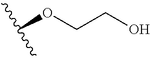 | 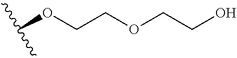 |
| 70 | 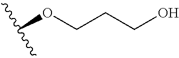 | 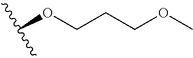 |
| 71 | 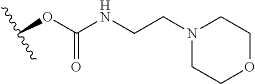 | 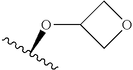 |
| 72 | 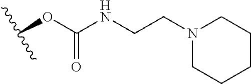 | 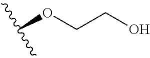 |
| 73 | 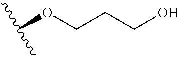 | 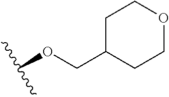 |
| 74 | 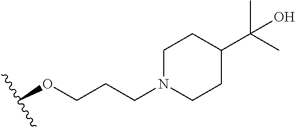 | 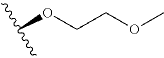 |
| 75 | 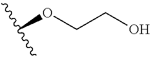 | 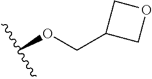 |
| 76 | 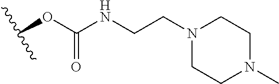 | 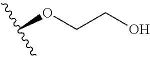 |
| 77 | 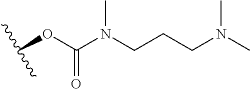 | 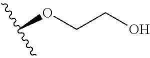 |
| 78 | 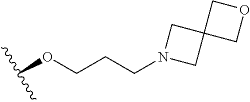 | 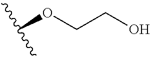 |
| 79 | 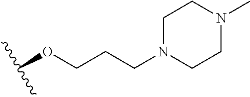 | 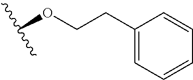 |
| 80 | 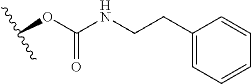 | 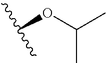 |
| 81 | 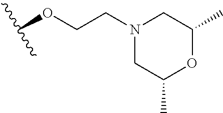 | 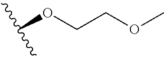 |
| 82 | 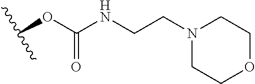 | 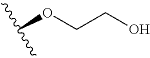 |
| 83 | 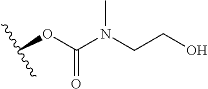 | 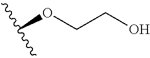 |
| 84 | 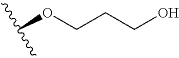 | 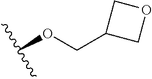 |
| 85 | 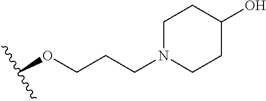 | 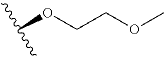 |
| 86 | 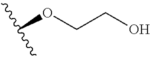 | 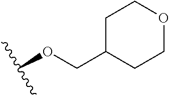 |
| 87 | 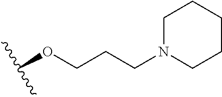 | 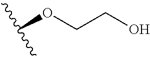 |
| 88 | 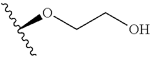 | 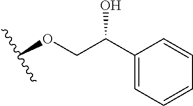 |
| 89 | 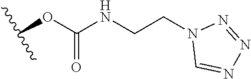 | 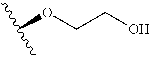 |
| 90 | 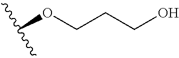 | 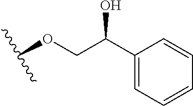 |
| 91 | 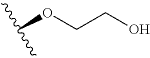 | 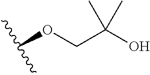 |
| 92 | 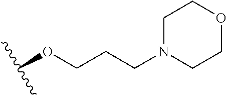 | 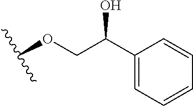 |
| 93 | 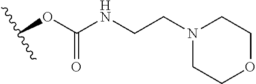 | 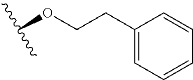 |
| 94 | 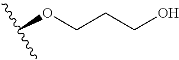 | 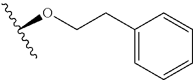 |
| 95 | 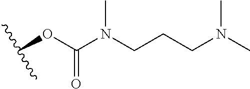 | 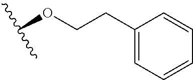 |
| 96 | 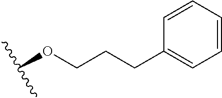 | 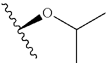 |
| 97 | 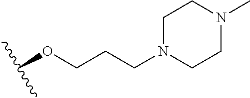 | 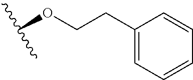 |
| 98 | 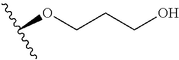 | 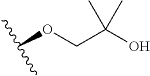 |
| 99 | 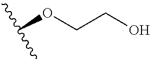 | 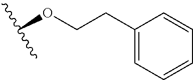 |
| 100 | 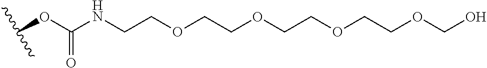 | 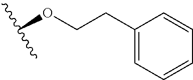 |
| 101 | 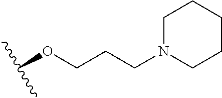 | 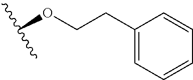 |
| 102 | 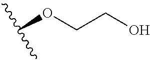 | 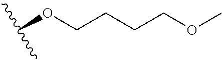 |
| 103 | 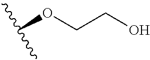 | 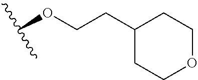 |
| 104 | 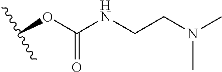 | 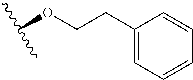 |
| 105 | 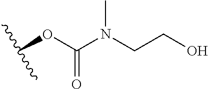 | 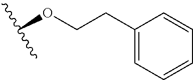 |
| 106 | 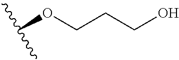 | 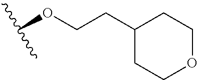 |
| 107 | 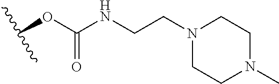 | 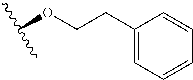 |
| 108 | 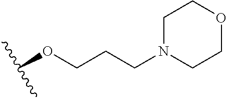 | 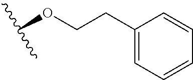 |
| 109 | 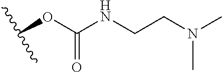 | 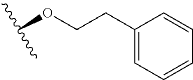 |
| 110 | 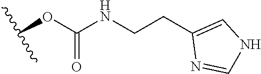 | 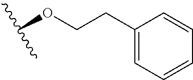 |
| 111 | 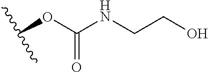 | 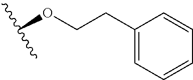 |
| 112 | 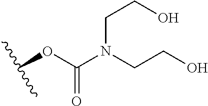 | 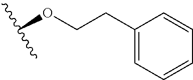 |
| 113 | 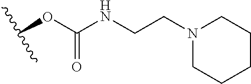 | 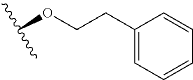 |
| 114 | 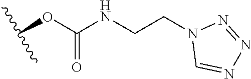 | 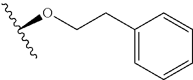 |
| 115 | 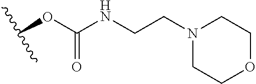 | 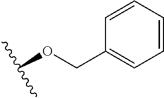 |
| 116 | 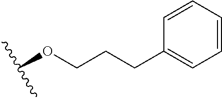 | 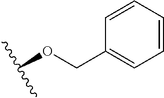 |
| 117 | 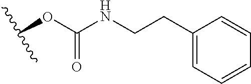 | 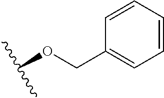 |
| 118 | 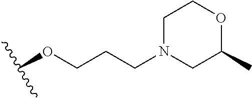 | 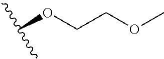 |
| 119 | 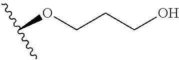 | 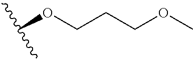 |
| 120 | 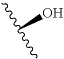 | 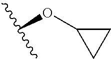 |
| 121 | 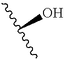 | 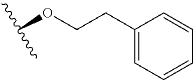 |
| 122 | 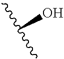 | 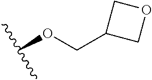 |
| 123 | 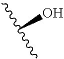 | 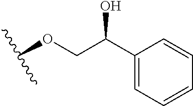 |
| 124 | 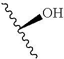 | 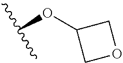 |
| 125 | 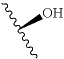 | 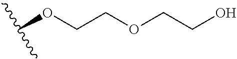 |
| 126 | 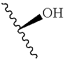 | 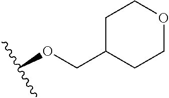 |
| 127 | 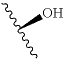 | 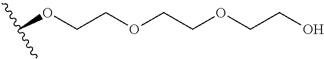 |
| 128 | 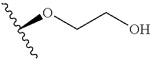 | 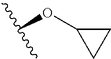 |
| 129 | 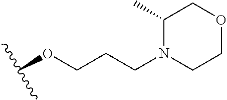 | 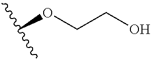 |
| 130 | 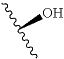 | 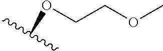 |

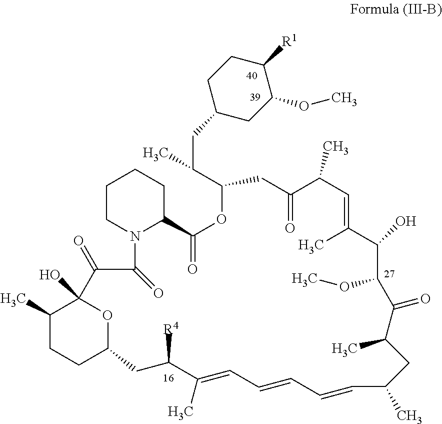

Table 2 with Formula (III-B) core:

| No. # | R¹ | R⁴ |
|---|---|---|
| 200 | 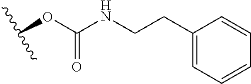 | 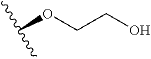 |
| 201 | 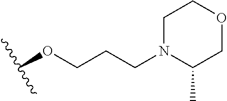 | 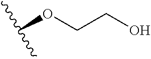 |
| 202 | 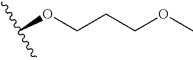 | 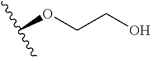 |
| 203 | 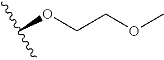 | 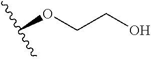 |
| 204 | 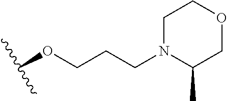 | 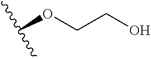 |
| 205 | 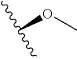 | 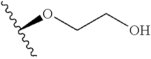 |
| 206 | 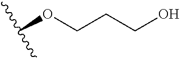 | 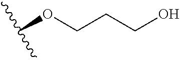 |
| 207 | 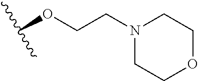 | 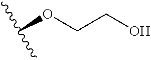 |
| 208 | 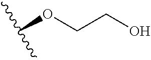 | 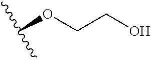 |
| 209 | 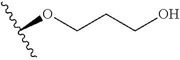 | 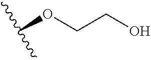 |
| 210 | 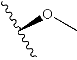 | 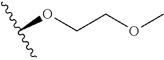 |
| 211 | 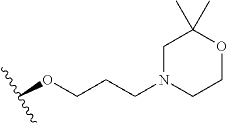 | 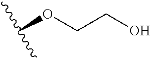 |
| 212 | 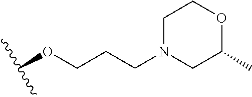 | 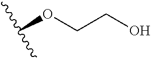 |
| 213 | 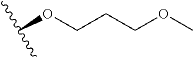 | 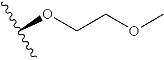 |
| 214 | 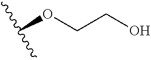 | 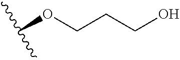 |
| 215 | 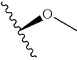 | 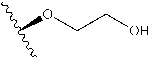 |
| 216 | 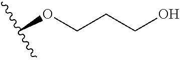 | 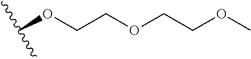 |
| 217 | 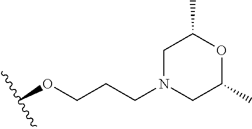 | 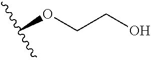 |
| 218 | 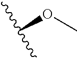 | 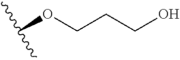 |
| 219 | 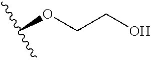 | 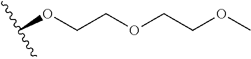 |
| 220 | 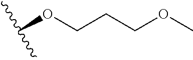 | 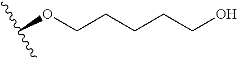 |
| 221 | 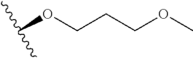 | 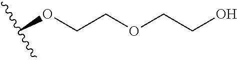 |
| 222 | 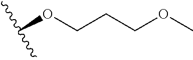 | 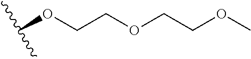 |
| 223 | 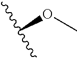 | 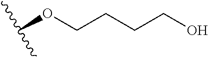 |
| 224 | 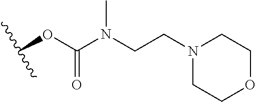 | 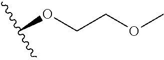 |
| 225 | 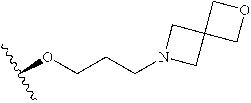 | 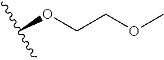 |
| 226 | 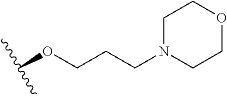 | 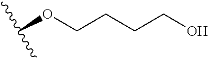 |
| 227 | 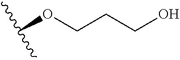 | 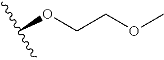 |
| 228 | 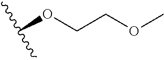 | 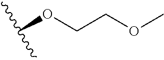 |
| 229 | 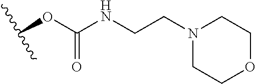 | 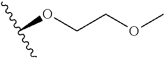 |
| 230 | 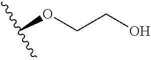 | 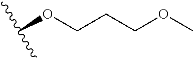 |
| 231 | 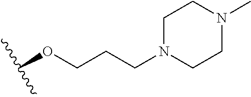 | 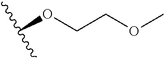 |
| 232 | 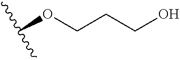 | 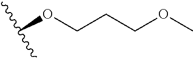 |
| 233 | 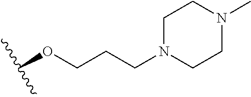 | 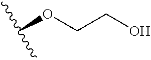 |
| 234 | 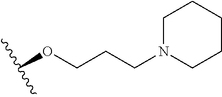 | 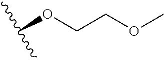 |
| 235 | 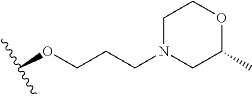 | 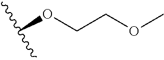 |
| 236 | 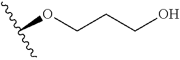 | 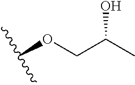 |
| 237 | 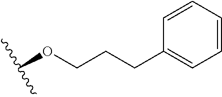 | 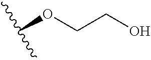 |
| 238 | 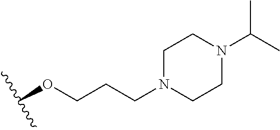 | 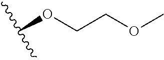 |
| 239 | 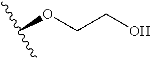 | 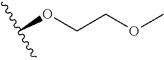 |
| 240 | 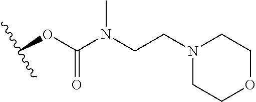 | 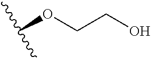 |
| 241 | 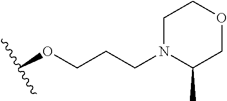 | 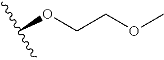 |
| 242 | 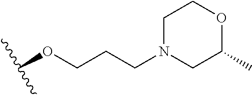 | 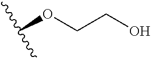 |
| 243 | 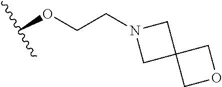 | 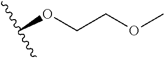 |
| 244 | 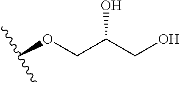 | 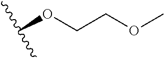 |
| 245 | 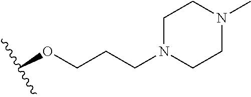 | 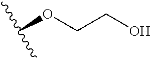 |
| 246 | 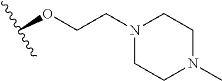 | 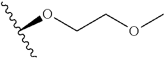 |
| 247 | 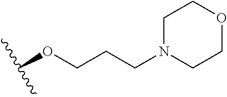 | 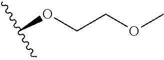 |
| 248 | 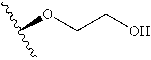 | 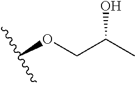 |
| 249 | 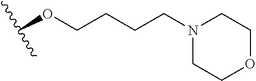 | 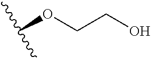 |
| 250 | 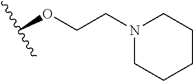 | 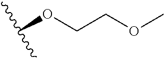 |
| 251 | 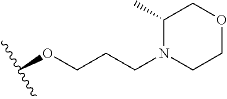 | 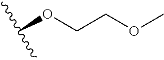 |
| 252 | 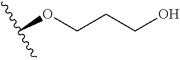 | 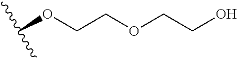 |
| 253 | 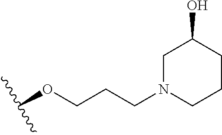 | 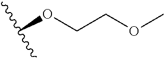 |
| 254 | 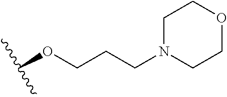 | 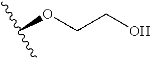 |
| 255 | 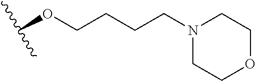 | 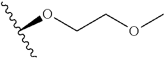 |
| 256 | 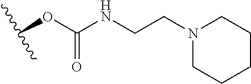 | 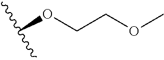 |
| 257 | 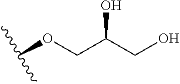 | 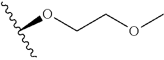 |
| 258 | 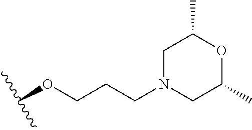 | 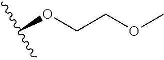 |
| 259 | 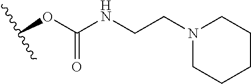 | 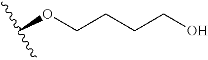 |
| 260 | 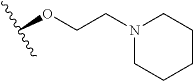 | 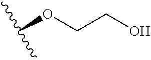 |
| 261 | 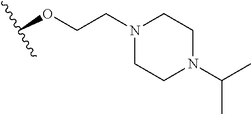 | 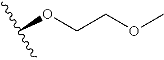 |
| 262 | 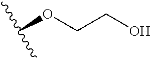 | 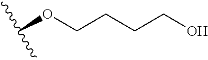 |
| 263 | 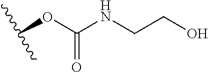 | 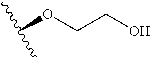 |
| 264 | 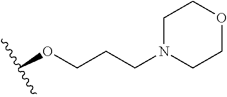 | 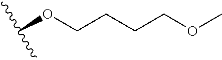 |
| 265 | 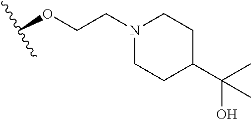 | 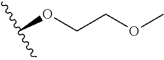 |
| 266 | 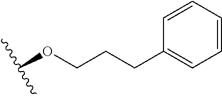 | 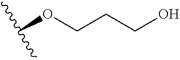 |
| 267 | 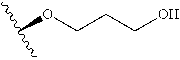 | 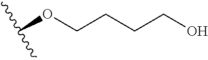 |
| 268 | 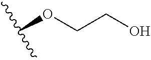 | 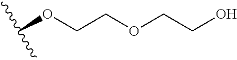 |
| 269 | 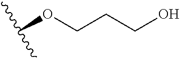 | 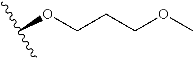 |
| 270 | 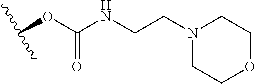 | 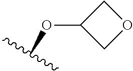 |
| 271 | 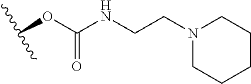 | 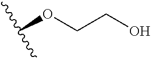 |
| 272 | 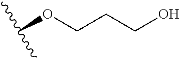 | 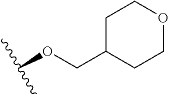 |
| 273 | 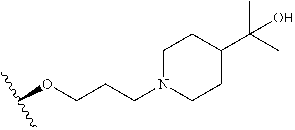 | 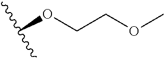 |
| 274 | 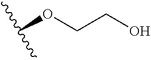 | 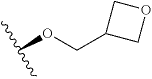 |
| 275 | 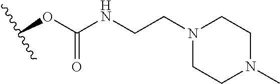 | 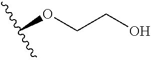 |
| 276 | 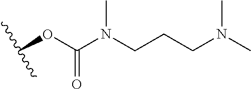 | 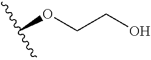 |
| 277 | 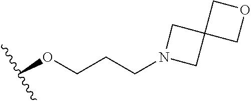 | 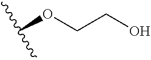 |
| 278 | 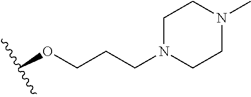 | 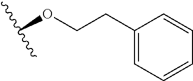 |
| 279 | 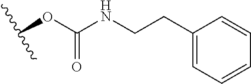 | 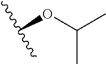 |
| 280 | 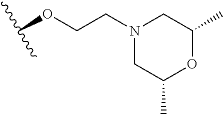 | 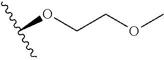 |
| 281 | 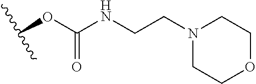 | 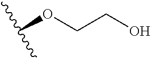 |
| 282 | 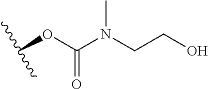 | 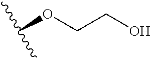 |
| 283 | 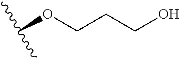 | 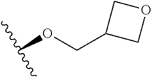 |
| 284 | 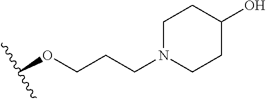 | 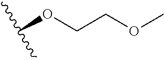 |
| 285 | 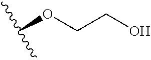 | 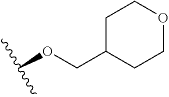 |
| 286 | 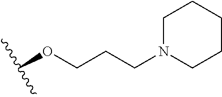 | 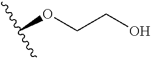 |
| 287 | 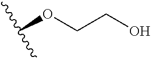 | 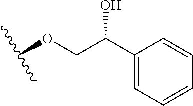 |
| 288 | 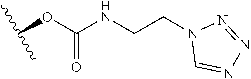 | 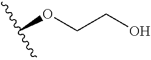 |
| 289 | 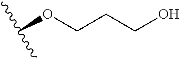 | 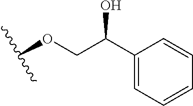 |
| 290 | 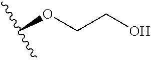 | 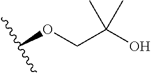 |
| 291 | 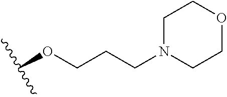 | 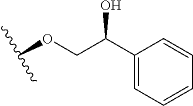 |
| 292 | 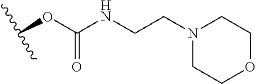 | 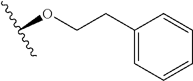 |
| 293 | 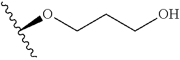 | 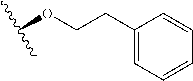 |
| 294 | 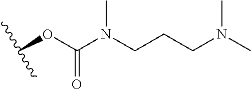 | 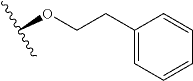 |
| 295 | 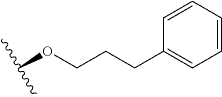 | 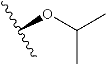 |
| 296 | 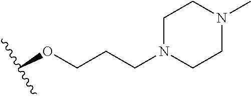 | 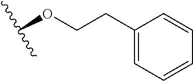 |
| 297 | 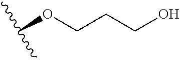 | 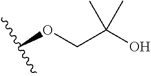 |
| 298 | 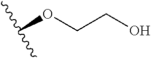 | 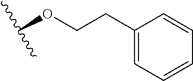 |
| 299 | 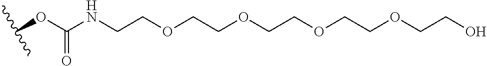 | 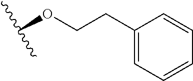 |
| 300 | 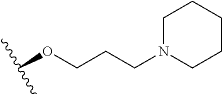 | 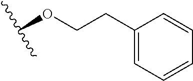 |
| 301 | 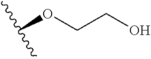 | 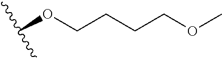 |
| 302 | 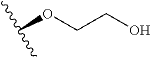 | 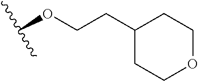 |
| 303 | 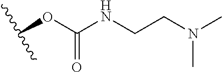 | 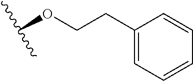 |
| 304 | 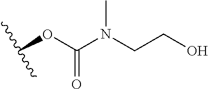 | 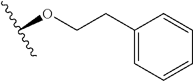 |
| 305 | 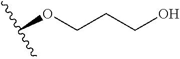 | 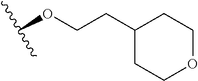 |
| 306 | 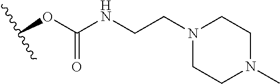 | 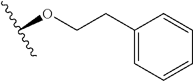 |
| 307 | 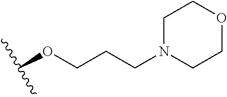 | 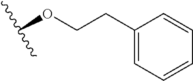 |
| 308 | 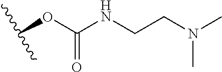 | 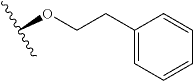 |
| 309 | 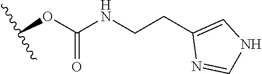 | 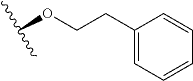 |
| 310 | 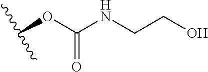 | 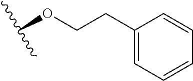 |
| 311 | 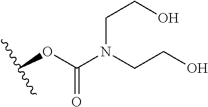 | 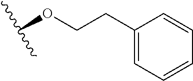 |
| 312 | 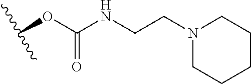 | 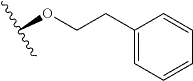 |
| 313 | 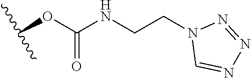 | 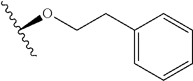 |
| 314 | 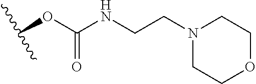 | 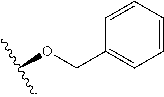 |
| 315 | 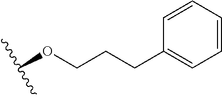 | 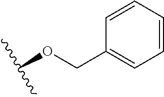 |
| 316 | 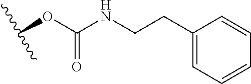 | 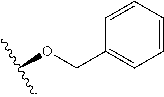 |
| 317 | 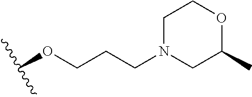 | 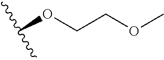 |
| 318 | 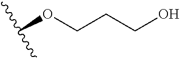 | 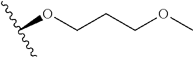 |
| 319 | 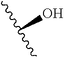 | 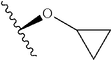 |
| 320 | 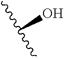 | 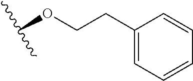 |
| 321 | 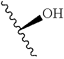 | 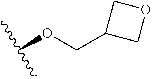 |
| 322 | 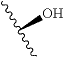 | 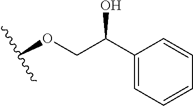 |
| 323 | 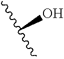 | 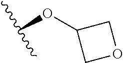 |
| 324 | 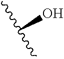 | 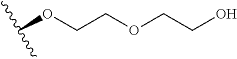 |
| 325 | 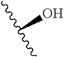 | 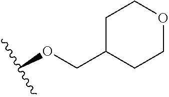 |
| 326 | 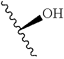 | 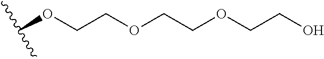 |
| 327 | 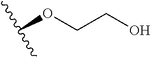 | 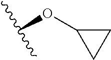 |
| 329 | 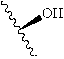 | 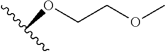 |
| 330 | 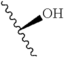 | 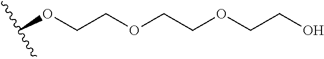 |

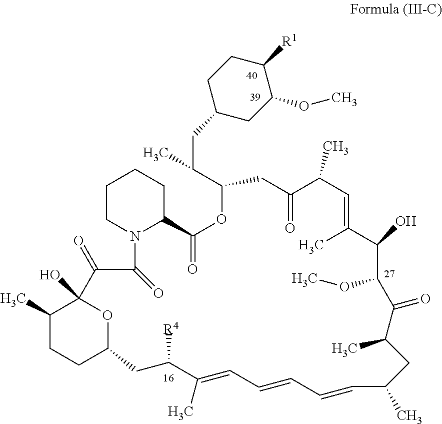

Table 3 with Formula (III-C) core:

| No. # | R¹ | R⁴ |
|---|---|---|
| 400 | 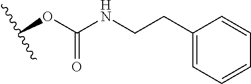 | 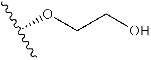 |
| 401 | 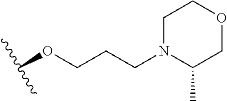 | 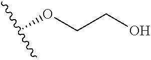 |
| 402 | 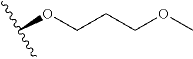 | 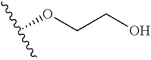 |
| 403 | 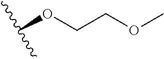 | 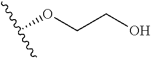 |
| 404 | 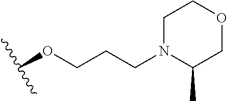 | 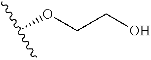 |
| 405 | 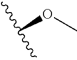 | 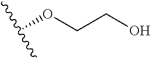 |
| 406 | 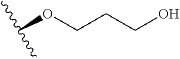 | 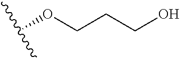 |
| 407 | 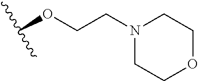 | 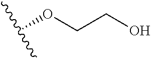 |
| 408 | 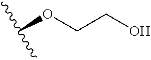 | 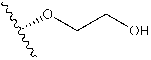 |
| 409 | 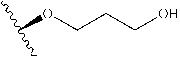 | 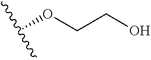 |
| 410 | 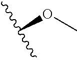 | 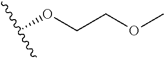 |
| 411 | 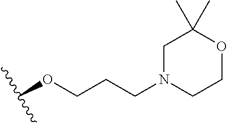 | 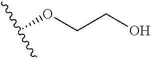 |
| 412 | 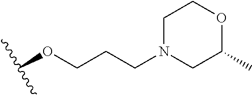 | 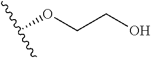 |
| 413 | 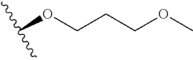 | 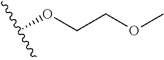 |
| 414 | 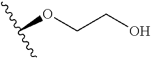 | 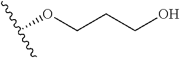 |
| 415 | 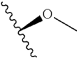 | 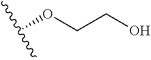 |
| 416 | 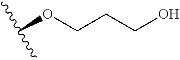 | 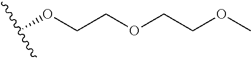 |
| 417 | 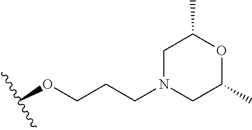 | 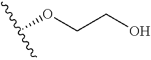 |
| 418 | 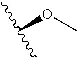 | 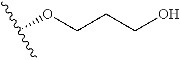 |
| 419 | 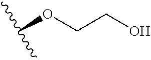 | 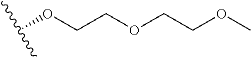 |
| 420 | 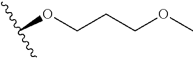 | 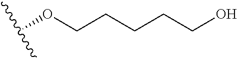 |
| 421 | 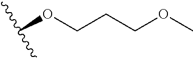 | 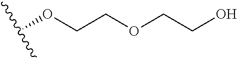 |
| 422 | 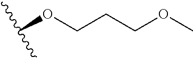 | 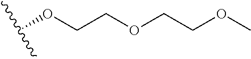 |
| 423 | 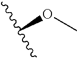 | 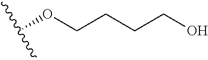 |
| 424 | 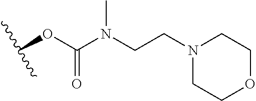 | 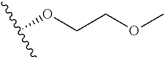 |
| 425 | 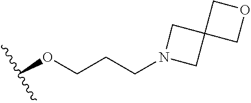 | 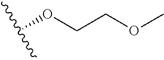 |
| 426 | 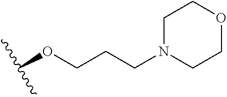 | 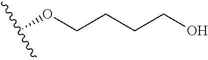 |
| 427 | 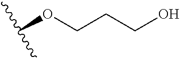 | 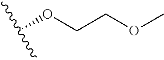 |
| 428 | 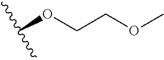 | 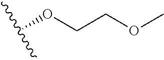 |
| 429 | 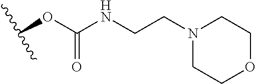 | 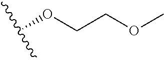 |
| 430 | 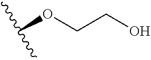 | 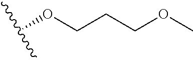 |
| 431 | 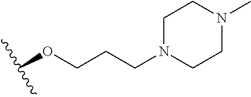 | 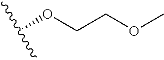 |
| 432 | 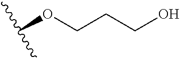 | 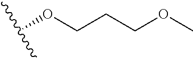 |
| 433 | 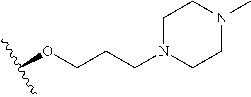 | 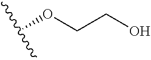 |
| 434 | 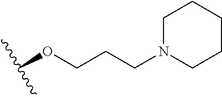 | 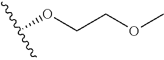 |
| 435 | 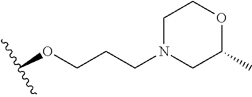 | 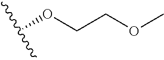 |
| 436 | 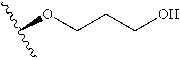 | 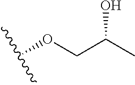 |
| 437 | 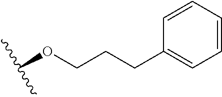 | 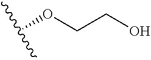 |
| 438 | 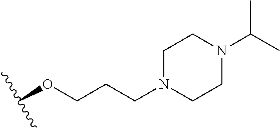 | 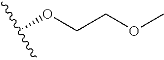 |
| 439 | 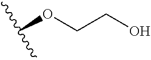 | 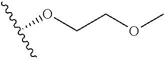 |
| 440 | 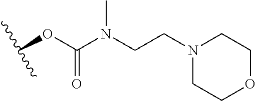 | 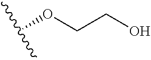 |
| 441 | 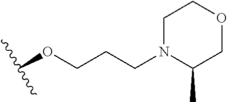 | 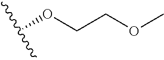 |
| 442 | 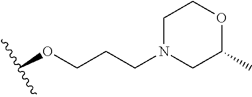 | 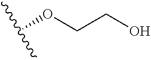 |
| 443 | 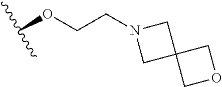 | 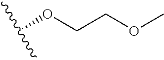 |
| 444 | 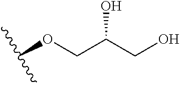 | 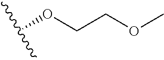 |
| 445 | 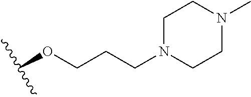 | 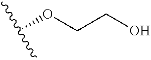 |
| 446 | 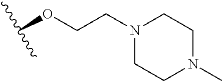 | 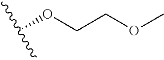 |
| 447 | 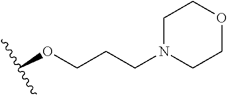 | 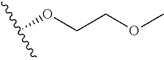 |
| 448 | 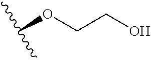 | 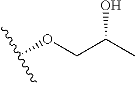 |
| 449 | 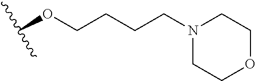 | 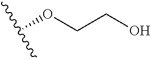 |
| 450 | 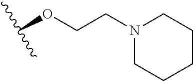 | 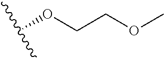 |
| 451 | 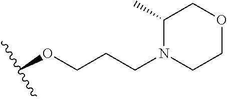 | 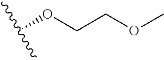 |
| 452 | 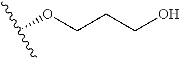 | 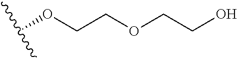 |
| 453 | 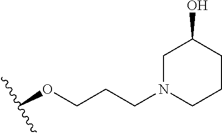 | 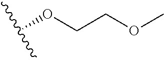 |
| 454 | 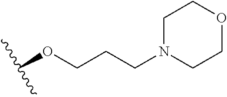 | 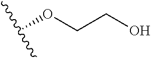 |
| 455 | 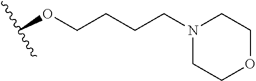 | 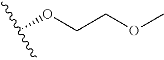 |
| 456 | 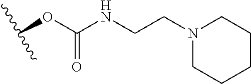 | 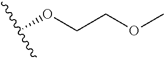 |
| 457 | 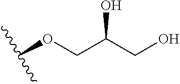 | 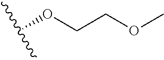 |
| 458 | 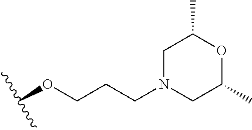 | 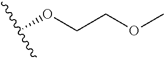 |
| 459 | 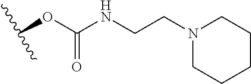 | 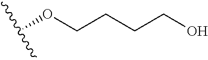 |
| 460 | 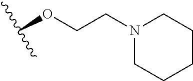 | 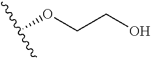 |
| 461 | 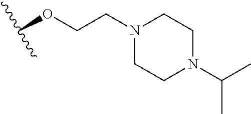 | 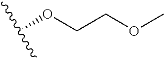 |
| 462 | 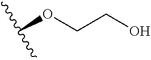 | 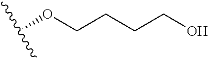 |
| 463 | 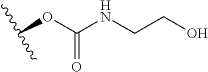 | 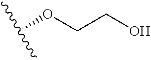 |
| 464 | 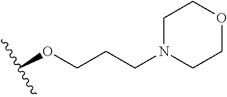 | 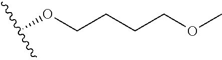 |
| 465 | 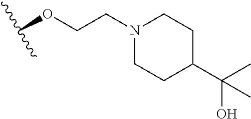 | 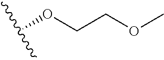 |
| 466 | 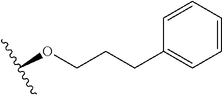 | 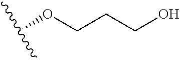 |
| 467 | 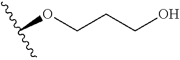 | 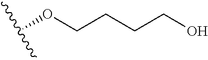 |
| 468 | 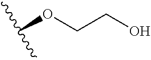 | 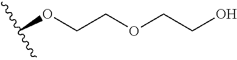 |
| 469 | 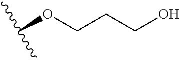 | 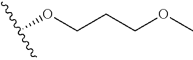 |
| 470 | 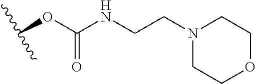 | 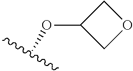 |
| 471 | 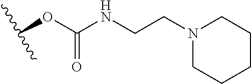 | 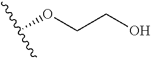 |
| 472 | 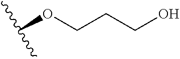 | 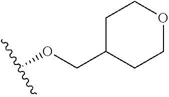 |
| 473 | 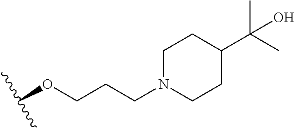 | 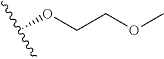 |
| 474 | 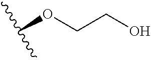 | 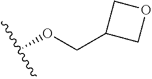 |
| 475 | 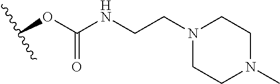 | 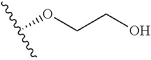 |
| 476 | 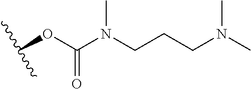 | 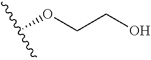 |
| 477 | 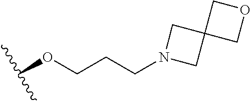 | 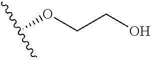 |
| 478 | 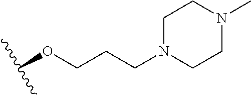 | 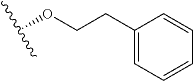 |
| 479 | 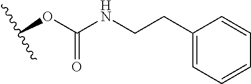 | 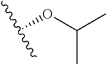 |
| 480 | 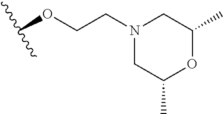 | 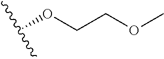 |
| 481 | 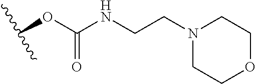 | 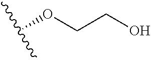 |
| 482 | 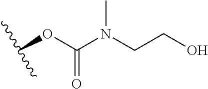 | 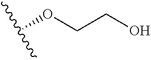 |
| 483 | 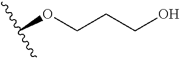 | 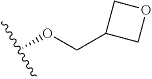 |
| 484 | 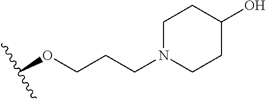 | 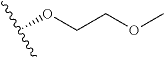 |
| 485 | 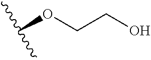 | 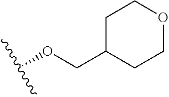 |
| 486 | 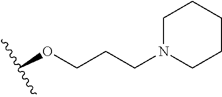 | 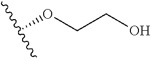 |
| 487 | 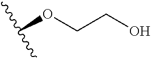 | 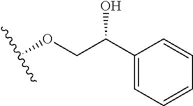 |
| 488 | 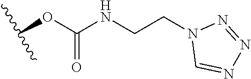 | 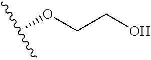 |
| 489 | 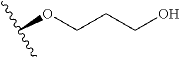 | 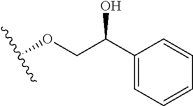 |
| 490 | 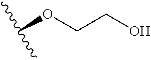 | 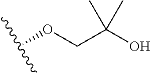 |
| 491 | 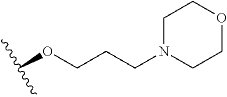 | 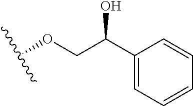 |
| 492 | 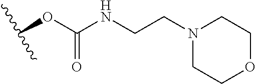 | 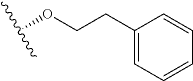 |
| 493 | 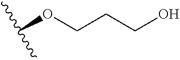 | 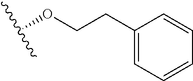 |
| 494 | 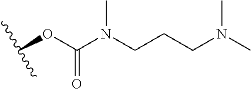 | 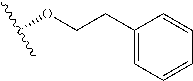 |
| 495 | 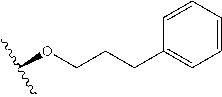 | 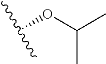 |
| 496 | 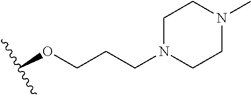 | 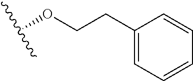 |
| 497 | 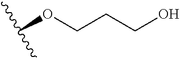 | 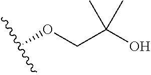 |
| 498 | 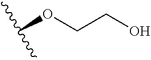 | 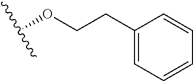 |
| 499 | 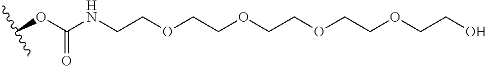 | 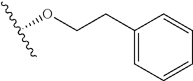 |
| 500 | 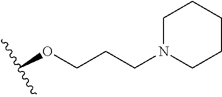 | 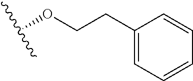 |
| 501 | 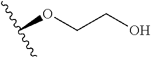 | 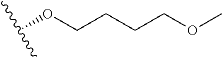 |
| 502 | 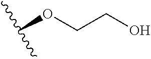 | 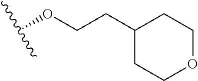 |
| 503 | 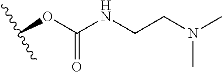 | 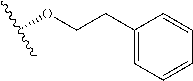 |
| 504 | 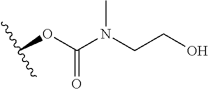 | 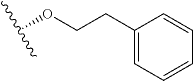 |
| 505 | 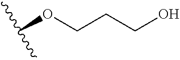 | 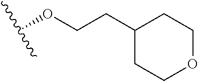 |
| 506 | 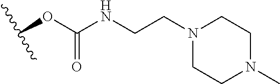 | 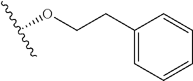 |
| 507 | 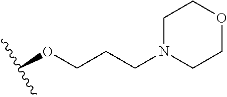 | 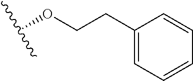 |
| 508 | 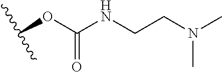 | 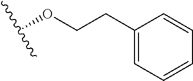 |
| 509 | 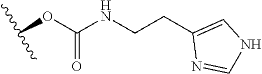 | 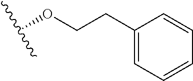 |
| 510 | 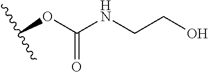 | 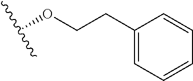 |
| 511 | 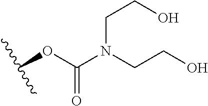 | 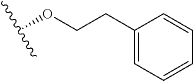 |
| 512 | 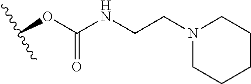 | 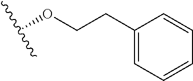 |
| 513 | 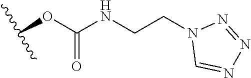 | 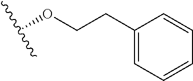 |
| 514 | 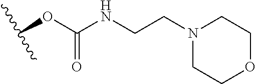 | 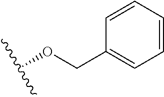 |
| 515 | 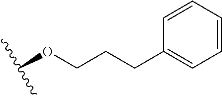 | 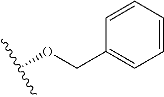 |
| 516 | 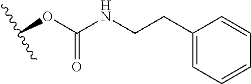 | 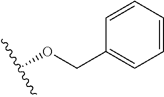 |
| 517 | 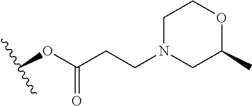 | 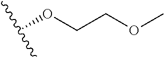 |
| 518 | 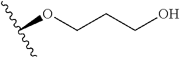 | 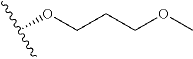 |
| 519 | 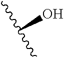 | 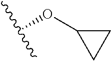 |
| 520 | 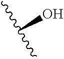 | 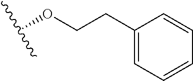 |
| 521 | 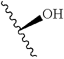 | 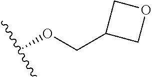 |
| 522 | 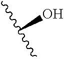 | 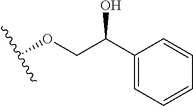 |
| 523 | 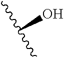 | 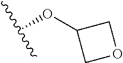 |
| 524 | 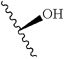 | 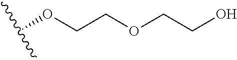 |
| 525 | 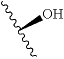 | 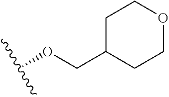 |
| 526 | 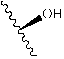 | 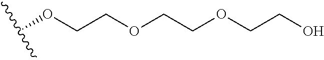 |
| 527 | 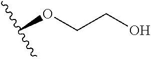 | 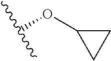 |
| 529 | 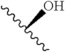 | 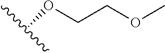 |

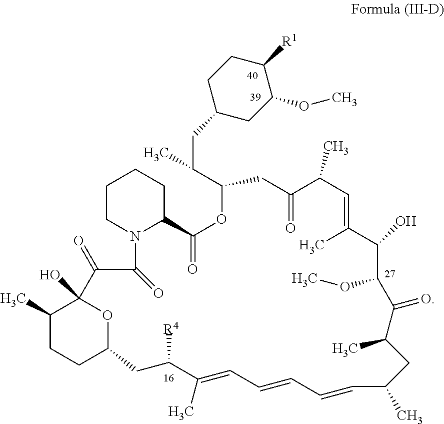

TABLE 4
with Formula (IIII-D) core:

| No. # | R¹ | R⁴ |
|---|---|---|
| 600 | 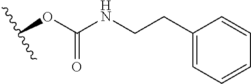 | 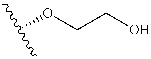 |
| 601 | 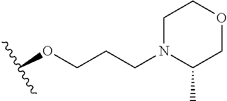 | 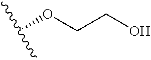 |
| 602 | 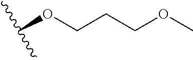 | 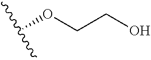 |
| 603 | 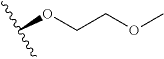 | 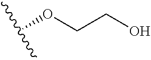 |
| 604 | 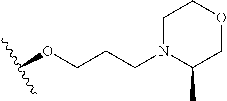 | 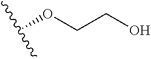 |
| 605 | 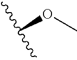 | 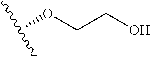 |
| 606 | 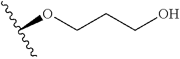 | 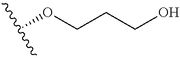 |
| 607 | 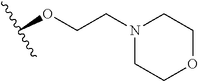 | 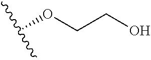 |
| 608 | 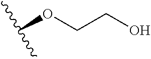 | 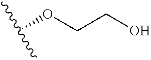 |
| 609 | 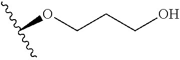 | 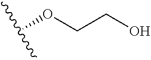 |
| 610 | 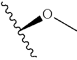 | 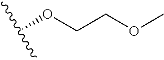 |
| 611 | 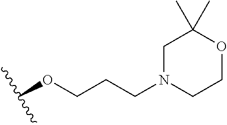 | 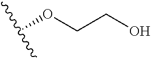 |
| 612 | 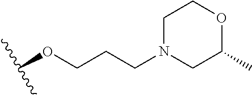 | 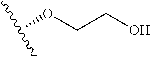 |
| 613 | 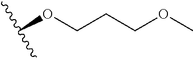 | 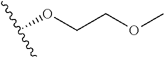 |
| 614 | 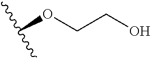 | 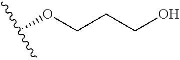 |
| 615 | 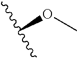 | 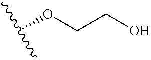 |
| 616 | 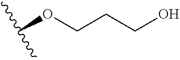 | 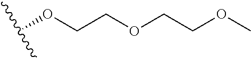 |
| 617 | 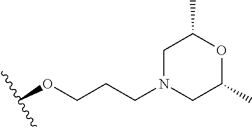 | 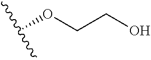 |
| 618 | 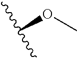 | 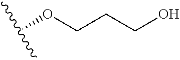 |
| 619 | 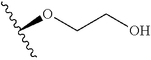 | 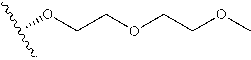 |
| 620 | 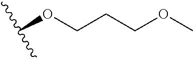 | 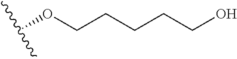 |
| 621 | 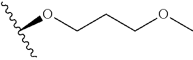 | 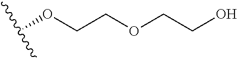 |
| 622 | 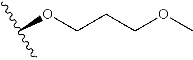 | 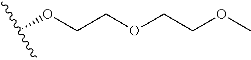 |
| 623 | 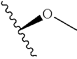 | 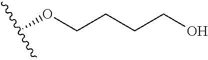 |
| 624 | 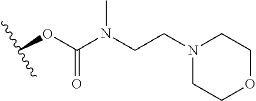 | 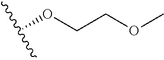 |
| 625 | 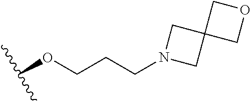 | 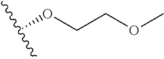 |
| 626 | 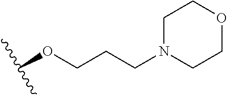 | 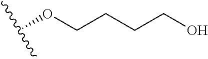 |
| 627 | 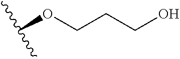 | 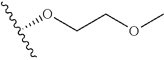 |
| 628 | 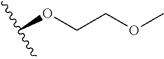 | 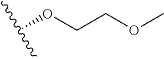 |
| 629 | 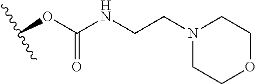 | 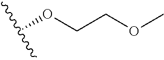 |
| 630 | 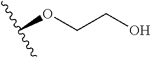 | 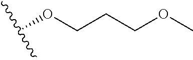 |
| 631 | 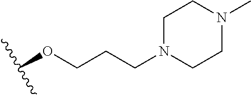 | 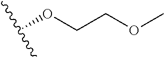 |
| 632 | 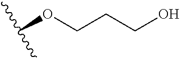 | 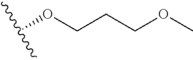 |
| 633 | 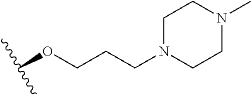 | 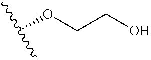 |
| 634 | 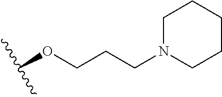 | 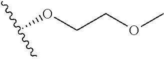 |
| 635 | 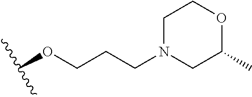 | 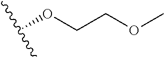 |
| 636 | 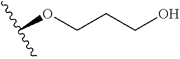 | 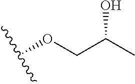 |
| 637 | 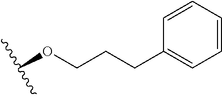 | 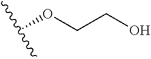 |
| 638 | 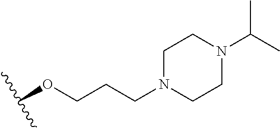 | 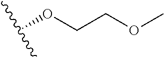 |
| 639 | 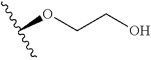 | 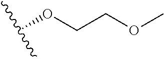 |
| 640 | 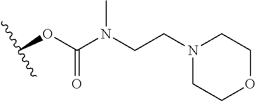 | 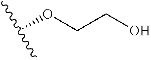 |
| 641 | 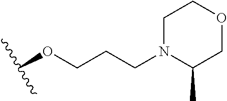 | 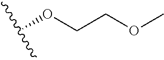 |
| 642 | 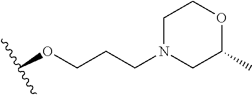 | 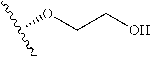 |
| 643 | 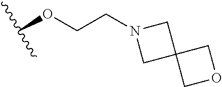 | 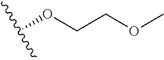 |
| 644 | 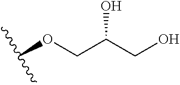 | 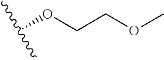 |
| 645 | 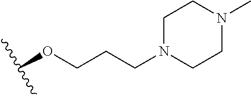 | 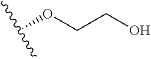 |
| 646 | 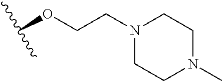 | 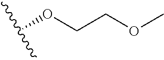 |
| 647 | 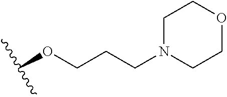 | 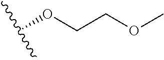 |
| 648 | 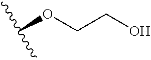 | 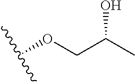 |
| 649 | 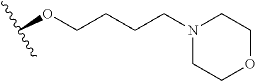 | 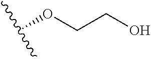 |
| 650 | 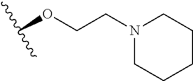 | 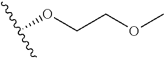 |
| 651 | 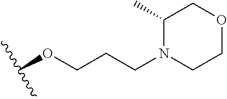 | 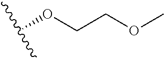 |
| 652 | 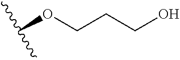 | 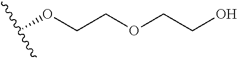 |
| 653 | 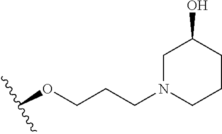 | 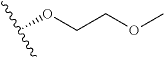 |
| 654 | 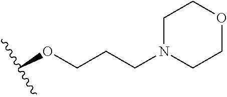 | 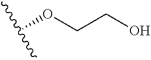 |
| 655 | 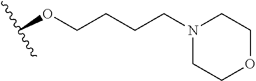 | 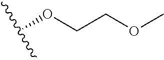 |
| 656 | 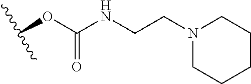 | 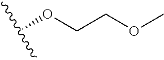 |
| 657 | 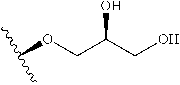 | 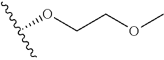 |
| 658 | 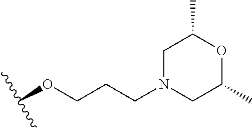 | 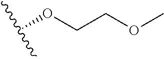 |
| 659 | 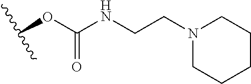 | 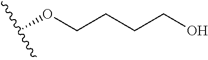 |
| 660 | 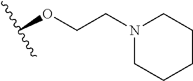 | 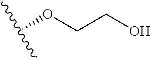 |
| 661 | 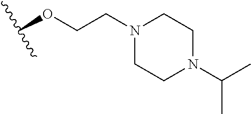 | 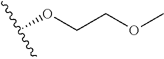 |
| 662 | 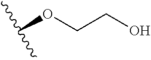 | 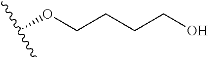 |
| 663 | 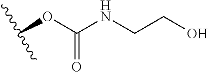 | 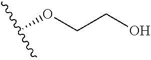 |
| 664 | 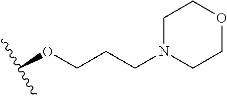 | 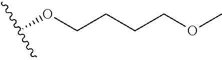 |
| 665 | 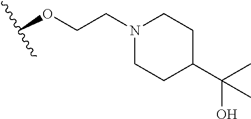 | 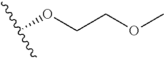 |
| 666 | 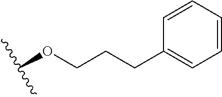 | 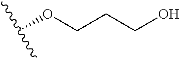 |
| 667 | 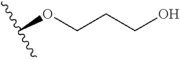 | 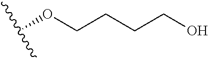 |
| 668 | 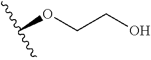 | 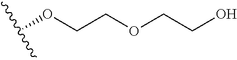 |
| 669 | 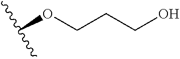 | 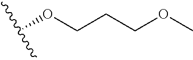 |
| 670 | 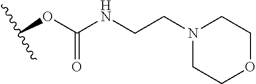 | 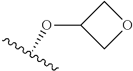 |
| 671 | 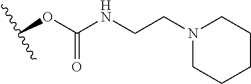 | 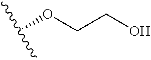 |
| 672 | 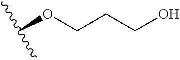 | 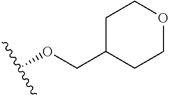 |
| 673 | 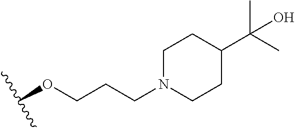 | 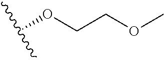 |
| 674 | 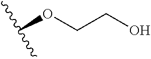 | 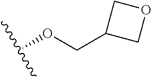 |
| 675 | 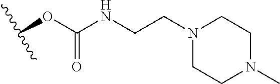 | 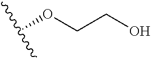 |
| 676 | 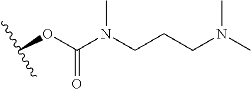 | 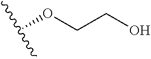 |
| 677 | 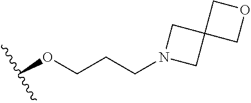 | 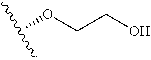 |
| 678 | 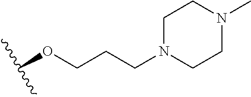 | 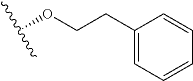 |
| 679 | 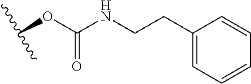 | 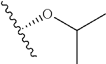 |
| 680 | 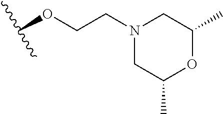 | 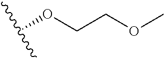 |
| 681 | 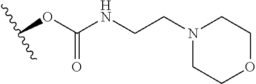 | 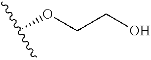 |
| 682 | 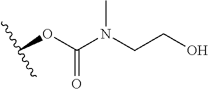 | 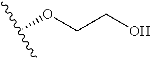 |
| 683 | 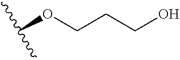 | 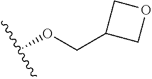 |
| 684 | 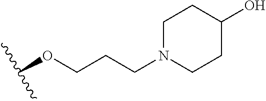 | 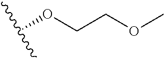 |
| 685 | 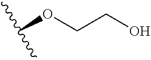 | 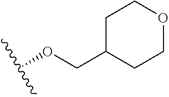 |
| 686 | 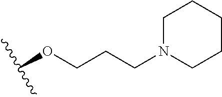 | 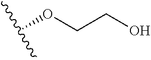 |
| 687 | 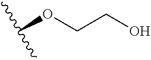 | 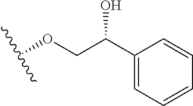 |
| 688 | 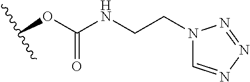 | 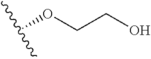 |
| 689 | 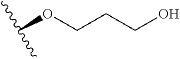 | 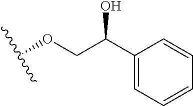 |
| 690 | 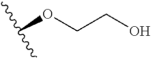 | 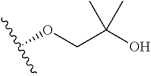 |
| 691 | 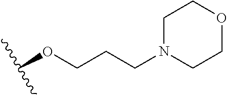 | 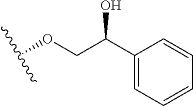 |
| 692 | 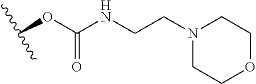 | 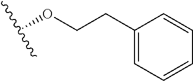 |
| 693 | 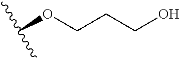 | 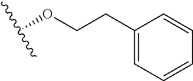 |
| 694 | 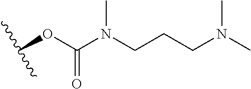 | 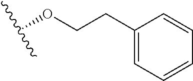 |
| 695 | 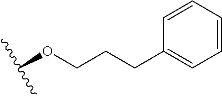 | 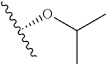 |
| 696 | 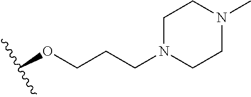 | 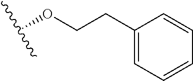 |
| 697 | 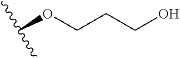 | 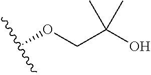 |
| 698 | 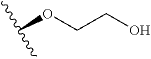 | 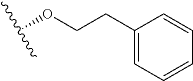 |
| 699 | 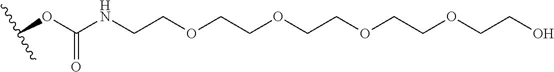 | 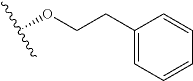 |
| 700 | 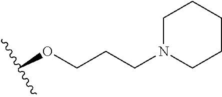 | 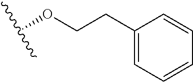 |
| 701 | 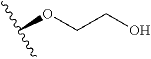 | 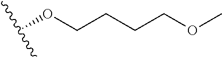 |
| 702 | 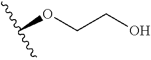 | 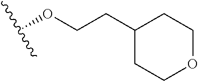 |
| 703 | 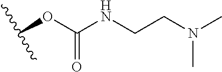 | 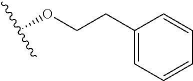 |
| 704 | 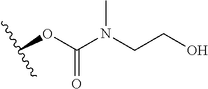 | 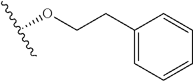 |
| 705 | 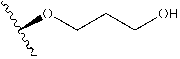 | 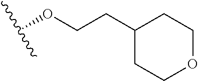 |
| 706 | 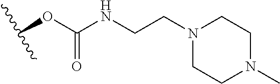 | 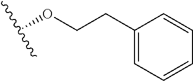 |
| 707 | 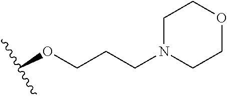 | 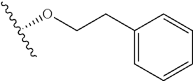 |
| 708 | 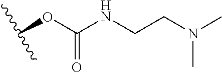 | 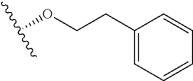 |
| 709 | 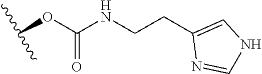 | 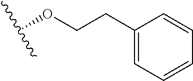 |
| 710 | 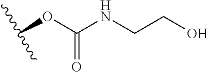 | 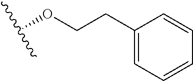 |
| 711 | 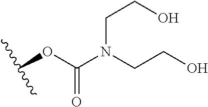 | 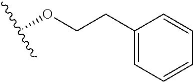 |
| 712 | 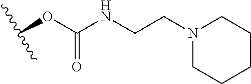 | 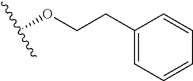 |
| 713 | 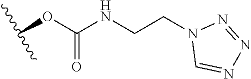 | 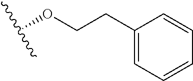 |
| 714 | 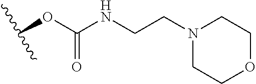 | 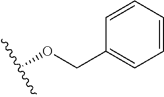 |
| 715 | 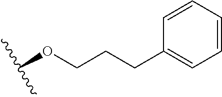 | 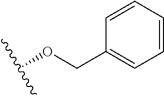 |
| 716 | 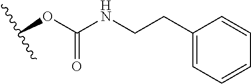 | 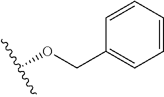 |
| 717 | 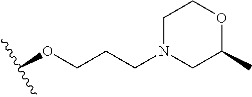 | 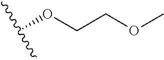 |
| 718 | 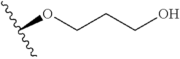 | 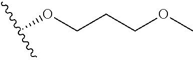 |
| 719 | 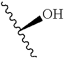 | 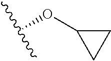 |
| 720 | 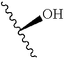 | 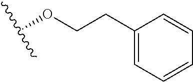 |
| 721 | 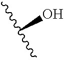 | 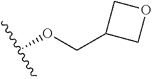 |
| 722 | 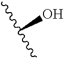 | 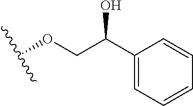 |
| 723 | 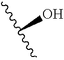 | 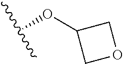 |
| 724 | 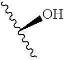 | 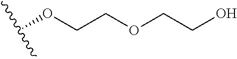 |
| 725 | 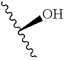 | 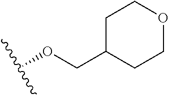 |
| 726 | 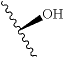 | 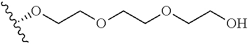 |
| 727 | 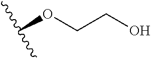 | 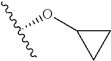 |
| 729 | 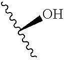 | 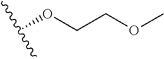 |
| 730 | 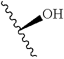 | 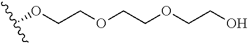 |

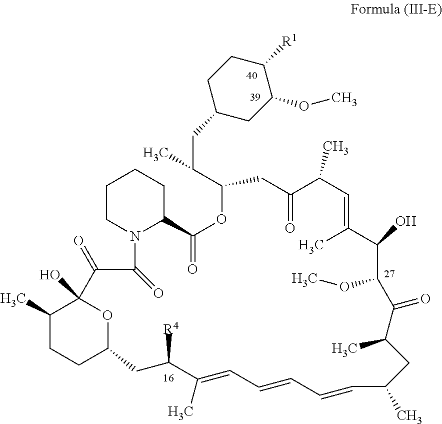

TABLE 5
with Formula (III-E) core:

| No. # | R¹ | R⁴ |
|---|---|---|
| 801 | 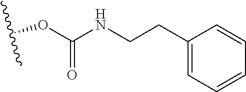 | 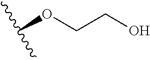 |
| 802 | 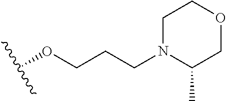 | 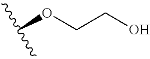 |
| 803 | 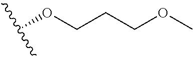 | 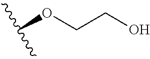 |
| 804 | 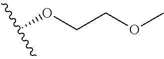 | 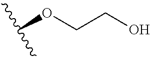 |
| 805 | 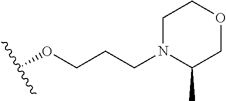 | 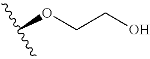 |
| 806 | 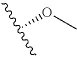 | 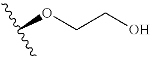 |
| 807 | 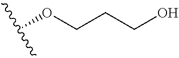 | 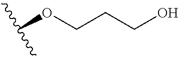 |
| 808 | 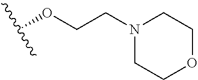 | 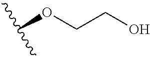 |
| 809 | 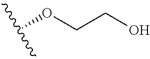 | 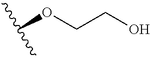 |
| 810 | 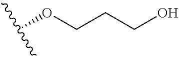 | 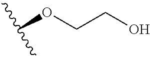 |
| 811 | 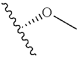 | 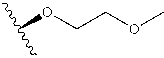 |
| 812 | 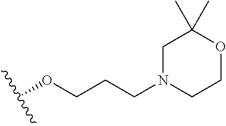 | 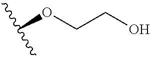 |
| 813 | 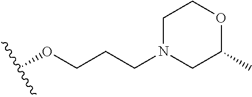 | 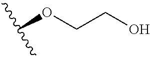 |
| 814 | 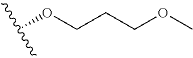 | 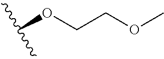 |
| 815 | 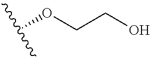 | 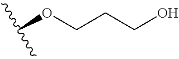 |
| 816 | 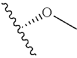 | 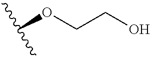 |
| 817 | 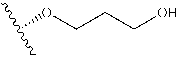 | 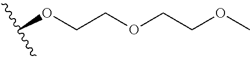 |
| 818 | 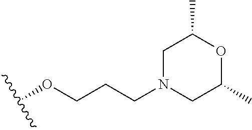 | 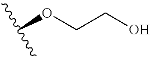 |
| 819 | 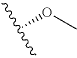 | 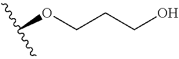 |
| 820 | 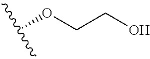 | 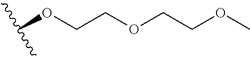 |
| 821 | 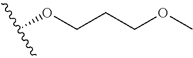 | 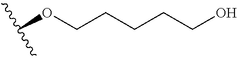 |
| 822 | 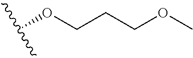 | 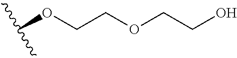 |
| 823 | 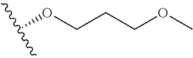 | 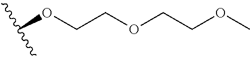 |
| 824 | 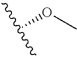 | 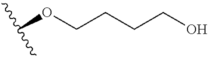 |
| 825 | 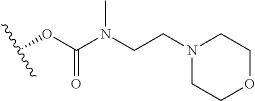 | 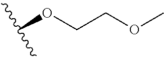 |
| 826 | 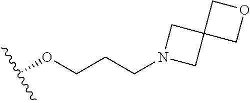 | 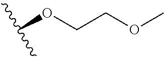 |
| 827 | 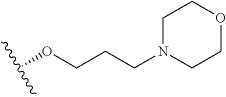 | 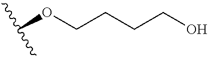 |
| 828 | 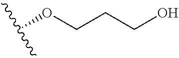 | 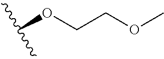 |
| 829 | 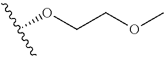 | 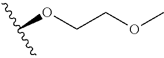 |
| 830 | 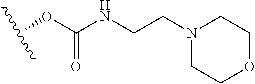 | 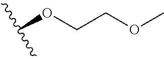 |
| 831 | 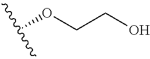 | 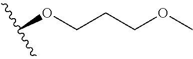 |
| 832 | 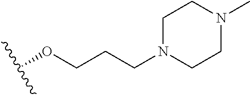 | 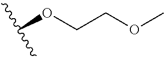 |
| 833 | 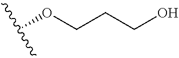 | 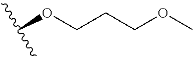 |
| 834 | 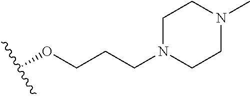 | 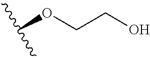 |
| 835 | 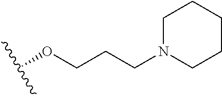 | 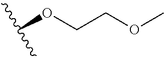 |
| 836 | 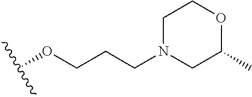 | 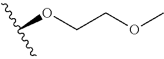 |
| 837 | 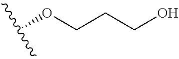 | 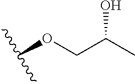 |
| 838 | 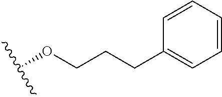 | 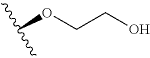 |
| 839 | 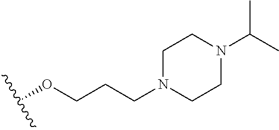 | 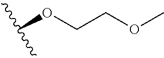 |
| 840 | 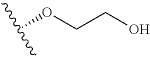 | 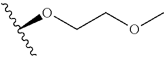 |
| 841 | 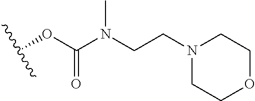 | 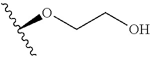 |
| 842 | 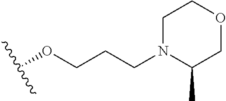 | 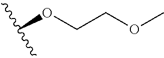 |
| 843 | 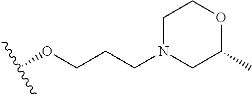 | 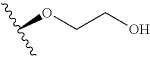 |
| 844 | 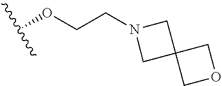 | 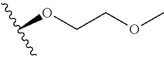 |
| 845 | 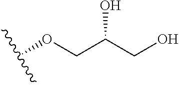 | 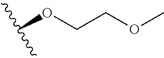 |
| 846 | 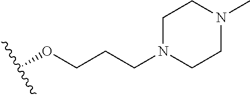 | 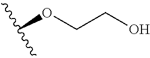 |
| 847 | 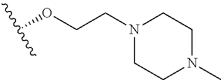 | 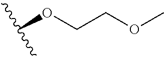 |
| 848 | 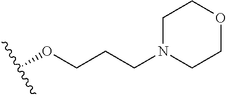 | 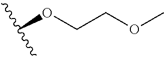 |
| 849 | 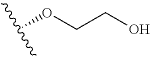 | 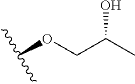 |
| 850 | 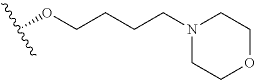 | 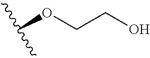 |
| 851 | 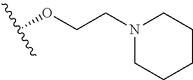 | 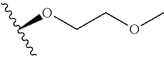 |
| 852 | 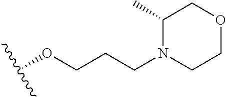 | 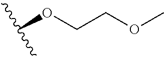 |
| 853 | 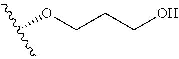 | 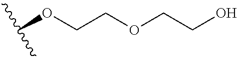 |
| 854 | 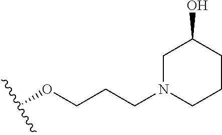 | 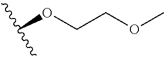 |
| 855 | 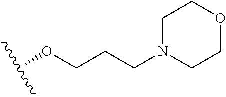 | 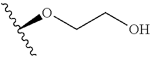 |
| 856 | 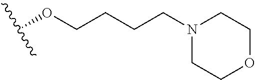 | 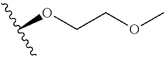 |
| 857 | 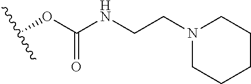 | 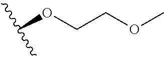 |
| 858 | 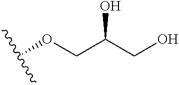 | 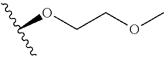 |
| 859 | 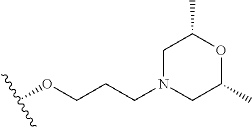 | 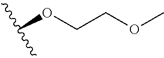 |
| 860 | 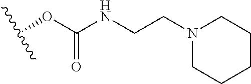 | 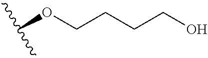 |
| 861 | 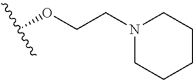 | 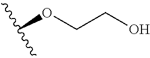 |
| 862 | 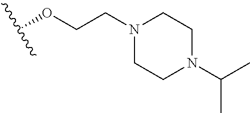 | 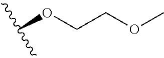 |
| 863 | 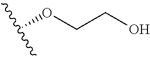 | 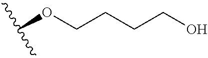 |
| 864 | 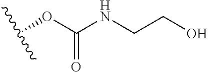 | 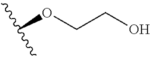 |
| 865 | 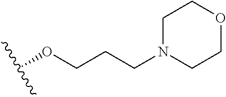 | 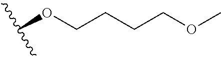 |
| 866 | 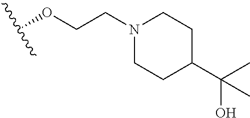 | 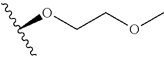 |
| 867 | 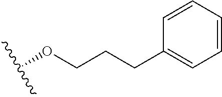 | 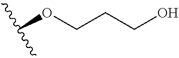 |
| 868 | 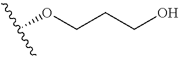 | 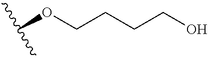 |
| 869 | 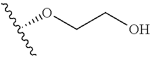 | 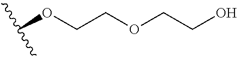 |
| 870 | 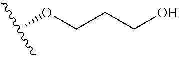 | 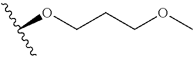 |
| 871 | 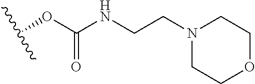 | 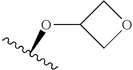 |
| 872 | 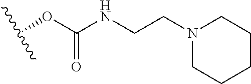 | 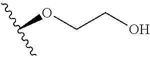 |
| 873 | 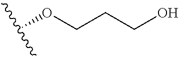 | 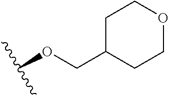 |
| 874 | 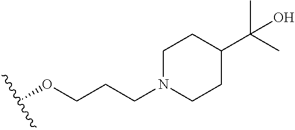 | 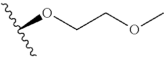 |
| 875 | 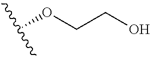 | 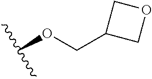 |
| 876 | 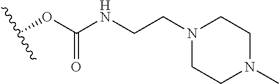 | 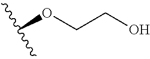 |
| 877 | 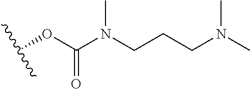 | 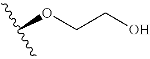 |
| 878 | 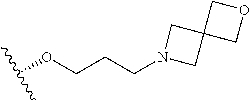 | 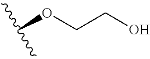 |
| 879 | 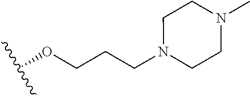 | 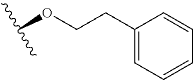 |
| 880 | 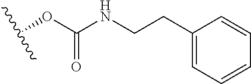 | 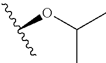 |
| 881 | 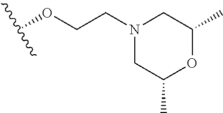 | 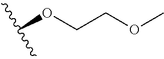 |
| 882 | 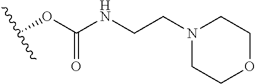 | 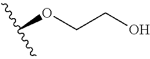 |
| 883 | 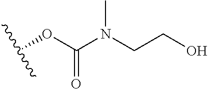 | 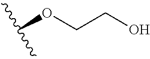 |
| 884 | 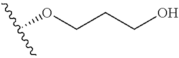 | 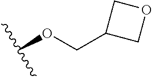 |
| 885 | 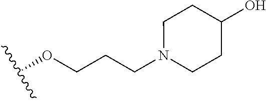 | 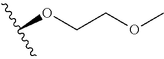 |
| 886 | 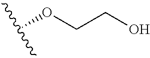 | 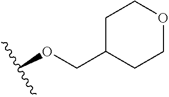 |
| 887 | 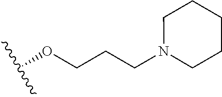 | 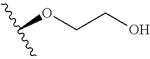 |
| 888 | 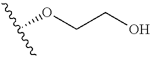 | 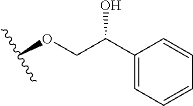 |
| 889 | 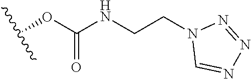 | 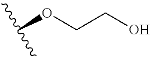 |
| 890 | 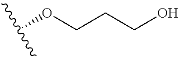 | 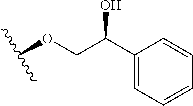 |
| 891 | 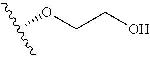 | 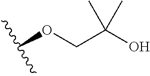 |
| 892 | 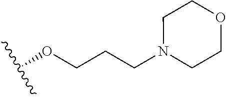 | 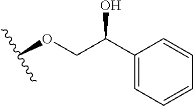 |
| 893 | 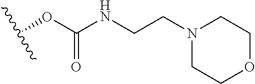 | 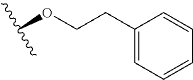 |
| 894 | 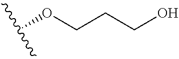 | 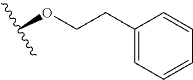 |
| 895 | 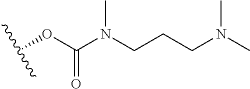 | 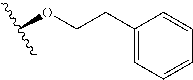 |
| 896 | 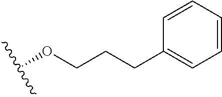 | 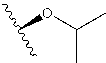 |
| 897 | 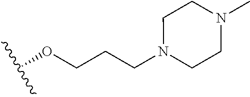 | 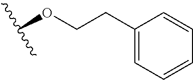 |
| 898 | 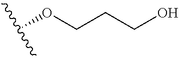 | 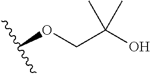 |
| 899 | 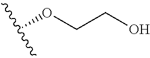 | 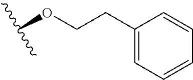 |
| 900 | 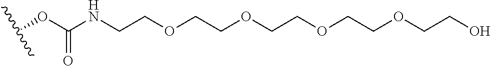 | 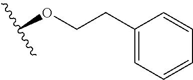 |
| 901 | 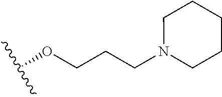 | 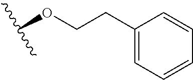 |
| 902 | 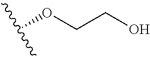 | 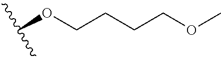 |
| 903 | 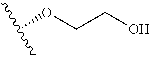 | 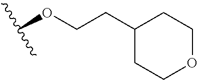 |
| 904 | 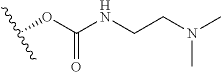 | 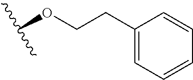 |
| 905 | 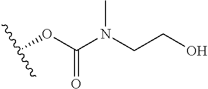 | 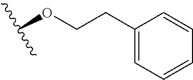 |
| 906 | 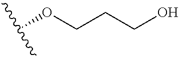 | 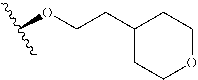 |
| 907 | 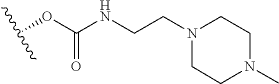 | 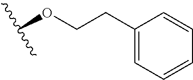 |
| 908 | 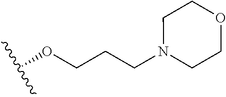 | 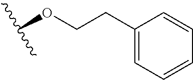 |
| 909 | 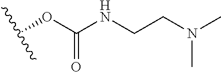 | 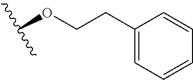 |
| 910 | 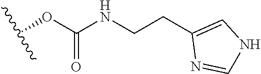 | 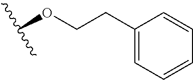 |
| 911 | 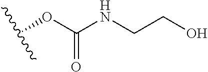 | 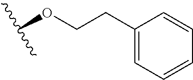 |
| 912 | 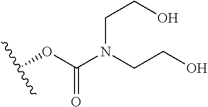 | 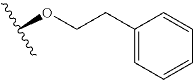 |
| 913 | 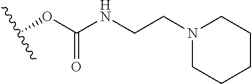 | 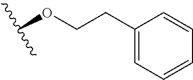 |
| 914 | 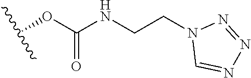 | 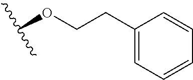 |
| 915 | 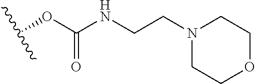 | 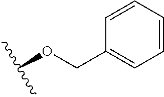 |
| 916 | 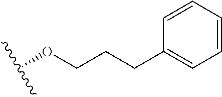 | 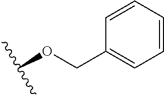 |
| 917 | 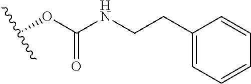 | 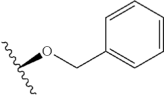 |
| 918 | 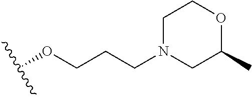 | 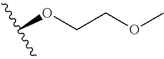 |
| 919 | 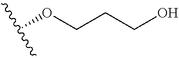 | 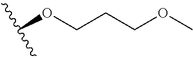 |
| 920 | 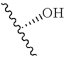 | 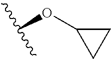 |
| 921 | 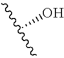 | 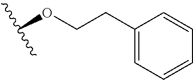 |
| 922 | 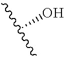 | 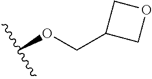 |
| 923 | 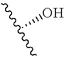 | 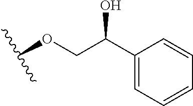 |
| 924 | 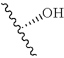 | 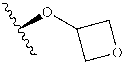 |
| 925 | 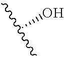 | 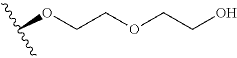 |
| 926 | 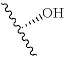 | 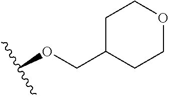 |
| 927 | 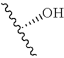 | 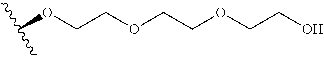 |
| 928 | 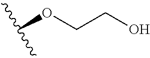 | 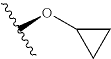 |
| 930 | 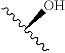 | 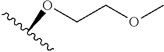 |
| 931 | 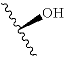 | 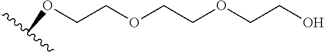 |

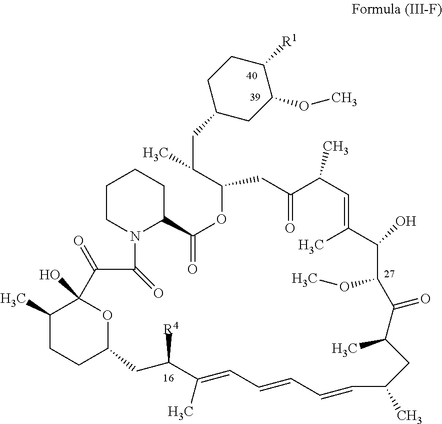

TABLE 6
with Formula (III-F) core:

| No. # | R¹ | R⁴ |
|---|---|---|
| 1001 | 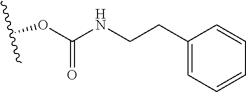 | 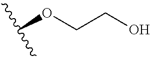 |
| 1002 | 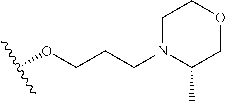 | 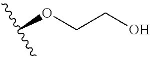 |
| 1003 | 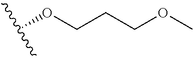 | 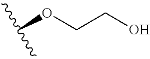 |
| 1004 | 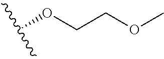 | 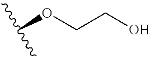 |
| 1005 | 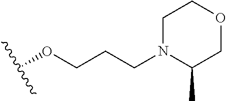 | 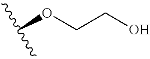 |
| 1006 | 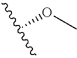 | 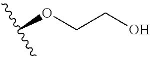 |
| 1007 | 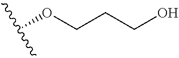 | 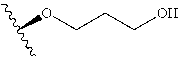 |
| 1008 | 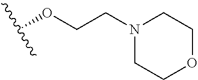 | 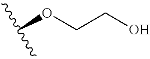 |
| 1009 | 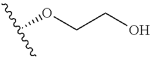 | 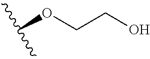 |
| 1000 | 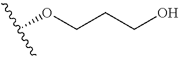 | 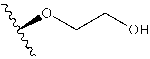 |
| 1011 | 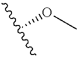 | 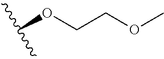 |
| 1012 | 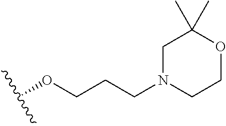 | 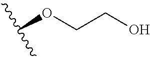 |
| 1013 | 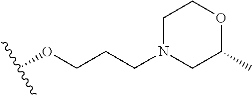 | 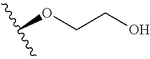 |
| 1014 | 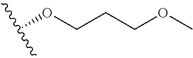 | 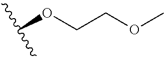 |
| 1015 | 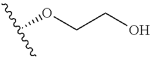 | 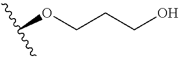 |
| 1016 | 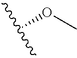 | 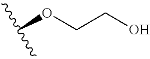 |
| 1017 | 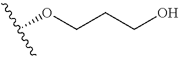 | 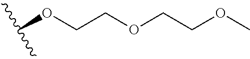 |
| 1018 | 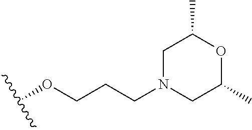 | 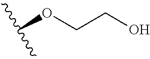 |
| 1019 | 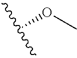 | 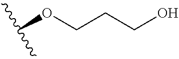 |
| 1020 | 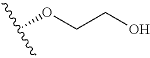 | 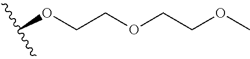 |
| 1021 | 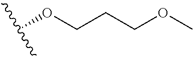 | 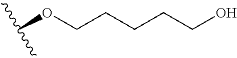 |
| 1022 | 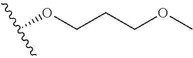 | 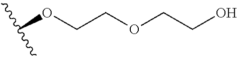 |
| 1023 | 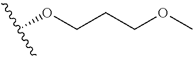 | 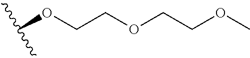 |
| 1024 | 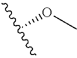 | 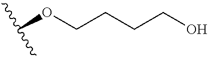 |
| 1025 | 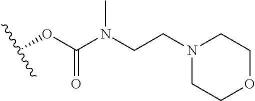 | 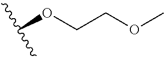 |
| 1026 | 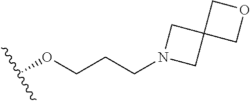 | 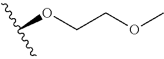 |
| 1027 | 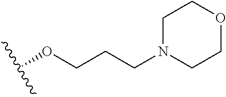 | 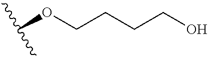 |
| 1028 | 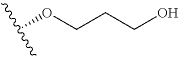 | 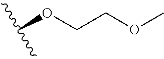 |
| 1029 | 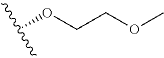 | 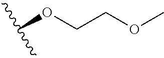 |
| 1030 | 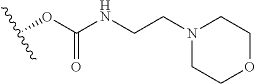 | 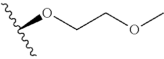 |
| 1031 | 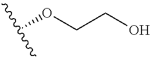 | 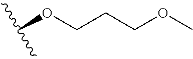 |
| 1032 | 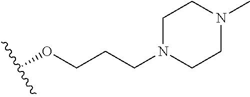 | 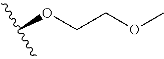 |
| 1033 | 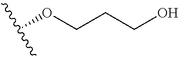 | 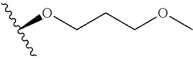 |
| 1034 | 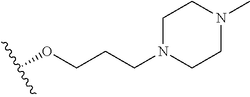 | 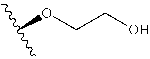 |
| 1035 | 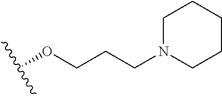 | 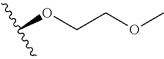 |
| 1036 | 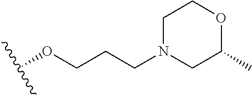 | 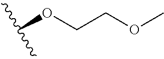 |
| 1037 | 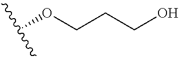 | 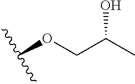 |
| 1038 | 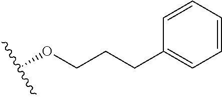 | 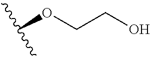 |
| 1039 | 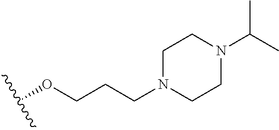 | 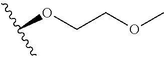 |
| 1040 | 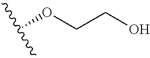 | 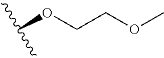 |
| 1041 | 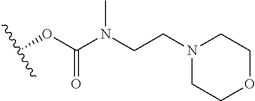 | 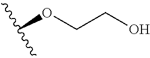 |
| 1042 | 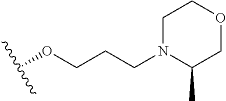 | 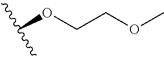 |
| 1043 | 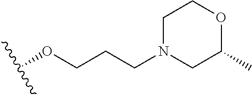 | 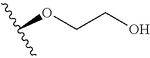 |
| 1044 | 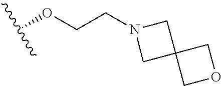 | 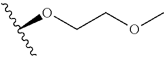 |
| 1045 | 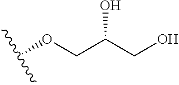 | 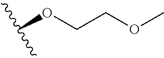 |
| 1046 | 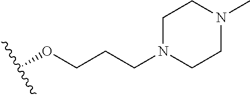 | 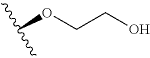 |
| 1047 | 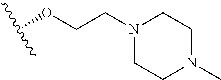 | 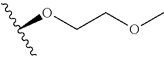 |
| 1048 | 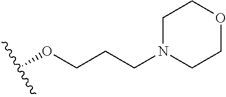 | 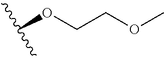 |
| 1049 | 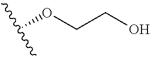 | 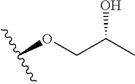 |
| 1050 | 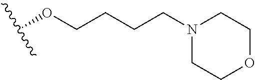 | 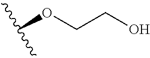 |
| 1051 | 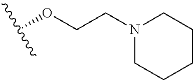 | 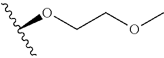 |
| 1052 | 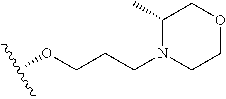 | 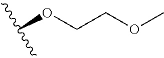 |
| 1053 | 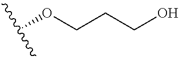 | 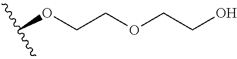 |
| 1054 | 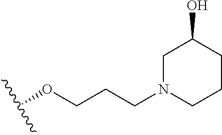 | 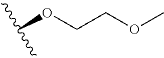 |
| 1055 | 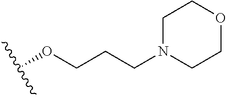 | 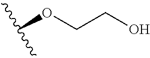 |
| 1056 | 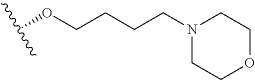 | 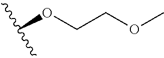 |
| 1057 | 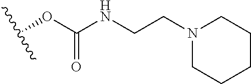 | 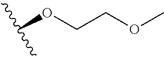 |
| 1058 | 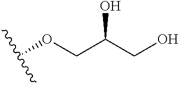 | 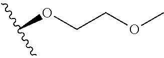 |
| 1059 | 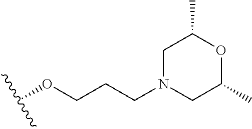 | 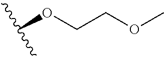 |
| 1060 | 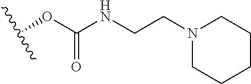 | 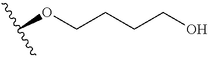 |
| 1061 | 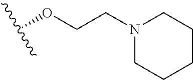 | 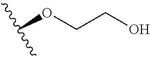 |
| 1062 | 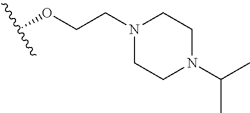 | 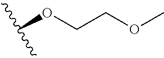 |
| 1063 | 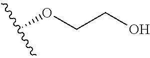 | 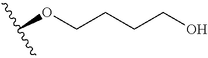 |
| 1064 | 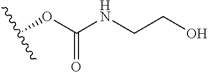 | 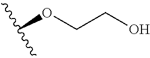 |
| 1065 | 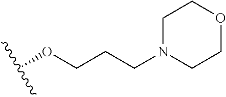 | 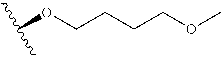 |
| 1066 | 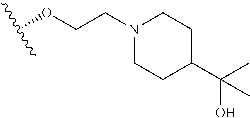 | 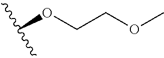 |
| 1067 | 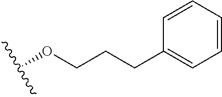 | 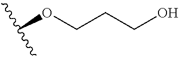 |
| 1068 | 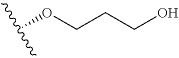 | 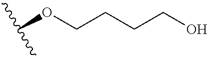 |
| 1069 | 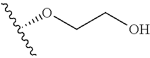 | 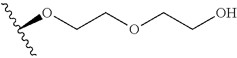 |
| 1070 | 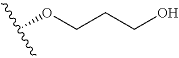 | 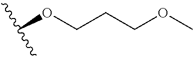 |
| 1071 | 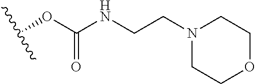 | 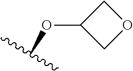 |
| 1072 | 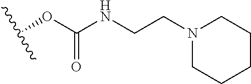 | 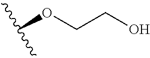 |
| 1073 | 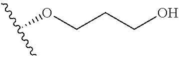 | 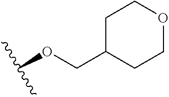 |
| 1074 | 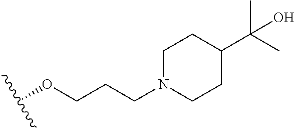 | 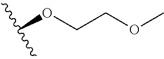 |
| 1075 | 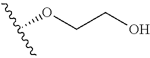 | 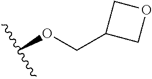 |
| 1076 | 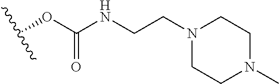 | 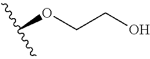 |
| 1077 | 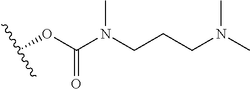 | 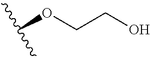 |
| 1078 | 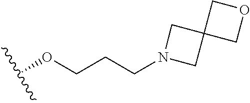 | 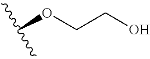 |
| 1079 | 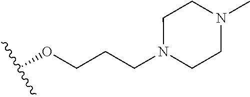 | 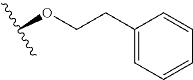 |
| 1080 | 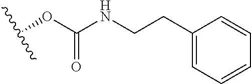 | 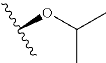 |
| 1081 | 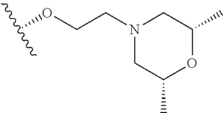 | 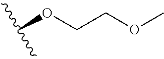 |
| 1082 | 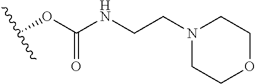 | 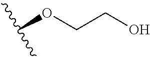 |
| 1083 | 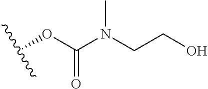 | 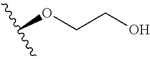 |
| 1084 | 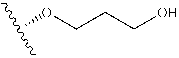 | 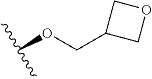 |
| 1085 | 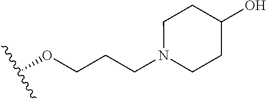 | 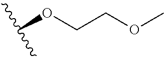 |
| 1086 | 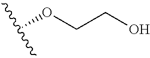 | 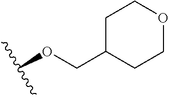 |
| 1087 | 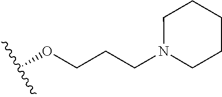 | 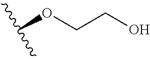 |
| 1088 | 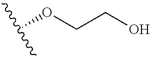 | 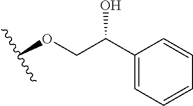 |
| 1089 | 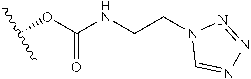 | 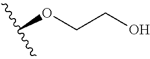 |
| 1090 | 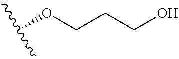 | 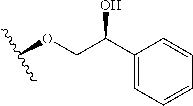 |
| 1091 | 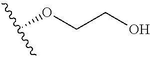 | 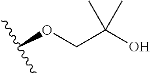 |
| 1092 | 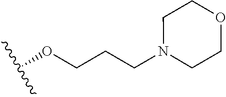 | 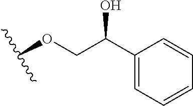 |
| 1993 | 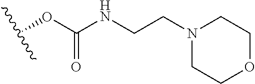 | 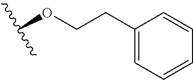 |
| 1094 | 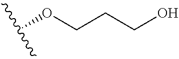 | 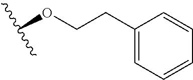 |
| 1095 | 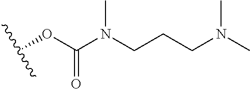 | 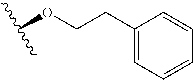 |
| 1096 | 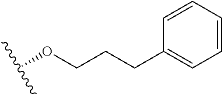 | 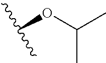 |
| 1097 | 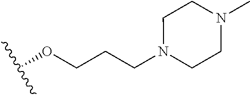 | 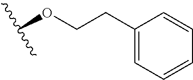 |
| 1098 | 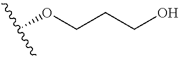 | 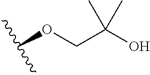 |
| 1099 | 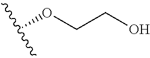 | 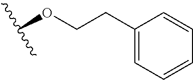 |
| 1100 | 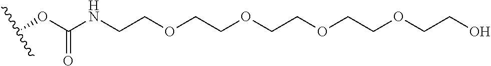 | 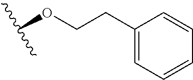 |
| 1101 | 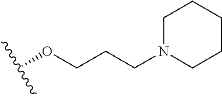 | 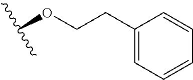 |
| 1102 | 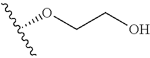 | 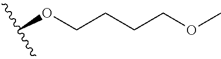 |
| 1103 | 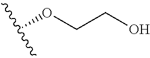 | 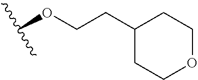 |
| 1104 | 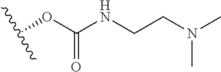 | 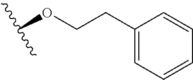 |
| 1105 | 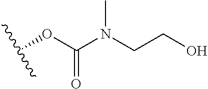 | 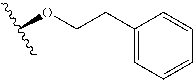 |
| 1106 | 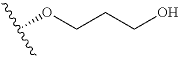 | 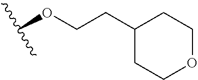 |
| 1107 | 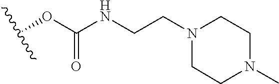 | 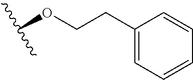 |
| 1108 | 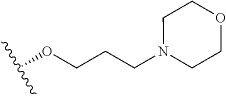 | 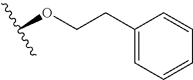 |
| 1109 | 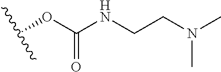 | 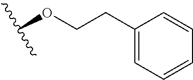 |
| 1110 | 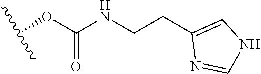 | 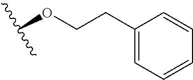 |
| 1111 | 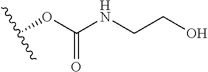 | 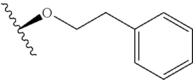 |
| 1112 | 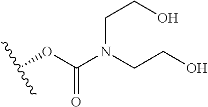 | 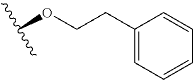 |
| 1113 | 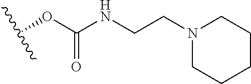 | 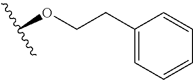 |
| 1114 | 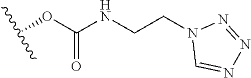 | 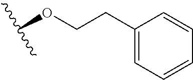 |
| 1115 | 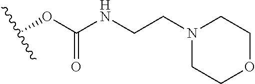 | 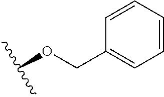 |
| 1116 | 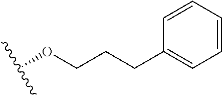 | 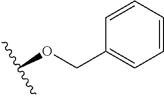 |
| 1117 | 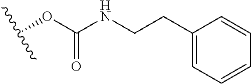 | 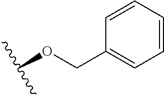 |
| 1118 | 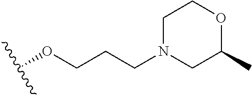 | 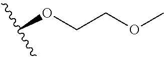 |
| 1119 | 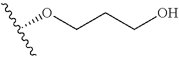 | 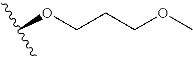 |
| 1120 | 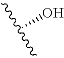 | 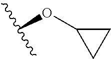 |
| 1121 | 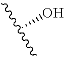 | 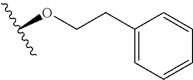 |
| 1122 | 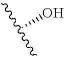 | 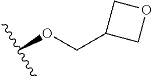 |
| 1123 | 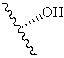 | 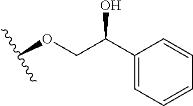 |
| 1124 | 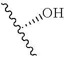 | 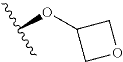 |
| 1125 | 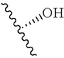 | 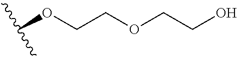 |
| 1126 | 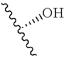 | 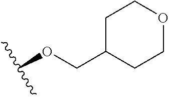 |
| 1127 | 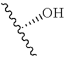 | 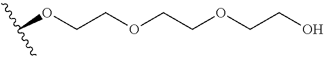 |
| 1128 | 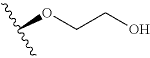 | 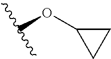 |
| 1130 | 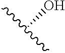 | 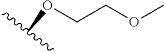 |
| 1131 | 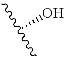 | 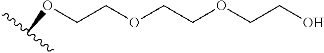 |

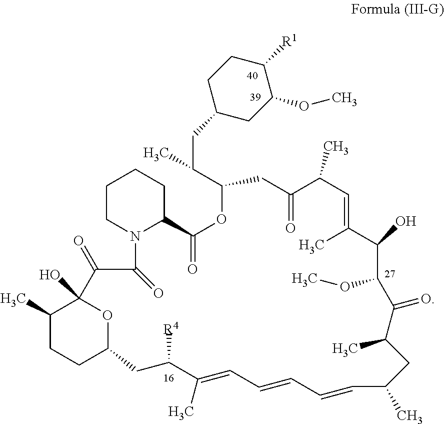

TABLE 7
with Formula (IIII-G) core:

| No. # | R¹ | R⁴ |
|---|---|---|
| 1201 | 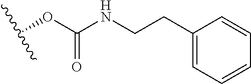 | 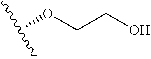 |
| 1202 | 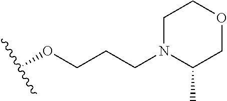 | 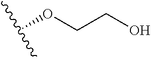 |
| 1203 | 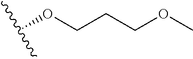 | 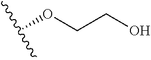 |
| 1204 | 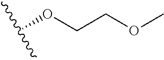 | 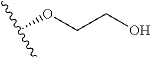 |
| 1205 | 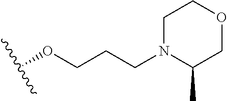 | 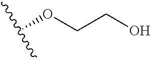 |
| 1206 | 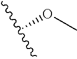 | 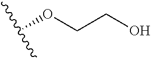 |
| 1207 | 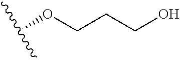 | 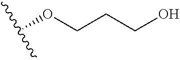 |
| 1208 | 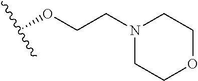 | 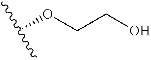 |
| 1209 | 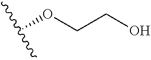 | 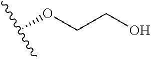 |
| 1210 | 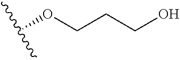 | 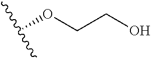 |
| 1211 | 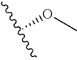 | 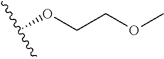 |
| 1212 | 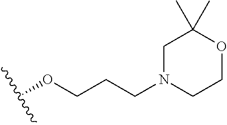 | 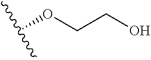 |
| 1213 | 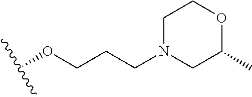 | 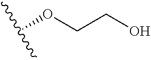 |
| 1214 | 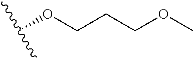 | 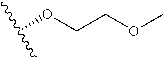 |
| 1215 | 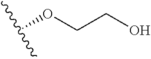 | 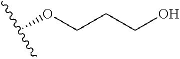 |
| 1216 | 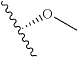 | 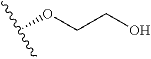 |
| 1217 | 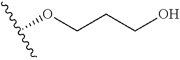 | 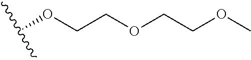 |
| 1218 | 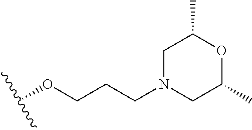 | 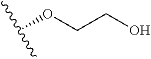 |
| 1219 | 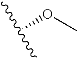 | 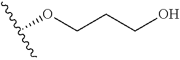 |
| 1220 | 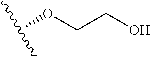 | 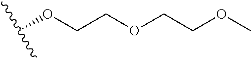 |
| 1221 | 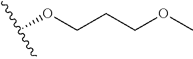 | 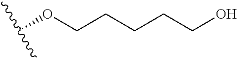 |
| 1222 | 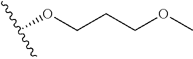 | 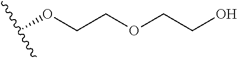 |
| 1223 | 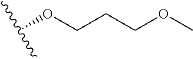 | 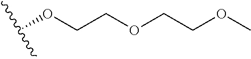 |
| 1224 | 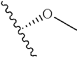 | 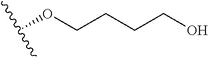 |
| 1225 | 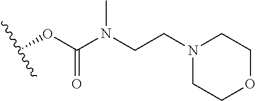 | 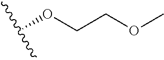 |
| 1226 | 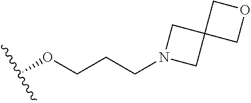 | 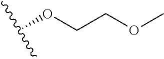 |
| 1227 | 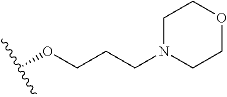 | 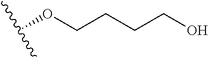 |
| 1228 | 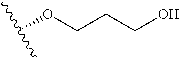 | 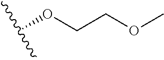 |
| 1229 | 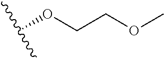 | 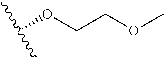 |
| 1230 | 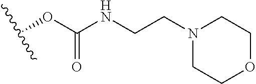 | 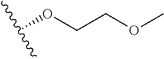 |
| 1231 | 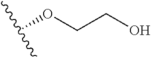 | 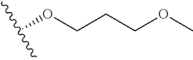 |
| 1232 | 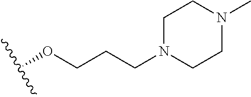 | 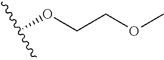 |
| 1233 | 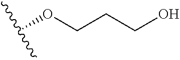 | 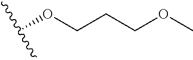 |
| 1234 | 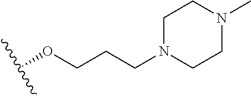 | 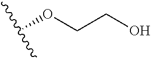 |
| 1235 | 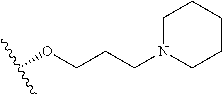 | 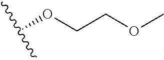 |
| 1236 | 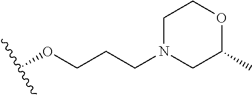 | 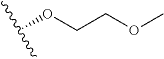 |
| 1237 | 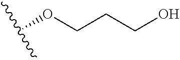 | 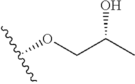 |
| 1238 | 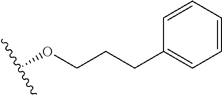 | 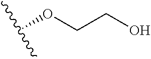 |
| 1239 | 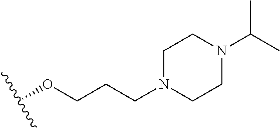 | 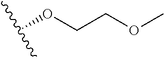 |
| 1240 | 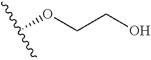 | 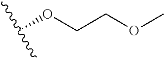 |
| 1241 | 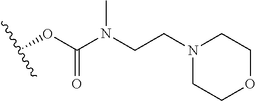 | 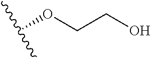 |
| 1242 | 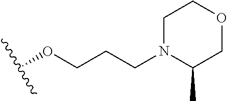 | 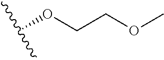 |
| 1243 | 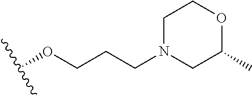 | 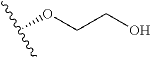 |
| 1244 | 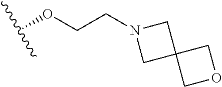 | 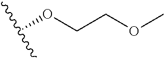 |
| 1245 | 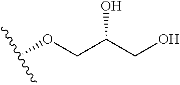 | 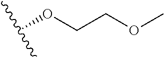 |
| 1246 | 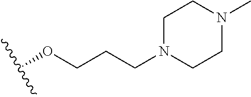 | 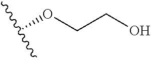 |
| 1247 | 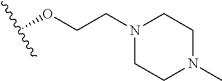 | 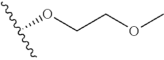 |
| 1248 | 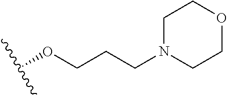 | 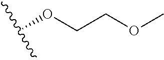 |
| 1249 | 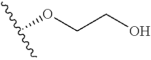 | 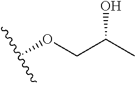 |
| 1250 | 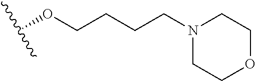 | 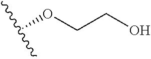 |
| 1251 | 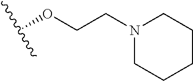 | 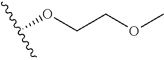 |
| 1252 | 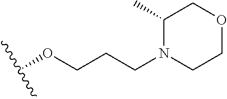 | 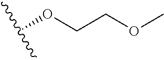 |
| 1253 | 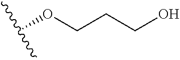 | 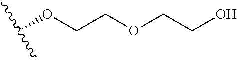 |
| 1254 | 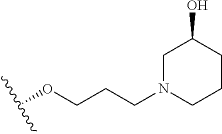 | 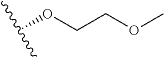 |
| 1255 | 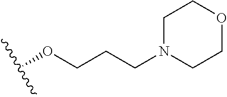 | 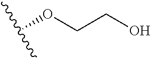 |
| 1256 | 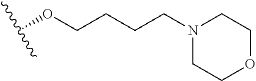 | 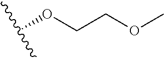 |
| 1257 | 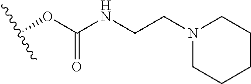 | 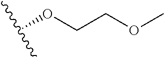 |
| 1258 | 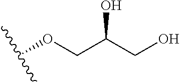 | 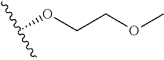 |
| 1259 | 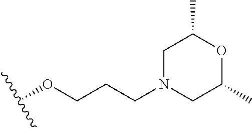 | 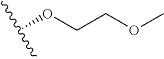 |
| 1260 | 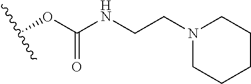 | 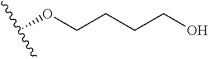 |
| 1261 | 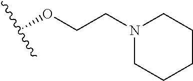 | 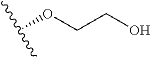 |
| 1262 | 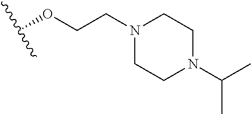 | 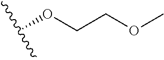 |
| 1263 | 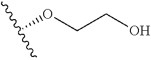 | 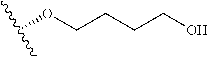 |
| 1264 | 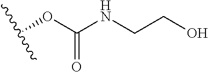 | 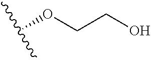 |
| 1265 | 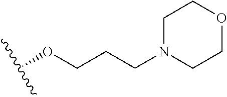 | 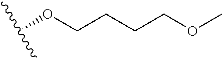 |
| 1266 | 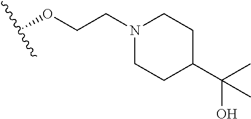 | 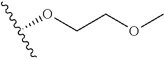 |
| 1267 | 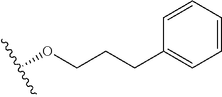 | 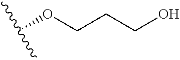 |
| 1268 | 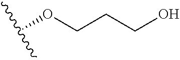 | 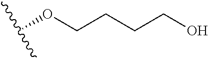 |
| 1269 | 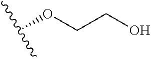 | 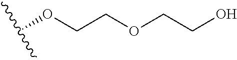 |
| 1270 | 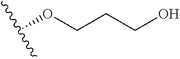 | 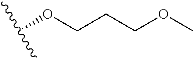 |
| 1271 | 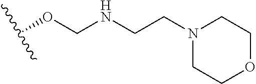 | 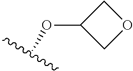 |
| 1272 | 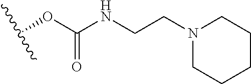 | 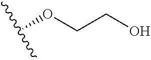 |
| 1273 | 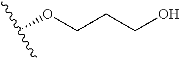 | 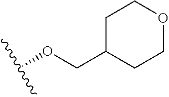 |
| 1274 | 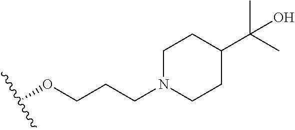 | 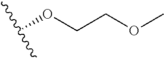 |
| 1275 | 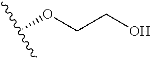 | 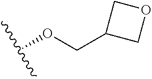 |
| 1276 | 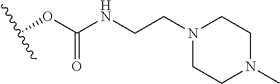 | 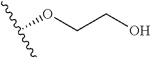 |
| 1277 | 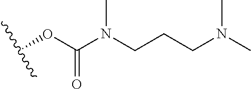 | 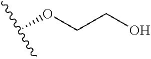 |
| 1278 | 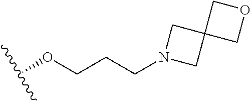 | 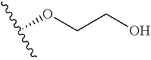 |
| 1279 | 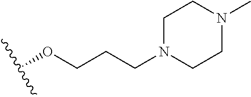 | 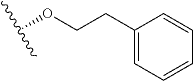 |
| 1280 | 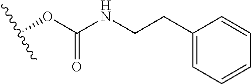 | 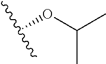 |
| 1281 | 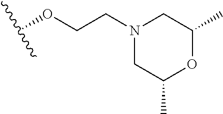 | 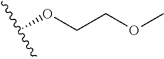 |
| 1282 | 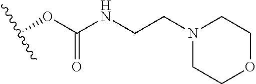 | 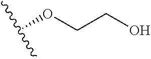 |
| 1283 | 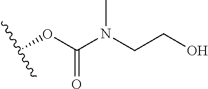 | 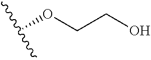 |
| 1284 | 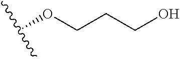 | 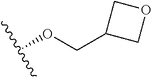 |
| 1285 | 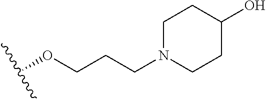 | 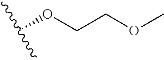 |
| 1286 | 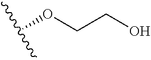 | 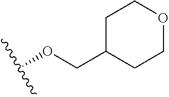 |
| 1287 | 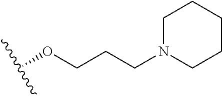 | 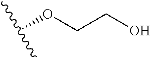 |
| 1288 | 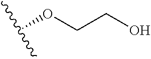 | 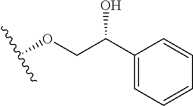 |
| 1289 | 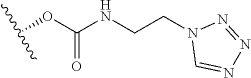 | 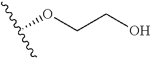 |
| 1290 | 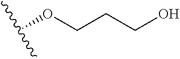 | 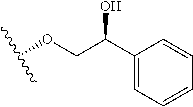 |
| 1291 | 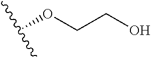 | 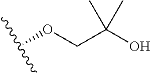 |
| 1292 | 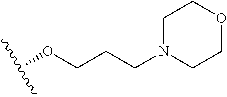 | 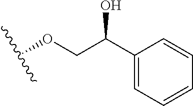 |
| 1293 | 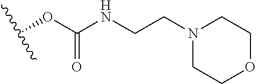 | 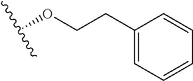 |
| 1294 | 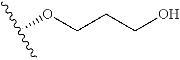 | 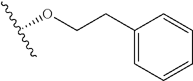 |
| 1295 | 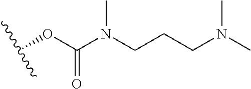 | 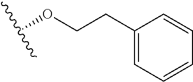 |
| 1296 | 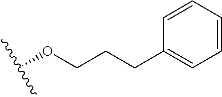 | 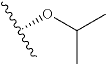 |
| 1297 | 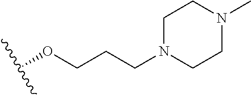 | 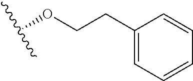 |
| 1298 | 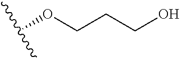 | 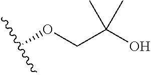 |
| 1299 | 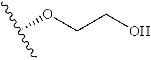 | 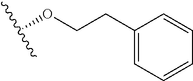 |
| 1300 | 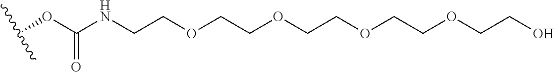 | 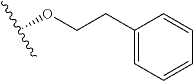 |
| 1301 | 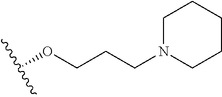 | 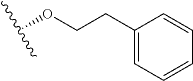 |
| 1302 | 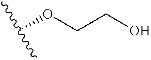 | 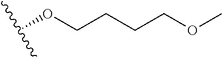 |
| 1303 | 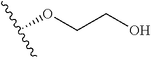 | 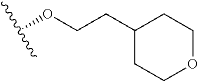 |
| 1304 | 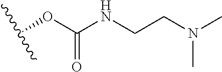 | 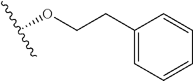 |
| 1305 | 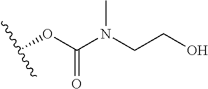 | 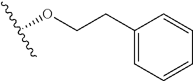 |
| 1306 | 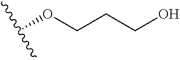 | 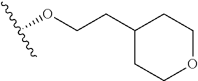 |
| 1307 | 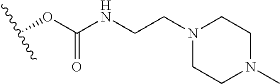 | 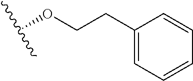 |
| 1308 | 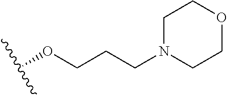 | 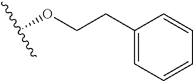 |
| 1309 | 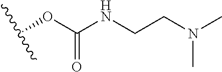 | 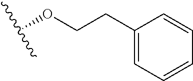 |
| 1310 | 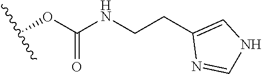 | 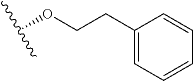 |
| 1311 | 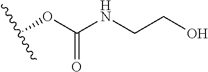 | 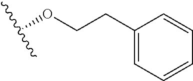 |
| 1312 | 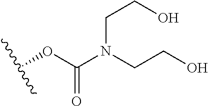 | 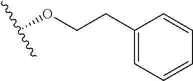 |
| 1313 | 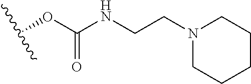 | 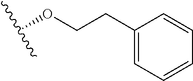 |
| 1314 | 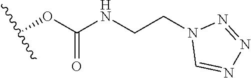 | 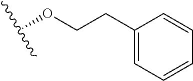 |
| 1315 | 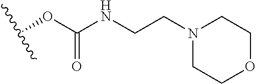 | 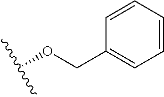 |
| 1316 | 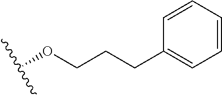 | 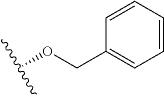 |
| 1317 | 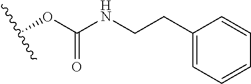 | 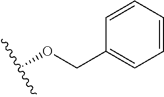 |
| 1318 | 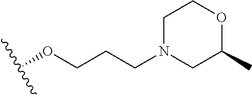 | 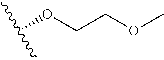 |
| 1319 | 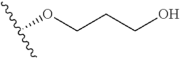 | 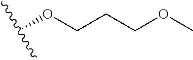 |
| 1320 | 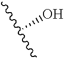 | 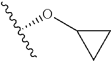 |
| 1321 | 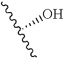 | 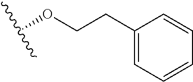 |
| 1322 | 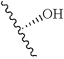 | 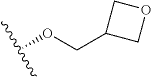 |
| 1323 | 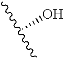 | 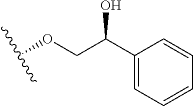 |
| 1324 | 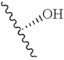 | 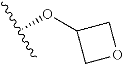 |
| 1325 | 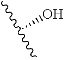 | 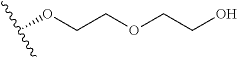 |
| 1326 | 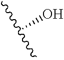 | 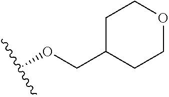 |
| 1327 | 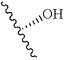 | 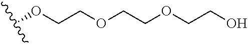 |
| 1328 | 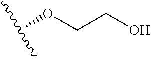 | 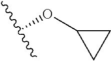 |
| 1330 | 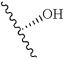 | 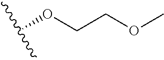 |
| 1331 | 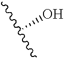 | 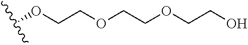 |

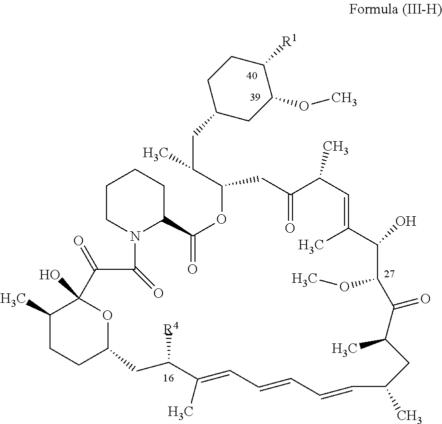

TABLE 8
with Formula (III-H) core:

| No. # | R¹ | R⁴ |
|---|---|---|
| 1401 | 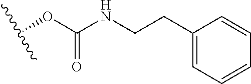 | 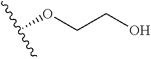 |
| 1402 | 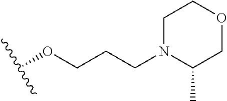 | 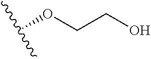 |
| 1403 | 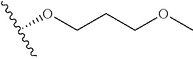 | 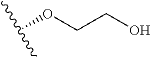 |
| 1404 | 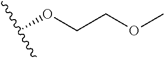 | 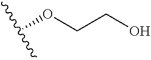 |
| 1405 | 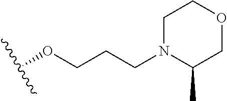 | 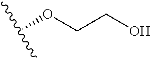 |
| 1406 | 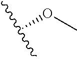 | 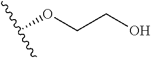 |
| 1407 | 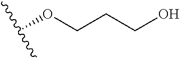 | 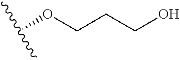 |
| 1408 | 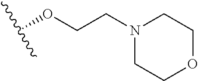 | 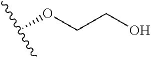 |
| 1409 | 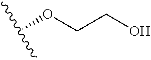 | 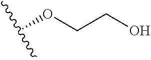 |
| 1410 | 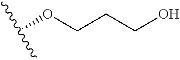 | 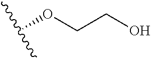 |
| 1411 | 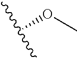 | 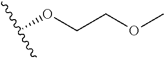 |
| 1412 | 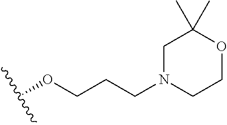 | 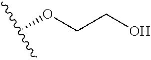 |
| 1413 | 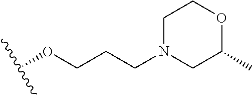 | 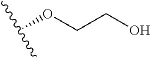 |
| 1414 | 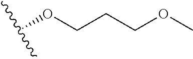 | 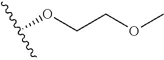 |
| 1415 | 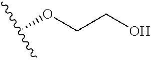 | 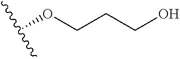 |
| 1416 | 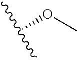 | 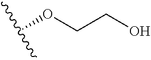 |
| 1417 | 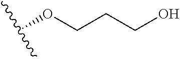 | 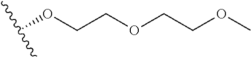 |
| 1418 | 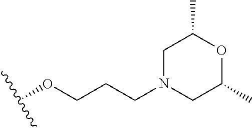 | 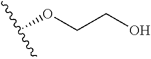 |
| 1419 | 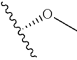 | 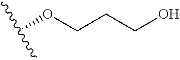 |
| 1420 | 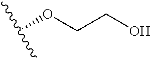 | 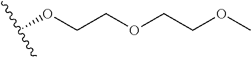 |
| 1421 | 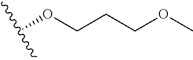 | 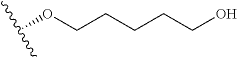 |
| 1422 | 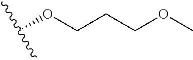 | 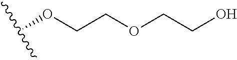 |
| 1423 | 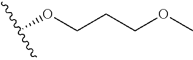 | 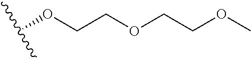 |
| 1424 | 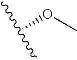 | 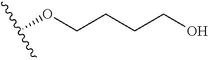 |
| 1425 | 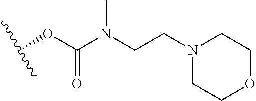 | 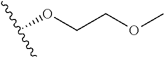 |
| 1426 | 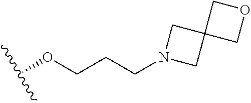 | 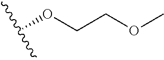 |
| 1427 | 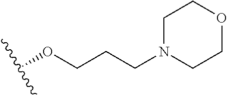 | 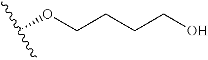 |
| 1428 | 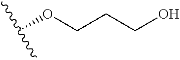 | 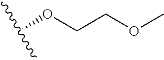 |
| 1429 | 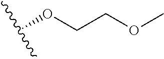 | 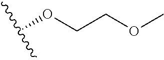 |
| 1430 | 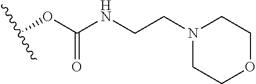 | 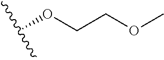 |
| 1431 | 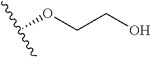 | 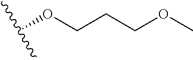 |
| 1432 | 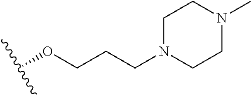 | 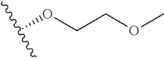 |
| 1433 | 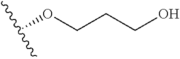 | 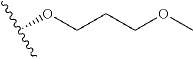 |
| 1434 | 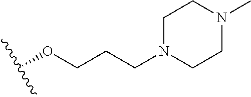 | 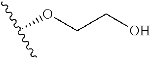 |
| 1435 | 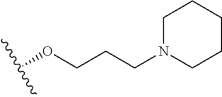 | 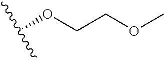 |
| 1436 | 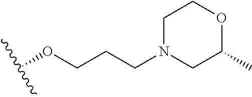 | 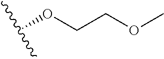 |
| 1437 | 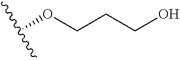 | 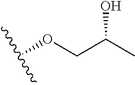 |
| 1438 | 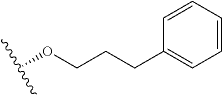 | 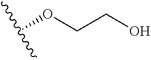 |
| 1439 | 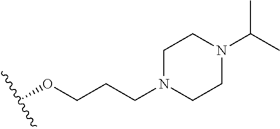 | 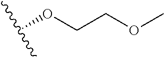 |
| 1440 | 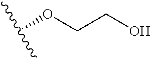 | 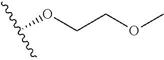 |
| 1441 | 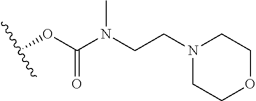 | 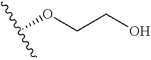 |
| 1442 | 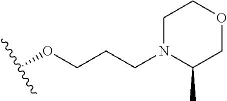 | 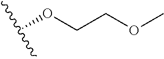 |
| 1443 | 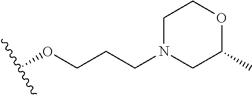 | 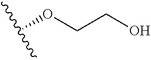 |
| 1444 | 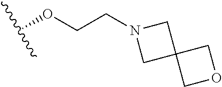 | 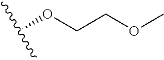 |
| 1445 | 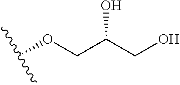 | 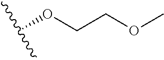 |
| 1446 | 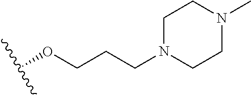 | 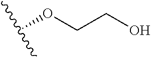 |
| 1447 | 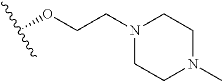 | 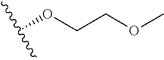 |
| 1448 | 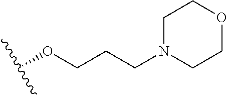 | 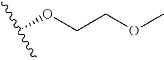 |
| 1449 | 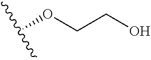 | 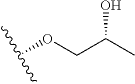 |
| 1450 | 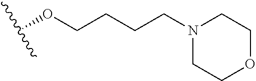 | 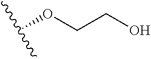 |
| 1451 | 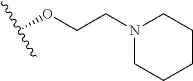 | 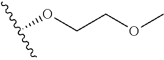 |
| 1452 | 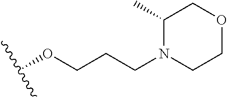 | 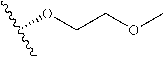 |
| 1453 | 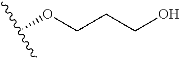 | 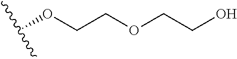 |
| 1454 | 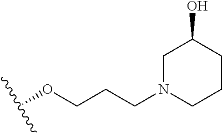 | 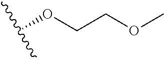 |
| 1455 | 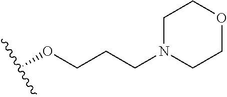 | 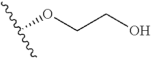 |
| 1456 | 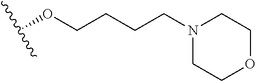 | 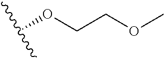 |
| 1457 | 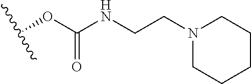 | 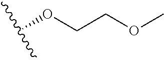 |
| 1458 | 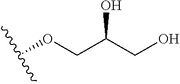 | 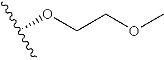 |
| 1459 | 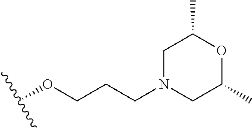 | 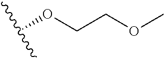 |
| 1460 | 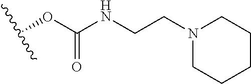 | 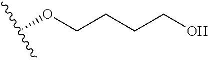 |
| 1461 | 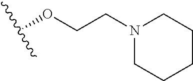 | 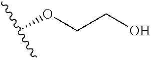 |
| 1462 | 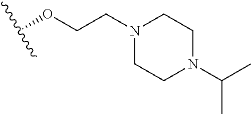 | 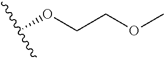 |
| 1463 | 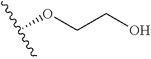 | 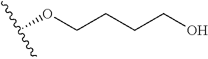 |
| 1464 | 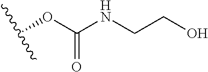 | 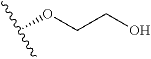 |
| 1465 | 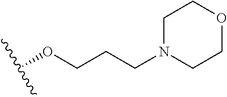 | 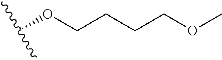 |
| 1466 | 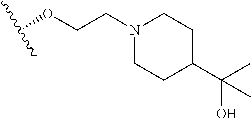 | 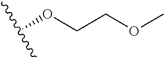 |
| 1467 | 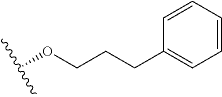 | 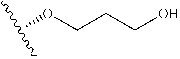 |
| 1468 | 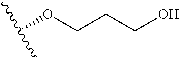 | 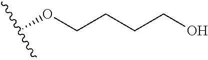 |
| 1469 | 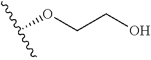 | 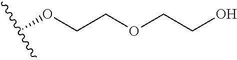 |
| 1470 | 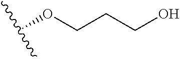 | 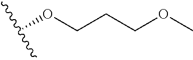 |
| 1471 | 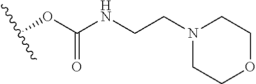 | 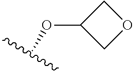 |
| 1472 | 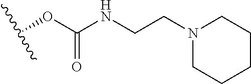 | 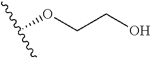 |
| 1473 | 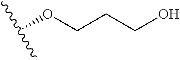 | 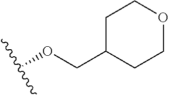 |
| 1474 | 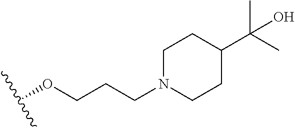 | 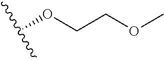 |
| 1475 | 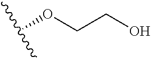 | 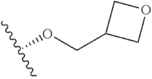 |
| 1476 | 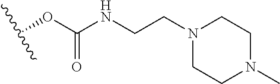 | 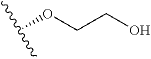 |
| 1477 | 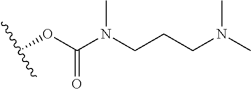 | 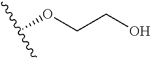 |
| 1478 | 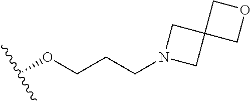 | 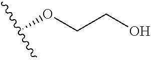 |
| 1479 | 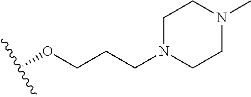 | 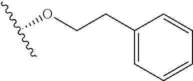 |
| 1480 | 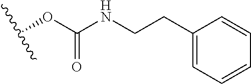 | 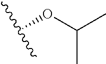 |
| 1481 | 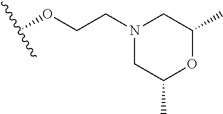 | 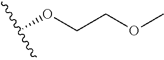 |
| 1482 | 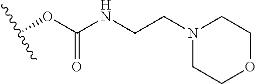 | 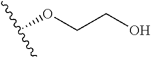 |
| 1483 | 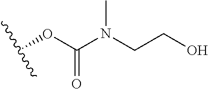 | 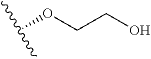 |
| 1484 | 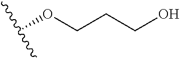 | 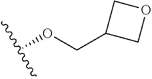 |
| 1485 | 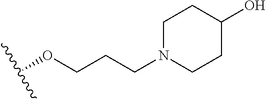 | 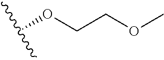 |
| 1486 | 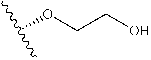 | 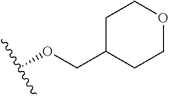 |
| 1487 | 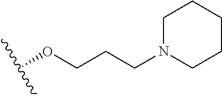 | 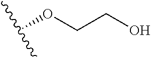 |
| 1488 | 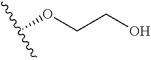 | 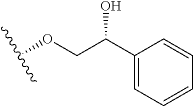 |
| 1489 | 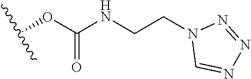 | 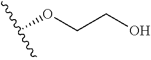 |
| 1490 | 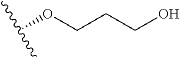 | 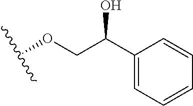 |
| 1491 | 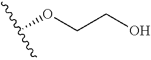 | 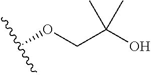 |
| 1492 | 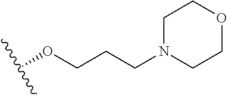 | 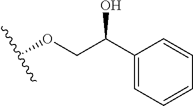 |
| 1493 | 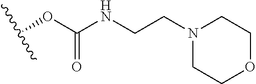 | 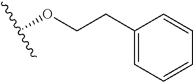 |
| 1494 | 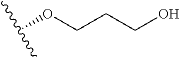 | 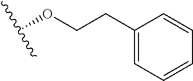 |
| 1495 | 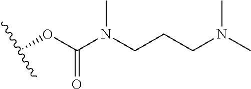 | 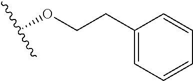 |
| 1496 | 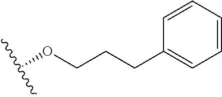 | 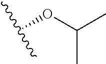 |
| 1497 | 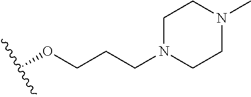 | 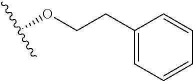 |
| 1498 | 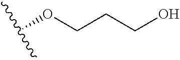 | 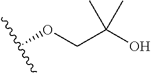 |
| 1499 | 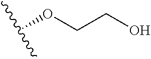 | 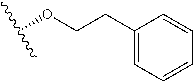 |
| 1500 | 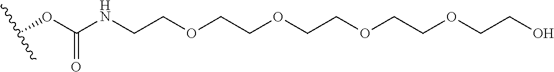 | 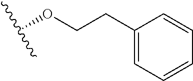 |
| 1501 | 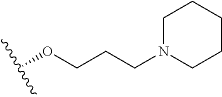 | 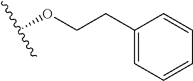 |
| 1502 | 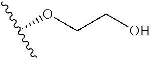 | 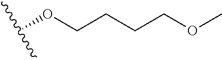 |
| 1503 | 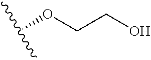 | 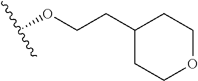 |
| 1504 | 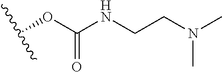 | 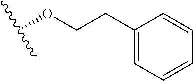 |
| 1505 | 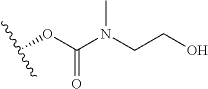 | 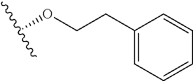 |
| 1506 | 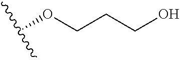 | 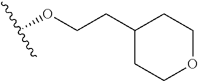 |
| 1507 | 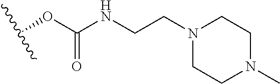 | 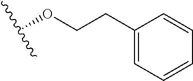 |
| 1508 | 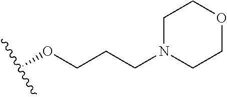 | 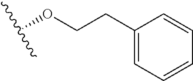 |
| 1509 | 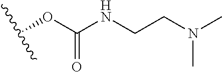 | 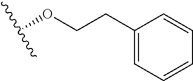 |
| 1510 | 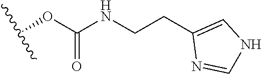 | 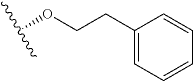 |
| 1511 | 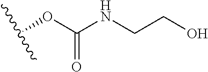 | 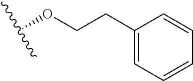 |
| 1512 | 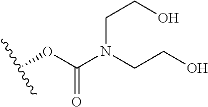 | 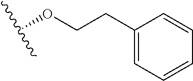 |
| 1513 | 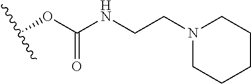 | 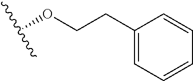 |
| 1514 | 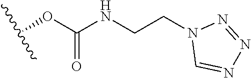 | 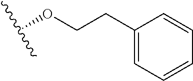 |
| 1515 | 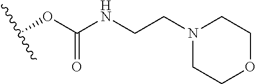 | 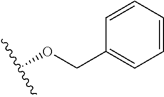 |
| 1516 | 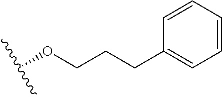 | 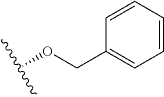 |
| 1517 | 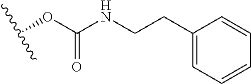 | 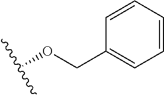 |
| 1518 | 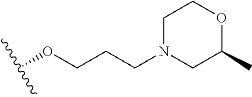 | 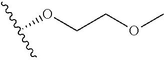 |
| 1519 | 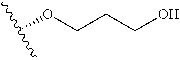 | 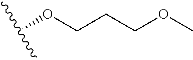 |
| 1520 | 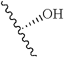 | 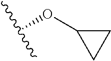 |
| 1521 | 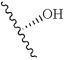 | 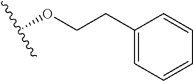 |
| 1522 | 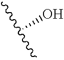 | 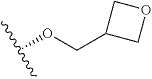 |
| 1523 | 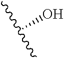 | 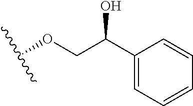 |
| 1524 | 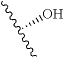 | 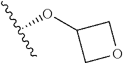 |
| 1525 | 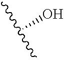 | 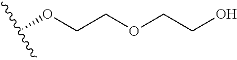 |
| 1526 | 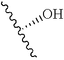 | 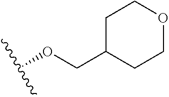 |
| 1527 | 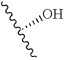 | 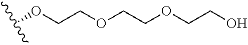 |
| 1528 | 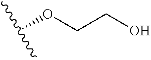 | 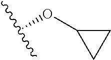 |
| 1530 | 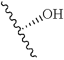 | 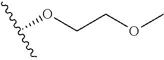 |
| 1531 | 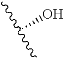 | 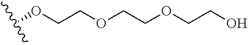 |

### Example 1: Supercritical Fluid Chromatography (SFC) Analytical Methods for Separating Various Diastereomers

TABLE 9
SFC analytical methods

| | Conditions |
|---|---|
| Analytical SFC conditions: | type |
| Instrument: Waters UPC2 | I |
| Stationary phase: Princeton 2 EthylPyridine | |
| 4.6 × 150 mm 5 μm | |
| Mobile phase: CO2/IpOH 80/20 | |
| Flowrate: 2.4 mL/min | |
| Pressure: 100 bar | |
| Temperature: 40° C. | |
| Detection: 277 nm | |
| Instrument: Waters UPC2 | II |
| Stationary phase: Princeton 2 EthylPyridine | |
| 4.6 × 150 mm 5 μm | |
| Mobile phase: CO2/IpOH 83/17 | |
| Flowrate: 2.4 mL/min | |
| Pressure: 100 bar | |
| Temperature: 40° C. | |
| Detection: 277 nm | |
| Instrument: Waters UPC2 | III |
| Stationary phase: Princeton 2 EthylPyridine | |
| 4.6 × 150 mm 5 μm | |
| Mobile phase: CO2/IpOH 85/15 | |
| Flowrate: 2.4 mL/min | |
| Pressure: 100 bar | |
| Temperature: 40° C. | |
| Detection: 277 nm | |
| Instrument: Waters UPC2 | IV |
| Stationary phase: Princeton 2 EthylPyridine | |
| 4.6 × 150 mm 5 μm | |
| Mobile phase: CO2/IpOH 75/25 | |
| Flowrate: 2.4 mL/min | |
| Pressure: 100 bar | |
| Temperature: 40° C. | |
| Detection: 277 nm | |

TABLE 10
Structures and Retention Time

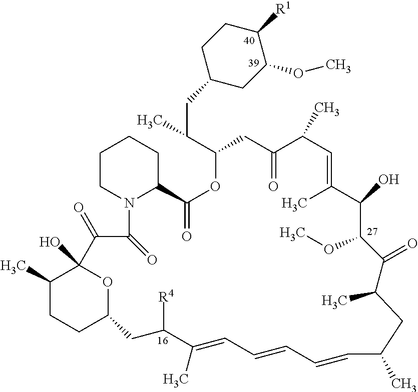

| | | Retention Time | Analytical |
|---|---|---|---|
| R¹ | R⁴ | (minutes) | Method |
| 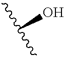 | 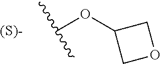 [SMILES: *OC1COC1] | 8.0 | I |
| 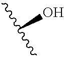 | 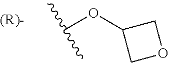 [SMILES: *OC1COC1] | 10.0 | I |
| 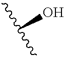 | 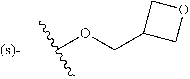 [SMILES: *OCC1COC1] | 9.4 | I |
| 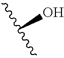 | 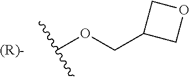 [SMILES: *OCC1COC1] | 10.5 | I |
| 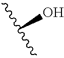 | 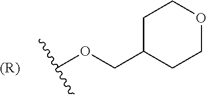 [SMILES: "(R)" ; *OCC1CCOCC1] | 15.6 | II |
| 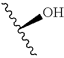 | 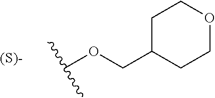 [SMILES: *OCC1CCOCC1] | 18.3 | II |
| 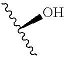 | 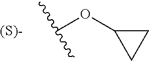 [SMILES: *OC1CC1] | 7.9 | I |
| 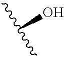 | 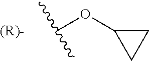 [SMILES: *OC1CC1] | 9.1 | I |
| 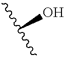 | 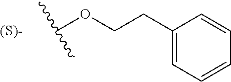 [SMILES: *OCCc1ccccc1] | 11.2 | I |
| 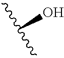 | 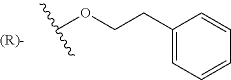 [SMILES: *OCCc1ccccc1] | 13.0 | I |
| 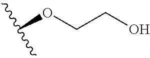 | 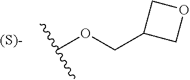 [SMILES: *OCC1COC1] | 10.3 | I |
| 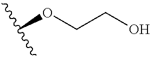 | 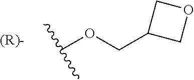 [SMILES: *OCC1COC1] | 11.7 | I |
| 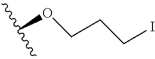 | 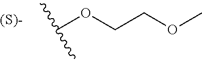 [SMILES: *OCCOC] | 7.9 | I |
| 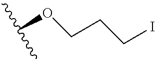 | 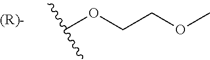 [SMILES: *OCCOC] | 8.6 | I |
| 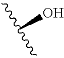 | 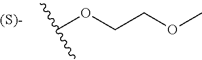 [SMILES: *OCCOC] | 11.0 | II |
| 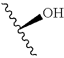 | 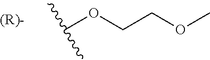 [SMILES: *OCCOC] | 10.8 | II |
| 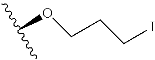 | 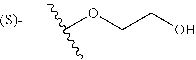 [SMILES: *OCCO] | 15.5 | I |
| 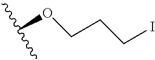 | 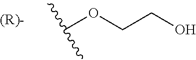 [SMILES: *OCCO] | 17.4 | I |
| 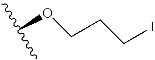 | 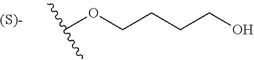 [SMILES: *OCCCCO] | 33.5 | II |
| 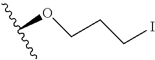 | 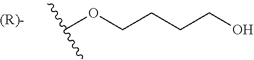 [SMILES: *OCCCCO] | 37.4 | II |
| 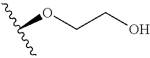 | 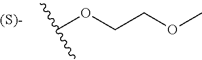 [SMILES: *OCCOC] | 19.6 | III |
| 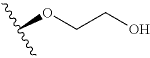 | 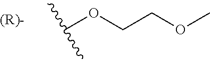 [SMILES: *OCCOC] | 23.6 | III |
| 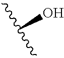 | 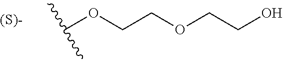 [SMILES: *OCCOCCO] | 15.1 | I |
| 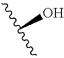 | 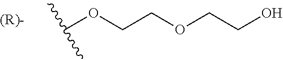 [SMILES: *OCCOCCO] | 16.3 | I |
| 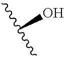 | 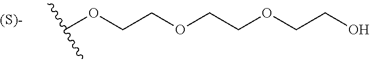 [SMILES: *OCCOCCOCCO] | 15.2 | I |
| 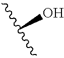 | 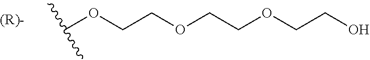 [SMILES: *OCCOCCOCCO] | 16.5 | I |
| 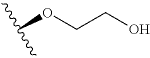 | 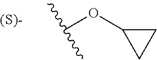 [SMILES: *OC1CC1] | 8.7 | I |
| 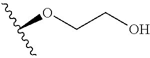 | 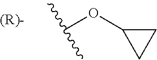 [SMILES: *OC1CC1] | 9.8 | I |
| 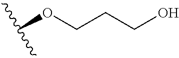 | 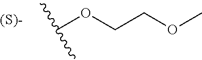 [SMILES: *OCCOC] | 10.2 | I |
| 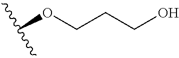 | 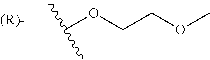 [SMILES: *OCCOC] | 11.9 | I |
| 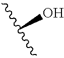 | 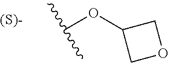 [SMILES: *OC1COC1] | 8.0 | I |
| 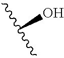 | 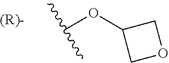 [SMILES: *OC1COC1] | 10.0 | I |
| 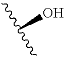 | 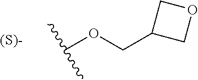 [SMILES: *OCC1COC1] | 9.4 | I |
| 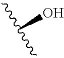 | 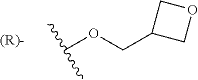 [SMILES: *OCC1COC1] | 10.5 | I |
| 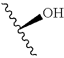 | 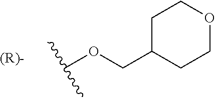 [SMILES: *OCC1CCOCC1] | 15.6 | II |
| 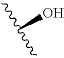 | 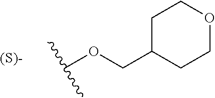 [SMILES: *OCC1CCOCC1] | 18.3 | II |
| 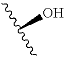 | 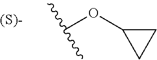 [SMILES: *OC1CC1] | 7.9 | I |
| 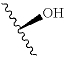 | 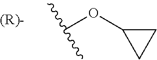 [SMILES: *OC1CC1] | 9.1 | I |
| 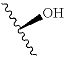 | 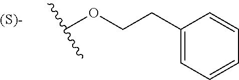 [SMILES: *OCCc1ccccc1] | 11.2 | I |
| 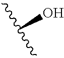 | 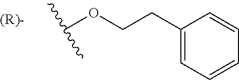 [SMILES: *OCCc1ccccc1] | 13.0 | I |
| 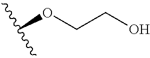 | 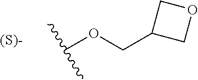 [SMILES: *OCC1COC1] | 10.3 | I |
| 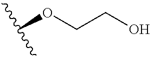 | 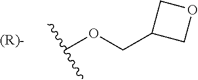 [SMILES: *OCC1COC1] | 11.7 | I |
| 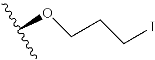 | 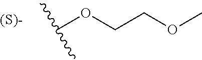 [SMILES: *OCCOC] | 7.9 | I |
| 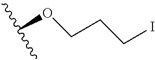 | 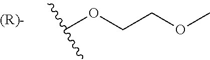 [SMILES: *OCCOC] | 8.6 | I |
| 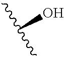 | 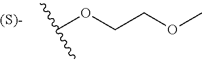 [SMILES: *OCCOC] | 11.0 | II |
| 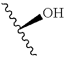 | 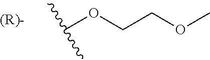 [SMILES: *OCCOC] | 10.8 | II |
| 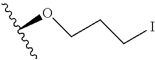 | 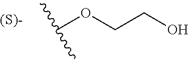 [SMILES: *OCCO] | 15.5 | I |
| 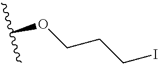 | 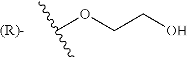 [SMILES: *OCCO] | 17.4 | I |
| 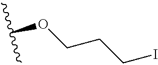 | 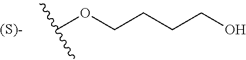 [SMILES: *OCCCCO] | 33.5 | II |
| 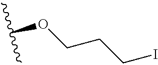 | 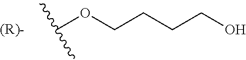 [SMILES: *OCCCCO] | 37.4 | II |
| 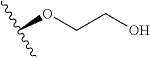 | 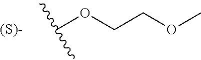 [SMILES: *OCCOC] | 19.6 | III |
| 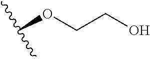 | 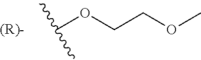 [SMILES: *OCCOC] | 23.6 | III |
| 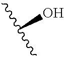 | 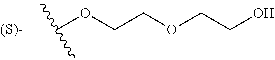 [SMILES: *OCCOCCO] | 15.1 | I |
| 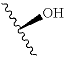 | 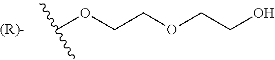 [SMILES: *OCCOCCO] | 16.3 | I |
| 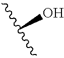 | 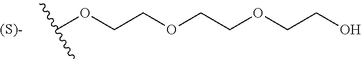 [SMILES: *OCCOCCOCCO] | 15.2 | I |
| 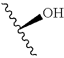 | 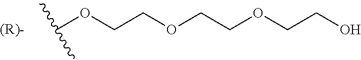 [SMILES: *OCCOCCOCCO] | 16.5 | I |
| 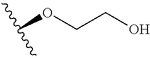 | 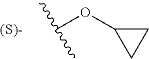 [SMILES: *OC1CC1] | 8.7 | I |
| 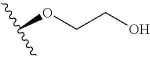 | 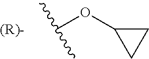 [SMILES: *OC1CC1] | 9.8 | I |
| 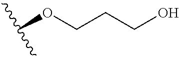 | 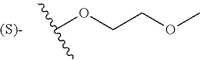 [SMILES: *OCCOC] | 10.2 | I |
| 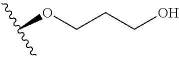 | 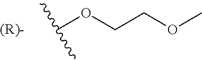 [SMILES: *OCCOC] | 11.9 | I |
| 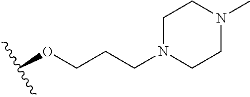 | 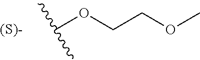 [SMILES: *OCCOC] | 6.49 | IV |
| 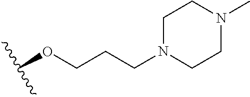 | 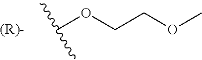 [SMILES: *OCCOC] | 7.41 | IV |
| 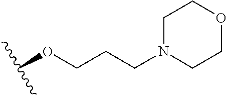 | 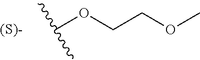 [SMILES: *OCCOC] | 18.44 | III |
| 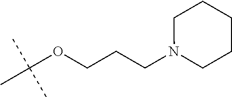 | 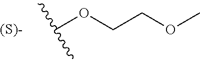 [SMILES: *OCCOC] | 9.60 | I |
| 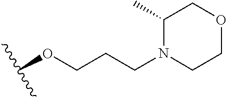 | 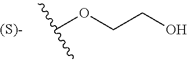 [SMILES: *OCCO] | 14.75 | I |
| 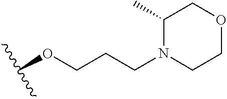 | 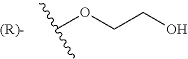 [SMILES: *OCCO] | 17.98 | I |
| 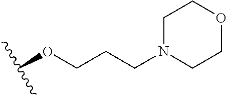 | 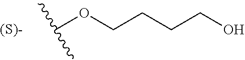 [SMILES: *OCCCCO] | 17.24 | I |

Differential pharmacology of compounds described herein in the following assays may be observed in different cell or tissue types depending on (1) the relative abundance of FKBP homologs in these cells/tissues and (2) the specificity of binding to these different FKBP homologs (Mol. Cell Biol. (2013) 33: 1357-1367). Various FKBP homologs are used in the following examples.

### Example 2

SPR Assay to Determine Binding Affinity to FKBP12.

Biotinylated avi-FKBP12 was immobilized on a streptavidin chip (Cytiva Series S SA) using a Biacore 8K or 8k+ (Cytiva). To achieve an immobilization level of 1000 RU, 2 μg/ml biotinylated avi-FKBP12 were injected for 100 sec at a flow rate of 10 l/min. Test compounds described in Table 11 were diluted in DMSO to 100× working concentration. Each test compound was 100-fold diluted in 50 mM HEPES pH 7.5, 150 mM NaCl, 2 mM MgCl₂, 1 mM DTT, 0.05% Tween-20 and a serial dilution prepared (9 concentrations, 3-fold dilutions, 0.08-500 nM). Rapamycin was used as reference sample (9 concentrations, 3-fold dilutions, 0.02-100 nM). The compound dilutions were then injected at 100 uL/min for 120 seconds contact time in sequence with increasing concentrations. Dissociation was monitored for 3600 seconds. 50 mM HEPES pH 7.5, 150 mM NaCl, 2 mM MgCl₂, 1 mM DTT, 0.05% Tween-20, 1% DMSO was used as running buffer. The single-cycle kinetics data were fit to a 1:1 binding model to measure the association rate ka (1/Ms), the dissociation rate kd (1/s) and the affinity Kd (M).

Table 11 includes FKBP12 direct binding K_{d} (nM) values of selected compounds; with compounds having a FKBP12 direct binding K_{d} of less than 0.3 nM as A, 0.3 nM to 1.0 nM as B, and greater than 1.0 nM as C.

TABLE 11
FKBP12 direct binding of various compounds of compounds represented by the Formula:

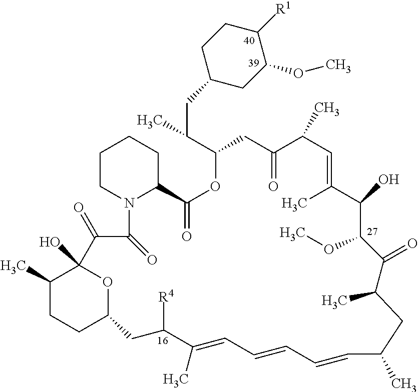

| Compound | | | FKBP12 Direct |
|---|---|---|---|
| Number | R¹ | R⁴ | Binding K_{d}, nM |
| 431 | 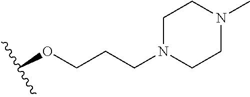 | 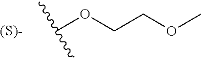 [SMILES: *OCCOC] | B |
| 32 | 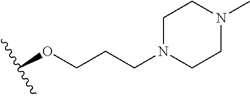 | 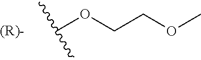 [SMILES: *OCCOC] | B |
| 521 | 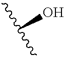 | 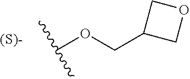 [SMILES: *OCC1COC1] | A |
| 122 | 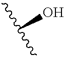 | 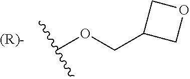 [SMILES: *OCC1COC1] | B |
| 474 | 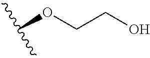 | 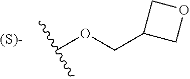 [SMILES: *OCC1COC1] | B |
| 75 | 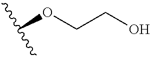 | 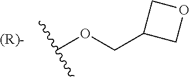 [SMILES: *OCC1COC1] | B |
| 524 | 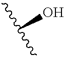 | 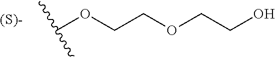 [SMILES: *OCCOCCO] | A |
| 125 | 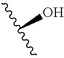 | 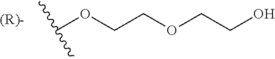 [SMILES: *OCCOCCO] | A |
| 523 | 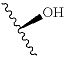 | 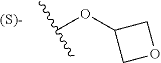 [SMILES: *OC1COC1] | A |
| 124 | 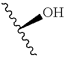 | 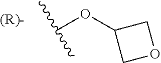 [SMILES: *OC1COC1] | A |
| 519 | 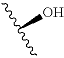 | 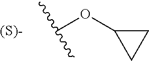 [SMILES: *OC1CC1] | A |
| 120 | 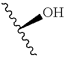 | 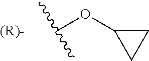 [SMILES: *OC1CC1] | B |
| 527 | 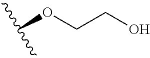 | 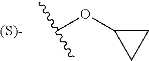 [SMILES: *OC1CC1] | B |
| 128 | 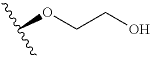 | 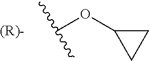 [SMILES: *OC1CC1] | C |
| 520 | 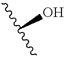 | 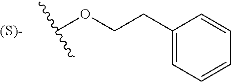 [SMILES: *OCCc1ccccc1] | C |
| 121 | 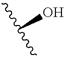 | 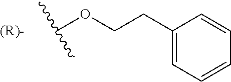 [SMILES: *OCCc1ccccc1] | C |
| 404 | 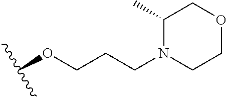 | 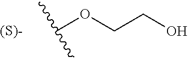 [SMILES: *OCCO] | A |
| 5 | 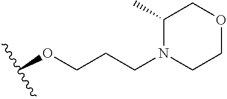 | 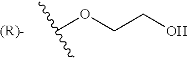 [SMILES: *OCCO] | |
| 426 | 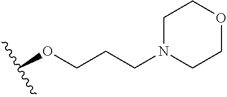 | 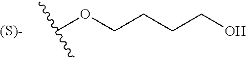 [SMILES: *OCCCCO] | A |
| 27 | 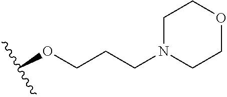 | 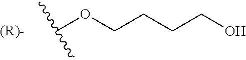 [SMILES: *OCCCCO] | A |
| 427 | 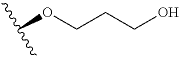 | 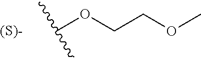 [SMILES: *OCCOC] | B |
| 28 | 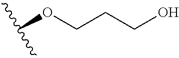 | 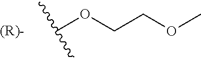 [SMILES: *OCCOC] | C |
| 439 | 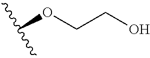 | 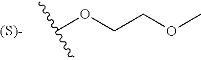 [SMILES: *OCCOC] | B |
| 40 | 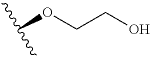 | 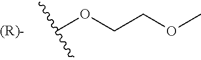 [SMILES: *OCCOC] | C |
| 529 | 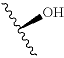 | 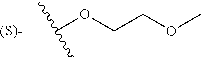 [SMILES: *OCCOC] | A |
| 130 | 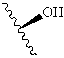 | 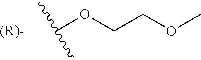 [SMILES: *OCCOC] | B |

| Compound | FKBP12 K_{d} (nM) |
|---|---|
| Rapamycin | A |

### Example 3: SPR Assay to Determine Binding Affinity to FKBP51

Biotinylated avi-FKBP51 is immobilized on a streptavidin chip (Cytiva Series S SA) using a Biacore 8K or 8k+ (Cytiva). To achieve an immobilization level of 2000 RU, 3 μg/ml biotinylated avi-FYBP51 are injected for 360 sec at a flow rate of 10 glu/min. Test compounds are diluted in DMSO to 100× working concentration. Each test compound is 100-fold diluted in 50 mM HEPES pH 7.5, 150 mM NaCl, 2 MM MgCl₂, 1 mM DTT, 0.05% Tween-20 and a serial dilution prepared (8 concentrations, 3-fold dilutions, 0.5-1000 nM). Rapamycin was used as reference sample (8 concentrations, 3-fold dilutions, 0.5-1000 nM). The compound dilutions were then injected at 100 uL/min for 120 seconds contact time and with 3600 seconds dissociation time with increasing concentrations. 50 mM HEPES pH 7.5, 150 mM NaCl, 2 mM MgCl₂, 1 mM DTT, 0.05% Tween-20, 1% DMSO was used as running buffer. Multi-cycle kinetics data were fit to a 1:1 binding model to measure the association rate ka (1/Ms), the dissociation rate kd (1/s) and the affinity Kd (M).

### Example 4: SPR Assay to Characterize Ternary Complex Formation with FKBP12

Biotinylated avi-FKBP12 was immobilized on a streptavidin chip (Cytiva Series S SA) using a Biacore 8K or 8k+ (Cytiva). To achieve an immobilization level of 100 RU, 0.3 μg/ml biotinylated avi-FKBP12 were injected for 80 sec at a flow rate of 10 μl/min. Serial dilution of FRB was prepared (12 concentrations, 3-fold dilutions, 0.00011-20 μM) and supplemented with 100 nM of a test compound. A-B-A injection mode was used to ensure saturation immobilized FKBP12 with respective test compound. 100 nM solution of the respective test compound was injected before FRB injection for 120 sec and during dissociation for 420 sec. The FRB dilutions were then injected 120 seconds contact time with increasing concentrations. Rapamycin was used as reference sample. 50 mM HEPES pH 7.5, 150 mM NaCl, 2 mM MgCl₂, 1 mM DTT, 0.05% Tween-20, 1% DMSO was used as running buffer at a flow rate of 30 μl/min. The multi-cycle kinetics data were fit to a 1:1 binding model to measure the association rate ka (1/Ms), the dissociation rate kd (1/s) and the affinity Kd (M). In case of fast association and dissociation, steady state affinity analysis following the law of mass action was used to determine the affinity Kd (M).

Table 12 includes FKBP12 ternary complex K_{d} (nM) values of selected compounds; with compounds having a FKBP12 ternary complex K_{d} of less than 500 nM as A, 500 nM to 1100 nM as B, and greater than 1100 nM as C.

TABLE 12
FKBP12 Ternary Complex of various compounds of compounds represented by the Formula:

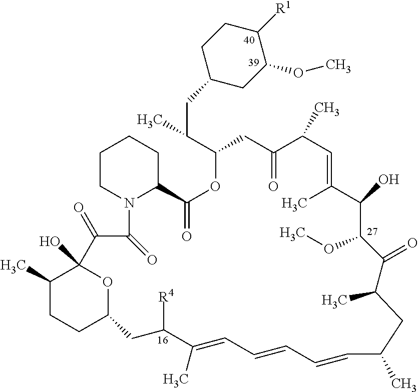

| | | | FKBP12 |
|---|---|---|---|
| | | | Ternary |
| Compound | | | Complex |
| Number | R¹ | R⁴ | K_{d}, nM |
| 431 | 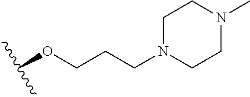 | 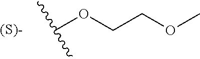 [SMILES: *OCCOC] | A |
| 32 | 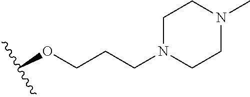 | 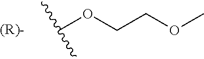 [SMILES: *OCCOC] | C |
| 521 | 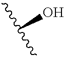 | 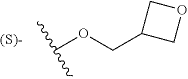 [SMILES: *OCC1COC1] | B |
| 122 | 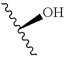 | 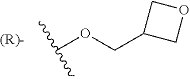 [SMILES: *OCC1COC1] | C |
| 474 | 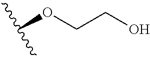 | 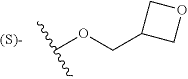 [SMILES: *OCC1COC1] | B |
| 75 | 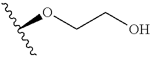 | 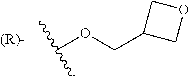 [SMILES: *OCC1COC1] | C |
| 524 | 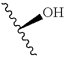 | 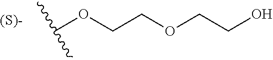 [SMILES: *OCCOCCO] | B |
| 125 | 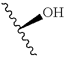 | 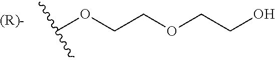 [SMILES: *OCCOCCO] | C |
| 523 | 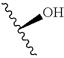 | 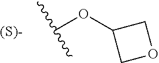 [SMILES: *OC1COC1] | C |
| 124 | 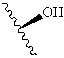 | 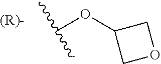 [SMILES: *OC1COC1] | C |
| 519 | 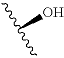 | 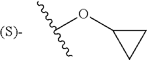 [SMILES: *OC1CC1] | A |
| 120 | 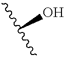 | 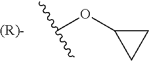 [SMILES: *OC1CC1] | A |
| 527 | 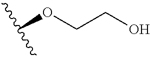 | 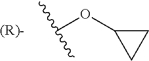 [SMILES: *OC1CC1] | A |
| 128 | 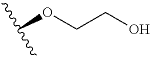 | 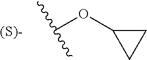 [SMILES: *OC1CC1] | A |
| 520 | 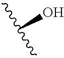 | 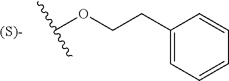 [SMILES: *OCCc1ccccc1] | A |
| 121 | 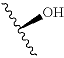 | 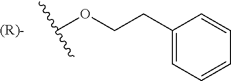 [SMILES: *OCCc1ccccc1] | A |
| 401 | 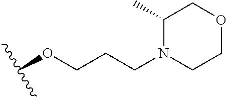 | 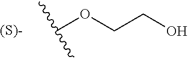 [SMILES: *OCCO] | A |
| 5 | 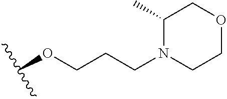 | 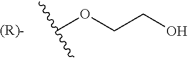 [SMILES: *OCCO] | |
| 426 | 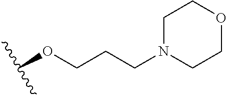 | 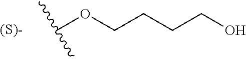 [SMILES: *OCCCCO] | B |
| 27 | 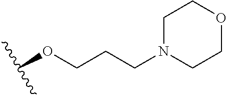 | 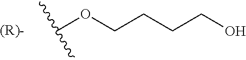 [SMILES: *OCCCCO] | B |
| 427 | 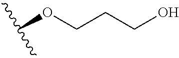 | 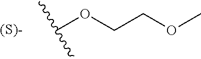 [SMILES: *OCCOC] | A |
| 28 | 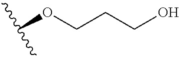 | 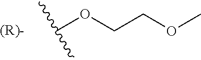 [SMILES: *OCCOC] | B |
| 439 | 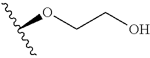 | 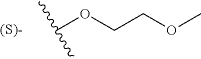 [SMILES: *OCCOC] | A |
| 40 | 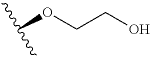 | 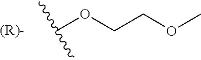 [SMILES: *OCCOC] | B |
| 529 | 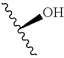 | 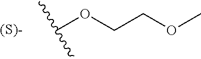 [SMILES: *OCCOC] | A |
| 130 | 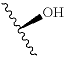 | 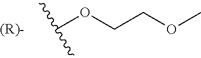 [SMILES: *OCCOC] | B |

### Example 5: SPR Assay to Characterize Ternary Complex Formation with FKBP51

Biotinylated avi-FKBP51 is immobilized on a streptavidin chip (Cytiva Series S SA) using a Biacore 8K or 8k+ (Cytiva). To achieve an immobilization level of 200 RU, 0.6 μg/ml biotinylated avi-FKBP51 is injected for 150 sec at a flow rate of 10 μl/min. Serial dilution of FRB is prepared (12 concentrations, 3-fold dilutions, 0.00011-20 μM) and supplemented with 100 nM of test compound. A-B-A injection mode is used to ensure saturation immobilized FKBP12 with respective test compound. 100 nM solution of the respective test compound is injected before FRB injection for 120 sec and during dissociation for 420 sec. The FRB dilutions are then injected 120 seconds contact time with increasing concentrations. Rapamycin is used as reference sample. 50 mM HEPES pH 7.5, 150 mM NaCl, 2 mM MgCl₂, 1 mM DTT, 0.05% Tween-20, 1% DMSO was is as running buffer at a flow rate of 30 μl/min. The multi-cycle kinetics data are fit to a 1:1 binding model to measure the association rate ka (1/Ms), the dissociation rate kd (1/s) and the affinity K_{d} (M). In case of fast association and dissociation, steady state affinity analysis following the law of mass action is used to determine the affinity Kd (M).

### Example 6: mTORC1 Inhibition, mTORC2 Inhibition, Cell Lysis, AlphaLISA Experiments, and Data Analysis

mTORC1 inhibition was determined via analysis of phosphorylation levels of Phospho-p70 S6 kinase (p70S6K pT389) and Phospho-S6 Ribosomal Protein (pRPS6 pS240/pS244) with the corresponding AlphaLISA kits (PerkinElmer Alpha SF Ultra™ Multiplex phospho (Thr389)/total p70 S6K Assay Kit (Eu/Tb) and AlphaLISA SF Ultra™ p-Ribosomal Protein S6 (Ser240/244) Assay Kit). Thus, PC-3 cells were plated on 96 well Corning clear bottom plates (Cat #3997) in growth medium (DMEM:Ham's F12, basic (CLS Cell Lines Service GmbH, Cat #820400a), supplemented with additional 5% fetal bovine serum (FBS; Gibco, Cat #10500064) at 1.20E+06 cells/mL and incubated over-night at 37° C., 5% CO₂. On the following day, cells were treated with growth medium containing increasing compound concentrations (12 points at 3-fold dilutions) and incubated for further 24 hours at 37° C., 5% CO₂ before cell lysis.

mTORC2 inhibition was determined via analysis of phosphorylation level of Phospho-AKT (pAKT pS473) with the corresponding AlphaLISA kit (PerkinElmer, Alpha SF Ultram Multiplex p-AKT1/2/3(Ser473)/Total AKT1). PC3 cells were plated on 96 well plates in assay medium (DMEM:Ham's F12, basic (CLS Cell Lines Service GmbH, Cat #820400a), supplemented with additional 10% FBS at 1.20E+06 cells/mL and incubated over-night at 37° C., 5% CO₂. On the following day, cells were treated with assay medium (10% FBS) containing increasing compound concentrations (12 points at 3-fold dilutions) and incubated for 6 hours at 37° C., 5% CO₂. Thereafter, medium was aspirated and cells were rinsed with PBS. In the following, cells were treated with compound dilutions in starvation medium (DMEM:Ham's F12, basic; without FBS) for further 18 h at 37° C., 5% CO₂. Then, immediately prior to cell lysis, cells were treated with 12% FBS for 15 min.

After performing experiments according to mTORC1 and mTORC2 protocols, cells were harvested in lysis buffer supplied with the AlphaLISA kits, additionally enriched with Roche cOmplete™ Protease Inhibitor Cocktail (Cat #CO-RO). Thus, cells were lysed using 50 μL of the lysis buffer and incubated for 60 min at 4° C. while shaking. After centrifugation at 4000 rpm for 5 min, experiments were performed according to the AlphaLISA manufacturer's protocol. Ten microliters of cell lysates were mixed with the acceptor mix. After incubation for 2 h at room temperature, the donor mix was added. After additional incubation at room temperature for 2 hours, AlphaLISA signal was read on PHERAstar® FSX (BMG Labtech) with AlphaPLEX module. Percent inhibition was calculated via ExcelFit standard algorithm, based on high control (cells incubated with vehicle/DMSO) and low control (mTORC1: cells incubated with 0.1 μM rapamycin; mTORC2: cells incubated with 1 μM rapamycin). All IC50 experiments were conducted in triplicates with rapamycin and vehicle controls.

### Data Analysis

Percentage activity/inhibition was calculated via application of the equations:

%−activity=100*((Sample−Low control)/(High control−Low control))

%−inhibition=100*(1−((Sample−Low control)/(High control−Low control)))
Sample=Assay signal (phospho-protein normalized to total protein) at respective compound concentration
High Control=Assay signal in presence of vehicle/DMSO
Low Control=Assay signal cells in presence of 0.1 μM (mTORC1) or 1 μM rapamycin (mTORC2)

EC50 values were calculated by ExcelFit standard algorithm. All IC50 experiments were conducted in triplicates with rapamycin and vehicle controls (six high/low controls per plate).

Table 13 includes IC₅₀ (nM) values for mTORC1 as measured by inhibition of p70S6K pT389 levels by selected compounds; with compounds having an IC₅₀ for mTORC1 of <0.8 nM as A, 0.8 nM to 1.5 nM as B, and greater than 1.5 as C.

When tested by the mTORC2 assay of Example 6, compounds of the disclosure typically display IC50s>1.0 uM whereas everolimus and rapamycin typically display IC50s of less than 10 nM

TABLE 13
mTORC 1 IC₅₀ of compounds represented by the Formula:

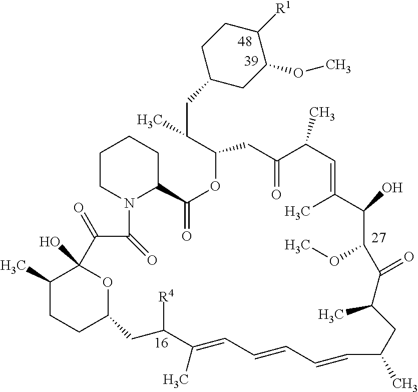

| Compound | | | |
|---|---|---|---|
| Number | R¹ | R⁴ | mTROC1 IC₅₀ (nM) |
| 431 | 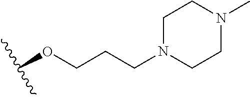 | 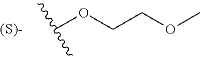 [SMILES: *OCCOC] | A |
| 32 | 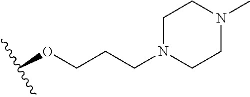 | 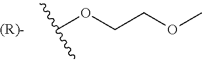 [SMILES: *OCCOC] | C |
| 521 | 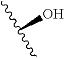 | 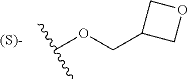 [SMILES: *OCC1COC1] | A |
| 122 | 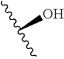 | 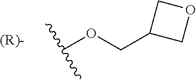 [SMILES: *OCC1COC1] | B |
| 474 | 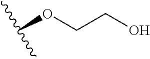 | 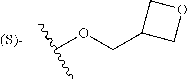 [SMILES: *OCC1COC1] | A |
| 75 | 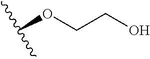 | 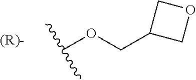 [SMILES: *OCC1COC1] | C |
| 524 | 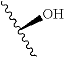 | 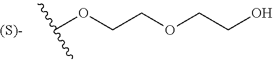 [SMILES: *OCCOCCO] | A |
| 125 | 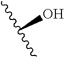 | 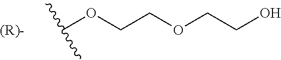 [SMILES: *OCCOCCO] | B |
| 523 | 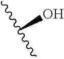 | 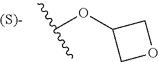 [SMILES: *OC1COC1] | A |
| 124 | 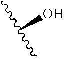 | 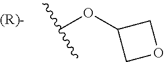 [SMILES: *OC1COC1] | C |
| 519 | 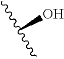 | 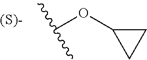 [SMILES: *OC1CC1] | A |
| 120 | 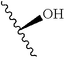 | 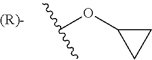 [SMILES: *OC1CC1] | A |
| 527 | 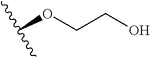 | 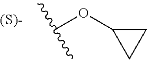 [SMILES: *OC1CC1] | A |
| 128 | 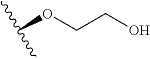 | 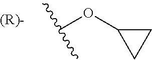 [SMILES: *OC1CC1] | B |
| 520 | 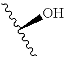 | 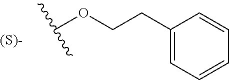 [SMILES: *OCCc1ccccc1] | A |
| 121 | 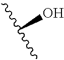 | 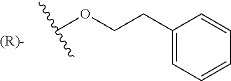 [SMILES: *OCCc1ccccc1] | B |
| 404 | 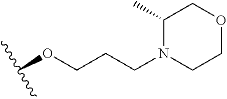 | 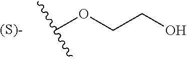 [SMILES: *OCCO] | A |
| 5 | 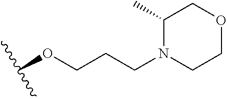 | 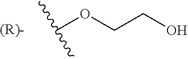 [SMILES: *OCCO] | |
| 426 | 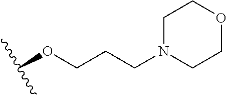 | 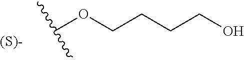 [SMILES: *OCCCCO] | B |
| 27 | 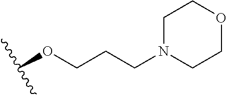 | 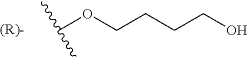 [SMILES: *OCCCCO] | B |
| 427 | 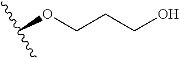 | 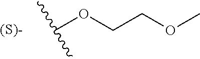 [SMILES: *OCCOC] | A |
| 28 | 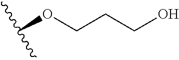 | 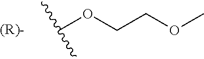 [SMILES: *OCCOC] | C |
| 439 | 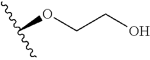 | 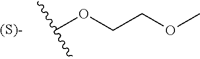 [SMILES: *OCCOC] | A |
| 40 | 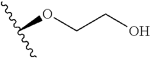 | 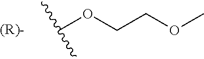 [SMILES: *OCCOC] | C |
| 529 | 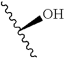 | 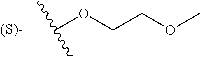 [SMILES: *OCCOC] | A |
| 130 | 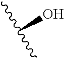 | 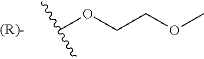 [SMILES: *OCCOC] | B |

| Compound | mTORC1 IC₅₀ (nM) |
|---|---|
| Rapamycin | A |

1-118. (canceled)

119. A compound represented by Formula (III-C): 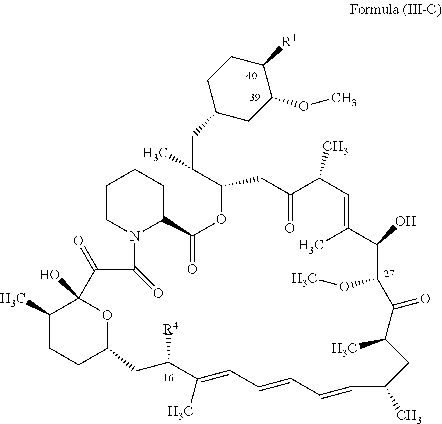
or a pharmaceutically acceptable salt thereof:
and wherein for Formula (III-C), R¹ and R⁴ are both selected from:

| Compound | | |
|---|---|---|
| No. # | R¹ | R⁴ |
| 413 | 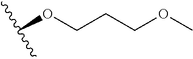 | 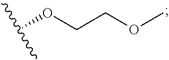 |
| 424 | 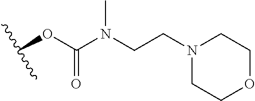 | 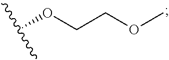 |
| 425 | 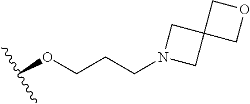 | 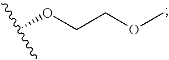 |
| 427 | 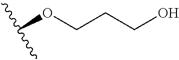 | 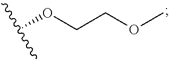 |
| 428 | 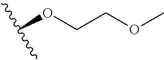 | 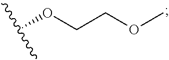 |
| 429 | 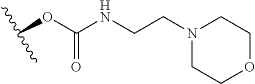 | 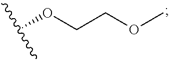 |
| 431 | 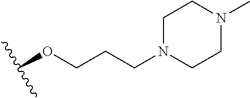 | 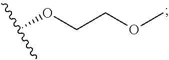 |
| 434 | 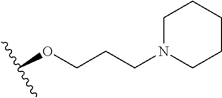 | 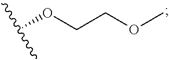 |
| 435 | 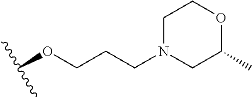 | 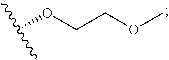 |
| 438 | 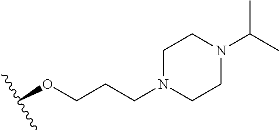 | 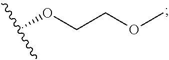 |
| 439 | 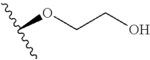 | 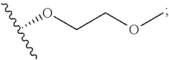 |
| 443 | 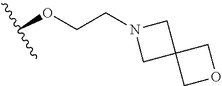 | 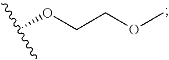 |
| 444 | 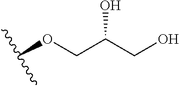 | 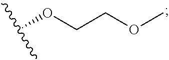 |
| 446 | 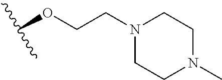 | 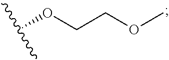 |
| 447 | 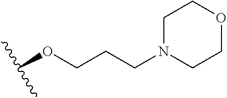 | 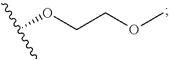 |
| 450 | 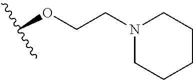 | 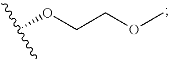 |
| 451 | 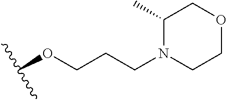 | 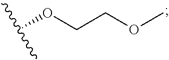 |
| 453 | 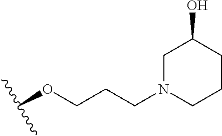 | 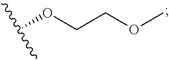 |
| 455 | 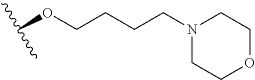 | 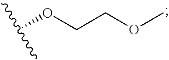 |
| 456 | 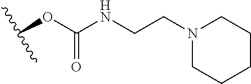 | 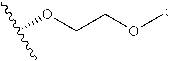 |
| 457 | 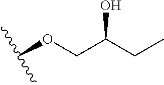 | 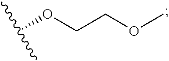 |
| 458 | 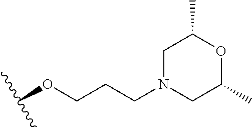 | 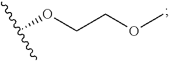 |
| 461 | 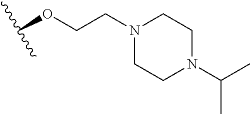 | 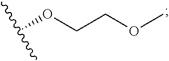 |
| 465 | 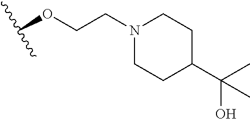 | 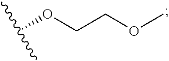 |
| 473 | 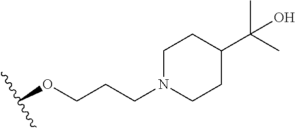 | 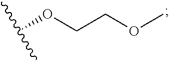 |
| 480 | 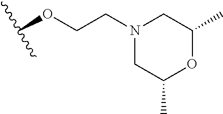 | 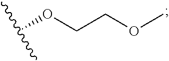 |
| 484 | 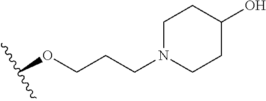 | 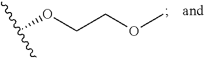 |
| 517 | 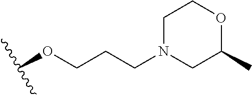 | 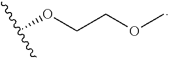 |

120. The compound or salt of claim 119, wherein for Formula (III-C), R¹ and R⁴ are both selected from:

| Compound | | |
|---|---|---|
| No. # | R¹ | R⁴ |
| 431 | 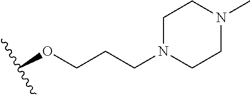 | 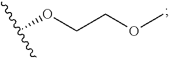 |
| 434 | 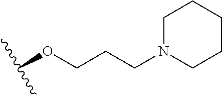 | 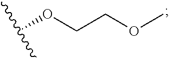 |
| 435 | 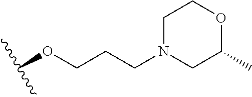 | 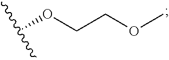 |
| 438 | 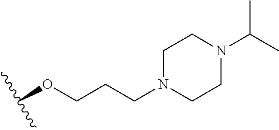 | 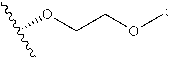 |
| 443 | 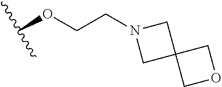 | 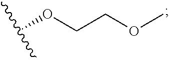 |
| 446 | 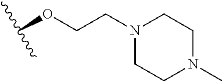 | 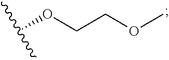 |
| 447 | 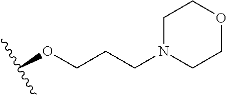 | 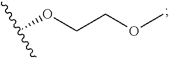 |
| 450 | 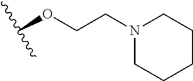 | 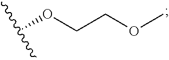 |
| 451 | 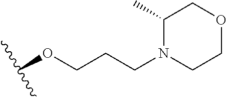 | 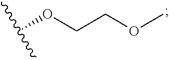 |
| 453 | 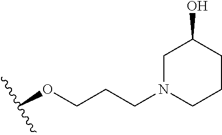 | 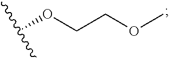 |
| 455 | 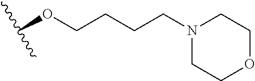 | 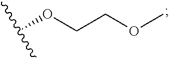 |
| 458 | 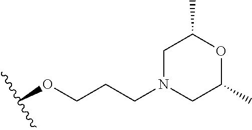 | 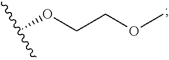 |
| 461 | 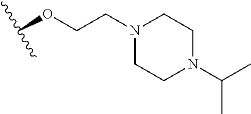 | 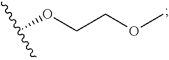 |
| 465 | 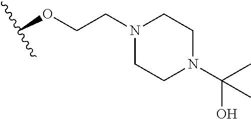 | 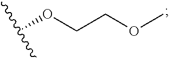 |
| 473 | 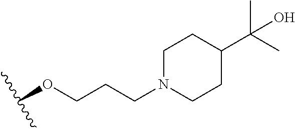 | 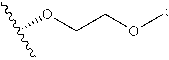 |
| 480 | 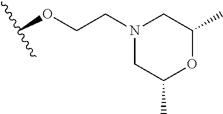 | 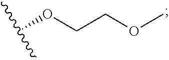 |
| 484 | 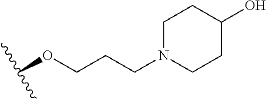 | 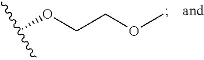 |
| 517 | 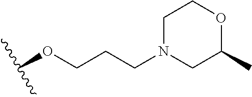 | 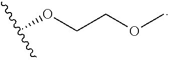 |

121. The compound or salt of claim 119, wherein for Formula (III-C), R¹ and R⁴ are both selected from:

| Compound No. # | R¹ | R⁴ |
|---|---|---|
| 431 | 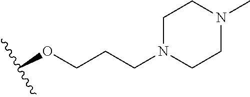 | 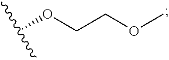 |
| 434 | 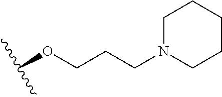 | 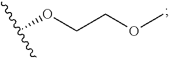 |
| 435 | 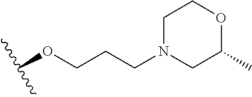 | 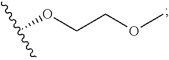 |
| 438 | 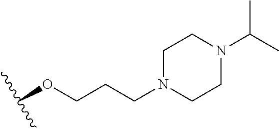 | 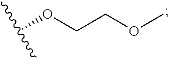 |
| 447 | 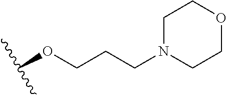 | 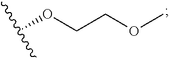 |
| 451 | 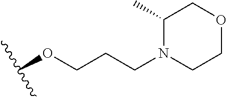 | 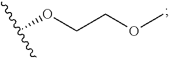 |
| 453 | 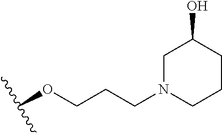 | 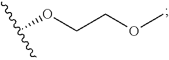 |
| 458 | 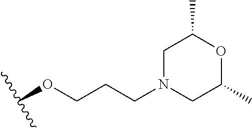 | 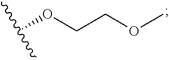 |
| 473 | 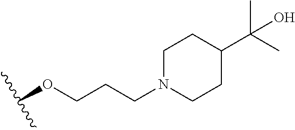 | 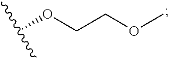 |
| 484 | 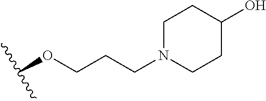 | 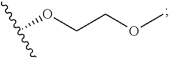 |
| 517 | 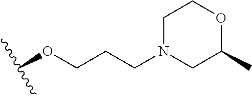 | 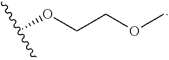 |

122. The compound or salt of claim 119, wherein for Formula (III-C), R¹ and R⁴ are both selected from:

| Compound No. # | R¹ | R⁴ |
|---|---|---|
| 431 | 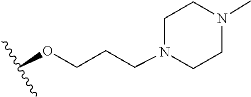 | 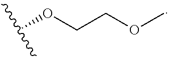 |

123. The compound or salt of claim 119, wherein for Formula (III-C), R¹ and R⁴ are both selected from:

| Compound No. # | R¹ | R⁴ |
|---|---|---|
| 447 | 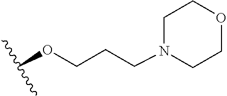 | 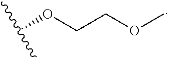 |

124. The compound or salt of claim 119, wherein for Formula (III-C), R¹ and R⁴ are both selected from:

| Compound No. # | R¹ | R⁴ |
|---|---|---|
| 451 | 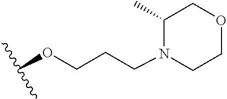 | 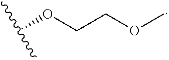 |

125. The compound or salt of claim 119, wherein for Formula (III-C), R¹ and R⁴ are both selected from:

| Com- | | |
|---|---|---|
| pound | | |
| No. # | R¹ | R⁴ |
| 413 | 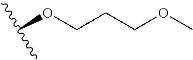 | 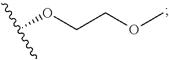 |
| 427 | 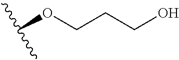 | 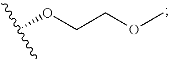 |
| 428 | 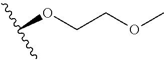 | 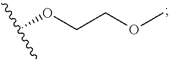 |
| 439 | 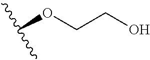 | 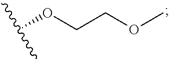 |
| 444 | 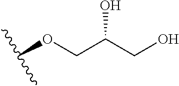 | 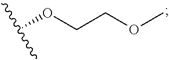 |
| 457 | 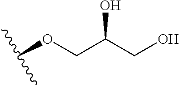 | 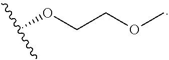 |

126. The compound or salt of claim 119, wherein for Formula (III-C), R¹ and R⁴ are both selected from.

| Com- | | |
|---|---|---|
| pound | | |
| No. # | R¹ | R⁴ |
| 413 | 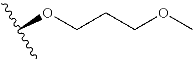 | 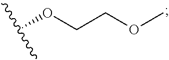 |
| 427 | 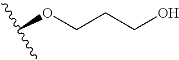 | 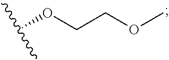 |
| 428 | 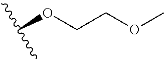 | 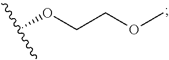 |
| 439 | 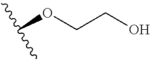 | 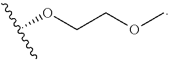 |

127. The compound or salt of claim 119, wherein for Formula (III-C), R¹ and R⁴ are both selected from:

| Com- | | |
|---|---|---|
| pound | | |
| No. # | R¹ | R⁴ |
| 413 | 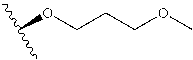 | 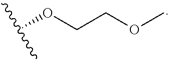 |

128. The compound or salt of claim 119, wherein for Formula (III-C), R¹ and R⁴ are both selected from.

| Compound | | |
|---|---|---|
| No. # | R¹ | R⁴ |
| 427 | 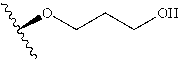 | 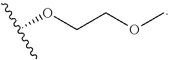 |

129. The compound or salt of claim 119, wherein for Formula (III-C), R¹ and R⁴ are both selected from:

| Compound | | |
|---|---|---|
| No. # | R¹ | R⁴ |
| 428 | 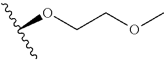 | 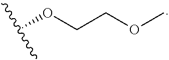 |

130. The compound or salt of claim 119, wherein for Formula (III-C), R¹ and R⁴ are both selected from:

| Compound | | |
|---|---|---|
| No. # | R¹ | R⁴ |
| 439 | 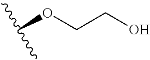 | 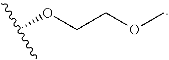 |

131. The compound or salt of claim 119, wherein for Formula (III-C), R¹ and R⁴ are both selected from:

| Compound No. # | R¹ | R⁴ |
|---|---|---|
| 424 | 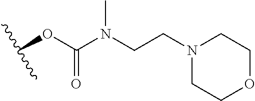 | 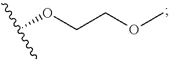 |
| 429 | 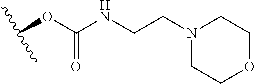 | 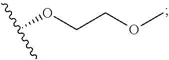 |
| 456 | 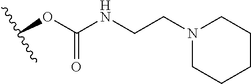 | 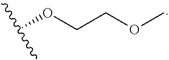 |

132. The compound or salt of claim 119, wherein for Formula (III-C), R¹ and R⁴ are both selected from:

| Compound No. # | R¹ | R⁴ |
|---|---|---|
| 424 | 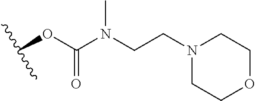 | 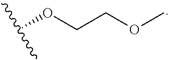 |

133. A pharmaceutical composition comprising a compound or salt of claim 119 and a pharmaceutically acceptable excipient.

134. A method of treating a disease, comprising administering to a subject in need thereof a pharmaceutical composition of claim 133.

135. The method of claim 134, wherein the disease is selected from a chronic disease.

136. The method of claim 135, wherein the chronic disease is selected from a disease wherein mTORC1 is hyperactivated or a disease that would benefit from inhibition of the activity of mTORC1.

137. The method of claim 136, wherein the chronic disease is selected from a neurodegenerative disease; a neurocutaneous disease; a neurodevelopmental disorder; mTORopathies, including tuberous sclerosis, focal cortical dysplasia, and a PTEN disease; tauopathies; cognitive disorders; epilepsies; autism spectrum disorders; autoimmune diseases; metabolic diseases; cancer; diseases of impaired autophagy; infectious diseases; cardiovascular diseases; muscular atrophy; inflammatory diseases; eye disorders; age related diseases that result in hyperactivation of mTORC1 or age related diseases that benefit from inhibition of the activity of mTORC1, including reduced immune activity in the elderly; a kidney disease, including polycystic kidney disease; and lymphangioleiomyomatosis.

138. The method of claim 137, wherein the chronic disease is an mTORopathy.
